(19) United States
(12) Patent Application Publication
(10) Pub. No.: US 2025/0295624 A1
(43) Pub. Date: Sep. 25, 2025

(54) MONOMETHYL FUMARATE-CARRIER CONJUGATES AND METHODS OF THEIR USE

(71) Applicant: Flagship Pioneering Innovations V, Inc., Cambridge, MA (US)

(72) Inventors: John Patrick CASEY, JR., Boston, MA (US); David Arthur BERRY, Newton, MA (US); Jessica Elizabeth ALEXANDER, Waltham, MA (US); Timothy F. BRIGGS, Waltham, MA (US); Leonard BUCKBINDER, East Greenwich, RI (US); Dinara Shashanka GUNASEKERA, Cambridge, MA (US); Afrand KAMALI SARVESTANI, Somerville, MA (US); Mi-Jeong KIM, Boston, MA (US); Bernard LANTER, Somerville, MA (US); Anna LIANG, Everett, MA (US); Kathleen NUDEL, Jamaica Plain, MA (US); Spencer Cory PECK, Watertown, MA (US); Cheri ROSS, Winthrop, MA (US); Steven John TAYLOR, Winchester, MA (US); Koji YASUDA, Boston, MA (US)

(21) Appl. No.: 19/172,883

(22) Filed: Apr. 8, 2025

Related U.S. Application Data
(63) Continuation of application No. 17/337,595, filed on Jun. 3, 2021, now abandoned.
Application No. 17/337,595 is a continuation of application No. PCT/US2019/064926, filed on Dec. 6, 2019.
(60) Provisional application No. 62/776,430, filed on Dec. 6, 2018.
(60) Provisional application No. 62/938,610, filed on Nov. 21, 2019.

(51) Int. Cl. A61K 31/225; A61K 47/54; A61P 25/28; A61P 37/06

(52) U.S. Cl. CPC A61K 31/225; A61K 47/549; A61P 25/28; A61P 37/06

(57) ABSTRACT

Disclosed are conjugates of monomethyl fumarate and a carrier group or aminocarrier group, or a pharmaceutically acceptable salt thereof. In the conjugates, monomethyl fumarate acyl is covalently bonded to the carrier group or aminocarrier group through a carbon-oxygen bond that is cleavable in vivo. The carrier group may include a core, e.g., a monosaccharide, a sugar acid (e.g., acid monosaccharide), a sugar alcohol, or a catechin polyphenol. The aminocarrier group may include a core, e.g., an aminomonosaccharide. The carrier group or aminocarrier group may include, e.g., at least one short chain fatty acid acyl, at least one tryptophan analogue, at least one ketone body, or at least one pre-ketone body. Also disclosed are pharmaceutical compositions containing the conjugates and methods of their use.

### FIELD OF THE INVENTION

The present invention relates to conjugates of monomethyl fumarate and a carrier or aminocarrier group. The present invention also features compositions containing the conjugates and methods of using the conjugates.

### BACKGROUND

The mammalian microbiota can engage in a bidirectional communication with the mammalian host system. While therapeutic approaches taking advantage of the mammalian microbiota have so far largely focused on probiotics (e.g., live microorganisms) as the active agents, combinations of small molecules leveraging the bidirectional communication remain largely underutilized.

There is a need for pharmaceutical applications leveraging the advantages of small molecule-based conjugates.

### SUMMARY OF THE INVENTION

The present invention provides conjugates consisting of monomethyl fumarate and a carrier group or aminocarrier group, pharmaceutical compositions containing them, and methods of modulating an autoimmunity marker in a subject or of treating an autoimmunity disorder in a subject.

In one aspect, the invention provides a conjugate, or a pharmaceutically acceptable salt thereof, of monomethyl fumarate covalently bonded to a carrier group or amino carrier group. In some embodiments, the conjugate includes monomethyl fumarate acyl covalently bonded to the carrier group or the aminocarrier group through a carbon-oxygen bond that is cleavable in vivo. In some embodiments, the carrier group or the aminocarrier group includes at least one short chain fatty acid acyl, at least one tryptophan analogue, at least one ketone body, or at least one pre-ketone body. In some embodiments, the cleavable in vivo carbon-oxygen bond is an ester bond or a glycosidic bond. In some embodiments, the cleavable in vivo carbon-oxygen bond is an ester bond. In some embodiments, the carbon-oxygen bond that is cleavable in vivo is a glycosidic bond attached to the anomeric carbon atom of the C₅₋₆ pyranose. In some embodiments, the carbon-oxygen bond that is cleavable in vivo is a bond attached to position 4 of the C₅₋₆ pyranose. In some embodiments, the carbon-oxygen bond that is cleavable in vivo is a bond attached to position 6 of the C₅₋₆ pyranose.

In some embodiments, the conjugate includes a carrier group including a core with one or more hydroxyls independently substituted with an acyl. In some embodiments, the acyl is a fatty acid acyl. In some embodiments, the conjugate includes a fatty acid acyl that is a short chain fatty acid acyl (e.g., propionyl or butyryl). In some embodiments, the conjugate includes a fatty acid acyl that is a medium chain fatty acyl. In some embodiments, the core is peracylated.

In other embodiments, the carrier group is monosaccharide, sugar alcohol, or sugar acid having one or more hydroxyls independently substituted with an alkyl, short chain fatty acid acyl, monomethyl fumarate acyl, tryptophan analogue acyl, ketone body acyl, optionally acylated ketone body, pre-ketone body acyl, or optionally acylated pre-ketone body; provided that at least one hydroxyl is substituted with a short chain fatty acid acyl, tryptophan analogue acyl, ketone body acyl, optionally acylated ketone body, pre-ketone body acyl, or optionally acylated pre-ketone body. When the substituted hydroxyl comprises an alcohol oxygen atom, the hydroxyl is substituted with an alkyl, short chain fatty acid acyl, monomethyl fumarate acyl, tryptophan analogue acyl, ketone body acyl, or pre-ketone body acyl, provided that at least one hydroxyl is substituted with a short chain fatty acid acyl, tryptophan analogue acyl, ketone body acyl, or pre-ketone body acyl, and when the substituted hydroxyl comprises a carboxylate oxygen atom, the hydroxyl is substituted with an alkyl, optionally acylated ketone body, or optionally acylated pre-ketone body. In some embodiments, the core is a monosaccharide. In some embodiments, the monosaccharide is selected from a group consisting of arabinose, fucose, galactose, glucose, mannose, rhamnose, ribose, tagatose, and xylose. In some embodiments, the monosaccharide is glucose or ribose.

In some embodiments, the core is a C₅₋₆ pyranose. In some embodiments, the C₅₋₆ pyranose is an alpha-anomer. In some embodiments, the C₅₋₆ pyranose core is a beta-anomer.

In particular embodiments, the carrier group is a monosaccharide having one or more hydroxyls independently substituted with an alkyl, short chain fatty acid acyl, monomethyl fumarate acyl, tryptophan analogue acyl, ketone body acyl, or pre-ketone body acyl; provided that at least one hydroxyl is substituted with a short chain fatty acid acyl, tryptophan analogue acyl, ketone body acyl, or pre-ketone body acyl. In certain embodiments, the monosaccharide is arabinose, xylose, fructose, galactose, glucose, ribose, tagatose, fucose, or rhamnose.

In further embodiments, the carrier group is a sugar acid having one or more hydroxyls independently substituted with an alkyl, short chain fatty acid acyl, monomethyl fumarate acyl, tryptophan analogue acyl, ketone body acyl, optionally acylated ketone body, pre-ketone body acyl, or optionally acylated pre-ketone body; provided that at least one hydroxyl is substituted with a short chain fatty acid acyl, tryptophan analogue acyl, ketone body acyl, optionally acylated ketone body, pre-ketone body acyl, or optionally acylated pre-ketone body. When the substituted hydroxyl comprises an alcohol oxygen atom, the hydroxyl is substituted with an alkyl, short chain fatty acid acyl, monomethyl fumarate acyl, tryptophan analogue acyl, ketone body acyl, or pre-ketone body acyl; provided that at least one hydroxyl is substituted with a short chain fatty acid acyl, tryptophan analogue acyl, ketone body acyl, or pre-ketone body acyl, and when the substituted hydroxyl comprises a carboxylate oxygen atom, the hydroxyl is substituted with an alkyl, optionally acylated ketone body, or optionally acylated pre-ketone body.

In particular embodiments, the sugar acid is aldonic acid, ulosonic acid, uronic acid, aldaric acid, xylonic acid, gluconic acid, glucuronic acid, galacturonic acid, tartaric acid, saccharic acid, or mucic acid.

In some embodiments, the core is an acid monosaccharide. In some embodiments, the acid monosaccharide is glucuronic acid. In other embodiments, sugar alcohol having one or more hydroxyls independently substituted with an alkyl, short chain fatty acid acyl, monomethyl fumarate acyl, tryptophan analogue acyl, ketone body acyl, or pre-ketone body acyl; provided that at least one hydroxyl is substituted with a short chain fatty acid acyl, tryptophan analogue acyl, ketone body acyl, or pre-ketone body acyl. In certain embodiments, the sugar alcohol is glycerol, erythritol, threitol, arabitol, xylitol, tibitol, mannitol, sorbitol, galactitol, fucitol, iditol, or inositol.

In some embodiments, the conjugate is a conjugate of monomethyl fumarate and a carrier group, or a pharmaceutically acceptable salt thereof, where monomethyl fumarate acyl is covalently bonded to the carrier group through a carbon-oxygen bond that is cleavable in vivo, where the carrier group includes a sugar alcohol core of the following structure:

HOCH₂(CHOH)ₙCH₂OH,
where n is 1, 2, 3, or 4; and one or more of the hydroxyl groups is independently substituted with an alkyl, acyl, or a bond to monomethyl fumarate.

In some embodiments, n is 1. In some embodiments, the sugar alcohol core has one or more hydroxyls independently substituted with a short chain fatty acyl (e.g., propionyl or butyryl).

In some embodiments, the conjugate includes an aminocarrier group including a core that is an aminomonosaccharide. In some embodiments, the aminomonosaccharide is glucosamine.

In further embodiments, the carrier group is an acylated aminomonosaccharide (e.g., an acylated aminomonosaccharide including glucosamine or galactosamine).

In yet further embodiments, the carrier group comprises an anomeric carbon atom bonded to monomethyl fumarate through a glycosidic bond.

In still further embodiments, the carrier group comprises an oxygen atom bonded to monomethyl fumarate through an ester bond. In other embodiments, the carrier group includes a C₅₋₆ pyranose or a C₅₋₆ aminopyranose core. In yet other embodiments, the oxygen atom bonded to monomethyl fumarate is covalently bonded to position 4 of the core. In still other embodiments, the oxygen atom bonded to monomethyl fumarate is covalently bonded to position 6 of the core.

In some embodiments, the carrier group is a stilbenoid having one or more hydroxyls independently substituted with an alkyl, short chain fatty acid acyl, monomethyl fumarate acyl, tryptophan analogue acyl, ketone body acyl, or pre-ketone body acyl; provided that at least one hydroxyl is substituted with a short chain fatty acid acyl, tryptophan analogue acyl, ketone body acyl, or pre-ketone body acyl. In particular embodiments, the stilbenoid is resveratrol.

In certain embodiments, the carrier group is a catechin polyphenol having one or more hydroxyls independently substituted with an alkyl, short chain fatty acid acyl, monomethyl fumarate acyl, tryptophan analogue acyl, ketone body acyl, or pre-ketone body acyl; provided that at least one hydroxyl is substituted with a short chain fatty acid acyl, tryptophan analogue acyl, ketone body acyl, or pre-ketone body acyl. In particular embodiments, the catechin polyphenol is quercetin.

In some embodiments, the conjugate is a conjugate of monomethyl fumarate and a carrier group, or a pharmaceutically acceptable salt thereof, where monomethyl fumarate acyl is covalently bonded to the carrier group through a carbon-oxygen bond that is cleavable in vivo, where the carrier group includes a catechin polyphenol core.

In some embodiments, the conjugate is a compound of the following structure:

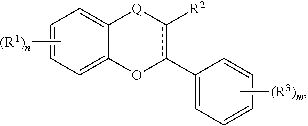

where
[symbol] is a single carbon-carbon bond or double carbon-carbon bond;
Q is —CH— or —C(O)—;
each R¹ and each R³ is independently H, halogen, —OR^{A};
R² is H or —OR^{A};
each R^{A} is independently H, alkyl, short chain fatty acid acyl, monomethyl fumarate acyl, or benzoyl optionally substituted with 1, 2, 3, or 4 substituents independently selected from the group consisting of H, hydroxy, halogen, optionally substituted alkyl, alkoxy, short chain fatty acid acyl, or monomethyl fumarate acyl; and
each of n and m is independently 1, 2, 3, or 4.

In some embodiments, each R¹ and each R³ is independently H or —OR^{A}. In some embodiments, each R^{A} is independently H or monomethyl fumarate acyl. In some embodiments, n is 2. In some embodiments, m is 1 or 2.

In other embodiments, the carrier group is a ketone body or a pre-ketone body having one or more hydroxyls substituted with a short chain fatty acid acyl, monomethyl fumarate acyl, tryptophan analogue acyl, ketone body acyl, or pre-ketone body acyl.

In still further embodiments, the carrier group is a bile acid having one or more hydroxyls substituted with an alkyl, short chain fatty acid acyl, monomethyl fumarate acyl, tryptophan analogue acyl, ketone body acyl, optionally acylated ketone body, pre-ketone body acyl, or optionally acylated pre-ketone body; provided that at least one hydroxyl is substituted with a short chain fatty acid acyl, tryptophan analogue acyl, ketone body acyl, optionally acylated ketone body, pre-ketone body acyl, or optionally acylated pre-ketone body. When the substituted hydroxyl comprises an alcohol oxygen atom, the hydroxyl is substituted with an alkyl, short chain fatty acid acyl, monomethyl fumarate acyl, tryptophan analogue acyl, ketone body acyl, or pre-ketone body acyl; provided that at least one hydroxyl is substituted with a short chain fatty acid acyl, tryptophan analogue acyl, ketone body acyl, or pre-ketone body acyl, and when the substituted hydroxyl comprises a carboxylate oxygen atom, the hydroxyl is substituted with an alkyl, optionally acylated ketone body, or optionally acylated pre-ketone body.

In certain embodiments, the bile acid is obeticholic acid. In some embodiments, each short chain fatty acid acyl is independently propionyl or butyryl. In particular embodiments, the carrier group includes propionyl. In further embodiments, the carrier group includes butyryl.

In some embodiments, the carrier group comprises one or more tryptophan analogue acyls. In certain embodiments, each tryptophan analogue acyl is independently indole3-acetic acid acyl, indole-3-acrylic acid acyl, indole-3-pyruvic acid acyl.

In particular embodiments, the carrier group is a tryptophan analogue. In certain embodiments, the tryptophan analogue is indole-3-carbinol.

In some embodiments, the conjugate is of the following structure:

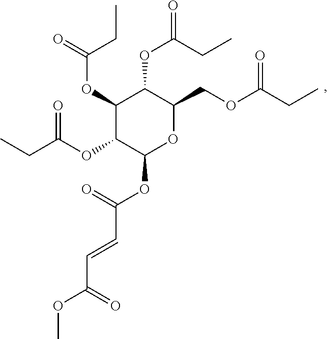 [SMILES: CCC(=O)OC[C@H]1O[C@@H](OC(=O)/C=C/C(=O)OC)[C@H](OC(=O)CC)[C@@H](OC(=O)CC)[C@@H]1OC(=O)CC]

or a pharmaceutically acceptable salt thereof.

In some embodiments, the conjugate is of the following structure:

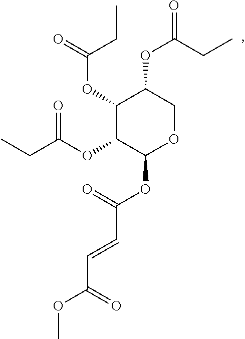 [SMILES: CCC(=O)O[C@H]1[C@H](OC(=O)/C=C/C(=O)OC)OC[C@@H](OC(=O)CC)[C@H]1OC(=O)CC]

or a pharmaceutically acceptable salt thereof.

In some embodiments, the conjugate is of the following structure:

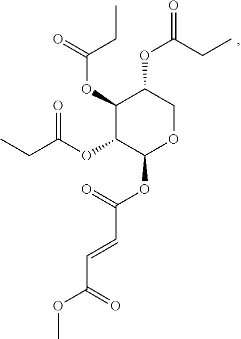 [SMILES: CCC(=O)O[C@@H]1[C@@H](OC(=O)CC)[C@H](OC(=O)/C=C/C(=O)OC)OC[C@H]1OC(=O)CC]

or a pharmaceutically acceptable salt thereof.

In some embodiments, the conjugate is of the following structure:

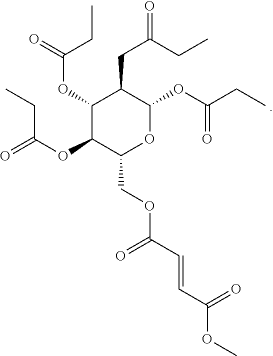 [SMILES: CCC(=O)C[C@H]1[C@H](OC(=O)CC)O[C@H](COC(=O)/C=C/C(=O)OC)[C@@H](OC(=O)CC)[C@@H]1OC(=O)CC]

or a pharmaceutically acceptable salt thereof.

Income embodiments, the conugate is of the following structure:

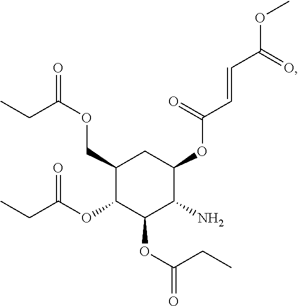 [SMILES: CCC(=O)OC[C@H]1C[C@@H](OC(=O)/C=C/C(=O)OC)[C@H](N)[C@@H](OC(=O)CC)[C@@H]1OC(=O)CC]

or a pharmaceutically acceptable salt thereof.

In one aspect, the invention provides a pharmaceutical composition consisting of a conjugate described herein, or a pharmaceutically acceptable salt thereof. Non-limiting examples of the conjugates include monomethyl fumarate covalently bonded to a carrier group having at least one short chain fatty acid acyl, at least one tryptophan analogue, at least one ketone body, or at least one pre-ketone body, through a carbon-oxygen bond that is cleavable in vivo, and a pharmaceutically acceptable carrier.

In another aspect, the invention provides a method of treating a subject in need thereof by administering to the subject in need thereof a therapeutically effective amount of a conjugate of the invention, a pharmaceutically acceptable salt thereof, or a pharmaceutical composition having a conjugate of the invention and a pharmaceutically acceptable carrier.

In some embodiments, the subject is suffering from an autoimmune disorder. In particular embodiments, the autoimmune disorder is multiple sclerosis, psoriasis, psoriatic arthritis, rheumatoid arthritis, systemic lupus erythematosus, Crohn's disease, Sjogren's syndrome, Behcet's disease, ulcerative colitis, or Guillain-Barré syndrome.

In certain embodiments, the subject is suffering from multiple sclerosis, e.g., primary progressive multiple sclerosis, secondary progressive multiple sclerosis, or relapsing-remitting multiple sclerosis. In other embodiments, the subject is suffering from primary progressive multiple sclerosis. In other embodiments, the subject is suffering from secondary progressive multiple sclerosis.

In yet other embodiments, the subject is suffering from obstructive sleep apnea, chronic lymphocytic leukemia, small lymphocytic leukemia, systemic sclerosis-pulmonary hypertension, glioblastoma multiforme, cutaneous T cell lymphoma, or progressive multifocal leukoencephalopathy.

In further embodiments, the subject is suffering from adrenoleukodystrophy, AGE-induced genome damage, Alexander's disease, Alper's disease, Alzheimer's disease, amyotrophic lateral sclerosis, angina pectoris, arthritis, asthma, balo concentric sclerosis, Canavan disease, cardiac insufficiency including left ventricular insufficiency, central nervous system vasculitis, Charcott-Marie-Tooth Disease, childhood ataxia with central nervous system hypomyelination, chronic idiopathic peripheral neuropathy, chronic obstructive pulmonary disease, diabetic retinopathy, graft-versus-host-disease, hepatitis C viral infection, herpes simplex viral infection, human immunodeficiency viral infection, Huntington's disease, irritable bowel syndrome, ischemia, Krabbe disease, lichen planus, macular degeneration, mitochondrial encephalomyopathy, monomelic amyotrophy, myocardial infarction, neurodegeneration with brain iron accumulation, neuromyelitis optica, neurosarcoidosis, optic neuritis, paraneoplastic syndrome, Parkinson's disease, Pelizaeus-Merzbacher disease, primary lateral sclerosis, progressive supranuclear palsy, reperfusion injury, retinopathia pigmentosa, Schilder's disease, subacute necrotizing myelopathy, susac syndrome, transverse myelitis, Zellweger's syndrome, granuloma annulare, pemphigus, bollus pemphigoid, contact dermatitis, acute dermatitis, chronic dermatitis, alopecia areata (totalis or universalis), sarcoidosis, cutaneous sarcoidosis, pyoderma gangrenosum, cutaneous lupus, or cutaneous Crohn's disease.

In particular embodiments, the subject is suffering from polyarthritis, juvenile-onset diabetes, type II diabetes, Hashimoto's thyroiditis, Grave's disease, pernicious anaemia, autoimmune hepatitis, or neurodermatitis.

In still further embodiments, the subject is suffering from retinopathia pigmentosa or forms of mitochondrial encephalomyopathy, progressive systemic sclerodermia, osteochondritis syphilitica (Wegener's disease), cutis marmorata (livedo reticularis), panarteriitis, vasculitis, osteoarthritis, gout, arteriosclerosis, Reiter's disease, pulmonary granulomatosis, endotoxic shock (septic-toxic shock), sepsis, pneumonia, encephalomyelitis, anorexia nervosa, acute hepatitis, chronic hepatitis, toxic hepatitis, alcohol-induced hepatitis, viral hepatitis, liver insufficiency, cytomegaloviral hepatitis, Rennert T-lymphomatosis, mesangial nephritis, post-angioplastic restenosis, reperfusion syndrome, cytomegaloviral retinopathy, adenoviral cold, adenoviral pharyngoconjunctival fever, adenoviral ophthalmia, AIDS, post-herpetic or post-zoster neuralgia, inflammatory demyelinating polyneuropathy, mononeuropathia multiplex, mucoviscidosis, Bechterew's disease, Barett oesophagus, Epstein-Barr virus infection, cardiac remodeling, interstitial cystitis, diabetes mellitus type II, human tumor radiosensitization, multidrug resistance in chemotherapy, mamma carcinoma, colon carcinoma, melanoma, primary liver cell carcinoma, adenocarcinoma, Kaposi's sarcoma, prostate carcinoma, leukaemia, acute myeloid leukaemia, multiple myeloma (plasmocytoma), Burkitt's lymphoma, Castleman tumor, cardiac insufficiency, myocardial infarct, angina pectoris, asthma, chronic obstructive pulmonary diseases, PDGF induced thymidine uptake of bronchial smooth muscle cells, bronchial smooth muscle cell proliferation, alcoholism, Alexander's disease, Alper's disease, Alzheimer's disease, ataxia telangiectasia, Batten disease (also known as Spielmeyer-Vogt-Sjögren-Batten disease), bovine spongiform encephalopathy (BSE), Cerebral palsy, Cockayne syndrome, corticobasal degeneration, Creutzfeldt-Jakob disease, familial fatal insomnia, frontotemporal lobar degeneration, Huntington's disease, HIV-associated dementia, Kennedy's disease, Krabbe's disease, Lewy body dementia, neuroborreliosis, Machado-Joseph disease (Spinocerebellar ataxia type 3), multiple system atrophy, narcolepsy, Niemann Pick disease, Pelizaeus-Merzbacher disease, Pick's disease, primary lateral sclerosis, prion disease, progressive supranuclear palsy, Refsum's disease, Sandhoff disease, subacute combined degeneration of spinal cord secondary to pernicious anaemia, spinocerebellar ataxia, spinal muscular atrophy, Steele-Richardson-Olszewski disease, Tabes dorsalis, toxic encephalopathy, LHON (Leber's Hereditary optic neuropathy), MELAS (Mitochondrial Encephalomyopathy; Lactic Acidosis; Stroke), MERRF (Myoclonic Epilepsy; Ragged Red Fibers), PEO (Progressive External Opthalmoplegia), Leigh's Syndrome, MNGIE (Myopathy and external ophthalmoplegia; Neuropathy; Gastro-Intestinal; Encephalopathy), Kearns-Sayre Syndrome (KSS), NARP, hereditary spastic paraparesis, mitochondrial myopathy, Friedreich Ataxia, optic neuritis, acute inflammatory demyelinating polyneuropathy (AIDP), chronic inflammatory demyelinating polyneuropathy (CIDP), acute transverse myelitis, acute disseminated encephalomyelitis (ADEM), or Leber's optic atrophy.

In another aspect, the invention provides a method of modulating an autoimmunity marker in a subject in need thereof by administering to the subject in need thereof a therapeutically effective amount of a conjugate of the invention, a pharmaceutically acceptable salt thereof, or a pharmaceutical composition having a conjugate of the invention and a pharmaceutically acceptable carrier.

In some embodiments, autoimmunity marker is for multiple sclerosis, psoriasis, psoriatic arthritis, rheumatoid arthritis, systemic lupus erythematosus, Crohn's disease, Sjogren's syndrome, Behcet's disease, ulcerative colitis, or Guillain-Barré syndrome.

In certain embodiments, a CYP1A1 mRNA level, intestinal motility, CD4⁺CD25⁺ Treg cell count, short chain fatty acids level, or mucus secretion is increased following the administration step. In other embodiments, abdominal pain, gastrointestinal inflammation, gastrointestinal permeability, gastrointestinal bleeding, intestinal motility, or frequency of bowel movements is reduced following the administration step. In further embodiments, an interleukin-8 (IL8) level, macrophage inflammatory protein 1α (MIP-1α) level, macrophage inflammatory protein 1β (MIP-1β) level, NFκB level, inducible nitric oxide synthase (iNOS) level, matrix metallopeptidase 9 (MMP9) level, interferon γ (IFNγ) level, interleukin-17 (IL17) level, intercellular adhesion molecule (ICAM) level, CXCL13 level, 8-iso-prostaglandin F_{2α} (8-iso-PGF2α) level IgA level, calprotectin level, lipocalin-2 level, or indoxyl sulfate level is reduced following the administration step.

In particular embodiments, an interleukin-8 (IL8) level, macrophage inflammatory protein 1α (MIP-1α) level, or macrophage inflammatory protein 1β (MIP-1β) level is reduced following the administration step.

In another aspect, the invention provides a method of modulating a multiple sclerosis marker in a subject in need thereof by administering to the subject in need thereof a therapeutically effective amount of a conjugate of the invention, a pharmaceutically acceptable salt thereof, or a pharmaceutical composition having a conjugate of the invention and a pharmaceutically acceptable carrier.

In certain embodiments, an Nrf2 expression level, citric acid level, serotonin level, β-hydroxybutyric acid level, docosahexaenoic acid level, putrescine level, N-methyl nicotinic acid level, lauric acid level, or arachidonic acid level is increased following the administration step. In further embodiments, a L-citrulline level, picolinic acid level, quinolinic acid level, 2-ketoglutaric acid level, L-kynurenine/L-tryptophan ratio, kyunurenic acid level, prostaglandin E2 level, leukotriene B4, linolenic acid level, linoleic acid level, CD8⁺ T cell count, memory B cell count, CD4⁺ EM cell count, or cumulative number of new Gd+ lesions, L-phenylalanine level, hippuric acid level, or eicosapentaenoic acid level is is reduced following the administration step.

In still another aspect, the invention provides a method of delivering a monomethyl fumarate moiety to a target site in a subject in need thereof by administering to the subject the conjugate described herein, or a pharmaceutically acceptable salt thereof, or the composition described herein.

In some embodiments, the target site is a small intestine (e.g., a proximal small intestine or a distal small intestine) of the subject. In some embodiments, the target site is a cecum of the subject. In some embodiments, the target site is a colon (e.g., a proximal colon or a distal colon) of the subject.

In some embodiments, a conjugate of the invention is administered to a subject in need there of orally or subcutaneously. In particular embodiments, a conjugate of the invention is administered to a subject in need thereof orally.

### Definitions

The term “acid monosaccharide,” as used herein, represents a sugar acid in its cyclic form (e.g., pyranose or furanose). When the core of a carrier group is an acid monosaccharide, each hydroxyl and acid group of the acid monosaccharide can be independently substituted. An acid monosaccharide that is an oxidized C₅₋₆ pyranose is a C₅₋₆ acid pyranose. Non-limiting examples of acid monosaccharides include glucuronic acid.

The term “acyl,” as used herein, represents a chemical substituent of formula —C(O)—R, where R is alkyl, alkenyl, aryl, arylalkyl, cycloalkyl, heterocyclyl, heterocyclyl alkyl, heteroaryl, or heteroaryl alkyl, or R combines with the carbonyl to which it is attached to form fatty acid acyl, ketone body acyl, pre-ketone body acyl, tryptophan analogue acyl, or monomethyl fumarate acyl.

The term “acylated aminomonosaccharide,” as used herein, refers to a compound or a monovalent group that is an aminomonosaccharide having one or more hydroxyls substituted with an alkyl, acyl (e.g., a fatty acid acyl, ketone body acyl, pre-ketone body acyl, tryptophan analogue acyl, or monomethyl fumarate acyl), optionally acylated ketone body, or optionally acylated pre-ketone body, provided that at least one of the hydroxyls is substituted with an acyl, optionally acylated ketone body, or optionally acylated pre-ketone body. Preferably, the fatty acid acyl is a short chain fatty acid acyl (e.g., propionyl or butyryl). When acylated sugar is a monovalent group, the valency is (i) on an oxygen atom of the aminomonosaccharide, or (ii) on an anomeric carbon atom of the aminomonosaccharide.

The term “acylated sugar,” as used herein, refers to a compound or a monovalent group that is a monosaccharide, sugar acid, or sugar alcohol having one or more hydroxyls substituted with an alkyl, acyl (e.g., a fatty acid acyl, ketone body acyl, pre-ketone body acyl, tryptophan analogue acyl, or monomethyl fumarate acyl), optionally acylated ketone body, or optionally acylated pre-ketone body, provided that at least one of the hydroxyls is substituted with an acyl, optionally acylated ketone body, or optionally acylated pre-ketone body. Preferably, the fatty acid acyl is a short chain fatty acid acyl (e.g., propionyl or butyryl). When acylated sugar is a monovalent group, the valency is (i) on an oxygen atom of the monosaccharide, sugar acid, or sugar alcohol, or (ii) on an anomeric carbon atom of the monosaccharide or sugar acid.

The term “acyloxy,” as used herein, represents a chemical substituent of formula —OR, where R is acyl.

The term “alcohol oxygen atom,” as used herein, refers to a divalent oxygen atom bonded to at least one sp³-hybridized carbon atom. A hydroxyl including an alcohol oxygen atom is an alcohol hydroxyl group.

The term “aldonyl,” as used herein, refers to a monovalent substituent that is aldonic acid in which a carboxylate hydroxyl is replaced with a valency.

The term “alkanoyl,” as used herein, represents a chemical substituent of formula —C(O)—R, where R is alkyl. An optionally substituted alkanoyl is an alkanoyl that is optionally substituted as described herein for alkyl.

The term “alkenyl,” as used herein, represents acyclic monovalent straight or branched chain hydrocarbon groups containing one, two, or three carbon-carbon double bonds. Alkenyl, when unsubstituted, has from 2 to 22 carbons, unless otherwise specified. In certain preferred embodiments, alkenyl, when unsubstituted, has from 2 to 12 carbon atoms (e.g., 1 to 8 carbons). Non-limiting examples of the alkenyl groups include ethenyl, prop-1-enyl, prop-2-enyl, 1-methylethenyl, but-1-enyl, but-2-enyl, but-3-enyl, 1-methylprop-1-enyl, 2-methylprop-1-enyl, and 1-methylprop-2-enyl. Alkenyl groups may be optionally substituted as defined herein for alkyl.

The term “alkoxy,” as used herein, represents a chemical substituent of formula —OR, where R is a C₁₋₆ alkyl group, unless otherwise specified. An optionally substituted alkoxy is an alkoxy group that is optionally substituted as defined herein for alkyl.

The term “alkyl,” as used herein, refers to an acyclic straight or branched chain saturated hydrocarbon group, which, when unsubstituted, has from 1 to 22 carbons (e.g., 1 to 20 carbons), unless otherwise specified. In certain preferred embodiments, alkyl, when unsubstituted, has from 1 to 12 carbons (e.g., 1 to 8 carbons). Alkyl groups are exemplified by methyl; ethyl; n- and iso-propyl; n-, sec-, iso- and tert-butyl; neopentyl, and the like, and may be optionally substituted, valency permitting, with one, two, three, or, in the case of alkyl groups of two carbons or more, four or more substituents independently selected from the group consisting of: alkoxy; acyloxy; alkylsulfenyl; alkylsulfinyl; alkylsulfonyl; amino; aryl; aryloxy; azido; cycloalkyl; cycloalkoxy; halo; heterocyclyl; heteroaryl; heterocyclylalkyl; heteroarylalkyl; heterocyclyloxy; heteroaryloxy; hydroxy; nitro; thioalkyl; thioalkenyl; thioaryl; thiol; silyl; cyano; ═O; ═S; and ═NR′, where R′ is H, alkyl, aryl, or heterocyclyl. Each of the substituents may itself be unsubstituted or, valency permitting, substituted with unsubstituted substituent(s) defined herein for each respective group.

The term “alkylated aminomonosaccharide,” as used herein, refers to a compound or a monovalent group that is an aminomonosaccharide having one or more hydroxyls substituted with an alkyl, acyl (e.g., a fatty acid acyl, ketone body acyl, pre-ketone body acyl, tryptophan analogue acyl, or monomethyl fumarate acyl), optionally acylated ketone body, or optionally acylated pre-ketone body, provided that at least one of the hydroxyls is substituted with an alkyl. When alkylated sugar is a monovalent group, the valency is (i) on an oxygen atom of the aminomonosaccharide, or (ii) on an anomeric carbon atom of the aminomonosaccharide.

The term “alkylated sugar,” as used herein, refers to a compound or a monovalent group that is a monosaccharide, sugar acid, or sugar alcohol having one or more hydroxyls substituted with an alkyl, acyl (e.g., a fatty acid acyl, ketone body acyl, pre-ketone body acyl, tryptophan analogue acyl, or monomethyl fumarate acyl), optionally acylated ketone body, or optionally acylated pre-ketone body, provided that at least one of the hydroxyls is substituted with an alkyl. When alkylated sugar is a monovalent group, the valency is (i) on an oxygen atom of the monosaccharide, sugar acid, or sugar alcohol, or (ii) on an anomeric carbon atom of the monosaccharide or sugar acid.

The term “aminocarrier,” as used herein, represents a carrier group, in which at least one hydroxyl is substituted with —NR₂, where each R is independently H or acyl. A non-limiting example of an aminocarrier group is an acylated aminomonosaccharide.

The term “aminomonosaccharide,” as used herein, represents a monosaccharide (e.g., a pyranose or furanose), in which at least one hydroxyl is replaced with —NR₂, where each R is independently H or acyl. An aminomonosaccharide that is a C₅₋₆ pyranose, in which at least one hydroxyl is replaced with —NR₂, is a C₅₋₆ aminopyranose. The aminomonosaccharide may be an aldose or ketose.

Non-limiting examples of aminomonosaccharides are glucosamine and galactosamine. In some embodiments, when the carrier group is an acylated aminomonosaccharide (e.g., acylated aminopyranose), one or more hydroxyls in the acylated aminomonosaccharide may be independently substituted with an alkyl, short chain fatty acid acyl, monomethyl fumarate acyl, tryptophan analogue acyl, ketone body acyl, or pre-ketone body acyl, and one and only one hydroxyl is substituted with a bond to monomethyl fumarate acyl, and one or more of the remaining hydroxyls are independently substituted as described herein. Preferably, the hydroxyl substituted with a bond to monomethyl fumarate acyl is attached to an anomeric carbon atom of the monosaccharide. Alternatively, the hydroxyl substituted with a bond to monomethyl fumarate acyl may be attached to position 4 or 6 of the aminomonosaccharide.

The term “aryl,” as used herein, is a monovalent or multivalent group consisting of one ring of carbon atoms or two, three, or four fused rings of carbon atoms, provided that at least one of the rings in aryl is n-aromatic. An unsubstituted aryl group typically contains from six to eighteen carbon atoms (e.g., from six to ten carbon atoms). An aryl group may be optionally substituted with 1, 2, 3, 4, or 5 substituents, where each of the substituents is independently alkyl, hydroxyl, protected hydroxyl, alkoxy, amino, protected amino, or heteroaryl.

The term “aryl alkyl,” as used herein, represents an alkyl group substituted with an aryl group. An optionally substituted aryl alkyl is an aryl alkyl, in which aryl and alkyl portions may be optionally substituted as the individual groups as described herein.

The term “aryloxy,” as used herein, represents a group —OR, where R is aryl. Aryloxy may be an optionally substituted aryloxy. An optionally substituted aryloxy is aryloxy that is optionally substituted as described herein for aryl.

The term “autoimmune disorder,” as used herein, refers to a group of diseases resulting from one's own immune system incorrectly attacking one's own tissue. Non-limiting examples of autoimmune disorders include multiple sclerosis, psoriasis, psoriatic arthritis, rheumatoid arthritis, systemic lupus erythematosus, Crohn's disease, Sjogren's syndrome, Behcet's disease, ulcerative colitis, and Guillain-Barré syndrome.

The term “autoimmunity marker,” as used herein, is an observable indication of the presence, absence, or risk of an autoimmune disorder (e.g., multiple sclerosis, psoriasis, psoriatic arthritis, rheumatoid arthritis, systemic lupus erythematosus, Crohn's disease, Sjogren's syndrome, Behcet's disease, ulcerative colitis, or Guillain-Barré syndrome).

The level of an autoimmunity marker may directly or inversely correlate with an autoimmune disorder state. Non-limiting examples of the autoimmunity markers are a CYP1A1 mRNA level, intestinal motility, CD4⁺CD25⁺ Treg cell (e.g., CD4⁺CD25⁺Foxp3⁺ Treg cell) count, mucus secretion, Tₕ1 cell count, interleukin-8 (IL8) level, macrophage inflammatory protein 1α (MIP-1α) level, macrophage inflammatory protein 1β (MIP-1β) level, NFκB level, inducible nitric oxide synthase (iNOS) level, matrix metallopeptidase 9 (MMP9) level, interferon γ (IFNγ) level, interleukin-17 (IL17) level, intercellular adhesion molecule (ICAM) level, CXCL13 level, 8-iso-prostaglandin F_{2α} (8-iso-PGF2α) level, IgA level, calprotectin level, lipocalin-2 level, short chain fatty acids level, and indoxyl sulfate level.

Autoimmunity markers may be measured using methods known in the art. For example, blood sample analyses may be used to measure a CD4⁺CD25⁺ Treg cell (e.g., CD4⁺CD25⁺Foxp3⁺ Treg cell) count, Tₕ1 cell count, NFκB level, inducible nitric oxide synthase (iNOS) level, matrix metallopeptidase 9 (MMP9) level, interferon γ (IFNγ) level, interleukin-17 (IL17) level, intercellular adhesion molecule (ICAM) level, CXCL13 level, and 8-iso-prostaglandin F_{2α} (8-iso-PGF2α) level. Stool sample analyses may be performed to measure an IgA level, calprotectin level, lipocalin-2 level, and short chain fatty acids level. Urine sample analysis may be performed to measure an indoxyl sulfate level.

The term “bile acid,” as used herein, represents a compound or monovalent group of formula:

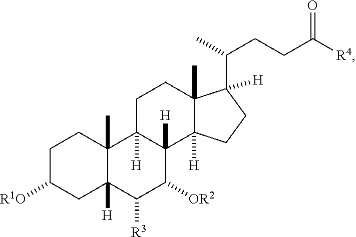

where
each of R¹ and R² is independently H, an alkyl, a bond to monomethyl fumarate acyl, short chain fatty acid acyl, monomethyl fumarate acyl, tryptophan analogue acyl, ketone body acyl, or pre-ketone body acyl;
R³ is H or alkyl (e.g., ethyl); and
R⁴ is hydroxyl, alkoxy, optionally acylated ketone body, or optionally acylated pre-ketone body.

When the carrier group is bile acid having one or more hydroxyls independently substituted with an alkyl, short chain fatty acid acyl, monomethyl fumarate acyl, tryptophan analogue acyl, optionally acylated ketone body, ketone body acyl, pre-ketone body acyl, or optionally acylated pre-ketone body; one and only one of R¹ and R² is a bond to monomethyl fumarate acyl, and the remaining one of R¹ and R² groups is independently an alkyl, short chain fatty acid acyl, monomethyl fumarate acyl, tryptophan analogue acyl, ketone body acyl, or pre-ketone body acyl, and/or one or both R^{B} groups are independently alkyl, optionally acylated ketone body, or optionally acylated pre-ketone body.

A non-limiting example of bile acid is obeticholic acid.

The term “carbonate linker,” as used herein, refers to a group R¹—(CO)—R², where R¹ and R² are bonds to two different oxygen atoms.

The term “carbonyl,” as used herein, refers to a divalent group —C(O)—.

The term “carboxylate,” as used herein, refers to a group —COOH or a salt thereof.

The term “carboxylate oxygen atom,” as used herein, refers to a divalent oxygen atom having one and only one valency bonded to the carbon atom of a carbonyl group. A hydroxyl including a carboxylate oxygen atom is a carboxylic hydroxyl group.

The term “carrier group,” as used herein, refers to (i) a monovalent group having a core and one or more substituents covalently bonded to the core, where each substituent is independently an acyl, alkyl, optionally acylated ketone body, optionally acylated pre-ketone body, or tryptophan analogue; provided that at least one substituent is an acyl, optionally acylated ketone body, optionally acylated pre-ketone body, or tryptophan analogue, or (ii) a tryptophan analogue having an alcohol oxygen atom substituted with a valency. The valency of the carrier group is on a carbon atom of a carbonyl group, on an anomeric carbon atom, on an alcohol oxygen atom, on a phenolic oxygen atom, or on a carboxylate oxygen atom. The core is a carbohydrate (e.g., monosaccharide), sugar acid, sugar alcohol, catechin polyphenol, ellagic acid, ellagic acid analogue, stilbenoid, curcuminoid, chalconoid, pyridoxine, bile acid, ketone body, or pre-ketone body. Preferably, the core is a monosaccharide. The one or more acyl groups are independently bonded to the core through a carbonate linker, ester bond, or glycosidic bond. In some embodiments, each substituent may be independently an alkyl, short chain fatty acid acyl, tryptophan analogue acyl, ketone body acyl, or pre-ketone body acyl. In some embodiments, the core is peracylated, i.e., all available hydroxyls on the core are substituted with acyls. In some embodiments, the carrier group is an acylated sugar. In some embodiments, a carrier group having a fatty acid acyl substituent is a group containing a short chain fatty acid. In some embodiments, a carrier group having a tryptophan analogue acyl substituent is a group containing a tryptophan analogue. In some embodiments, a carrier group having a ketone body core, a pre-ketone body core, a ketone body acyl substituent, pre-ketone body acyl substituent, optionally acylated ketone body, or optionally acylated pre-ketone body is a group containing a ketone body or pre-ketone body.

The term “catechin polyphenol,” as used herein, refers to a compound, a carrier group, or a core of formula:

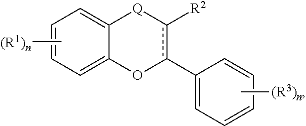

where
[symbol] is a single carbon-carbon bond or double carbon-carbon bond;
Q is —CH₂— or —C(O)—;
each R¹ and each R³ is independently H, halogen, —OR^{A};
R² is H or —OR^{A};
each R^{A} is independently H, alkyl, a bond to monomethyl fumarate acyl, short chain fatty acid acyl, monomethyl fumarate acyl, tryptophan analogue acyl, ketone body acyl, pre-ketone body acyl, or benzoyl optionally substituted with 1, 2, 3, or 4 substituents independently selected from the group consisting of H, hydroxy, halogen, optionally substituted alkyl, alkoxy, a bond to monomethyl fumarate acyl, short chain fatty acid acyl, monomethyl fumarate acyl, tryptophan analogue acyl, ketone body acyl, or pre-ketone body acyl; and
each of n and m is independently 1, 2, 3, or 4.

Preferably, n is 2. Preferably, m is 2 or 3.

Non-limiting examples of catechin polyphenols include epigallocatechin gallate, apigenin, naringenin, genistein, quercetin, luteolin, daidzein, equol, or hesperetin.

When the carrier group is a catechin polyphenol having one or more hydroxyls independently substituted with an alkyl, short chain fatty acid acyl, monomethyl fumarate acyl, tryptophan analogue acyl, ketone body acyl, or pre-ketone body acyl, one and only one R^{A} is a bond to monomethyl fumarate acyl, and one or more of the remaining R^{A} groups are independently an alkyl, short chain fatty acid acyl, monomethyl fumarate acyl, tryptophan analogue acyl, ketone body acyl, or pre-ketone body acyl.

The term “chalconoid,” as used herein, refers to a compound or monovalent group of the structure:

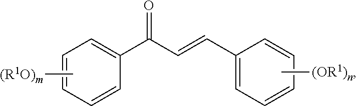

where
each of n and m is independently 0, 1, 2, or 3;
each R¹ is independently H, hydroxy, alkoxy, a bond to monomethyl fumarate acyl, short chain fatty acid acyl, monomethyl fumarate acyl, tryptophan analogue acyl, ketone body acyl, or pre-ketone body acyl;
provided that at least one R¹ is present.

When the carrier group is a chalconoid having one or more hydroxyls independently substituted with an alkyl, short chain fatty acid acyl, monomethyl fumarate acyl, tryptophan analogue acyl, ketone body acyl, or pre-ketone body acyl, one and only one R¹ is a bond to monomethyl fumarate acyl, and one or more of the remaining R¹ groups are independently an alkyl, short chain fatty acid acyl, monomethyl fumarate acyl, tryptophan analogue acyl, ketone body acyl, or pre-ketone body acyl. A non-limiting example of a chalconoid is:

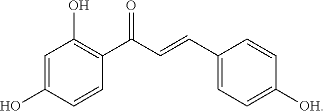 [SMILES: O=C(/C=C/c1ccc(O)cc1)c1ccc(O)cc1O]

The term “cleavable in vivo,” as used herein, refers to a property of a compound or a bond within a compound that is broken down in vivo to produce at least two separate compounds. In some embodiments, the cleavage process is hydrolysis. Thus, a compound that is cleavable in vivo may be a compound hydrolyzable in vivo. Cleavage of a compound or bond can be mediated by an enzyme or may proceed spontaneously under conditions present in a given in vivo compartment (e.g., a portion of the gastrointestinal tract (e.g., the duodenum)).

The term “conjugate of monomethyl fumarate”, as used herein, refers to a compound of the following formula:

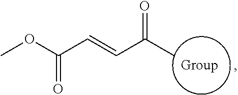

where Group is a monovalent substituent bonded to the monomethyl fumarate acyl through a carbon-oxygen bond as described herein.

The term “curcuminoid,” as used herein, refers to a compound or monovalent group of the structure:

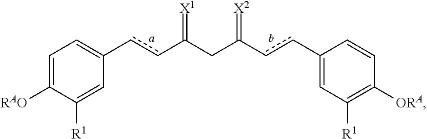

or a tautomer thereof,
where
each or a and b is independently a single or a double bond;
each of X¹ and X², together with the carbon atom to which each is attached, is independently a carbonyl or —(CH(OR^{A}))—;
each R^{A} is independently H, a bond to monomethyl fumarate acyl, short chain fatty acid acyl, monomethyl fumarate acyl, tryptophan analogue acyl, ketone body acyl, or pre-ketone body acyl; and
each R¹ is independently H or OMe.

When the carrier group is a curcuminoid having one or more hydroxyls independently substituted with an alkyl, short chain fatty acid acyl, monomethyl fumarate acyl, tryptophan analogue acyl, ketone body acyl, or pre-ketone body acyl, one and only one R^{A} is a bond to monomethyl fumarate acyl, and one or more of the remaining R^{5A} groups are independently an alkyl, short chain fatty acid acyl, monomethyl fumarate acyl, tryptophan analogue acyl, ketone body acyl, or pre-ketone body acyl.

Non-limiting examples of curcuminoids include:

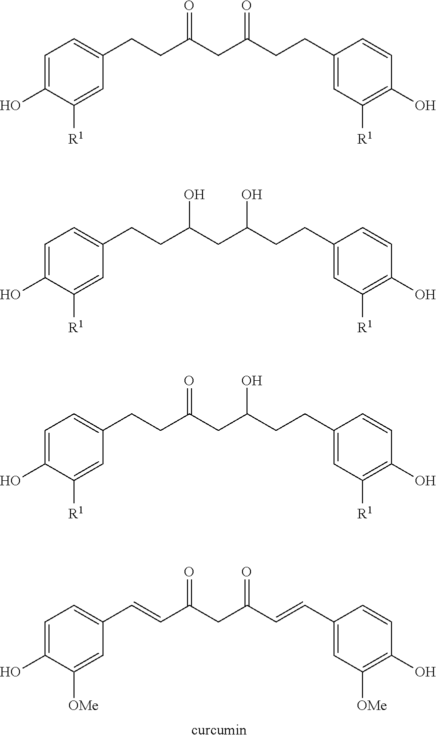

The terms “ellagic acid” and “ellagic acid analogue,” as used herein, collectively refer to a compound or monovalent group of the structure:

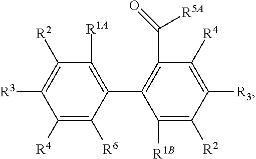

where
each of R², R³, and R′ is independently H or —OR^{A};
R⁶ is H or —(CO)—R^{5B};
R^{1A} is H or —OR^{A}, and R^{5A} is —OH or —OR^{B}; or R^{1A} and R^{5A} combine to form —O—;
R^{1B} is H or —OR^{A}, and R^{5B} is absent, —OH, or —OR^{B}; or R^{1B} and R^{5B} combine to form —O—;
each R^{A} is independently H, O-protecting group, a bond to monomethyl fumarate acyl, short chain fatty acid acyl, monomethyl fumarate acyl, tryptophan analogue acyl, ketone body acyl, or pre-ketone body acyl; and
each R^{B} is independently H, alkyl, optionally acylated ketone body, or optionally acylated pre-ketone body.

When the carrier group is an ellagic acid or an analogue thereof having one or more hydroxyls independently substituted with an alkyl, short chain fatty acid acyl, monomethyl fumarate acyl, tryptophan analogue acyl, optionally acylated ketone body, ketone body acyl, pre-ketone body acyl, or optionally acylated pre-ketone body; one and only one R^{A} is a bond to monomethyl fumarate acyl, and one or more of the remaining R^{5A} groups are independently an alkyl, short chain fatty acid acyl, monomethyl fumarate acyl, tryptophan analogue acyl, ketone body acyl, or pre-ketone body acyl, and/or one or both R^{B} groups are independently alkyl, optionally acylated ketone body, or optionally acylated pre-ketone body. The term “ellagic acid analogue,” refers to the compounds and groups of the above structure that are not ellagic acid. The term “ellagic acid” refers to the following two compounds:

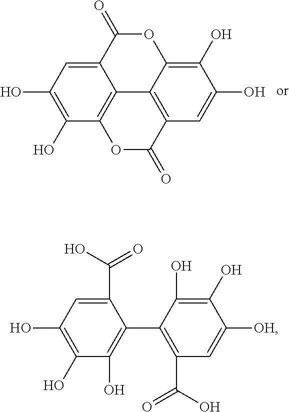 [SMILES: O=c1oc2c(O)c(O)cc3c(=O)oc4c(O)c(O)cc1c4c23 ; "or" ; O=C(O)c1cc(O)c(O)c(O)c1-c1c(C(=O)O)cc(O)c(O)c1O]

or these compounds within the structure of a conjugate.

Non-limiting examples of ellagic acid analogues include urolithin A, urolithin B, urolithin C, urolithin D, urolithin E, and urolithin M5.

The term “ester bond,” as used herein, refers to a covalent bond between an alcohol or phenolic oxygen atom and the carbon atom of carbonyl group that is further bonded to a carbon atom.

The term “fatty acid,” as used herein, refers to a short-chain fatty acid, a medium chain fatty acid, a long chain fatty acid, a very long chain fatty acid, or an unsaturated analogue thereof, or a phenyl-substituted analogue thereof. Short chain fatty acids contain from 1 to 6 carbon atoms, medium chain fatty acids contain from 7 to 13 carbon atoms, long-chain fatty acids contain from 14 to 22 carbon atoms, and a very long-chain fatty acid contains 23 to 26 carbon atoms. Fatty acids described herein are saturated fatty acids. Non-limiting examples of short-chain fatty acids include propionic acid and butyric acid. For the avoidance of doubt, the term “fatty acid,” as used herein, includes isotopically enriched fatty acids, e.g., fatty acids, in which one or more hydrogen atom positions carries deuterium. Non-limiting examples of deuterated short-chain fatty acids include deuterated propionic acid (e.g., d3-propionic acid) and deuterated butyric acid (e.g., d5-butyric acid).

D3-propionic acid is of the following structure:

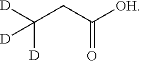

D5-butyric acid is of the following structure:

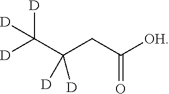 [SMILES: [2H]C([2H])([2H])C([2H])([2H])CC(=O)O]

The term “fatty acid acyl,” as used herein, refers to a fatty acid, in which the carboxyl hydroxyl group is replaced with a valency. Non-limiting examples of short-chain fatty acid acyls include propionyl and butyryl. Non-limiting examples of deuterated short-chain fatty acid acyls include deuterated propionyl (e.g., d3-propionyl) and deuterated butyryl (e.g., d5-butyryl).

D3-propionyl is of the following structure:

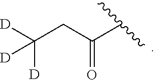 [SMILES: *C(=O)CC([2H])([2H])[2H]]

D5-butyryl is of the following structure:

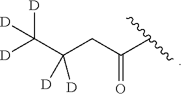 [SMILES: *C(=O)CC([2H])([2H])C([2H])([2H])[2H]]

The term “fatty acid acyloxy,” as used herein, refers to group —OR, where R is a fatty acid acyl.

The term “glycosidic bond,” as used herein, refers to a covalent bond between an oxygen atom and an anomeric carbon atom in a pyranose ring or furanose ring. In some embodiments, the anomeric carbon is in position 1.

The term “halogen,” as used herein, represents a halogen selected from bromine, chlorine, iodine, and fluorine.

The term “heteroaryl,” as used herein, represents a monocyclic 5⁻, 6⁻, 7⁻, or 8-membered ring system, or a fused or bridging bicyclic, tricyclic, or tetracyclic ring system; the ring system contains one, two, three, or four heteroatoms independently selected from the group consisting of nitrogen, oxygen, and sulfur; and at least one of the rings is an aromatic ring. Non-limiting examples of heteroaryl groups include benzimidazolyl, benzofuryl, benzothiazolyl, benzothienyl, benzoxazolyl, furyl, imidazolyl, indolyl, isoindazolyl, isoquinolinyl, isothiazolyl, isothiazolyl, isoxazolyl, oxadiazolyl, oxazolyl, purinyl, pyrrolyl, pyridinyl, pyrazinyl, pyrimidinyl, quinazolinyl, quinolinyl, thiadiazolyl (e.g., 1,3,4-thiadiazole), thiazolyl, thienyl, triazolyl, tetrazolyl, dihydroindolyl, tetrahydroquinolyl, tetrahydroisoquinolyl, etc. The term bicyclic, tricyclic, and tetracyclic heteroaryls include at least one ring having at least one heteroatom as described above and at least one aromatic ring. For example, a ring having at least one heteroatom may be fused to one, two, or three carbocyclic rings, e.g., an aryl ring, a cyclohexane ring, a cyclohexene ring, a cyclopentane ring, a cyclopentene ring, or another monocyclic heterocyclic ring. Examples of fused heteroaryls include 1,2,3,5,8,8a-hexahydroindolizine; 2,3-dihydrobenzofuran; 2,3-dihydroindole; and 2,3-dihydrobenzothiophene. Heteroaryl may be optionally substituted with one, two, three, four, or five substituents independently selected from the group consisting of: alkyl; alkenyl; alkoxy; acyloxy; aryloxy; alkylsulfenyl; alkylsulfinyl; alkylsulfonyl; amino; arylalkoxy; cycloalkyl; cycloalkoxy; halogen; heterocyclyl; heterocyclyl alkyl; heteroaryl; heteroaryl alkyl; heterocyclyloxy; heteroaryloxy; hydroxyl; nitro; thioalkyl; thioalkenyl; thioaryl; thiol; cyano; ═O; —NR₂, where each R is independently hydrogen, alkyl, acyl, aryl, arylalkyl, cycloalkyl, heterocyclyl, or heteroaryl; —COOR^{A}, where R^{A} is hydrogen, alkyl, aryl, arylalkyl, cycloalkyl, heterocyclyl, or heteroaryl; and —CON(R^{B})₂, where each R^{B} is independently hydrogen, alkyl, aryl, arylalkyl, cycloalkyl, heterocyclyl, or heteroaryl. Each of the substituents may itself be unsubstituted or substituted with unsubstituted substituent(s) defined herein for each respective group.

The term “heteroaryl alkyl,” as used herein, represents an alkyl group substituted with a heteroaryl group. An optionally substituted heteroaryl alkyl is a heteroaryl alkyl, in which heteroaryl and alkyl portions may be optionally substituted as the individual groups as described herein. The term “heteroaryloxy,” as used herein, refers to a structure —OR, in which R is heteroaryl. Heteroaryloxy can be optionally substituted as defined for heteroaryl.

The term “heterocyclyl,” as used herein, represents a monocyclic, bicyclic, tricyclic, or tetracyclic non-aromatic ring system having fused or bridging 4⁻, 5⁻, 6⁻, 7⁻, or 8-membered rings, unless otherwise specified, the ring system containing one, two, three, or four heteroatoms independently selected from the group consisting of nitrogen, oxygen, and sulfur. Non-aromatic 5-membered heterocyclyl has zero or one double bonds, non-aromatic 6- and 7-membered heterocyclyl groups have zero to two double bonds, and non-aromatic 8-membered heterocyclyl groups have zero to two double bonds and/or zero or one carbon-carbon triple bond. Heterocyclyl groups have a carbon count of 1 to 16 carbon atoms unless otherwise specified. Certain heterocyclyl groups may have a carbon count up to 9 carbon atoms. Non-aromatic heterocyclyl groups include pyrrolinyl, pyrrolidinyl, pyrazolinyl, pyrazolidinyl, imidazolinyl, imidazolidinyl, piperidinyl, homopiperidinyl, piperazinyl, pyridazinyl, oxazolidinyl, isoxazolidinyl, morpholinyl, thiomorpholinyl, thiazolidinyl, isothiazolidinyl, thiazolidinyl, tetrahydrofuranyl, dihydrofuranyl, tetrahydrothienyl, dihydrothienyl, pyranyl, dihydropyranyl, dithiazolyl, etc. The term “heterocyclyl” also represents a heterocyclic compound having a bridged multicyclic structure in which one or more carbons and/or heteroatoms bridges two non-adjacent members of a monocyclic ring, e.g., quinuclidine, tropanes, or diaza-bicyclo[2.2.2]octane. The term “heterocyclyl” includes bicyclic, tricyclic, and tetracyclic groups in which any of the above heterocyclic rings is fused to one, two, or three carbocyclic rings, e.g., a cyclohexane ring, a cyclohexene ring, a cyclopentane ring, a cyclopentene ring, or another heterocyclic ring. Examples of fused heterocyclyls include 1,2,3,5,8,8a-hexahydroindolizine; 2,3-dihydrobenzofuran; 2,3-dihydroindole; and 2,3-dihydrobenzothiophene. The heterocyclyl group may be unsubstituted or substituted with one, two, three, four or five substituents independently selected from the group consisting of: alkyl; alkenyl; alkoxy; acyloxy; alkylsulfenyl; alkylsulfinyl; alkylsulfonyl; aryloxy; amino; arylalkoxy; cycloalkyl; cycloalkoxy; halogen; heterocyclyl; heterocyclyl alkyl; heteroaryl; heteroaryl alkyl; heterocyclyloxy; heteroaryloxy; hydroxyl; nitro; thioalkyl; thioalkenyl; thioaryl; thiol; cyano; ═O; ═S; —NR₂, where each R is independently hydrogen, alkyl, acyl, aryl, arylalkyl, cycloalkyl, heterocyclyl, or heteroaryl; —COOR^{A}, where R^{A} is hydrogen, alkyl, aryl, arylalkyl, cycloalkyl, heterocyclyl, or heteroaryl; and —CON(R^{B})₂, where each R^{B} is independently hydrogen, alkyl, aryl, arylalkyl, cycloalkyl, heterocyclyl, or heteroaryl.

The term “heterocyclyl alkyl,” as used herein, represents an alkyl group substituted with a heterocyclyl group. The heterocyclyl and alkyl portions of an optionally substituted heterocyclyl alkyl are optionally substituted as the described for heterocyclyl and alkyl, respectively.

The term “heterocyclylene,” as used herein, represents a heterocyclyl, in which one hydrogen atom is replaced with a valency. An optionally substituted heterocyclylene is a heterocyclylene that is optionally substituted as described herein for heterocyclyl.

The term “heterocyclyloxy,” as used herein, refers to a structure —OR, in which R is heterocyclyl. Heterocyclyloxy can be optionally substituted as described for heterocyclyl.

The terms “hydroxyl” and “hydroxy,” as used interchangeably herein, represent —OH. A hydroxyl substituted with an acyl is an acyloxy. A hydroxyl substituted with an alkyl is an alkoxy. A protected hydroxyl is a hydroxyl in which the hydrogen atom is replaced with an O-protecting group.

The term “ketone body,” as used herein, refers to (i) β-hydroxybutyric acid, or (ii) a group that is β-hydroxybutyric acid, where at least one hydroxyl hydrogen atom is replaced with a valency or a carboxylate —OH is replaced with a valency. An optionally acylated ketone body has an alcohol hydroxyl optionally substituted with short chain fatty acid acyl, monomethyl fumarate acyl, or tryptophan analogue acyl.

The term “ketone body acyl,” as used herein, refers to a β-hydroxybutyric acid, in which the carboxylate —OH group is replaced with a valency.

The term “4-methyl-1,3-dioxan-2-yl,” as used herein, refers to the monovalent group of formula:

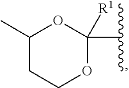

where R¹ is optionally substituted C₁₋₆ alkyl (e.g., methyl).

The term “modulating,” as used herein, refers to an observable change, for example, in the level of a marker in a subject, as measured using techniques and methods known in the art for such a measurement. Modulating the marker level in a subject may result in a change of at least 1% relative to prior to administration (e.g., at least 5%, 10%, 15%, 20%, 25%, 30%, 35%, 40%, 45%, 50%, 55%, 60%, 65%, 70%, 75%, 80%, 85%, 90%, 95% or at least 98% or more relative to prior to administration; e.g., up to 100% relative to prior to administration). In some embodiments, modulating is increasing the level of a marker in a subject. Increasing the marker level in a subject may result in an increase of at least 1% relative to prior to administration (e.g., at least 5%, 10%, 15%, 20%, 25%, 30%, 35%, 40%, 45%, 50%, 55%, 60%, 65%, 70%, 75%, 80%, 85%, 90%, 95% or at least 98% or more relative to prior to administration; e.g., up to 100% relative to prior to administration). In other embodiments, modulating is decreasing the level of a marker in a subject. Decreasing the marker level in a subject may result in a decrease of at least 1% relative to prior to administration (e.g., at least 5%, 10%, 15%, 20%, 25%, 30%, 35%, 40%, 45%, 50%, 55%, 60%, 65%, 70%, 75%, 80%, 85%, 90%, 95% or at least 98% or more relative to prior to administration; e.g., up to 100% relative to prior to administration). In embodiments in which a parameter is increased or decreased (or reduced) in a subject following a step of administering a composition described herein, the increase or decrease may take place and/or be detectable within a range of time following the administration (e.g., within six hours, 24 hours, 3 days, a week or longer), and may take place and/or be detectable after one or more administrations (e.g., after 2, 3, 4, 5, 6, 7, 8, 9, 10 or more administrations, e.g., as part of a dosing regimen for the subject).

The term “monomethyl fumarate acyl,” as used herein, refers to a group of the following structure:

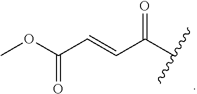 [SMILES: *C(=O)/C=C/C(=O)OC]

The term “monosaccharide,” as used herein, represents C₅₋₆ pyranoses and C₄₋₆ furanoses. The monosaccharide may be an aldose (e.g., an aldopyranose) or ketose (e.g., a ketopyranose). Non-limiting examples of monosaccharides are arabinose, xylose, fructose, galactose, glucose, ribose, tagatose, fucose, mannose, and rhamnose. In some embodiments, the monosaccharide is L-arabinose, D-xylose, fructose, galactose, D-glucose, D-ribose, D-tagatose, L-fucose, or L-rhamnose. When the core of a carrier group is a monosaccharide, each hydroxyl group of the monosaccharide can be independently substituted. For example, when the carrier group is a monosaccharide having one or more hydroxyls independently substituted with an alkyl, short chain fatty acid acyl, monomethyl fumarate acyl, tryptophan analogue acyl, ketone body acyl, or pre-ketone body acyl, one and only one hydroxyl is substituted with a bond to monomethyl fumarate acyl, and one or more of the remaining hydroxyls are independently substituted with an alkyl, short chain fatty acid acyl, monomethyl fumarate acyl, tryptophan analogue acyl, ketone body acyl, or pre-ketone body acyl. Preferably, the hydroxyl substituted with a bond to monomethyl fumarate acyl is attached to an anomeric carbon atom of the monosaccharide. Alternatively, the hydroxyl substituted with a bond to monomethyl fumarate acyl may be attached to, e.g., position 4 or 6 of the monosaccharide. For the avoidance of doubt, the position enumeration in monosaccharides that are pyranoses is as follows:

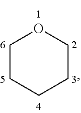

where position 2 designates an anomeric carbon atom.

The term “multiple sclerosis marker,” as used herein, is an observable indication of the presence, absence, or risk of multiple sclerosis (e.g., primary progressive multiple sclerosis, secondary progressive multiple sclerosis, or relapsing-remitting multiple sclerosis). Non-limiting examples of multiple sclerosis markers include an Nrf2 expression level, citric acid level, serotonin level, β-hydroxybutyric acid level, docosahexaenoic acid level, a L-citrulline level, picolinic acid level, quinolinic acid level, 2-ketoglutaric acid level, L-kynurenine/L-tryptophan ratio, kyunurenic acid level, prostaglandin E2 level, leukotriene B4, linolenic acid level, linoleic acid level, CD8⁺ T cell count, memory B cell count, CD4⁺ EM cell count, cumulative number of new Gd+ lesions, L-phenylalanine level, hippuric acid level, eicosapentaenoic acid level, putrescine level, N-methyl nicotinic acid level, lauric acid level, arachidonic acid level, and 2-hydroxyisovaleric acid level. 2-hydroxyisovaleric acid level may increase or decrease. For example, reduction of the 2-hydroxyisovaleric acid level in subject's urine is an improvement in the multiple sclerosis marker. Increase in the 2-hydroxyisovaleric acid level in subject's cerebrospinal fluid is also an improvement in the multiple sclerosis marker. The level of 2-hydroxyisovaleric acid in subject's urine is typically measured using gas chromatography, and the level of 2-hydroxyisovaleric acid in subject's cerebrospinal fluid is measured using NMR.

The term “oxo,” as used herein, represents a divalent oxygen atom (e.g., the structure of oxo may be shown as ═O).

The term “pharmaceutical composition,” as used herein, represents a composition containing a compound described herein, formulated with a pharmaceutically acceptable excipient, and manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other formulation described herein.

The term “pharmaceutically acceptable salt,” as use herein, represents those salts which are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting the free base group with a suitable organic acid. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

The term “phenolic oxygen atom,” as used herein, refers to a divalent oxygen atom bonded to an sp²-hybridized carbon atom within a n-aromatic ring. The phenolic oxygen may be further bonded to an sp³-hybridized carbon atom or an sp²-hybridized carbon atom.

The term “pre-ketone body,” as used herein, represents (i) a ketone body having hydroxymethyl instead of a carboxylate, or (ii) a group that is a ketone body having hydroxymethyl instead of a carboxylate, where at least one hydroxyl is replaced with +OR, where R is a valency. A non-limiting example of a pre-ketone body is butane-1,3-diol or 4-hydroxybutan-2-one. The term “pre-ketone body,” as used herein, also represents (4-methyl-1,3-dioxan-2-yl-alkylene)ₙ—CO—R^{A}, where n is 0 or 1, and R^{A} is —OH, if the pre-ketone body is not part of a conjugate, or a valency if the pre-ketone body is part of a group including a pre-ketone body (e.g., a pre-ketone body acyl). A non-limiting example of a pre-ketone body is butane-1,3-diol or 4-hydroxybutan-2-one. An optionally acylated pre-ketone body has an alcohol hydroxyl optionally substituted with short chain fatty acid acyl, monomethyl fumarate acyl, or tryptophan analogue acyl.

The term “pre-ketone body acyl,” as used herein, refers to a pre-ketone body, in which the carboxylate —OH group is replaced with a valency.

The term “subject,” as used herein, represents a human or non-human animal (e.g., a mammal) that is suffering from, or is at risk of, disease, disorder, or condition, as determined by a qualified professional (e.g., a doctor or a nurse practitioner) with or without known in the art laboratory test(s) of sample(s) from the subject. Non-limiting examples of diseases, disorders, and conditions include autoimmune disorders (e.g., multiple sclerosis, psoriasis, psoriatic arthritis, rheumatoid arthritis, systemic lupus erythematosus, Crohn's disease, Sjogren's syndrome, Behcet's disease, ulcerative colitis, or Guillain-Barré syndrome), adrenoleukodystrophy, AGE-induced genome damage, Alexander's disease, Alper's disease, Alzheimer's disease, amyotrophic lateral sclerosis, angina pectoris, arthritis, asthma, balo concentric sclerosis, Canavan disease, cardiac insufficiency including left ventricular insufficiency, central nervous system vasculitis, Charcott-Marie-Tooth Disease, childhood ataxia with central nervous system hypomyelination, chronic idiopathic peripheral neuropathy, chronic obstructive pulmonary disease, diabetic retinopathy, graft-versus-host-disease, hepatitis C viral infection, herpes simplex viral infection, human immunodeficiency viral infection, Huntington's disease, irritable bowel syndrome, ischemia, Krabbe disease, lichen planus, macular degeneration, mitochondrial encephalomyopathy, monomelic amyotrophy, myocardial infarction, neurodegeneration with brain iron accumulation, neuromyelitis optica, neurosarcoidosis, optic neuritis, paraneoplastic syndrome, Parkinson's disease, Pelizaeus-Merzbacher disease, primary lateral sclerosis, progressive supranuclear palsy, reperfusion injury, retinopathia pigmentosa, Schilder's disease, subacute necrotizing myelopathy, susac syndrome, transverse myelitis, Zellweger's syndrome, granuloma annulare, pemphigus, bollus pemphigoid, contact dermatitis, acute dermatitis, chronic dermatitis, alopecia areata (totalis or universalis), sarcoidosis, cutaneous sarcoidosis, pyoderma gangrenosum, cutaneous lupus, cutaneous Crohn's disease, obstructive sleep apnea, chronic lymphocytic leukemia, small lymphocytic leukemia, systemic sclerosis-pulmonary hypertension, glioblastoma multiforme, cutaneous T cell lymphoma, progressive multifocal leukoencephalopathy, polyarthritis, juvenile-onset diabetes, type II diabetes, Hashimoto's thyroiditis, Grave's disease, pernicious anaemia, autoimmune hepatitis, neurodermatitis, retinopathia pigmentosa or forms of mitochondrial encephalomyopathy, progressive systemic sclerodermia, osteochondritis syphilitica (Wegener's disease), cutis marmorata (livedo reticularis), panarteriitis, vasculitis, osteoarthritis, gout, arteriosclerosis, Reiter's disease, pulmonary granulomatosis, endotoxic shock (septic-toxic shock), sepsis, pneumonia, encephalomyelitis, anorexia nervosa, acute hepatitis, chronic hepatitis, toxic hepatitis, alcohol-induced hepatitis, viral hepatitis, liver insufficiency, cytomegaloviral hepatitis, Rennert T-lymphomatosis, mesangial nephritis, post-angioplastic restenosis, reperfusion syndrome, cytomegaloviral retinopathy, adenoviral cold, adenoviral pharyngoconjunctival fever, adenoviral ophthalmia, AIDS, post-herpetic or post-zoster neuralgia, inflammatory demyelinating polyneuropathy, mononeuropathia multiplex, mucoviscidosis, Bechterew's disease, Barett oesophagus, Epstein-Barr virus infection, cardiac remodeling, interstitial cystitis, diabetes mellitus type II, human tumor radiosensitization, multidrug resistance in chemotherapy, mamma carcinoma, colon carcinoma, melanoma, primary liver cell carcinoma, adenocarcinoma, Kaposi's sarcoma, prostate carcinoma, leukaemia, acute myeloid leukaemia, multiple myeloma (plasmocytoma), Burkitt's lymphoma, Castleman tumor, cardiac insufficiency, myocardial infarct, angina pectoris, asthma, chronic obstructive pulmonary diseases, PDGF induced thymidine uptake of bronchial smooth muscle cells, bronchial smooth muscle cell proliferation, alcoholism, Alexander's disease, Alper's disease, Alzheimer's disease, ataxia telangiectasia, Batten disease (also known as Spielmeyer-Vogt-Sjögren-Batten disease), bovine spongiform encephalopathy (BSE), Cerebral palsy, Cockayne syndrome, corticobasal degeneration, Creutzfeldt-Jakob disease, familial fatal insomnia, frontotemporal lobar degeneration, Huntington's disease, HIV-associated dementia, Kennedy's disease, Krabbe's disease, Lewy body dementia, neuroborreliosis, Machado-Joseph disease (Spinocerebellar ataxia type 3), multiple system atrophy, narcolepsy, Niemann Pick disease, Pelizaeus-Merzbacher disease, Pick's disease, primary lateral sclerosis, prion disease, progressive supranuclear palsy, Refsum's disease, Sandhoff disease, subacute combined degeneration of spinal cord secondary to pernicious anaemia, spinocerebellar ataxia, spinal muscular atrophy, Steele-Richardson-Olszewski disease, Tabes dorsalis, toxic encephalopathy, LHON (Lebor's Hereditary optic neuropathy), MELAS (Mitochondrial Encephalomyopathy; Lactic Acidosis; Stroke), MERRF (Myoclonic Epilepsy; Ragged Red Fibers), PEO (Progressive External Opthalmoplegia), Leigh's Syndrome, MNGIE (Myopathy and external ophthalmoplegia; Neuropathy; Gastro-Intestinal; Encephalopathy), Kearns-Sayre Syndrome (KSS), NARP, hereditary spastic paraparesis, mitochondrial myopathy, Friedreich Ataxia, optic neuritis, acute inflammatory demyelinating polyneuropathy (AIDP), chronic inflammatory demyelinating polyneuropathy (CIDP), acute transverse myelitis, acute disseminated encephalomyelitis (ADEM), and Leber's optic atrophy.

The term “sugar acid,” as used herein, refers to an oxidized monosaccharide having a carboxylic acid moiety. For example, in the linear form of a sugar acid, one or both terminal positions may be oxidized to a carboxylic acid. Sugar acids have a carbon count of three to six. There are four classes of sugar acids: aldonic acid, ulosonic acid, uronic acid, and aldaric acid. Non-limiting examples of sugar acids include xylonic acid, gluconic acid, glucuronic acid, galacturonic acid, tartaric acid, saccharic acid, or mucic acid. When the core of a carrier group is a sugar acid, each hydroxyl group of the sugar acid can be independently substituted. For example, when the carrier group is a sugar acid having one or more hydroxyls independently substituted with an alkyl, short chain fatty acid acyl, monomethyl fumarate acyl, tryptophan analogue acyl, optionally acylated ketone body, ketone body acyl, pre-ketone body acyl, or optionally acylated pre-ketone body; one and only one alcohol hydroxyl group is substituted with a bond to monomethyl fumarate acyl, and one or more of the remaining alcohol hydroxyl groups are independently an alkyl, short chain fatty acid acyl, monomethyl fumarate acyl, tryptophan analogue acyl, ketone body acyl, or pre-ketone body acyl, and/or one or more of the carboxylic hydroxyl groups are independently alkyl, optionally acylated ketone body, or optionally acylated pre-ketone body.

The term “sugar acid acyl,” as used herein, refers to a monovalent group that is a sugar acid having a carboxylate, in which —OH is replaced with a valency.

The term “sugar alcohol,” as used herein, refers to inositol or a compound of formula HOCH₂(CHOH)ₙCH₂OH, where n is 1, 2, 3, or 4. Non-limiting examples of sugar alcohols include glycerol, erythritol, threitol, arabitol, xylitol, tibitol, mannitol, sorbitol, galactitol, fucitol, iditol, and inositol.

When the core of a carrier group is a sugar alcohol, each hydroxyl group of the sugar alcohol can be independently substituted. For example, when the carrier group is a sugar alcohol having one or more hydroxyls independently substituted with an alkyl, short chain fatty acid acyl, monomethyl fumarate acyl, tryptophan analogue acyl, ketone body acyl, or pre-ketone body acyl, one and only one hydroxyl is substituted with a bond to monomethyl fumarate acyl, and one or more of the remaining hydroxyls are independently substituted with an alkyl, short chain fatty acid acyl, monomethyl fumarate acyl, tryptophan analogue acyl, ketone body acyl, or pre-ketone body acyl.

The term “sulfate,” as used herein, represents group —OSO₃H or a salt thereof.

“Treatment” and “treating,” as used herein, refer to the medical management of a subject with the intent to improve, ameliorate, stabilize, prevent or a disease, disorder, or condition (e.g., an autoimmune disorder). This term includes active treatment (treatment directed to improve the multiple sclerosis); causal treatment (treatment directed to the cause of the associated multiple sclerosis); palliative treatment (treatment designed for the relief of symptoms of the multiple sclerosis); preventative treatment (treatment directed to minimizing or partially or completely inhibiting the development of the associated multiple sclerosis); and supportive treatment (treatment employed to supplement another therapy).

The term “tryptophan analogue,” as used herein, refers to a compound of formula R^{T}—L^{T}—(CO)ₙ—OH, where n is 0 or 1; R^{T} is indol-3-yl; and L^{T} is —CH₂—, —CH₂CH₂—, —CH₂CH₂CH₂—, —CH₂(CO)—, or —CH—CH—. Preferably, L^{T} is —CH₂—, —CH₂CH₂—, —CH₂(CO)—, or —CH—CH—. Non-limiting examples of tryptophan analogues include indole-3-carbinol, indole-3-acetic acid, indole-3-propionic acid, indole-3-butyric acid, indole-3-acrylic acid, and indole-3-pyruvic acid.

The term “tryptophan analogue acyl,” as used herein, refers to a monovalent group that is a tryptophan analogue having a carboxylate (n is 1), in which —OH is replaced with a valency.

The compounds described herein, unless otherwise noted, encompass isotopically enriched compounds (e.g., deuterated compounds), tautomers, and all stereoisomers and conformers (e.g. enantiomers, diastereomers, (unless otherwise specified) E/Z isomers, atropisomers, etc.), as well as racemates thereof and mixtures of different proportions of enantiomers or diastereomers, or mixtures of any of the foregoing forms as well as salts (e.g., pharmaceutically acceptable salts).

### BRIEF DESCRIPTION OF THE DRAWINGS

FIG. 1 is a series of mass spectra presenting the biotransformation and detection of monomethyl fumarate in vitro. The release of monomethyl fumaric acid was monitored at 0 h and 2 h timepoints and was compared to neat solutions of monomethyl fumaric acid. The presence of monomethyl fumaric acid is seen at the 2 h timepoint.

FIG. 2A is a graph depicting the results of a treatment course of propionate or butyrate in an autoimmune encephalomyelitis (EAE) model of multiple sclerosis in mice. Data are shown on a 5-point score scale and each treatment group contained 10-12 mice. Mice treated with 200 mM propionate (down arrow) and 200 mM butyrate (diamond) received lower EAE scores when compared to control (vehicle only) mice.

FIG. 2B is a graph depicting the ratio of splenic T_{H}17 cell (CD3⁺, IL7⁺)/regulatory T cells (CD3⁺, FoxP3⁺) based on FACS analysis at the end of the EAE model multiple sclerosis study (n=8 mice per group). Data are presented as mean±SEM; **p<0.05, were considered to be statistically significant.

FIG. 2C is a graph depicting the results of a treatment course using dimethyl fumarate, compound 1, compound 6, compound 15, or compound 20 in an autoimmune encephalomyelitis (EAE) model of multiple sclerosis in mice. Data are shown on a 5-point score scale. Mice treated with compound 1, 6, 15, or 20 received lower EAE scores when compared to control (vehicle only) mice.

FIG. 2D is a graph depicting the results of a treatment course using dimethyl fumarate, compound 3, or compound 24 in an autoimmune encephalomyelitis (EAE) model of multiple sclerosis in mice. Data are shown on a 5-point score scale. Mice treated with compound 3 or 24 received lower EAE scores when compared to control (vehicle only) mice. Mice treated with compound 3 received lower or similar EAE scores when compared to mice treated with dimethyl fumarate.

FIG. 3A is a graph depicting mean monomethyl fumarate concentration (ng/mL) measured in blood samples from rats collected at 15 min, 30 min, 1 h, 2 h, 4 h, or 8 h following administration of dimethyl fumarate, compound 1, compound 6, compound 10, or compound 15.

FIG. 3B is a graph depicting mean monomethyl fumarate concentration (ng/mL) measured in blood samples from rats collected at 15 min, 30 min, 1 h, 2 h, 4 h, and 8 h following administration of dimethyl fumarate, compound 3, compound 11, compound 20, compound 27, or compound 28.

FIG. 3C is a graph depicting mean monomethyl fumarate concentration (ng/mL) measured in blood samples from rats collected at 15 min, 30 min, 1 h, 2 h, 4 h, and 8 h following administration of dimethyl fumarate, compound 7, compound 24, compound 25, or compound 26.

FIG. 3D is a graph depicting mean monomethyl fumarate concentration (ng/mL) measured in blood samples from rats collected at 15 min, 30 min, 1 h, 2 h, 4 h, and 8 h following administration of dimethyl fumarate, compound 22, compound 23, compound 29, or diroximel fumarate.

FIG. 3E is a graph depicting mean deuterated propionate (d3) concentration (μM) measured in blood samples from rats collected at 15 min, 30 min, 1 h, 2 h, 4 h, and 8 h following administration of sodium propionate-d3, compound 1-d9, compound 6-d9, or compound 20-d9.

FIG. 3F is a graph depicting mean deuterated butyrate (d5) concentration (μM) measured in blood samples from rats collected at 15 min, 30 min, 1 h, 2 h, 4 h, and 8 h following administration of sodium butyrate-d5 or compound 15-d15.

FIG. 3G is a graph depicting mean deuterated propionate (d3) concentration (μM) measured in blood samples from rats collected at 15 min, 30 min, 1 h, 2 h, 4 h, and 8 h following administration of sodium propionate-d3 or compound 3-d12.

FIG. 3H is a graph depicting mean deuterated butyrate (d5) concentration (μM) measured in blood samples from rats collected at 15 min, 30 min, 1 h, 2 h, 4 h, and 8 h following administration of sodium butyrate-d5 or compound 24-d15.

FIG. 4A is a graph depicting deuterated propionate (d3) concentration (nmol/g) measured in stomach tissue from mice collected at 15 min, 30 min, 1 h, 2 h, 4 h, 8 h, and 12 h following administration of sodium propionate-d3, compound 3-d12, or compound 6-d9.

FIG. 4B is a graph depicting deuterated propionate (d3) concentration (nmol/g) measured in proximal small intestine tissue from mice collected at 15 min, 30 min, 1 h, 2 h, 4 h, 8 h, and 12 h following administration of sodium propionate-d3, compound 3-d12, or compound 6-d9.

FIG. 4C is a graph depicting deuterated propionate (d3) concentration (nmol/g) measured in distal small intestine tissue from mice collected at 15 min, 30 min, 1 h, 2 h, 4 h, 8 h, and 12 h following administration of sodium propionate-d3, compound 3-d12, or compound 6-d9.

FIG. 4D is a graph depicting deuterated propionate (d3) concentration (nmol/g) measured in distal cecum tissue from mice collected at 15 min, 30 min, 1 h, 2 h, 4 h, 8 h, and 12 h following administration of sodium propionate-d3, compound 3-d12, or compound 6-d9.

FIG. 4E is a graph depicting deuterated propionate (d3) concentration (nmol/g) measured in proximal colon tissue from mice collected at 15 min, 30 min, 1 h, 2 h, 4 h, 8 h, and 12 h following administration of sodium propionate-d3, compound 3-d12, or compound 6-d9.

FIG. 4F is a graph depicting deuterated propionate (d3) concentration (nmol/g) measured in distal colon tissue from mice collected at 15 min, 30 min, 1 h, 2 h, 4 h, 8 h, and 12 h following administration of sodium propionate-d3, compound 3-d12, or compound 6-d9.

FIG. 4G is a graph depicting deuterated propionate (d3) concentration (nmol/g) measured in blood plasma from mice collected at 15 min, 30 min, 1 h, 2 h, 4 h, 8 h, and 12 h following administration of sodium propionate-d3, compound 3-d12, or compound 6-d9.

FIG. 4H is a graph depicting deuterated propionate (d3) concentration (nmol/g) measured in brain tissue from mice collected at 15 min, 30 min, 1 h, 2 h, 4 h, 8 h, and 12 h following administration of sodium propionate-d3, compound 3-d12, or compound 6-d9.

FIG. 5A is a graph depicting deuterated propionate (d3) concentration (nmol/g) measured in stomach, proximal small intestine, distal small intestine, cecum, proximal colon, and distal colon tissues from mice collected at 15 min, 30 min, 1 h, 2 h, 4 h, 8 h, and 12 h following administration of sodium propionate-d3.

FIG. 5B is a graph depicting deuterated propionate (d3) concentration (nmol/g) measured in stomach, proximal small intestine, distal small intestine, cecum, proximal colon, and distal colon tissues from mice collected at 15 min, 30 min, 1 h, 2 h, 4 h, 8 h, and 12 h following administration of compound 3-d12.

FIG. 5C is a graph depicting deuterated propionate (d3) concentration (nmol/g) measured in stomach, proximal small intestine, distal small intestine, cecum, proximal colon, and distal colon tissues from mice collected at 15 min, 30 min, 1 h, 2 h, 4 h, 8 h, and 12 h following administration of compound 6-d9.

FIG. 6A is a graph depicting deuterated propionate (d3) concentration (nmol/g) and monomethyl fumarate concentration (nmol/g) measured in stomach tissue from mice collected at 15 min, 30 min, 1 h, 2 h, 4 h, 8 h, and 12 h following administration of compound 3-d12.

FIG. 6B is a graph depicting deuterated propionate (d3) concentration (nmol/g) and monomethyl fumarate concentration (nmol/g) measured in proximal small intestine tissue from mice collected at 15 min, 30 min, 1 h, 2 h, 4 h, 8 h, and 12 h following administration of compound 3-d12.

FIG. 6C is a graph depicting deuterated propionate (d3) concentration (nmol/g) and monomethyl fumarate concentration (nmol/g) measured in distal small intestine tissue from mice collected at 15 min, 30 min, 1 h, 2 h, 4 h, 8 h, and 12 h following administration of compound 3-d12.

FIG. 6D is a graph depicting deuterated propionate (d3) concentration (nmol/g) and monomethyl fumarate concentration (nmol/g) measured in distal cecum tissue from mice collected at 15 min, 30 min, 1 h, 2 h, 4 h, 8 h, and 12 h following administration of compound 3-d12.

FIG. 6E is a graph depicting deuterated propionate (d3) concentration (nmol/g) and monomethyl fumarate concentration (nmol/g) measured in proximal colon tissue from mice collected at 15 min, 30 min, 1 h, 2 h, 4 h, 8 h, and 12 h following administration of compound 3-d12.

FIG. 6F is a graph depicting deuterated propionate (d3) concentration (nmol/g) and monomethyl fumarate concentration (nmol/g) measured in distal colon tissue from mice collected at 15 min, 30 min, 1 h, 2 h, 4 h, 8 h, and 12 h following administration of compound 3-d12.

FIG. 7A is a graph depicting monomethyl fumarate concentration (nmol/g) measured in stomach tissue from mice collected at 15 min, 30 min, 1 h, 2 h, 4 h, 8 h, and 12 h following administration of compound 3-d12, compound 6-d9, dimethyl fumarate, or diroximel fumarate.

FIG. 7B is a graph depicting monomethyl fumarate concentration (nmol/g) measured in proximal small intestine tissue from mice collected at 15 min, 30 min, 1 h, 2 h, 4 h, 8 h, and 12 h following administration of compound 3-d12, compound 6-d9, dimethyl fumarate, or diroximel fumarate.

FIG. 7C is a graph depicting monomethyl fumarate concentration (nmol/g) measured in distal small intestine tissue from mice collected at 15 min, 30 min, 1 h, 2 h, 4 h, 8 h, and 12 h following administration of compound 3-d12, compound 6-d9, dimethyl fumarate, or diroximel fumarate.

FIG. 7D is a graph depicting monomethyl fumarate concentration (nmol/g) measured in distal cecum tissue from mice collected at 15 min, 30 min, 1 h, 2 h, 4 h, 8 h, and 12 h following administration of compound 3-d12, compound 6-d9, dimethyl fumarate, or diroximel fumarate.

FIG. 7E is a graph depicting monomethyl fumarate concentration (nmol/g) measured in proximal colon tissue from mice collected at 15 min, 30 min, 1 h, 2 h, 4 h, 8 h, and 12 h following administration of compound 3-d12, compound 6-d9, dimethyl fumarate, or diroximel fumarate.

FIG. 7F is a graph depicting monomethyl fumarate concentration (nmol/g) measured in distal colon tissue from mice collected at 15 min, 30 min, 1 h, 2 h, 4 h, 8 h, and 12 h following administration of compound 3-d12, compound 6-d9, dimethyl fumarate, or diroximel fumarate.

### DETAILED DESCRIPTION

The invention provides conjugates, compositions, and methods that may be used in the treatment of multiple sclerosis. A conjugate contains monomethyl fumarate covalently linked to a carrier group through a carbon-oxygen bond which is cleavable in vivo. The carrier group includes a core having one or more hydroxyls independently substituted with at least one acyl (e.g., at least one short chain fatty acid acyl, at least one tryptophan analogue, at least one ketone body, or at least one pre-ketone body).

Administration of conjugates that are stable under a range of physiological pH levels and cleaved selectively at a desired site of absorption/action (for example, in the GI tract (e.g., in the stomach, small intestine, or large intestine)) can increase bioavailability and produce beneficial effects in subjects having a disease, disorder, or condition described herein.

The components of the conjugates described herein (e.g., an acylated carrier group (e.g., short chain fatty acid acyl) and monomethyl fumarate) may act synergistically to modulate an autoimmunity marker, e.g., upon hydrolysis in the GI tract of the subject receiving the conjugate.

Advantageously, the conjugates disclosed herein may have superior organoleptic properties (e.g., palatability). This provides an important advantage as the individual components (e.g., monomethyl fumarate or short chain fatty acid acyl) may exhibit less desirable organoleptic properties (e.g., palatability). Improved organoleptic properties facilitate oral administration, and are particularly advantageous for delivery of high unit dosages.

Advantageously, conjugates disclosed herein (e.g., an acylated carrier group (e.g., short chain fatty acid acyl) and monomethyl fumarate), in addition to delivering a therapeutically active moiety (e.g., monomethyl fumarate), may deliver a second therapeutically active moiety (e.g., short chain fatty acid) to the brain to impart superior bioavailability of the active for the treatment of, e.g., multiple sclerosis (e.g., primary or secondary progressive multiple sclerosis).

### Conjugates

In some embodiments, compounds of the invention are conjugates of monomethyl fumarate (MMF) and a carrier group, or a pharmaceutically acceptable salt thereof, wherein monomethyl fumarate is covalently bonded to the carrier group through a carbon-oxygen bond that is cleavable in vivo. In some embodiments, a carrier group includes a core and one or more substituents covalently bonded to the core, where each substituent is independently an acyl.

### Core: Monosaccharides, Aminomonosacchandes, Sugar Adds, and Sugar Alcohols

In some embodiments, a core is selected from the group consisting of: monosaccharide, aminomonosaccharide, acid monosaccharide, catechin polyphenol, sugar alcohol, and sugar acid.

In some embodiments, a core is monosaccharide. In some embodiments, a monosaccharide core is a C₅₋₆ pyranose core. In some embodiments, a monosaccharide core is a C₄₋₅ furanose core. In some embodiments, a C₅₋₆ pyranose is the alpha-anomer of the C₅₋₆ pyranose. In some embodiments, a C₅₋₆ pyranose is the beta-anomer of the C₅₋₆ pyranose. In some embodiments, a monosaccharide core is selected from the group consisting of: arabinose, fucose, galactose, glucose, mannose, rhamnose, ribose, tagatose, and xylose. In some embodiments, a monosaccharide core is selected from either glucose or ribose. In some embodiments, a monosaccharide is glucose.

In some embodiments, a core is aminomonosaccharide. In some embodiments, an aminomonosaccharide core is a C₅₋₆ aminopyranose core. In some embodiments, a C₅₋₆ aminopyranose is the alpha-anomer of the C₅₋₆ aminopyranose. In some embodiments, a C₅₋₆ aminopyranose is the beta-anomer of the C₅₋₆ aminopyranose. In some embodiments, an aminomonosaccharide core is glucosamine.

In some embodiments, a core is an acid monosaccharide. In some embodiments, an acid monosaccharide core is a C₅₋₆ acid pyranose core. In some embodiments, a C₅₋₆ acid pyranose is the alpha-anomer of the C₅₋₆ acid pyranose. In some embodiments, a C₅₋₆ acid pyranose is the beta-anomer of the C₅₋₆ acid pyranose. In some embodiments, an acid monosaccharide core is glucuronic acid.

When a core is C₅₋₆ pyranose (e.g. C₅₋₆ monosaccharide pyranose monosaccharide, C₅₋₆ aminomonosaccharide, C₅₋₆ acid monosaccharide), in some embodiments, the in vivo cleavable carbon-oxygen bond between monomethyl fumarate and C₅₋₆ pyranose includes an oxygen atom bonded to the anomeric carbon (i.e. the 1 carbon) of C₅₋₆ pyranose. In some embodiments, the in vivo cleavable carbon-oxygen bond between monomethyl fumarate and C₅₋₆ pyranose includes an oxygen atom bonded to the 2 carbon of C₅₋₆ pyranose. In some embodiments, the in vivo carbon-oxygen bond between monomethyl fumarate and C₅₋₆ pyranose includes an oxygen atom bonded to the 3 carbon of C₅₋₆ pyranose. In some embodiments, the in vivo cleavable carbon-oxygen bond between monomethyl fumarate and C₅₋₆ pyranose includes an oxygen atom bonded to the 4 carbon of C₅₋₆ pyranose. In some embodiments, the in vivo cleavable carbon-oxygen bond between monomethyl fumarate and C₅₋₆ pyranose includes an oxygen atom bonded to the 5 carbon of C₅₋₆ pyranose. In some embodiments, the in vivo cleavable carbon-oxygen bond between monomethyl fumarate and Ce pyranose includes an oxygen atom bonded to the 6 carbon of Ce pyranose.

In some embodiments, a core is a sugar alcohol of the following structure:

HOCH₂(CHOH)ₙCH₂OH,

where n is 1, 2, 3, or 4, and one or more of the hydroxyl groups is independently substituted with an alkyl, acyl, or a bond to monomethyl fumarate.

In some embodiments, n is 1.

### Core: Catechin Polyphenols

In some embodiments, a core or a conjugate is a catechin polyphenol of the following structure:

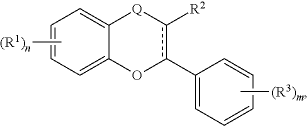

wherein
[symbol] is a single carbon-carbon bond or double carbon-carbon bond;
Q is —CH₂— or —C(O)—;
each R¹ and each R³ is independently H, halogen, or —OR^{A};
R² is H or —OR^{A};
each R^{A} is independently H, alkyl, short chain fatty acid acyl, monomethyl fumarate acyl, a bond to monomethyl fumarate acyl, or benzoyl optionally substituted with 1, 2, 3, or 4 substituents independently selected from the group consisting of H, hydroxy, halogen, optionally substituted alkyl, alkoxy, short chain fatty acid acyl, monomethyl fumarate acyl, or a bond to monomethyl fumarate acyl; and
each of n and m is independently 1, 2, 3, or 4.

In some embodiments, each R¹ and each R³ is independently H or —OR^{A}. In some embodiments, each R^{A} is independently H or monomethyl fumarate acyl. In some embodiments, n is 1. In some embodiments, n is 2. In some embodiments, m is 1 or 2. In some embodiments, m is 1. In some embodiments, m is 2.

### Acyls

In some embodiments, a core is peracylated, i.e., all available hydroxyls on the core are substituted with acyls. In some embodiments, a core is not peracylated. In some embodiments, the carrier group is an acylated sugar. In some embodiments, the carrier group is an alkylated sugar.

### Acyls: Monosaccharides, Aminomonosaccharides, Sugar Acids, and Sugar Alcohols

When the core of a carrier group is a monosaccharide, in some embodiments, each hydroxyl group of the monosaccharide can be independently substituted as described herein.

When the core of a carrier group is an aminomonosaccharide, in some embodiments, each hydroxyl and amine group of the aminomonosaccharide can be independently substituted. In some embodiments, when the core of a carrier group is an aminomonosaccharide, each hydroxyl group of the aminomonosaccharide can be independently substituted as described herein.

When the core of a carrier group is an acid monosaccharide, in some embodiments, each hydroxyl and acid group of the acid monosaccharide can be independently substituted. In some embodiments, when the core of a carrier group is an acid monosaccharide, each hydroxyl group of the acid monosaccharide can be independently substituted as described herein.

When a core is an acylated sugar (e.g. acylated monosaccharide, acid monosaccharide, or sugar alcohol), in some embodiments, the acylated sugar includes one or more hydroxyls independently substituted with fatty acid acyl group. In some embodiments, an acylated sugar includes one or more hydroxyls independently substituted with fatty acid acyl. In some embodiments, an acylated sugar includes one or more hydroxyls independently substituted with short chain fatty acid acyl. In some embodiments, an acylated sugar includes one or more hydroxyls independently substituted with propionyl. In some embodiments, an acylated sugar includes one or more hydroxyls independently substituted with butyryl. In some embodiments, an acylated sugar includes one or more hydroxyls independently substituted with medium chain fatty acid.

### Acyls: Catechin Polyphenols

When the core of a carrier group is a catechin polyphenol, in some embodiments, each catechin hydroxyl group of the catechin polyphenol can be independently substituted. In some embodiments, when the core of a group is a catechin polyphenol, each hydroxyl group can be independently substituted with monomethyl fumarate acyl or fatty acyl. In some embodiments, when the core of a group is a catechin polyphenol, each hydroxyl group can be independently substituted with monomethyl fumarate acyl.

### Conjugates

The conjugates described herein, or pharmaceutically acceptable salts thereof, contain monomethyl fumarate bonded through a carbon-oxygen bond to a carrier group. The carbon-oxygen bond may be cleavable in vivo. The carbon-oxygen bond may be an ester bond or a glycosidic bond.

The conjugate may be, e.g., a compound of formula (A):

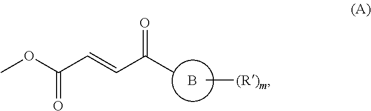

or a pharmaceutically acceptable salt thereof,
where
n is 0 or 1;
group B is a monosaccharide, aminomonosaccharide, sugar acid (e.g., acid monosaccharide), sugar alcohol, catechin polyphenol, ellagic acid, ellagic acid analogue, stilbenoid, curcuminoid, chalconoid, pyridoxine, bile acid, ketone body, or pre-ketone body;
each R′ is independently an alkyl or acyl (e.g., short chain fatty acid acyl, tryptophan analogue acyl, ketone body acyl, or pre-ketone body acyl); and
m is an integer from 0 to the total number of available hydroxyl groups in group B (e.g., 0, 1, 2, 3, 4, or 5);
provided that group B is bonded to the monomethyl fumarate acyl through a carbon-oxygen bond.

One of skill in the art will recognize that the linkage between monomethyl fumarate and group B does not include peroxide.

A conjugate of monomethyl fumarate and an acylated sugar may be a compound of formula (A), in which group B is a monosaccharide, sugar acid (e.g., acid monosaccharide), or sugar alcohol, and at least one R′ is acyl. A conjugate of monomethyl fumarate and an acylated sugar may be a compound of formula (A), in which group B is an aminomonosaccharide, and at least one R′ is an alkyl.

In some embodiments, group B is a monosaccharide, sugar acid, sugar alcohol, catechin polyphenol, ellagic acid, ellagic acid analogue, stilbenoid, curcuminoid, chalconoid, pyridoxine, bile acid, ketone body, or pre-ketone body. In some embodiments, group B is a monosaccharide, aminomonosaccharide, sugar acid (e.g., acid monosaccharide), or sugar alcohol. In some embodiments, each R′ is alkyl, short chain fatty acid acyl, tryptophan analogue acyl, ketone body acyl, or pre-ketone body acyl. In some embodiments, when B is a monosaccharide, aminomonosaccharide, sugar acid (e.g., acid monosaccharide), or sugar alcohol, each R′ is independently a short chain fatty acid acyl. In some embodiments, when B is a catechin polyphenol, each R′ is independently a monomethyl fumarate acyl or a short chain fatty acid acyl. In some embodiments, when B is a catechin polyphenol, each R′ is independently a monomethyl fumarate acyl.

In certain embodiments, the group of formula (A) includes at least one fatty acid acyl.

In some embodiments, the fatty acid acyl(s) are individually short chain fatty acid acyls (e.g., acetyl, propionyl, butyryl, or valeryl).

Non-limiting examples of a carrier group include:

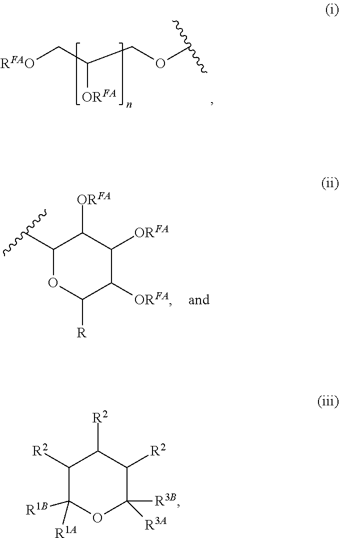

where
n is 1, 2, 3, or 4 (e.g., n is 1);
R is H, —CH₃, —CH₂OR^{FA}, or —COOR^{C};
each R^{FA} is independently H, a fatty acid acyl (e.g., a short chain fatty acid acyl or medium chain fatty acid acyl), a ketone body acyl (e.g., β-hydroxybutyrate acyl), a pre-ketone body acyl, or a tryptophan analogue acyl (e.g., indole-3-acetyl, indole-3-acyloyl, or indole-3-pyruvyl);
each of R^{1A} and R^{1B} is independently H, OR^{A}, or a bond to the monomethylfumarate moiety;
each R² is independently H, OR^{A}, NHR^{A}, or a bond to the monomethylfumarate moiety;
each of R^{3A} and R^{3B} is independently H, OR^{A}, CH₂R^{B}, or —COOR^{C};
each R^{A} is independently H, alkyl, a fatty acid acyl, a ketone body acyl, a pre-ketone body acyl, or a tryptophan analogue acyl; and
each R^{B} is independently H, OR^{A}, or a bond to the monomethylfumarate moiety; and
each R^{C} is independently H or alkyl; and
provided that the carrier group of formula (iii) includes a bond to the monomethylfumarate moiety and OR^{A}.

In certain embodiments, the carrier group is a group of formula (i). In particular embodiments, the carrier group is a group of formula (ii). In other embodiments, the carrier group is a group of formula (iii).

In some embodiments, at least one R^{FA} is a fatty acid acyl, a ketone body acyl, a pre-ketone body acyl, or a tryptophan analogue acyl. In some embodiments of a group containing a fatty acid acyl, at least one R^{FA} is a fatty acid acyl. In some embodiments of a group containing a ketone body or a pre-ketone body, at least one R^{FA} is a ketone body acyl a pre-ketone body acyl. In some embodiments of a group containing an amino acid metabolite acyl, at least one R^{FA} is a tryptophan analogue acyl. In some embodiments, one of R^{3A} and R^{3B} is H.

The carrier group may be, e.g., a monosaccharide having one or more hydroxyls independently substituted with an alkyl, short chain fatty acid acyl, monomethyl fumarate acyl, tryptophan analogue acyl, ketone body acyl, or pre-ketone body acyl; provided that at least one hydroxyl is substituted with a short chain fatty acid acyl, tryptophan analogue acyl, ketone body acyl, or pre-ketone body acyl. The monosaccharide may be, e.g., arabinose, xylose, fructose, galactose, glucose, ribose, tagatose, fucose, or rhamnose.

The carrier group may be, e.g., a sugar acid having one or more hydroxyls independently substituted with an alkyl, short chain fatty acid acyl, monomethyl fumarate acyl, tryptophan analogue acyl, ketone body acyl, optionally acylated ketone body, pre-ketone body acyl, or optionally acylated pre-ketone body; provided that at least one hydroxyl is substituted with a short chain fatty acid acyl, tryptophan analogue acyl, ketone body acyl, optionally acylated ketone body, pre-ketone body acyl, or optionally acylated pre-ketone body. When the substituted hydroxyl includes an alcohol oxygen atom, the hydroxyl is substituted with an alkyl, short chain fatty acid acyl, monomethyl fumarate acyl, tryptophan analogue acyl, ketone body acyl, or pre-ketone body acyl; provided that at least one hydroxyl is substituted with a short chain fatty acid acyl, tryptophan analogue acyl, ketone body acyl, or pre-ketone body acyl. When the substituted hydroxyl includes a carboxylate oxygen atom, the hydroxyl is substituted with an alkyl, optionally acylated ketone body, or optionally acylated pre-ketone body. The sugar acid may be, e.g., aldonic acid, ulosonic acid, uronic acid, or aldaric acid. The sugar acid may be, e.g., xylonic acid, gluconic acid, glucuronic acid, galacturonic acid, tartaric acid, saccharic acid, or mucic acid.

The carrier group may be, e.g., a sugar alcohol having one or more hydroxyls independently substituted with an alkyl, short chain fatty acid acyl, monomethyl fumarate acyl, tryptophan analogue acyl, ketone body acyl, or pre-ketone body acyl; provided that at least one hydroxyl is substituted with a short chain fatty acid acyl, tryptophan analogue acyl, ketone body acyl, or pre-ketone body acyl. The sugar alcohol may be, e.g., glycerol, erythritol, threitol, arabitol, xylitol, tibitol, mannitol, sorbitol, galactitol, fucitol, iditol, or inositol.

The conjugate may be, e.g., a compound of formula (B):

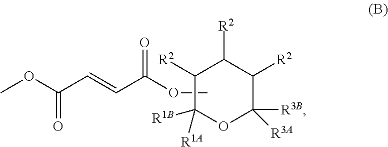

where
each of R^{1A} and R^{1B} is independently H, OR^{A}, or a bond to the monomethylfumarate moiety;
each R² is independently H, OR^{A}, NHR^{A}, or a bond to the monomethylfumarate moiety;
each of R^{3A} and R^{3B} is independently H, OR^{A}, CH₂R^{B}, or —COOR^{C};
each R^{A} is independently H, alkyl, a fatty acid acyl, a ketone body acyl, a pre-ketone body acyl, or a tryptophan analogue acyl;
each R^{B} is independently H, OR^{A}, or a bond to the monomethylfumarate moiety; and
each R^{C} is independently H or alkyl; and
provided that the compound of formula (B) includes a bond to monomethylfumarate moiety and OR^{A}.

In some embodiments compounds of the invention are selected from the group consisting of: methyl((2S,3S,4R,5R,6S)-6-methyl-3,4,5-tris(propionyloxy)tetrahydro-2H-pyran-2-yl) fumarate, methyl((2S,3R,4R,5S,6S)-6-methyl-3,4,5-tris(propionyloxy)tetrahydro-2H-pyran-2-yl) fumarate, methyl((2S,3R,4S,5R,6R)-3,4,5-tris(propionyloxy)-6-((propionyloxy)methyl)tetrahydro-2H-pyran-2-yl) fumarate, methyl((2R,3R,4S,5R,6R)-3,4,5-tris(propionyloxy)-6-((propionyloxy)methyl)tetrahydro-2H-pyran-2-yl) fumarate, methyl((2S,3R,4S,5S)-3,4,5-tris(propionyloxy)tetrahydro-2H-pyran-2-yl) fumarate, methyl((2S,3R,4R,5R)-3,4,5-tris(propionyloxy)tetrahydro-2H-pyran-2-yl) fumarate, methyl((2S,3R,4S,5R)-3,4,5-tris(butyryloxy)tetrahydro-2H-pyran-2-yl) fumarate, methyl((2R,3R,4S,5R)-3,4,5-tris(butyryloxy)tetrahydro-2H-pyran-2-yl) fumarate, methyl((2R,3R,4R,5R)-3,4,5-tris(propionyloxy)tetrahydro-2H-pyran-2-yl) fumarate, methyl((2S,3R,4S,5R,6R)-3,4,5-tris(butyryloxy)-6-((butyryloxy)methyl)tetrahydro-2H-pyran-2-yl) fumarate, methyl((2R,3R,4S,5R,6R)-3,4,5-tris(butyryloxy)-6-((butyryloxy)methyl)tetrahydro-2H-pyran-2-yl) fumarate, methyl((2R,3R,4S,5S)-3,4,5-tris(butyryloxy)tetrahydro-2H-pyran-2-yl) fumarate, methyl((2S,3S,4R,5R,6S)-3,4,5-tris(butyryloxy)-6-methyltetrahydro-2H-pyran-2-yl) fumarate, methyl((2R,3R,4R,5S,6S)-3,4,5-tris(butyryloxy)-6-methyltetrahydro-2H-pyran-2-yl) fumarate, methyl((2S,3R,4R,5S,6S)-3,4,5-tris(butyryloxy)-6-methyltetrahydro-2H-pyran-2-yl) fumarate, methyl((2R,3R,4S,5R)-3,4,5-tris(propionyloxy)tetrahydro-2H-pyran-2-yl) fumarate, (R)-2,3-bis(propionyloxy)propyl methyl fumarate, (S)-2,3-bis(propionyloxy)propyl methyl fumarate, (S)-2,3-bis(butyryloxy)propyl methyl fumarate, methyl((2S,3R,4S,5R)-3,4,5-tris(propionyloxy)tetrahydro-2H-pyran-2-yl) fumarate, methyl((2R,3S,4R,5R,6S)-6-methyl-3,4,5-tris(propionyloxy)tetrahydro-2H-pyran-2-yl) fumarate, methyl(((2R,3R,4S,5R,6S)-3,4,5,6-tetrakis(propionyloxy)tetrahydro-2H-pyran-2-yl)methyl) fumarate, methyl(((2R,3R,4S,5R,6S)-3,4,5,6-tetrakis(butyryloxy)tetrahydro-2H-pyran-2-yl)methyl) fumarate, methyl((2S,3R,4R,5R)-3,4,5-tris(butyryloxy)tetrahydro-2H-pyran-2-yl) fumarate, (2S,3S,4S,5R,6R)-3,4,5-tris(butyryloxy)-6-(((E)-4-methoxy-4-oxobut-2-enoyl)oxy)tetrahydro-2H-pyran-2-carboxylic acid, (2S,3S,4S,5R,6R)-6-(((E)-4-methoxy-4-oxobut-2-enoyl)oxy)-3,4,5-tris(propionyloxy)tetrahydro-2H-pyran-2-carboxylic acid, (2S,3R,4R,5S,6R)-2-(((E)-4-methoxy-4-oxobut-2-enoyl)oxy)-4,5-bis(propionyloxy)-6-((propionyloxy)methyl)tetrahydro-2H-pyran-3-aminium chloride, (2R,3R,4R,5S,6R)-4,5-bis(butyryloxy)-6-((butyryloxy)methyl)-2-(((E)-4-methoxy-4-oxobut-2-enoyl)oxy)tetrahydro-2H-pyran-3-aminium chloride, methyl((2R,3R,4R,5S,6R)-3-propionamido-4,5-bis(propionyloxy)-6-((propionyloxy)methyl)tetrahydro-2H-pyran-2-yl) fumarate, methyl((2S,3R,4R,5S)-3,4,5-tris(butyryloxy)-2-((butyryloxy)methyl)tetrahydro-2H-pyran-2-yl) fumarate, methyl((2R,3S,4S,5R,6R)-3,4,5-tris(butyryloxy)-6-((butyryloxy)methyl)tetrahydro-2H-pyran-2-yl) fumarate, methyl((2S,3R,4S,5S,6R)-3,4,5-tris(butyryloxy)-6-((butyryloxy)methyl)tetrahydro-2H-pyran-2-yl) fumarate, (2R,3R,4R,5S,6R)-3-butyramido-4,5-bis(butyryloxy)-6-((butyryloxy)methyl)tetrahydro-2H-pyran-2-yl methyl fumarate, methyl((2S,3R,4S,5S,6R)-3,4,5-tris(propionyloxy)-6-((propionyloxy)methyl)tetrahydro-2H-pyran-2-yl), methyl((2R,3S,4S,5R,6R)-3,4,5-tris(propionyloxy)-6-((propionyloxy)methyl)tetrahydro-2H-pyran-2-yl) fumarate, 1-methyl(2S,3R,4S,5S)-3,4,5-tris(butanoyloxy)oxan-2-yl(2E)-but-2-enedioate, -methyl(2R,3S,4R,5R,6S)-3,4,5-tris(butanoyloxy)-6-methyloxan-2-yl(2E)-but-2-enedioate, 1-methyl(2S,3R,4S,5R,6R)-3,4,5-tris(butanoyloxy)-6-(hydroxymethyl)oxan-2-yl(2E)-but-2-enedioate, 1-methyl(2R,3R,4S,5R,6R)-3,4,5-tris(butanoyloxy)-6-(hydroxymethyl)oxan-2-yl(2E)-but-2-enedioate, 1-methyl 4-[(2R,3R,4S,5R,6R)-3,4,5,6-tetrakis(butanoyloxy)oxan-2-yl]methyl(2E)-but-2-enedioate, 1-methyl 4-[(2R,3S,4S,5R,6S)-3,4,5,6-tetrakis(butanoyloxy)oxan-2-yl]methyl(2E)-but-2-enedioate, (2R,3R,4S,5R,6R)-6-(hydroxymethyl)-3,4,5-tris(propanoyloxy)oxan-2-yl 1-methyl(2E)-but-2-enedioate, 1-methyl 4-[(2R,3S,4S,5R,6R)-3,4,5,6-tetrakis(butanoyloxy)oxan-2-yl]methyl(2E)-but-2-enedioate, 1-methyl(2S,3R,4S,5S,6R)-3,4,5-tris(butanoyloxy)-6-(hydroxymethyl)oxan-2-yl(2E)-but-2-enedioate, 1-methyl(2R,3R,4S,5S,6R)-3,4,5-tris(butanoyloxy)-6-(hydroxymethyl)oxan-2-yl(2E)-but-2-enedioate, (2S,3R,4S,5S,6R)-6-(hydroxymethyl)-3,4,5-tris(propanoyloxy)oxan-2-yl 1-methyl(2E)-but-2-enedioate, (2S,3R,4S,5S,6R)-5-hydroxy-3,4-bis(propanoyloxy)-6-[(propanoyloxy)methyl]oxan-2-yl 1-methyl(2E)-but-2-enedioate, (2R,3R,4S,5S,6R)-6-(hydroxymethyl)-3,4,5-tris(propanoyloxy)oxan-2-yl 1-methyl(2E)-but-2-enedioate, (2R,3R,4S,5S,6R)-5-hydroxy-3,4-bis(propanoyloxy)-6-[(propanoyloxy)methyl]oxan-2-yl 1-methyl(2E)-but-2-enedioate, 1-methyl 4-[(2R,3R,4S,5R,6R)-3,4,5,6-tetrakis(propanoyloxy)oxan-2-yl]methyl(2E)-but-2-enedioate, 1-methyl(2R,3S,4S,5R,6S)-4,5,6-tris(propanoyloxy)-2-[(propanoyloxy)methyl]oxan-3-yl(2E)-but-2-enedioate, and 1-methyl(2R,3S,4S,5R,6R)-4,5,6-tris(propanoyloxy)-2-[(propanoyloxy)methyl]oxan-3-yl(2E)-but-2-enedioate.

In some embodiments compounds of the invention are selected from the group consisting of: O4-[2-[(E)-4-methoxy-4-oxo-but-2-enoyl]oxy-4-[(2R,3R)-3,5,7-tris[[(E)-4-methoxy-4-oxo-but-2-enoyl]oxy]chroman-2-yl]phenyl]O1-methyl(E)-but-2-enedioate, O1-methyl O4-[4-[3,5,7-tris[[(E)-4-methoxy-4-oxo-but-2-enoyl]oxy]-4-oxo-chromen-2-yl]phenyl](E)-but-2-enedioate, O4-[2-[(E)-4-methoxy-4-oxo-but-2-enoyl]oxy-4-[3,5,7-tris[[(E)-4-methoxy-4-oxo-but-2-enoyl]oxy]-4-oxo-chromen-2-yl]phenyl]O1-methyl(E)-but-2-enedioate, and O4-[4-[3-hydroxy-5,7-bis[[(E)-4-methoxy-4-oxo-but-2-enoyl]oxy]-4-oxo-chromen-2-yl]phenyl]O1-methyl(E)-but-2-enedioate.

### Methods

The conjugates described herein may be used to treat a disease, disorder, or condition (e.g., an autoimmune disorder) in a subject in need thereof.

Without wishing to be bound by theory, metabolic products of the microbiome can interact with the host's immune system in several ways. The metabolites can have effects remote to the gastrointestinal tract, for example, through bidirectional interactions with the central nervous system.

Examples include SCFA interacting with free fatty acid reporters. Short-chain fatty acids may impact autoimmunity by expanding regulatory T cells and by suppressing the JNK1/P38 pathway. A conjugate described herein can biodegrade, for example, in the distal small intestine or colon, thereby providing high levels of monomethyl fumarate and fatty acids (e.g., short chain fatty acids) in the distal gut, where these compounds can interact with the immune system.

A method of treating multiple sclerosis in a subject in need thereof may include administering a conjugate described herein (e.g., a pharmaceutical composition containing the conjugate) to a subject in need thereof. Non-limiting examples of multiple sclerosis include primary progressive multiple sclerosis, secondary progressive multiple sclerosis, or relapsing-remitting multiple sclerosis. Preferably, multiple sclerosis is primary progressive multiple sclerosis.

A method of treating an autoimmune disorder in a subject in need thereof may include administering a conjugate described herein (e.g., a pharmaceutical composition containing the conjugate) to a subject in need thereof. Non-limiting examples of diseases, disorders, and conditions include autoimmune disorders, as described herein, e.g., autoimmune disorders (e.g., multiple sclerosis, psoriasis, psoriatic arthritis, rheumatoid arthritis, systemic lupus erythematosus, Crohn's disease, Sjogren's syndrome, Behcet's disease, ulcerative colitis, or Guillain-Barré syndrome), adrenoleukodystrophy, AGE-induced genome damage, Alexander's disease, Alper's disease, Alzheimer's disease, amyotrophic lateral sclerosis, angina pectoris, arthritis, asthma, balo concentric sclerosis, Canavan disease, cardiac insufficiency including left ventricular insufficiency, central nervous system vasculitis, Charcott-Marie-Tooth Disease, childhood ataxia with central nervous system hypomyelination, chronic idiopathic peripheral neuropathy, chronic obstructive pulmonary disease, diabetic retinopathy, graft-versus-host-disease, hepatitis C viral infection, herpes simplex viral infection, human immunodeficiency viral infection, Huntington's disease, irritable bowel syndrome, ischemia, Krabbe disease, lichen planus, macular degeneration, mitochondrial encephalomyopathy, monomelic amyotrophy, myocardial infarction, neurodegeneration with brain iron accumulation, neuromyelitis optica, neurosarcoidosis, optic neuritis, paraneoplastic syndrome, Parkinson's disease, Pelizaeus-Merzbacher disease, primary lateral sclerosis, progressive supranuclear palsy, reperfusion injury, retinopathia pigmentosa, Schilder's disease, subacute necrotizing myelopathy, susac syndrome, transverse myelitis, Zellweger's syndrome, granuloma annulare, pemphigus, bollus pemphigoid, contact dermatitis, acute dermatitis, chronic dermatitis, alopecia areata (totalis or universalis), sarcoidosis, cutaneous sarcoidosis, pyoderma gangrenosum, cutaneous lupus, cutaneous Crohn's disease, obstructive sleep apnea, chronic lymphocytic leukemia, small lymphocytic leukemia, systemic sclerosis-pulmonary hypertension, glioblastoma multiforme, cutaneous T cell lymphoma, progressive multifocal leukoencephalopathy, polyarthritis, juvenile-onset diabetes, type II diabetes, Hashimoto's thyroiditis, Grave's disease, pernicious anaemia, autoimmune hepatitis, neurodermatitis, retinopathia pigmentosa or forms of mitochondrial encephalomyopathy, progressive systemic sclerodermia, osteochondritis syphilitica (Wegener's disease), cutis marmorata (livedo reticularis), panarteriitis, vasculitis, osteoarthritis, gout, arteriosclerosis, Reiter's disease, pulmonary granulomatosis, endotoxic shock (septic-toxic shock), sepsis, pneumonia, encephalomyelitis, anorexia nervosa, acute hepatitis, chronic hepatitis, toxic hepatitis, alcohol-induced hepatitis, viral hepatitis, liver insufficiency, cytomegaloviral hepatitis, Rennert T-lymphomatosis, mesangial nephritis, post-angioplastic restenosis, reperfusion syndrome, cytomegaloviral retinopathy, adenoviral cold, adenoviral pharyngoconjunctival fever, adenoviral ophthalmia, AIDS, post-herpetic or post-zoster neuralgia, inflammatory demyelinating polyneuropathy, mononeuropathia multiplex, mucoviscidosis, Bechterew's disease, Barett oesophagus, Epstein-Barr virus infection, cardiac remodeling, interstitial cystitis, diabetes mellitus type II, human tumor radiosensitization, multidrug resistance in chemotherapy, mamma carcinoma, colon carcinoma, melanoma, primary liver cell carcinoma, adenocarcinoma, Kaposi's sarcoma, prostate carcinoma, leukaemia, acute myeloid leukaemia, multiple myeloma (plasmocytoma), Burkitt's lymphoma, Castleman tumor, cardiac insufficiency, myocardial infarct, angina pectoris, asthma, chronic obstructive pulmonary diseases, PDGF induced thymidine uptake of bronchial smooth muscle cells, bronchial smooth muscle cell proliferation, alcoholism, Alexander's disease, Alper's disease, Alzheimer's disease, ataxia telangiectasia, Batten disease (also known as Spielmeyer-Vogt-Sjögren-Batten disease), bovine spongiform encephalopathy (BSE), Cerebral palsy, Cockayne syndrome, corticobasal degeneration, Creutzfeldt-Jakob disease, familial fatal insomnia, frontotemporal lobar degeneration, Huntington's disease, HIV-associated dementia, Kennedy's disease, Krabbe's disease, Lewy body dementia, neuroborreliosis, Machado-Joseph disease (Spinocerebellar ataxia type 3), multiple system atrophy, narcolepsy, Niemann Pick disease, Pelizaeus-Merzbacher disease, Pick's disease, primary lateral sclerosis, prion disease, progressive supranuclear palsy, Refsum's disease, Sandhoff disease, subacute combined degeneration of spinal cord secondary to pernicious anaemia, spinocerebellar ataxia, spinal muscular atrophy, Steele-Richardson-Olszewski disease, Tabes dorsalis, toxic encephalopathy, LHON (Leber's Hereditary optic neuropathy), MELAS (Mitochondrial Encephalomyopathy; Lactic Acidosis; Stroke), MERRF (Myoclonic Epilepsy; Ragged Red Fibers), PEO (Progressive External Opthalmoplegia), Leigh's Syndrome, MNGIE (Myopathy and external ophthalmoplegia; Neuropathy; Gastro-Intestinal; Encephalopathy), Kearns-Sayre Syndrome (KSS), NARP, hereditary spastic paraparesis, mitochondrial myopathy, Friedreich Ataxia, optic neuritis, acute inflammatory demyelinating polyneuropathy (AIDP), chronic inflammatory demyelinating polyneuropathy (CIDP), acute transverse myelitis, acute disseminated encephalomyelitis (ADEM), and Leber's optic atrophy.

In some embodiments, the components of the conjugate (e.g., monomethyl fumarate and one or more carrier group components) may act synergistically to treat a disease, disorder, or condition (e.g., multiple sclerosis), e.g., upon hydrolysis in the GI tract of the subject receiving the conjugate.

Additionally or alternatively, the conjugates described herein may be used for modulating an autoimmunity marker in a subject in need thereof. A method of modulating an autoimmunity marker in a subject in need thereof may include administering a conjugate described herein (e.g., a pharmaceutical composition containing the conjugate) to a subject in need thereof.

Non-limiting examples of autoimmunity markers include markers for an inflammatory bowel disease, Addison's disease, alopecia areata, ankylosing spondylitis, antiphospholipid syndrome, hemolytic anemia, autoimmune hepatitis, Behcet's disease, Berger's disease, bullous pemphigoid, cardiomyopathy, celiac sprue, chronic fatigue immune dysfunction syndrome (CFIDS), chronic inflammatory demyelinating polyneuropathy, Churg-Strauss syndrome, cicatricial pemphigoid, cold agglutinin disease, type 1 diabetes, discoid lupus, essential mixed cryoglobulinemia, Graves' disease, Guillain-Barré syndrome, Hashimoto's thyroiditis, hypothyroidism, autoimmune lymphoproliferative syndrome (ALPS), idiopathic pulmonary fibrosis, idiopathic thrombocytopenia purpura (ITP), juvenile arthritis, lichen planus, lupus erythematosus, Ménière's disease, mixed connective tissue disease, multiple sclerosis, myasthenia gravis, pemphigus vulgaris, pernicious anemia, polychondritis, autoimmune polyglandular syndromes, polymyalgia rheumatica, polymyositis, dermatomyositis, primary agammaglobulinemia, primary biliary cirrhosis, psoriasis, psoriatic arthritis, Raynaud's phenomenon, Reiter's syndrome, rheumatic fever, rheumatoid arthritis, sarcoidosis, scleroderma, Sjögren's syndrome, stiff-man syndrome, Takayasu arteritis, giant cell arteritis, ulcerative colitis, uveitis, vasculitis, and granulomatosis with polyangiitis. In some embodiments, the autoimmunity marker is a marker for an inflammatory bowel disease (e.g., Crohn's disease or ulcerative colitis).

The autoimmunity markers include, for example, a CYP1A1 mRNA level, intestinal motility, mucus secretion, CD4⁺CD25⁺ Treg cell (e.g., CD4⁺CD25⁺Foxp3⁺ Treg) count, Tₕ1 cell count, interleukin-8 (IL8) level, macrophage inflammatory protein 1α (MIP-1α) level, macrophage inflammatory protein 1β (MIP-1β) level, NFκB level, inducible nitric oxide synthase (iNOS) level, matrix metallopeptidase 9 (MMP9) level, interferon γ (IFNγ) level, interleukin-17 (IL17) level, intercellular adhesion molecule (ICAM) level, CXCL13 level, 8-iso-prostaglandin F_{2α} (8-iso-PGF2α) level, IgA level, calprotectin level, lipocalin-2 level, short chain fatty acids level, and indoxyl sulfate level.

The autoimmunity markers can be measured in a sample from a subject using methods known in the art. For example, CD4⁺CD25⁺ Treg cell (e.g., CD4⁺CD25⁺Foxp3⁺ Treg) count and Tₕ1 cell count are measured via routine blood test, followed by flow cytometry analysis of cell markers and/or cytokines (e.g., CD4, CD25, Foxp3, IFNγ, IL2, and/or IL4). NFκB and iNOS levels can be measured using routine blood tests. Stool sample analyses may be performed to measure an IgA level, calprotectin level, lipocalin-2 level, and short chain fatty acids level. Urine sample analysis may be performed to measure an indoxyl sulfate level. Mucus secretion can be assessed through biopsy or by analysis of fecal matter content. Mucus secretion can be measured using HT-29 cell counts or by measuring mucin gene expression in biopsy samples, e.g., by PCR (Recio, The impact of Food Bioactive on Health: In vitro and ex vivo models, Chapter 11, HT29 Cell line, (2015)). Intestinal motility can be assessed using gastrointestinal scintigrapghy (e.g., wireless pH and motility capsules) or by examining effect of a test article on its ability to improve transepithelial electrical resistance (TEER) in either a cell line (e.g., CACO-2) or on a co-culture complex system (e.g., MATEK epi-intestinal) (Kickman, J. Lab. Autom., 20:107-126, 2015). Gastrointestinal permeability can be measured using a dual sugar absorption test known in the art. For example, dual sugar absorption test involves administering a predetermined amount of a drink containing lactulose and mannitol, and measuring absorption of these two sugars over six hours.

Abdominal pain is typically assessed by a survey. Gastrointestinal bleeding may be assessed by the presence or absence of blood in a stool sample from a subject. Gastrointestinal inflammation can be assessed by biopsy.

In some embodiments, upon administration to a subject in need thereof, a conjugate described herein increases an autoimmunity marker, e.g., intestinal motility, CD4⁺CD25⁺ Treg cell count, short chain fatty acids level, or mucus secretion in a subject (e.g., at least 5%, 10%, 15%, 20%, 25%, 30%, 35%, 40%, 45%, 50%, 55%, 60%, 65%, 70%, 75%, 80%, 85%, 90%, 95% or 98% or more relative to prior to administration). In some embodiments, upon administration to a subject in need thereof, a conjugate described herein increases an autoimmunity marker, e.g., a CYP1A1 mRNA level in a subject (e.g., at least 5%, 10%, 15%, 20%, 25%, 30%, 35%, 40%, 45%, 50%, 55%, 60%, 65%, 70%, 75%, 80%, 85%, 90%, 95% or 98% or more relative to prior to administration). In certain embodiments, upon administration to a subject in need thereof, a conjugate described herein decreases an autoimmunity marker, e.g., iNOS, MMP9, IFNγ, IL17, ICAM, CXCL13, 8-iso-PGF2α in a subject (e.g., at least 5%, 10%, 15%, 20%, 25%, 30%, 35%, 40%, 45%, 50%, 55%, 60%, 65%, 70%, 75%, 80%, 85%, 90%, 95% or 98% or more relative to prior to administration). In certain embodiments, upon administration to a subject in need thereof, a conjugate described herein decreases an interleukin-8 (IL8) level in a subject (e.g., at least 5%, 10%, 15%, 20%, 25%, 30%, 35%, 40%, 45%, 50%, 55%, 60%, 65%, 70%, 75%, 80%, 85%, 90%, 95% or 98% or more relative to prior to administration). In certain embodiments, upon administration to a subject in need thereof, a conjugate described herein decreases a macrophage inflammatory protein 1α (MIP-1α) level in a subject (e.g., at least 5%, 10%, 15%, 20%, 25%, 30%, 35%, 40%, 45%, 50%, 55%, 60%, 65%, 70%, 75%, 80%, 85%, 90°/%, 95% or 98% or more relative to prior to administration). In certain embodiments, upon administration to a subject in need thereof, a conjugate described herein decreases macrophage inflammatory protein 1β (MIP-1β) level in a subject (e.g., at least 5%, 10%, 15%, 20%, 25%, 30%, 35%, 40%, 45%, 50%, 55%, 60%, 65%, 70%, 75%, 80%%, 85%, 90%, 95% or 98% or more relative to prior to administration). In further embodiments, upon administration to a subject in need thereof, a conjugate described herein modulates (increases or decreases) an autoimmunity marker, e.g., Tₕ1 cell count in a subject (e.g., at least 5%, 10%, 15%, 20%, 25%, 30%, 35%, 40%, 45%, 50%, 55%, 60%, 65%, 70%, 75%, 80%, 85%, 90%, 95% or 98% or more relative to prior to administration). The Tₕ1 cell count increase or decrease may be desirable depending on the particular condition and its state. An attendant doctor or nurse practitioner can determine whether an increase or a decrease in the Tₕ1 cell count is desired.

In some embodiments, a conjugate described herein decreases gastrointestinal inflammation (upper intestine, cecum, ileum, colon, rectum) in a subject (e.g., at least 5%, 10%, 15%, 20%, 25%, 30%, 35%, 40%, 45%, 50%, 55%, 60%, 65%, 70%, 75%, 80%, 85%, 90%, 95% or 98% or more relative to prior to administration)). In certain embodiments, a conjugate described herein decreases abdominal pain (e.g., incidence and/or intensity) in a subject (e.g., at least 5%, 10%, 15%, 20%, 25%, 30%, 35%, 40%, 45%, 50%, 55%, 60%, 65%, 70%, 75%, 80%, 85%, 90%, 95% or 98% or more relative to prior to administration). In particular embodiments, a conjugate described herein decreases gastrointestinal permeability in a subject (e.g., at least 5%, 10%, 15%, 20%, 25%, 30%, 35%, 40%, 45%, 50%, 55%, 60%, 65%, 70%, 75%, 80%, 85%, 90%, 95% or 98% or more relative to prior to administration). In further embodiments, a conjugate described herein increases intestinal motility or frequency of bowel movements in a subject (e.g., at least 5%, 10%, 15%, 20%, 25%, 30%, 35%, 40%, 45%, 50%, 55%, 60%%, 65%, 70%, 75%, 80%, 85%, 90%, 95% or 98% or more relative to prior to administration). In yet further embodiments, a conjugate described herein decreases intestinal motility or frequency of bowel movements in a subject (e.g., at least 5%, 10%, 15%, 20%, 25%, 30%, 35%, 40%, 45%, 50%, 55%, 60%%, 65%, 70%, 75%, 80%, 85%, 90%, 95% or 98% or more relative to prior to administration). In still further embodiments, a conjugate described herein decreases gastrointestinal bleeding in a subject (e.g., at least 5%, 10%, 15%, 20%, 25%, 30%, 35%, 40%, 45%, 50%, 55%, 60%, 65%, 70%, 75%, 80%, 85%, 90%, 95% or 98% or more relative to prior to administration). In other embodiments, a conjugate described herein decreases or increases mucus secretion or improves mucosal health in a gastrointestinal cell, tissue or in a subject (e.g., at least 5%, 10%, 15%, 20%, 25%, 30%, 35%, 40%, 45%, 50%, 55%, 60%, 65%, 70%, 75%, 80%, 85%, 90%, 95% or 98% or more relative to prior to administration).

Additionally or alternatively, the conjugates described herein may be used for modulating a multiple sclerosis marker in a subject in need thereof. A method of modulating a multiple sclerosis marker in a subject in need thereof may include administering a conjugate described herein (e.g., a pharmaceutical composition containing the conjugate) to a subject in need thereof.

Non-limiting examples of multiple sclerosis markers include an Nrf2 expression level, citric acid level, serotonin level, β-hydroxybutyric acid level, docosahexaenoic acid level, a L-citrulline level, picolinic acid level, quinolinic acid level, 2-ketoglutaric acid level, L-kynurenine/L-tryptophan ratio, kyunurenic acid level, prostaglandin E2 level, leukotriene B4, linolenic acid level, linoleic acid level, CD8⁺ T cell count, memory B cell count, CD4⁺ EM cell count, cumulative number of new Gd+ lesions, L-phenylalanine level, hippuric acid level, eicosapentaenoic acid level, putrescine level, N-methyl nicotinic acid level, lauric acid level, and arachidonic acid level.

In some embodiments, upon administration to a subject in need thereof, a conjugate described herein increases a multiple sclerosis marker in a subject, e.g., an Nrf2 expression level, citric acid level, serotonin level, β-hydroxybutyric acid level, or docosahexaenoic acid level (e.g., at least 5%, 10%, 15%, 20%, 25%, 30%, 35%, 40%, 45%, 50%, 55%, 60%, 65%, 70%, 75%, 80%, 85%, 90%, 95% or 98% or more relative to prior to administration).

In some embodiments, an conjugate described herein decreases a multiple sclerosis in a subject, e.g., a L-citrulline level, picolinic acid level, quinolinic acid level, 2-ketoglutaric acid level, L-kynurenine/L-tryptophan ratio, kyunurenic acid level, prostaglandin E2 level, leukotriene B4, linolenic acid level, linoleic acid level, CD8⁺ T cell count, memory B cell count, CD4⁺ EM cell count, or cumulative number of new Gd+ lesions (e.g., at least 5%, 10%, 15%, 20%, 25%, 30%, 35%, 40%, 45%, 50%, 55%, 60%, 65%, 70%, 75%, 80%, 85%, 90%, 95% or 98% or more relative to prior to administration)).

### Pharmaceutical Compositions

The conjugates disclosed herein may be formulated into pharmaceutical compositions for administration to human subjects in a biologically compatible form suitable for administration in vivo.

Pharmaceutical compositions typically include a conjugate as described herein and a physiologically acceptable excipient (e.g., a pharmaceutically acceptable excipient).

The conjugate described herein can also be used in the form of the free acid/base, in the form of salts, zwitterions, or as solvates. All forms are within the scope of the invention. The conjugates, salts, zwitterions, solvates, or pharmaceutical compositions thereof, may be administered to a subject in a variety of forms depending on the selected route of administration, as will be understood by those skilled in the art. The conjugates described herein may be administered, for example, by oral, parenteral, buccal, sublingual, nasal, rectal, patch, pump, or transdermal administration, and the pharmaceutical compositions formulated accordingly. Parenteral administration includes intravenous, intraperitoneal, subcutaneous, intramuscular, transepithelial, nasal, intrapulmonary, intrathecal, rectal, and topical modes of administration. Parenteral administration may be by continuous infusion over a selected period of time.

For human use, a conjugate disclosed herein can be administered alone or in admixture with a pharmaceutical carrier selected regarding the intended route of administration and standard pharmaceutical practice. Pharmaceutical compositions for use in accordance with the present invention thus can be formulated in a conventional manner using one or more physiologically acceptable carriers having excipients and auxiliaries that facilitate processing of conjugates disclosed herein into preparations which can be used pharmaceutically.

This disclosure also includes pharmaceutical compositions which can contain one or more physiologically acceptable carriers. In making the pharmaceutical compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semisolid, or liquid material (e.g., normal saline), which acts as a vehicle, carrier or medium for the active ingredient. Thus, the compositions can be in the form of tablets, powders, lozenges, sachets, cachets, elixirs, suspensions, emulsions, solutions, syrups, and soft and hard gelatin capsules. As is known in the art, the type of diluent can vary depending upon the intended route of administration. The resulting compositions can include additional agents, e.g., preservatives.

The excipient or carrier is selected on the basis of the mode and route of administration. Suitable pharmaceutical carriers, as well as pharmaceutical necessities for use in pharmaceutical formulations, are described in Remington: The Science and Practice of Pharmacy, 21st Ed., Gennaro, Ed., Lippencott Williams & Wilkins (2005), a well-known reference text in this field, and in the USP/NF (United States Pharmacopeia and the National Formulary). Examples of suitable excipients are lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents, e.g., talc, magnesium stearate, and mineral oil; wetting agents; emulsifying and suspending agents; preserving agents, e.g., methyl- and propylhydroxy-benzoates; sweetening agents; and flavoring agents. Other exemplary excipients are described in Handbook of Pharmaceutical Excipients, 6th Edition, Rowe et al., Eds., Pharmaceutical Press (2009).

These pharmaceutical compositions can be manufactured in a conventional manner, e.g., by conventional mixing, dissolving, granulating, dragee-making, levigating, emulsifying, encapsulating, entrapping, or lyophilizing processes. Methods well known in the art for making formulations are found, for example, in Remington: The Science and Practice of Pharmacy, 21st Ed., Gennaro, Ed., Lippencott Williams & Wilkins (2005), and Encyclopedia of Pharmaceutical Technology, eds. J. Swarbrick and J. C. Boylan, 1988-1999, Marcel Dekker, New York. Proper formulation is dependent upon the route of administration chosen. The formulation and preparation of such compositions is well-known to those skilled in the art of pharmaceutical formulation. In preparing a formulation, the conjugates can be milled to provide the appropriate particle size prior to combining with the other ingredients. If the conjugate is substantially insoluble, it can be milled to a particle size of less than 200 mesh. If the conjugate is substantially water soluble, the particle size can be adjusted by milling to provide a substantially uniform distribution in the formulation, e.g., about 40 mesh.

### Dosages

The dosage of the conjugate used in the methods described herein, or pharmaceutically acceptable salts or prodrugs thereof, or pharmaceutical compositions thereof, can vary depending on many factors, e.g., the pharmacodynamic properties of the conjugate; the mode of administration; the age, health, and weight of the recipient; the nature and extent of the symptoms; the frequency of the treatment, and the type of concurrent treatment, if any; and the clearance rate of the conjugate in the subject to be treated. One of skill in the art can determine the appropriate dosage based on the above factors. The conjugates used in the methods described herein may be administered initially in a suitable dosage that may be adjusted as required, depending on the clinical response. In general, a suitable daily dose of a conjugate disclosed herein will be that amount of the conjugate that is the lowest dose effective to produce a therapeutic effect. Such an effective dose will generally depend upon the factors described above.

A conjugate disclosed herein may be administered to the subject in a single dose or in multiple doses. When multiple doses are administered, the doses may be separated from one another by, for example, 1-24 hours, 1-7 days, or 1-4 weeks. The conjugate may be administered according to a schedule, or the conjugate may be administered without a predetermined schedule. It is to be understood that, for any particular subject, specific dosage regimes should be adjusted over time according to the individual need and the professional judgment of the person administering or supervising the administration of the compositions.

The conjugates may be provided in a dosage form. In some embodiments, the unit dosage form may be an oral unit dosage form (e.g., a tablet, capsule, suspension, liquid solution, powder, crystals, lozenge, sachet, cachet, elixir, syrup, and the like) or a food product serving (e.g., the active agents may be included as food additives or dietary ingredients). In certain embodiments, the dosage form is designed for administration of at least one conjugate disclosed herein, where the total amount of an administered conjugate is from 0.1 g to 10 g (e.g., 0.5 g to 9 g, 0.5 g to 8 g, 0.5 g to 7 g, 0.5 g to 6 g, 0.5 g to 5 g, 0.5 g to 1 g, 0.5 g to 1.5 g, 0.5 g to 2 g, 0.5 g to 2.5 g, 1 g to 1.5 g, 1 g to 2 g, 1 g to 2.5 g, 1.5 g to 2 g, 1.5 g to 2.5 g, or 2 g to 2.5 g). In other embodiments, the conjugate is consumed at a rate of 0.1 g to 10 g per day (e.g., 0.5 g to 9 g, 0.5 g to 8 g, 0.5 g to 7 g, 0.5 g to 6 g, 0.5 g to 5 g, 0.5 g to 1 g per day, 0.5 g to 1.5 g per day, 0.5 g to 2 g per day, 0.5 g to 2.5 g per day, 1 g to 1.5 g per day, 1 g to 2 g per day, 1 g to 2.5 g per day, 1.5 g to 2 g per day, 1.5 g to 2.5 g per day, or 2 g to 2.5 g per day) or more. The attending physician ultimately will decide the appropriate amount and dosage regimen, an effective amount of the conjugate disclosed herein may be, for example, a total daily dosage of, e.g., between 0.5 g and 5 g (e.g., 0.5 to 2.5 g) of any of the conjugate described herein. Alternatively, the dosage amount can be calculated using the body weight of the subject. Preferably, when daily dosages exceed 5 g/day, the dosage of the conjugate may be divided across two or three daily administration events.

In the methods of the invention, the time period during which multiple doses of a conjugate disclosed herein are administered to a subject can vary. For example, in some embodiments doses of the conjugates are administered to a subject over a time period that is 1-7 days; 1-12 weeks; or 1-3 months. In other embodiments, the conjugates are administered to the subject over a time period that is, for example, 4-11 months or 1-30 years. In yet other embodiments, the conjugates disclosed herein are administered to a subject at the onset of symptoms. In any of these embodiments, the amount of the conjugate that is administered may vary during the time period of administration. When a conjugate is administered daily, administration may occur, for example, 1, 2, 3, or 4 times per day.

### Formulations

A conjugate described herein may be administered to a subject with a pharmaceutically acceptable diluent, carrier, or excipient, in unit dosage form. Conventional pharmaceutical practice may be employed to provide suitable formulations or compositions to administer the conjugate to subjects suffering from a disorder. Administration may begin before the subject is symptomatic.

Exemplary routes of administration of the conjugates disclosed herein or pharmaceutical compositions thereof, used in the present invention include oral, sublingual, buccal, transdermal, intradermal, intramuscular, parenteral, intravenous, intra-arterial, intracranial, subcutaneous, intraorbital, intraventricular, intraspinal, intraperitoneal, intranasal, inhalation, and topical administration. The conjugates desirably are administered with a physiologically acceptable carrier (e.g., a pharmaceutically acceptable carrier). Pharmaceutical formulations of the conjugates described herein formulated for treatment of the disorders described herein are also part of the present invention. In some preferred embodiments, the conjugates disclosed herein are administered to a subject orally. In other preferred embodiments, the conjugates disclosed herein are administered to a subject topically.

### Formulations for Oral Administration

The pharmaceutical compositions contemplated by the invention include those formulated for oral administration (“oral dosage forms”). Oral dosage forms can be, for example, in the form of tablets, capsules, a liquid solution or suspension, a powder, or liquid or solid crystals, which contain the active ingredient(s) in a mixture with physiologically acceptable excipients (e.g., pharmaceutically acceptable excipients). These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other physiologically acceptable excipients (e.g., pharmaceutically acceptable excipients) can be colorants, flavoring agents, plasticizers, humectants, buffering agents, and the like.

Formulations for oral administration may also be presented as chewable tablets, as hard gelatin capsules where the active ingredient is mixed with an inert solid diluent (e.g., potato starch, lactose, microcrystalline cellulose, calcium carbonate, calcium phosphate or kaolin), or as soft gelatin capsules where the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Controlled release compositions for oral use may be constructed to release the active drug by controlling the dissolution and/or the diffusion of the active drug substance. Any of a number of strategies can be pursued in order to obtain controlled release and the targeted plasma concentration versus time profile. In one example, controlled release is obtained by appropriate selection of various formulation parameters and ingredients, including, e.g., various types of controlled release compositions and coatings. Examples include single or multiple unit tablet or capsule compositions, oil solutions, suspensions, emulsions, microcapsules, microspheres, nanoparticles, patches, and liposomes. In certain embodiments, compositions include biodegradable, pH, and/or temperature-sensitive polymer coatings.

Dissolution or diffusion controlled release can be achieved by appropriate coating of a tablet, capsule, pellet, or granulate formulation of conjugates, or by incorporating the conjugate into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, d-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

The liquid forms in which the conjugates and compositions of the present invention can be incorporated for administration orally include aqueous solutions, suitably flavored syrups, aqueous or oil suspensions, and flavored emulsions with edible oils, e.g., cottonseed oil, sesame oil, coconut oil, or peanut oil, as well as elixirs and similar pharmaceutical vehicles.

### Formulations for Buccal Administration

Dosages for buccal or sublingual administration typically are 0.1 to 500 mg per single dose as required. In practice, the physician determines the actual dosing regimen which is most suitable for an individual subject, and the dosage varies with the age, weight, and response of the particular subject. The above dosages are exemplary of the average case, but individual instances exist where higher or lower dosages are merited, and such are within the scope of this invention.

For buccal administration, the compositions may take the form of tablets, lozenges, etc. formulated in a conventional manner. Liquid drug formulations suitable for use with nebulizers and liquid spray devices and electrohydrodynamic (EHD) aerosol devices will typically include a conjugate disclosed herein with a pharmaceutically acceptable carrier. Preferably, the pharmaceutically acceptable carrier is a liquid, e.g., alcohol, water, polyethylene glycol, or a perfluorocarbon. Optionally, another material may be added to alter the aerosol properties of the solution or suspension of conjugates disclosed herein.

Desirably, this material is liquid, e.g., an alcohol, glycol, polyglycol, or a fatty acid. Other methods of formulating liquid drug solutions or suspension suitable for use in aerosol devices are known to those of skill in the art (see, e.g., U.S. Pat. Nos. 5,112,598 and 5,556,611, each of which is herein incorporated by reference).

### Formulations for Nasal or Inhalation Administration

The conjugates may also be formulated for nasal administration. Compositions for nasal administration also may conveniently be formulated as aerosols, drops, gels, and powders. The formulations may be provided in a single or multidose form. In the case of a dropper or pipette, dosing may be achieved by the subject administering an appropriate, predetermined volume of the solution or suspension. In the case of a spray, this may be achieved, for example, by means of a metering atomizing spray pump.

The conjugates may further be formulated for aerosol administration, particularly to the respiratory tract by inhalation and including intranasal administration. The conjugates for nasal or inhalation administration will generally have a small particle size for example on the order of five (5) microns or less. Such a particle size may be obtained by means known in the art, for example by micronization. The active ingredient is provided in a pressurized pack with a suitable propellant, e.g., a chlorofluorocarbon (CFC), for example, dichlorodifluoromethane, trichlorofluoromethane, or dichlorotetrafluoroethane, or carbon dioxide, or other suitable gas. The aerosol may conveniently also contain a surfactant, e.g., lecithin. The dose of drug may be controlled by a metered valve. Alternatively, the active ingredients may be provided in a form of a dry powder, e.g., a powder mix of the conjugate in a suitable powder base, e.g., lactose, starch, and starch derivatives, e.g., hydroxypropylmethyl cellulose, and polyvinylpyrrolidine (PVP). The powder carrier will form a gel in the nasal cavity. The powder composition may be presented in unit dose form for example in capsules or cartridges of e.g., gelatin or blister packs from which the powder may be administered by means of an inhaler.

Aerosol formulations typically include a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device, e.g., a single dose nasal inhaler or an aerosol dispenser fitted with a metering valve which is intended for disposal after use. Where the dosage form comprises an aerosol dispenser, it will contain a propellant, which can be a compressed gas, e.g., compressed air or an organic propellant, e.g., fluorochlorohydrocarbon. The aerosol dosage forms can also take the form of a pump-atomizer.

### Formulations for Parenteral Administration

The conjugates described herein for use in the methods of the invention can be administered in a pharmaceutically acceptable parenteral (e.g., intravenous or intramuscular) formulation as described herein. The pharmaceutical formulation may also be administered parenterally (intravenous, intramuscular, subcutaneous or the like) in dosage forms or formulations containing conventional, nontoxic pharmaceutically acceptable carriers and adjuvants. In particular, formulations suitable for parenteral administration include aqueous and non-aqueous sterile injection solutions which may contain anti-oxidants, buffers, bacteriostats, and solutes which render the formulation isotonic with the blood of the intended recipient; and aqueous and non-aqueous sterile suspensions which may include suspending agents and thickening agents. For example, to prepare such a composition, the conjugates disclosed herein may be dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution and isotonic sodium chloride solution. The aqueous formulation may also contain one or more preservatives, for example, methyl, ethyl or n-propyl p-hydroxybenzoate. Additional information regarding parenteral formulations can be found, for example, in the United States Pharmacopeia-National Formulary (USP-NF), herein incorporated by reference.

The parenteral formulation can be any of the five general types of preparations identified by the USP-NF as suitable for parenteral administration:
(1) “Drug Injection:” a liquid preparation that is a drug substance (e.g., a conjugate disclosed herein or a solution thereof);
(2) “Drug for Injection:” the drug substance (e.g., a conjugate disclosed herein) as a dry solid that will be combined with the appropriate sterile vehicle for parenteral administration as a drug injection;
(3) “Drug Injectable Emulsion:” a liquid preparation of the drug substance (e.g., a conjugate disclosed herein) that is dissolved or dispersed in a suitable emulsion medium;
(4) “Drug Injectable Suspension:” a liquid preparation of the drug substance (e.g., a conjugate disclosed herein) suspended in a suitable liquid medium; and
(5) “Drug for Injectable Suspension:” the drug substance (e.g., a conjugate disclosed herein) as a dry solid that will be combined with the appropriate sterile vehicle for parenteral administration as a drug injectable suspension.

Exemplary formulations for parenteral administration include solutions of the conjugates prepared in water suitably mixed with a surfactant, e.g., hydroxypropylcellulose. Dispersions can also be prepared in glycerol, liquid polyethylene glycols, DMSO and mixtures thereof with or without alcohol, and in oils. Under ordinary conditions of storage and use, these preparations may contain a preservative to prevent the growth of microorganisms. Conventional procedures and ingredients for the selection and preparation of suitable formulations are described, for example, in Remington: The Science and Practice of Pharmacy, 21st Ed., Gennaro, Ed., Lippencott Williams & Wilkins (2005) and in The United States Pharmacopeia: The National Formulary (USP 36 NF31), published in 2013.

Formulations for parenteral administration may, for example, contain excipients, sterile water, or saline, polyalkylene glycols, e.g., polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the conjugates or biologically active agents within the conjugates. Other potentially useful parenteral delivery systems for conjugates include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

The parenteral formulation can be formulated for prompt release or for sustained/extended release of the conjugate. Exemplary formulations for parenteral release of the conjugate include: aqueous solutions, powders for reconstitution, cosolvent solutions, oil/water emulsions, suspensions, oil-based solutions, liposomes, microspheres, and polymeric gels.

### Preparation of Conjugates

Compounds can be prepared using synthetic methods and reaction conditions known in the art. Optimum reaction conditions and reaction times may vary depending on the reactants used. Unless otherwise specified, solvents, temperatures, pressures, and other reaction conditions may be selected by one of ordinary skill in the art.

### Glycosidic Preparation Strategy #1: (Substitution)

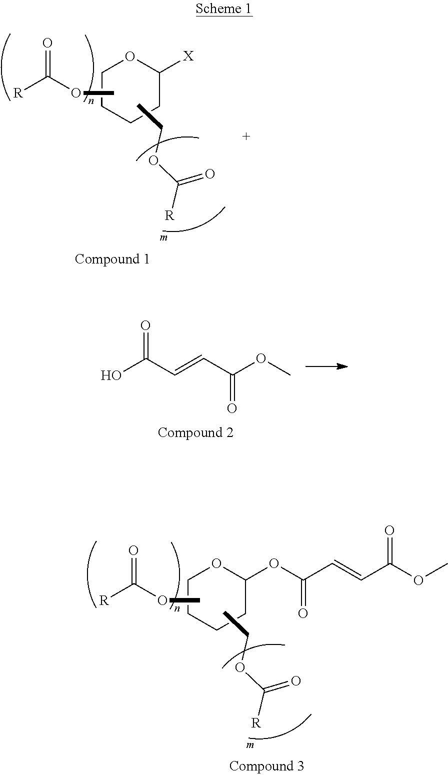

In Scheme 1, a polyacylated sugar, compound 1 where n represents an integer from 1 to 3 m represents an integer from 0 to 1, R is equal to C₁₋₁₀ alkyl is treated with monomethyl fumarate compound 2, in an appropriate solvent, optionally in the presence of a catalyst. Suitable catalysts include pyridine, dimethylaminopyridine, trimethylamine and the like. The catalyst can be used in quantities ranging from 0.01 to 1.1 equivalents, relative to compound 2. Suitable solvents include methylene chloride, ethyl acetate, diethyl ether, tetrahydrofuran, 1,4-dioxane, 1,2-dimethoxyethane, toluene, combinations thereof and the like. Reaction temperatures range from −10° C. to the boiling point of the solvent used; reaction completion times range from 1 to 96 h. The monomethyl fumarate can be used in quantities ranging from 0.5 to 15 equivalents relative to compound 1.

The product, compound 3, can be purified by methods known to those of skill in the art

### Glycosidic Preparation Strategy #2: (Mitsunobu Reaction)

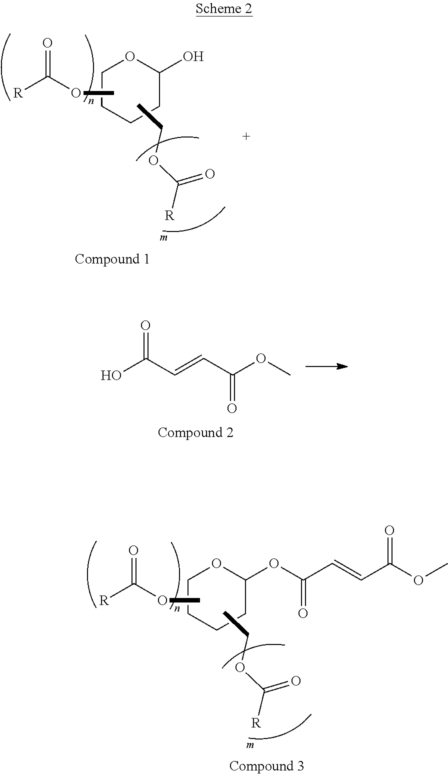

In Scheme 2 a polyacylated sugar, compound 1 where n represents an integer from 1 to 3, m represents an integer from 0 to 1, R is equal to C₁₋₁₀ alkyl is treated with triphenylphosphine and a diazo compound such as diethylazodicarboxylate (DEAD) and the like in an appropriate solvent. Suitable solvents include methylene chloride, THF, acetonitrile, toluene, diethyl ether, combinations thereof and the like. Reaction temperatures range from −10° C. to the boiling point of the solvent used; reaction completion times range from 1 to 96 h. After a time range compound 2 is added in the same solvent used in the prior transformation. Reaction temperatures range from −10° C. to the boiling point of the solvent used; reaction completion times range from 1 to 96 h. The product, compound 3 can be purified by methods known to those of skill in the art.

### Glycosidic Preparation Strategy #3: (Acylation)

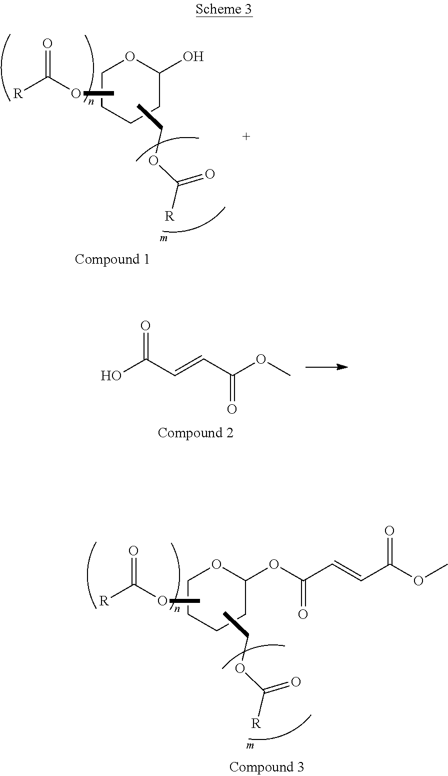

In Scheme 3 Step compound 1 is treated with an compound 2, in an appropriate solvent, optionally in the presence of a catalyst. Suitable catalysts include pyridine, dimethylaminopyridine, trimethylamine and the like. The catalyst can be used in quantities ranging from 0.01 to 1.1 equivalents, relative to compound 2. Suitable solvents include methylene chloride, ethyl acetate, diethyl ether, tetrahydrofuran, 1,4-dioxane, 1,2-dimethoxyethane, toluene, combinations thereof and the like. Reaction temperatures range from −10° C. to the boiling point of the solvent used; reaction completion times range from 1 to 96 h. A suitable acylating agent may also be generated in situ by a reaction of a carboxylic acid with an activating reagent such as EDC, DCC, or EEDQ or the like. The acylating agents can be used in quantities ranging from 0.5 to 15 equivalents relative to compound 1.

### Ester Preparation Strategy #1 (Acylation)

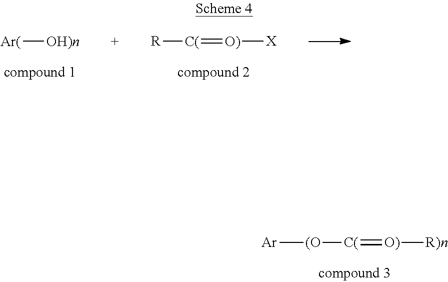

In Scheme 4, a polyphenolic compound, compound 1 where n represents an integer from 1 to 15, is treated with an acylating agent, compound 2, in an appropriate solvent, optionally in the presence of a catalyst. Suitable catalysts include pyridine, dimethylaminopyridine, trimethylamine and the like. The catalyst can be used in quantities ranging from 0.01 to 1.1 equivalents, relative to compound 2. Suitable solvents include methylene chloride, ethyl acetate, diethyl ether, tetrahydrofuran, 1,4-dioxane, 1,2-dimethoxyethane, toluene, combinations thereof and the like. Reaction temperatures range from −10° C. to the boiling point of the solvent used; reaction completion times range from 1 to 96 h. Suitable acylating agents include acyl chlorides, acyl fluorides, acyl bromides, carboxylic acid anhydrides whether symmetrical or not. A suitable acylating agent may also be generated in situ by prior reaction of a carboxylic acid with an activating reagent such as EDC or EEDQ or the like. The acylating agents can be used in quantities ranging from 0.5 to 15 equivalents relative to compound 1.

The product, compound 3, can be purified by methods known to those of skill in the art.

### Ester Preparation Strategy #2 (Acylation)

In some cases, the polyphenolic compound 1 may contain a functional group, Y, required to remain unreacted in the course of ester formation. In this case, it is appropriate to protect the functional group, Y, in the polyphenolic compound from acylation. This functional group may be an amino group or a hydroxyl group or other functionality with a labile hydrogen attached to a heteroatom. Such polyphenol esters can be prepared according to Scheme 5.

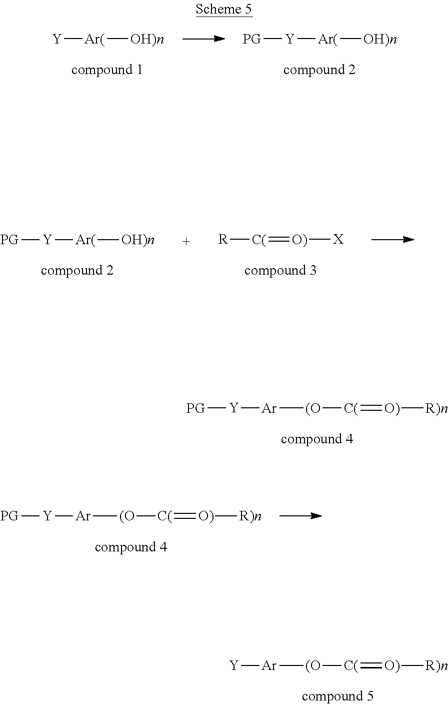

In Scheme 5 Step 1, compound 1, a polyphenolic compound containing a functional group Y with a labile hydrogen in need of protection, is treated with a protecting reagent such as BOC anhydride, benzyoxycarbonyl chloride, FMOC chloride, benzyl bromide and the like in an appropriate solvent, optionally in the presence of a catalyst to provide compound 2 scheme 2. Compound 2 can be purified by methods known to those of skill in the art.

In Scheme 5 Step 2, compound 2 is treated with an acylating agent, compound 3, in an appropriate solvent, optionally in the presence of a catalyst. Suitable catalysts include pyridine, dimethylaminopyridine, trimethylamine and the like. The catalyst can be used in quantities ranging from 0.01 to 1.1 equivalents, relative to compound 2. Suitable solvents include methylene chloride, ethyl acetate, diethyl ether, tetrahydrofuran, 1,4-dioxane, 1,2-dimethoxyethane, toluene, combinations thereof and the like. Reaction temperatures range from −10° C. to the boiling point of the solvent used; reaction completion times range from 1 to 96 h. Suitable acylating agents include acyl chlorides, acyl fluorides, acyl bromides, carboxylic acid anhydrides whether symmetrical or not. A suitable acylating agent may also be generated in situ by prior reaction of a carboxylic acid with an activating reagent such as EDC or EEDQ or the like. The acylating agents can be used in quantities ranging from 0.5 to 15 equivalents, relative to compound 3. Compound 4 can be purified by methods known to those of skill in the art.

In Scheme 5 Step 3, compound 4 is subjected to conditions that cleave the protecting group, PG.

In the case of a BOC protecting group, the protecting group of compound 4 is removed under acidic conditions to give compound 5 of the invention. Suitable acids include trifluoroacetic acid, hydrochloric acid, p-toluenesulfonic acid and the like.

In the case of an FMOC protecting group, the protecting group of compound 4 is removed under basic conditions to give compound 5 of the invention. Suitable bases include piperidine, triethylamine and the like. Suitable solvents include DMF, NMP dichloromethane and the like. The FMOC group is also removed under non-basic conditions such as by treatment with tetrabutylammonium fluoride trihydrate in a suitable solvent such as DMF. The FMOC group is also removed by catalytic hydrogenation. Suitable catalysts for hydrogenation include 10% palladium-on-charcoal and palladium (II) acetate and the like. Suitable solvents for hydrogenation include DMF, ethanol, and the like

In the case of a benzyloxycarbonyl or benzyl protecting group the protecting group of compound 4 is removed by hydrogenation to give compound 5. Suitable catalysts for hydrogenation include 10% Palladium-on-charcoal and Palladium acetate and the like. Suitable solvents for hydrogenation include DMF, ethanol, methanol, ethyl acetate, and the like. The product, compound 5, can be purified by methods known to those of skill in the art.

### Ester Preparation Strategy #3 (Acylation)

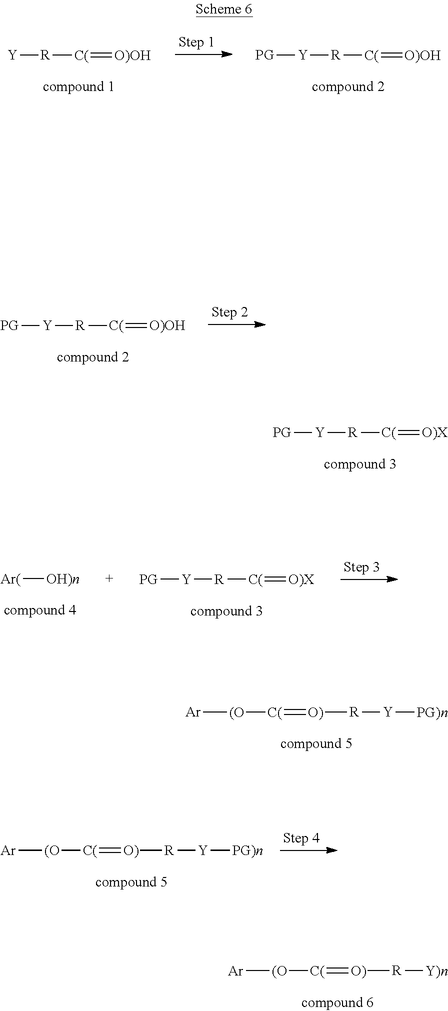

In Scheme 6 Step 1, compound 1, an acyl compound containing a functional group Y with a labile hydrogen in need on protection, is treated with a protecting reagent such as BOC anhydride, benzyoxycarbonyl chloride, FMOC chloride, benzyl bromide and the like in an appropriate solvent, optionally in the presence of a catalyst to provide compound 2 scheme 3. Compound 2 can be purified by methods known to those of skill in the art.

In Scheme 6 Step 2, compound 2 is treated with an activating reagent such as thionyl chloride, phosphorus oxychloride, EDC or EEDQ or the like to generate the activated acyl compound 3.

In Scheme 6 Step 3, the polyphenol compound 4 is treated with the activated acyl compound 3, in an appropriate solvent, optionally in the presence of a catalyst. Suitable catalysts include pyridine, dimethylaminopyridine, trimethylamine and the like to generate compound 5. The catalyst can be used in quantities ranging from 0.01 to 1.1 equivalents, relative to compound 3. Suitable solvents include methylene chloride, ethyl acetate, diethyl ether, tetrahydrofuran, 1,4-dioxane, 1,2-dimethoxyethane, toluene, combinations thereof and the like. Reaction temperatures range from −10° C. to the boiling point of the solvent used; reaction completion times range from 1 to 96 h. The activated acyl compound 3 can be used in quantities ranging from 0.5 to 15 equivalents relative to compound 4.

In Scheme 6 Step 4, compound 5 is subjected to conditions designed to cleave the protecting group, PG, illustrated in Scheme 2 above. The product, compound 6, can be purified by methods known to those of skill in the art.

### Ester Preparation Strategy #4 (Acylation)

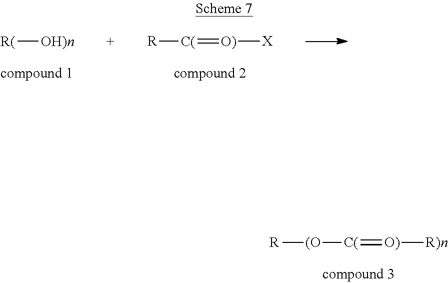

In Scheme 7 Step 1 a poly-ol compound, compound 1, where R represents a non-aromatic cyclic or acyclic moiety and n represents an integer from 1 to 15, is treated with an acylating agent, compound 2, in an appropriate solvent, optionally in the presence of a catalyst. Suitable catalysts include pyridine, dimethylaminopyridine, trimethylamine and the like. The catalyst can be used in quantities ranging from 0.01 to 1.1 equivalents, relative to compound 2. Suitable solvents include methylene chloride, ethyl acetate, diethyl ether, tetrahydrofuran, 1,4-dioxane, 1,2-dimethoxyethane, toluene, combinations thereof and the like. Reaction temperatures range from −10° C. to the boiling point of the solvent used; reaction completion times range from 1 to 96 h. Suitable acylating agents include acyl chlorides, acyl fluorides, acyl bromides, carboxylic acid anhydrides whether symmetrical or not. A suitable acylating agent may also be generated in situ by prior reaction of a carboxylic acid with an activating reagent such as EDC or EEDQ or the like. The acylating agents can be used in quantities ranging from 0.5 to 15 equivalents, relative to compound 1. The product, compound 3, can be purified by methods known to those of skill in the art.

### Ester Preparation Strategy #5 (Baeyer-Villiger Oxidation)

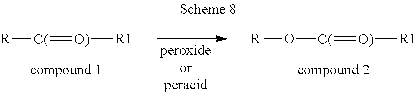

In Scheme 8 Step 1, a ketone compound, compound 1, where R and R1 represent non-aromatic cyclic or acyclic moieties, is treated with a peroxide or peroxyacid agent, such as meta-chloroperbenzoic acid, performic acid, peracetic acid, hydrogen peroxide, tert-butyl hydroperoxide and the like in an appropriate solvent, optionally in the presence of a catalyst. Suitable solvents include methylene chloride, diethyl ether, combinations thereof and the like. Suitable catalysts include BF₃, carboxylic acids and the like. Reaction temperatures range from −10° C. to the boiling point of the solvent used; reaction completion times range from 1 to 96 h. The product, compound 2, can be purified by methods known to those of skill in the art.

The R and R1 groups of compound 1 in Scheme 5 may optionally include additional ketone functionality that can undergo reaction. In addition the R and R1 groups of compound 1 may form a ring.

### Ester Preparation Strategy #6 (Mitsunobu Reaction)

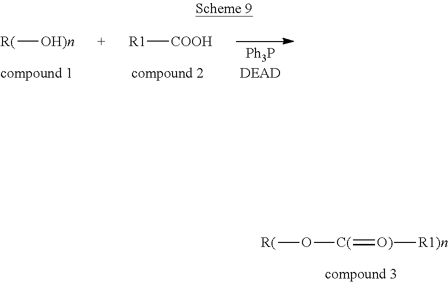

In Scheme 9 Step 1, a mixture of an alcohol compound, compound 1, where R represents a non-aromatic cyclic or acyclic moiety, and a carboxylic acid, compound 2 where R1 represents an alkanoyl group optionally substituted with one or more protected hydroxyl groups or oxo is treated with triphenylphosphine and a diazo compound such as diethylazodicarboxylate (DEAD) and the like in an appropriate solvent. Suitable solvents include methylene chloride, THF, acetonitrile, toluene, diethyl ether, combinations thereof and the like. Reaction temperatures range from −10° C. to the boiling point of the solvent used; reaction completion times range from 1 to 96 h. The product, compound 3 can be purified by methods known to those of skill in the art.

Where compound 3 is optionally substituted by one or more protected alcohol groups deprotection is accomplished by the methods illustrated in Scheme 2 above.

Ester preparation strategy #7 (Nucleophilic Alkylation)

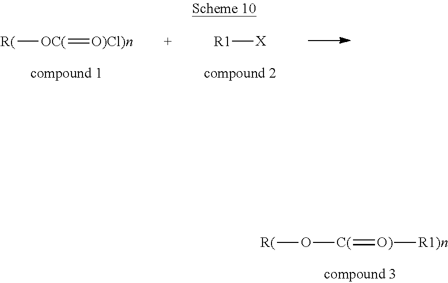

In Scheme 10 Step 1, a chloroformate compound, compound 1, where R represents an aromatic moiety or a non-aromatic cyclic or acyclic moiety, is treated, in an appropriate solvent, with an organometallic compound, compound 2 where R¹ represents an alkyl group optionally substituted with one or more protected hydroxyl groups and X represents a metal such as Cu, Zn, Mg which is optionally coordinated by one or more counterions, such as chloride. Suitable solvents include methylene chloride, THF, acetonitrile, toluene, diethyl ether, combinations thereof, and the like. Reaction temperatures range from −10° C. to the boiling point of the solvent used; reaction completion times range from 1 to 96 h. The product, compound 3, can be purified by methods known to those of skill in the art.

Compound 1 can be prepared from the corresponding alcohol or polyol compounds by standard methods familiar to one skilled in the art.

Where compound 2 is optionally substituted by one or more protected alcohol groups deprotection is accomplished by the methods illustrated in Scheme 2 above.

Further modification of the initial product by methods known in the art and illustrated in the examples below, may be used to prepare additional compounds of this invention.

### Ester Preparation Strategy #8 (Acylation)

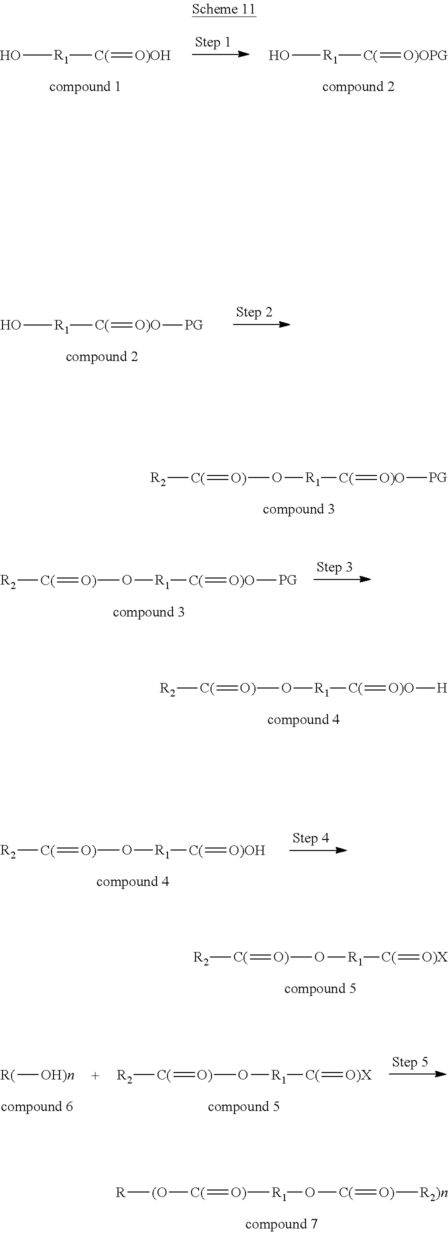

In Scheme 11 step 1, compound 1, an acyl compound containing a hydroxyl group to be acylated, is treated with a protecting reagent such as benzyl bromide and the like in an appropriate solvent, optionally in the presence of a catalyst to provide compound 2 scheme 8. Compound 2 can be purified by methods known to those of skill in the art.

In Scheme 11 Step 2, compound 2 is treated with an acylating agent, in an appropriate solvent, optionally in the presence of a catalyst. Suitable catalysts include pyridine, dimethylaminopyridine, trimethylamine and the like. The catalyst can be used in quantities ranging from 0.01 to 1.1 equivalents, relative to compound 2. Suitable solvents include methylene chloride, ethyl acetate, diethyl ether, tetrahydrofuran, 1,4-dioxane, 1,2-dimethoxyethane, toluene, combinations thereof and the like. Reaction temperatures range from −10° C. to the boiling point of the solvent used; reaction completion times range from 1 to 96 h. Suitable acylating agents include acyl chlorides, acyl fluorides, acyl bromides, carboxylic acid anhydrides whether symmetrical or not. A suitable acylating agent may also be generated in situ by a reaction of a carboxylic acid with an activating reagent such as EDC or EEDQ or the like. The acylating agents can be used in quantities ranging from 0.5 to 15 equivalents relative to compound 1.

In Scheme 11 Step 3, compound 3 is subjected to conditions that cleave the protecting group, PG. In the case of a benzyl protecting group, the protecting group of compound 3 is removed by hydrogenation to give compound 4. Suitable catalysts for hydrogenation include 10% palladium-on-charcoal and palladium acetate and the like. Suitable solvents for hydrogenation include, DMF, ethanol, methanol, ethyl acetate and the like. The product, compound 4, can be purified by methods known to those of skill in the art.

In Scheme 11 Step 4, compound 4 is treated with an activating reagent such as thionyl chloride, phosphorus oxychloride, EDO or EEDQ or the like to generate the activated acyl compound 5.

In Scheme 11 Step 5, the poly-hydroxyl compound, compound 6, where R represents an aromatic or an aliphatic cyclic or acyclic core, is treated with the activated acyl compound 5, in an appropriate solvent, optionally in the presence of a catalyst. Suitable catalysts include pyridine, dimethylaminopyridine, trimethylamine and the like to generate compound 5. The catalyst can be used in quantities ranging from 0.01 to 1.1 equivalents, relative to compound 3. Suitable solvents include methylene chloride, ethyl acetate, diethyl ether, tetrahydrofuran, 1,4-dioxane, 1,2-dimethoxyethane, toluene, combinations thereof and the like. Reaction temperatures range from −10° C. to the boiling point of the solvent used; reaction completion times range from 1 to 96 h. The activated acyl compound 5 can be used in quantities ranging from 0.5 to 15 equivalents relative to compound 6.

The product, compound 7, can be purified by methods known in the art.

The following examples are meant to illustrate the invention. They are not meant to limit the invention in any way.

### EXAMPLES

### Example 1: Preparation of Exemplary Conjugates of the Invention

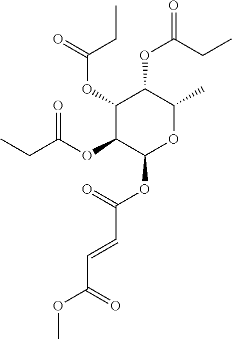 [SMILES: CCC(=O)O[C@H]1[C@H](OC(=O)CC)[C@H](OC(=O)/C=C/C(=O)OC)O[C@@H](C)[C@H]1OC(=O)CC]

### Compound 1: methyl((2S,3S,4R,5R,6S)-6-methyl-3,4,5-tris(propionyloxy)tetrahydro-2H-pyran-2-yl) fumarate

To a mixture of [(2S,3R,4R,5S)-6-hydroxy-2-methyl-4,5-di(propanoyloxy)tetrahydropyran-3-yl]propanoate (0.5 g, 1.50 mmol, 1 equiv.) and (E)-4-methoxy-4-oxo-but-2-enoic acid (234.87 mg, 1.81 mmol, 1.2 equiv.) in THF (5 mL) was added DCC (620.82 mg, 3.01 mmol, 2 equiv.) and DMAP (91.90 mg, 752.23 μmol, 0.5 equiv.) in one portion at 20° C. under N₂. The mixture was stirred at 20° C. for 12 h. LC-MS showed [(2S,3R,4R,5S)-6-hydroxy-2-methyl-4,5-di(propanoyloxy)tetrahydropyran-3-yl]propanoate was consumed completely and one main peak with desired m/z was detected. The reaction mixture was filtered and concentrated under reduced pressure to give a residue. The residue was purified by prep-HPLC (water+0.04% (v/v) HCl/MeOH), and methyl((2S,3S,4R,5R,6S)-6-methyl-3,4,5-tris(propionyloxy)tetrahydro-2H-pyran-2-yl) fumarate (0.1 g, 222.76 μmol, 14.81% yield, 99% purity) was obtained as colorless oil. LCMS: (M+Na)⁺: 467.1. ¹H NMR (400 MHz, CDCl₃) 6.9 (s, 2H), 6.4 (s, 1H), 5.3 (m, 3H), 4.3 (m, 1H), 3.8 (s, 3H), 2.5 (m, 2H), 2.2 (m, 4H), 1.2 (m, 6H) 1.0 (m, 6H) ppm.

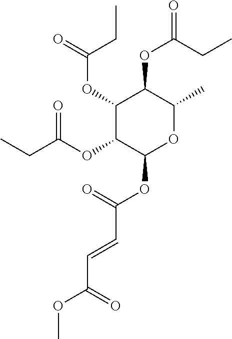 [SMILES: CCC(=O)O[C@@H]1[C@@H](OC(=O)CC)[C@H](C)O[C@@H](OC(=O)/C=C/C(=O)OC)[C@@H]1OC(=O)CC]

### Compound 2: methyl((2S,3R,4R,5S,6S)-6-methyl-3,4,5-tris(propionyloxy)tetrahydro-2H-pyran-2-yl) fumarate

To a solution of (2S,3S,4R,5R,6R)-5-acetoxy-6-hydroxy-2-methyltetrahydro-2H-pyran-3,4-diyl dipropionate (500 mg, 1.50 mmol, 1 equiv.), DCC (464.24 mg, 2.25 mmol, 455.14 μL, 1.5 equiv.) and DMAP (54.98 mg, 450.00 μmol, 0.3 equiv.) in THF (10 mL) was added (E)-4-methoxy-4-oxo-but-2-enoic acid (292.72 mg, 2.25 mmol, 1.5 equiv.) and the mixture was stirred at 25° C. for 12 h. LCMS showed the starting reactant was consumed. The mixture reaction was concentrated under reduced pressure. The residue was purified by prep-HPLC (column: Waters Xbridge Prep OBD C18 150×40 10p; mobile phase: water+10 mM NH₄HCO₃/ACN; B %: 40%-55%, 11 min) to afford the title compound (water+10 mM NH₄HCO₃/ACN)(50 mg, 106.88 μmol, 7.13% yield, 95% purity) as colorless oil. ¹H NMR (CDCl₃, 400 MHz): δ 6.9 (m, 2H), 6.1 (s, 1H) 5.3 (m, 2H), 5.1 (m, 1H), 3.9 (m, 1H), 3.8 (s, 3H), 2.4 (m, 6H), 1.5 (m, 3H), 1.3 (m, 3H), 1.1 (m, 3H), 1.0 (m, 3H) ppm LCMS: (M+Na)⁺467.1.

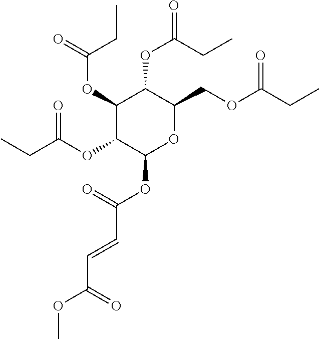 [SMILES: CCC(=O)OC[C@H]1O[C@@H](OC(=O)/C=C/C(=O)OC)[C@H](OC(=O)CC)[C@@H](OC(=O)CC)[C@@H]1OC(=O)CC]

### Compound 3: methyl((2S,3R,4S,5R,6R)-3,4,5-tris(propionyloxy)-6-((propionyloxy)methyl)tetrahydro-2H-pyran-2-yl) fumarate

To a mixture of (2R,3R,4S,5R,6R)-2-hydroxy-6-((propionyloxy)methyl)tetrahydro-2H-pyran-3,4,5-triyl tripropionate (0.5 g, 1.24 mmol, 1 equiv.) and (E)-4-methoxy-4-oxo-but-2-enoic acid (193.02 mg, 1.48 mmol, 1.2 equiv.) in THF (5 mL) was added DCC (510.20 mg, 2.47 mmol, 2 equiv.) and DMAP (75.52 mg, 618.19 μmol, 0.5 equiv.) in one portion at 20° C. under N₂. The mixture was stirred at 20° C. for 12 hours. LC-MS showed starting material was consumed completely and one main peak with desired m/z was detected. The reaction mixture was filtered and concentrated under reduced pressure to give a residue. The residue was purified by prep-HPLC (water+10 mM NH₄HCO₃/ACN). Then, the residue was separated by SFC (H₂O, 1% (v/v) NH₃, EtOH) to afford the title compound (0.006 g, 10.46 μmol, 11.74% yield, 90% purity) and its anomer (0.012 g, 21.84 μmol, 24.52%% yield, 94% purity) as colorless oil. ¹H NMR (CDCl₃, 400 MHz): δ 7.0 (m, 2H), 6.6 (d, 1H), 5.5 (dd 1H), 5.1 (m, 2H), 3.8 (s, 3H), 2.3 (m, 9H), 1.1 (m, 12H)ppm LCMS: (M+Na)⁺539.1.

Compound 3-d12 was synthesized in a similar manner as described herein, with the exception that d3-propionic acid was used in combination with the EDCI coupling conditions.

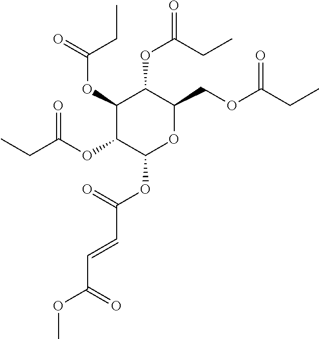 [SMILES: CCC(=O)OC[C@H]1O[C@H](OC(=O)/C=C/C(=O)OC)[C@H](OC(=O)CC)[C@@H](OC(=O)CC)[C@@H]1OC(=O)CC]

### Compound 4: methyl((2R,3R,4S,5R,6R)-3,4,5-tris(propionyloxy)-6-((propionyloxy)methyl)tetrahydro-2H-pyran-2-yl) fumarate

To a mixture of (2S,3R,4S,5R,6R)-2-hydroxy-6-((propionyloxy)methyl)tetrahydro-2H-pyran-3,4,5-triyl tripropionate (0.5 g, 1.24 mmol, 1 equiv.) and (E)-4-methoxy-4-oxo-but-2-enoic acid (193.02 mg, 1.48 mmol, 1.2 equiv.) in THF (5 mL) was added DCC (510.20 mg, 2.47 mmol, 2 equiv.) and DMAP (75.52 mg, 618.19 μmol, 0.5 equiv.) in one portion at 20° C. under N₂. The mixture was stirred at 20° C. for 12 hours. LC-MS showed starting material was consumed completely and one main peak with desired m/z was detected. The reaction mixture was filtered and concentrated under reduced pressure to give a residue. The residue was purified by prep-HPLC (water+10 mM NH₄HCO₃/ACN). Then the residue was separated by SFC (H₂O, 0.1% (v/v) NH₃, EtOH) to afford the title compound (0.006 g, 11.74% yield) and its anomer (0.012 g, 24.52%) as colorless oil. LCMS: (M+18)⁺: 534.1. ¹H NMR (CDCl₃, 400 MHz): δ 7.0 (s, 2H), 6.4 (s, 1H), 5.3 (t, 1H), 5.5 (m, 2H), 4.1 (dd, 3H), 3.8 (s, 3H), 2.3 (m, 9H), 1.0 (m, 12H) ppm.

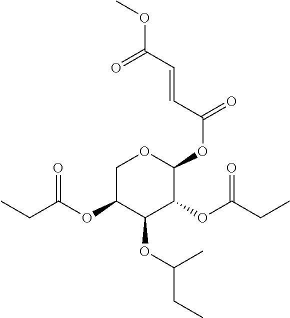 [SMILES: CCC(=O)O[C@H]1[C@H](OC(=O)/C=C/C(=O)OC)OC[C@H](OC(=O)CC)[C@@H]1OC(C)CC]

### Compound 5: methyl((2S,3R,4S,5S)-3,4,5-tris(propionyloxy)tetrahydro-2H-pyran-2-yl) fumarate

This compound was synthesized in the same manner as compound 2. ¹H NMR (400 MHz, chloroform-d): δ 7.07-6.73 (m, 1H), 5.78 (d, J=6.4 Hz, 1H), 5.39-5.27 (m, 1H), 5.20 (dd, J=8.4, 3.5 Hz, 1H), 4.06 (dd, J=12.8, 4.5 Hz, 1H), 3.82 (s, 3H), 2.56-2.19 (m, 6H), 1.28-0.97 (m, 9H) ppm.

LCMS: (M+Na)⁺: 453.1.

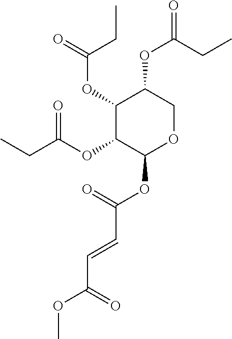 [SMILES: CCC(=O)O[C@H]1[C@H](OC(=O)/C=C/C(=O)OC)OC[C@@H](OC(=O)CC)[C@H]1OC(=O)CC]

### Compound 6: methyl((2S,3R,4R,5R)-3,4,5-tris(propionyloxy)tetrahydro-2H-pyran-2-yl) fumarate

To a solution of (2R,3R,4R)-2,3,4,5-tetrahydroxypentanal (5 g, 33.30 mmol, 1 equiv.) in pyridine (50 mL) was added propanoyl propanoate (26.01 g, 199.83 mmol, 25.75 mL, 6 equiv.) at 25° C. The mixture was stirred at 25° C. for 16 h. TLC indicated formation of new spots. The reaction mixture was concentrated under reduced pressure to give a residue. The residue was purified by column chromatography (SiO₂, petroleum ether/ethyl acetate=20/1 to 10/1) to give [(3R,4R,5R)-4,5,6-tri(propanoyloxy)tetrahydropyran-3-yl]propanoate (9 g, 24.04 mmol, 72.18% yield, 100% purity) as a colourless oil. To a solution of [(3R,4R,5R)-4,5,6-tri(propanoyloxy)tetrahydropyran-3-yl]propanoate (8.95 g, 23.91 mmol, 1 equiv.) in THF (100 mL) was added MeNH₂ (2.78 g, 35.86 mmol, 40% purity in H₂O, 1.5 equiv.) at 25° C. The mixture was stirred at 25° C. for 16 h. TLC indicated new spots formed. The reaction mixture was concentrated under reduced pressure to give a residue. The residue was purified by column chromatography (SiO₂, petroleum ether/ethyl acetate=10/1 to 1/1) to give [(3R,4R,5R)-6-hydroxy-4,5-di(propanoyloxy)tetrahydropyran-3-yl]propanoate (3 g, 8.01 mmol, 33.51% yield, 85% purity) as a yellow oil. To a solution of [(3R,4R,5R)-6-hydroxy-4,5-di(propanoyloxy)tetrahydropyran-3-yl]propanoate (300 mg, 942.45 μmol, 1 equiv.) in DCM (5 mL) was added DCC (291.68 mg, 1.41 mmol, 285.96 μL, 1.5 equiv.), DMAP (57.57 mg, 471.23 μmol, 0.5 equiv.) and (E)-4-methoxy-4-oxo-but-2-enoic acid (183.92 mg, 1.41 mmol, 1.5 equiv.) at 25° C. The mixture was stirred at 25° C. for 5 h. LCMS showed the desired compound was detected. The reaction mixture was filtered and concentrated under reduced pressure to give a residue. The residue was purified by prep-HPLC (column: Luna C18 100×30 5 μm; mobile phase: water+0.05% (v/v) HCl/ACN; B %: 40%-65%, 11 min) to give desired compound (200 mg) as a white solid, which was further separated by SFC (column: DAICEL CHIRALPAK IC 250 mm×30 mm, 5 μm; mobile phase: 0.1% NH₃, H₂O, IPA; B %: 25%-25%, 5.1 min) (104 mg, 217.47 μmol, 23.08% yield). LCMS: (M+18)⁺& (M+Na)⁺448.1 & 453. ¹H NMR (CDCl₃, 400 MHz): 6.8 (dd, 2H), 6.1 (d, 1H), 5.5 (m, 1H), 5.1 (m, 2H), 4.0 (dd, 2H), 3.8 (s, 3H), 2.3 (m, 6H), 1.1 (t, 9H) ppm.

Compound 6-d9 was synthesized in a similar manner as described herein, with the exception that d3-propionic acid was used in combination with the EDCI coupling conditions.

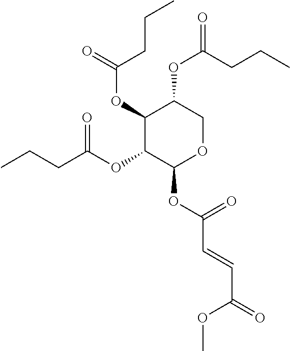 [SMILES: CCCC(=O)O[C@@H]1[C@@H](OC(=O)CCC)[C@H](OC(=O)/C=C/C(=O)OC)OC[C@H]1OC(=O)CCC]

### Compound 7: methyl((2S,3R,4S,5R)-3,4,5-tris(butyryloxy)tetrahydro-2H-pyran-2-yl) fumarate

### Preparation 1

### methyl((2S,3R,4S,5R)-3,4,5-tris(butyryloxy)tetrahydro-2H-pyran-2-yl) fumarate

To a solution of (3R,4S,5R)-tetrahydropyran-2,3,4,5-tetrol (10.00 g, 66.61 mmol, 1 equiv.) in pyridine (100 mL) was added butyric anhydride (84.30 g, 532.87 mmol, 87.17 mL, 8 equiv.). The mixture was stirred at 15° C. for 12 h. TLC indicated (3R,4S,5R)-tetrahydropyran-2,3,4,5-tetrol was consumed completely. The reaction mixture was concentrated under reduced pressure to give a residue. The residue was purified by column chromatography (SiO₂, petroleum ether/ethyl acetate=30/1 to 3/1). [(3R,4S,5R)-4,5,6-tri(butanoyloxy)tetrahydropyran-3-yl]butanoate (22 g, crude) was obtained as colorless liquid. To a solution of [(3R,4S,5R)-4,5,6-tri(butanoyloxy)tetrahydropyran-3-yl]butanoate (22 g, 51.10 mmol, 1 equiv.) in THF (150 mL) was added MeNH₂/H₂O (7.14 g, 91.99 mmol, 40% purity, 1.8 equiv.). The mixture was stirred at 15° C. for 12 h. LCMS showed the desired compound was detected. The reaction mixture was concentrated under reduced pressure to give a residue. The residue was purified by column chromatography (SiO₂, petroleum ether/ethyl acetate=30/1 to 3/1). Compound [(3R,4S,5R)-4,5-di(butanoyloxy)-6-hydroxy-tetrahydropyran-3-yl]butanoate (8 g, crude) was obtained as colorless oil. To a solution of [(3R,4S,5R)-4,5-di(butanoyloxy)-6-hydroxy-tetrahydropyran-3-yl]butanoate (4 g, 11.10 mmol, 1 equiv.), DCC (3.43 g, 16.65 mmol, 1.5 equiv.) and DMAP (406.78 mg, 3.33 mmol, 0.3 equiv.) in THF (50 mL) was added (E)-4-methoxy-4-oxo-but-2-enoic acid (2.17 g, 16.65 mmol, 1.5 equiv.). The mixture was stirred at 15° C. for 12 h. LCMS showed the desired compound was detected. The reaction mixture was filtered and concentrated under reduced pressure to give a residue. The residue was purified by prep-HPLC (water+0.05% (v/v) HCl/ACN) to give 2 g of the racemate as a black oil, which was further separated by SFC (0.1% NH₃, H₂O IPA). methyl((2S,3R,4S,5R)-3,4,5-tris(butyryloxy)tetrahydro-2H-pyran-2-yl) fumarate, 220 mg, 460.97 μmol, 21.78% yield, 99% purity) was obtained as a white solid. 1H NMR (400 MHz, methanol-d4): δ 6.85-6.64 (m, 2H), 5.78 (d, J=6.9 Hz, 1H), 5.23 (t, J=8.3 Hz, 1H), 5.05-4.84 (m, 2H), 4.05 (dd, J=11.9, 5.0 Hz, 1H), 3.71 (s, 3H), 3.55 (dd, J=12.0, 8.5 Hz, 1H), 2.19 (dtt, J=9.4, 5.1, 2.3 Hz, 6H), 1.61-1.39 (m, 6H), 0.91-0.66 (m, 9H) ppm. LCMS: (M+Na)⁺: 495.2.

### Preparation 2

To a solution of (2R,3R,4S,5R)-2-hydroxytetrahydro-2H-pyran-3,4,5-triyl tributyrate (500 mg, 1.39 mmol, 1 equiv.), DCC (429.38 mg, 2.08 mmol, 420.96 μL, 1.5 equiv.) and DMAP (50.85 mg, 416.21 μmol, 0.3 equiv.) in THF (10 mL) was added (E)-4-methoxy-4-oxo-but-2-enoic acid (270.74 mg, 2.08 mmol, 1.5 equiv.) and the mixture was stirred at 25° C. for 12 h. LCMS showed the starting reactant was consumed. The mixture reaction was concentrated. The residue was purified by prep-HPLC (water+10 mM NH₄HCO₃/ACN). to afford the title compound (104 mg, 18% yield). ¹H NMR (CDCl₃, 400 MHz): δ 6.8 (m, 2H), 5.8 (m, 1H), 5.3 (m, 3H), 4.0 (dd 2H), 3.7 (s, 3H), 2.2 (m, 6H), 1.6 (m, 6H), 0.9 (m, 9H) ppm. LCMS: (M+Na)⁺495.1.

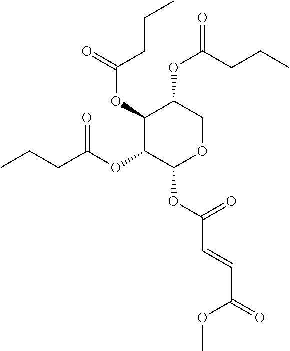 [SMILES: CCCC(=O)O[C@@H]1[C@@H](OC(=O)CCC)[C@@H](OC(=O)/C=C/C(=O)OC)OC[C@H]1OC(=O)CCC]

### Compound 8: methyl((2R,3R,4S,5R)-3,4,5-tris(butyryloxy)tetrahydro-2H-pyran-2-yl) fumarate

To a solution of (2S,3R,4S,5R)-2-hydroxytetrahydro-2H-pyran-3,4,5-triyl tributyrate (500 mg, 1.39 mmol, 1 equiv.), DCC (429.38 mg, 2.08 mmol, 420.96 μL, 1.5 equiv.) and DMAP (50.85 mg, 416.21 μmol, 0.3 equiv.) in THF (10 mL) was added (E)-4-methoxy-4-oxo-but-2-enoic acid (270.74 mg, 2.08 mmol, 1.5 equiv.) and the mixture was stirred at 25° C. for 12 h. LCMS showed the starting reactant was consumed. The mixture reaction was concentrated. The residue was purified by prep-HPLC (water+10 mM NH₄HCOS)/ACN). The title compound (206 mg, 414.20 μmol, 31% yield, 95% purity) was obtained as colorless oil. LCMS: (M+Na)⁺: 495.1 ¹H NMR (d4-methanol, 400 MHz): δ 6.9 (d, 2H), 6.4 (d, 1H), 5.3 (m, 3H), 4.2 (m, 1H), 3.8 (m, 4H), 2.4 (t, 3H), 2.2 (t, 3H), 1.6 m, 6H), 0.91 (m, 9H) ppm.

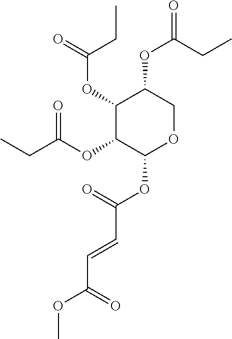 [SMILES: CCC(=O)O[C@H]1[C@@H](OC(=O)/C=C/C(=O)OC)OC[C@@H](OC(=O)CC)[C@H]1OC(=O)CC]

### Compound 9: methyl((2R,3R,4R,5R)-3,4,5-tris(propionyloxy)tetrahydro-2H-pyran-2-yl) fumarate

Compound 9 was synthesized in the same manner as compound 8. ¹H NMR (400 MHz, chloroform-d): δ 7.09-6.79 (m, 1H), 6.26 (d, J=3.8 Hz, 1H), 5.69 (t, J=3.3 Hz, 1H), 5.34-5.00 (m, 1H), 4.05 (t, J=10.7 Hz, 1H), 3.87 (s, 2H), 3.83-3.73 (m, 1H), 2.61-2.11 (m, 6H), 1.31-1.02 (m, 9H) ppm. LCMS: (M+Na)⁺: 453.1.

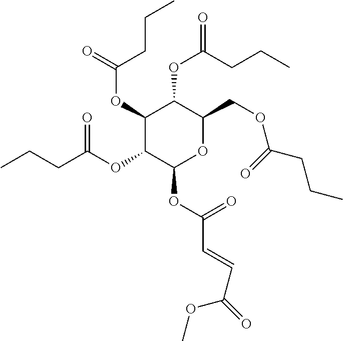 [SMILES: CCCC(=O)OC[C@H]1O[C@@H](OC(=O)/C=C/C(=O)OC)[C@H](OC(=O)CCC)[C@@H](OC(=O)CCC)[C@@H]1OC(=O)CCC]

### Compound 10: methyl((2S,3R,4S,5R,6R)-3,4,5-tris(butyryloxy)-6-((butyryloxy)methyl)tetrahydro-2H-pyran-2-yl) fumarate

D-(+)-glucose was dissolved to 0.5M in a mixture of dichloromethane and pyridine (50% mixture) and butyric anhydride (7 equiv.) was added to the solution at 0° C. The mixture was stirred at room temperature for 8 h. The mixture was neutralized with 1M HCl and purified by flash column chromatography. The resulting oil was dissolved in 0.1 M dry THF and treated with 1.5 eq of methyl amine in THF. The mixture was stirred at room temperature for 5 h, concentrated in vacuo and purified by column chromatography over silica gel using ethyl acetate-n-hexane (50/50) as eluent. The resulting viscus oil was dissolved in dry tetrahydrofuran (THF), and then dicyclohexylcarbodiimide (DCC) (1.2 equiv.), and (E)-4-methoxy-4-oxo-but-2-enoic acid (1.5 equiv.) was added to the solution at 0° C. The mixture was stirred at room temperature for 5 h. The resulting mixture was filtered and concentrated in vacuo. The crude product was purified by column chromatography over silica gel using ethyl acetate-n-hexane (30/70) as eluent to give the title compound as a waxy solid. ¹H NMR (400 MHz, Chloroform-d) δ 7.01-6.68 (m, 2H), 5.79 (d, J=8.1 Hz, 1H), 5.40-5.12 (m, 3H), 4.25 (dd, J=12.5, 4.7 Hz, 1H), 4.13 (dd, J=12.6, 2.2 Hz, 1H), 3.91-3.85 (m, 1H), 3.81 (s, 3H), 2.40-2.15 (m, 8H), 1.74-1.48 (m, 8H), 0.90 (ddt, J=17.5, 10.1, 7.4 Hz, 12H) ppm.

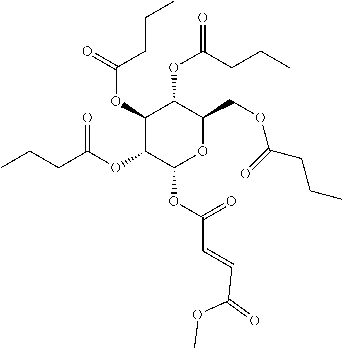 [SMILES: CCCC(=O)OC[C@H]1O[C@H](OC(=O)/C=C/C(=O)OC)[C@H](OC(=O)CCC)[C@@H](OC(=O)CCC)[C@@H]1OC(=O)CCC]

### Compound 11: methyl((2R,3R,4S,5R,6R)-3,4,5-tris(butyryloxy)-6-((butyryloxy)methyl)tetrahydro-2H-pyran-2-yl) fumarate

To a mixture of (2R,3R,4S,5R,6S)-2-((butyryloxy)methyl)-6-hydroxytetrahydro-2H-pyran-3,4,5-triyl tributyrate (1 g, 2.17 mmol, 1 equiv.) and (E)-4-methoxy-4-oxo-but-2-enoic acid (339.01 mg, 2.61 mmol, 1.2 equiv.) in THF (20 mL) was added DCC (896.07 mg, 4.34 mmol, 2 equiv.) and DMAP (132.64 mg, 1.09 mmol, 0.5 equiv.) in one portion at 20° C. under N₂. The mixture was stirred at 20° C. for 12 h. LCMS showed the starting reactant consumed completely. The reaction mixture was filtered and concentrated under reduced pressure to give a residue. The residue was purified by prep-HPLC (water+0.1% (v/v) TFA/ACN) to give 100 mg as a white solid, which was further separated by SFC (0.1% NH₃, H₂O, MeOH; B %: 20%-20%, 5 min). The title compound (0.030 g, 50.82 μmol, 2.34% yield, 97% purity) was obtained as colorless oil. LCMS: (M+Na)⁺: 595. ¹H NMR (CDCl₃, 400 MHz): 6.9 (m, 2H), 6.4 (s, 1H), 5.5 (m, 1H), 5.1 (m, 2H), 4.1 (m, 3H), 3.8, (s, 3H), 2.2 (m, 8H), 1.5 (m, 8H), 0.93 (m, 12H) ppm.

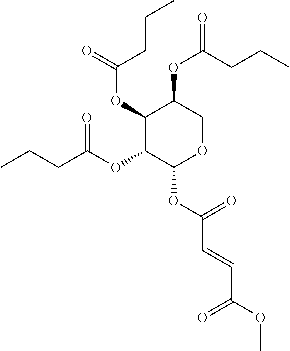 [SMILES: CCCC(=O)O[C@@H]1[C@@H](OC(=O)CCC)[C@@H](OC(=O)/C=C/C(=O)OC)OC[C@@H]1OC(=O)CCC]

### Compound 12: methyl((2R,3R,4S,5S)-3,4,5-tris(butyryloxy)tetrahydro-2H-pyran-2-yl) fumarate

To a solution of (2S,3R,4S,5S)-tetrahydro-2H-pyran-2,3,4,5-tetraol (3 g, 19.98 mmol, 1 equiv.) in pyridine (30 mL) was added butanoyl butanoate (25.29 g, 159.86 mmol, 26.15 mL, 8 equiv.) at 25° C. The mixture was stirred at 25° C. for 12 h. LCMS showed the desired compound was detected. The reaction mixture was concentrated under reduced pressure to give a residue. The residue was purified by column chromatography (SiO₂, petroleum ether/ethyl acetate=10/1 to 3/1). The compound (2R,3R,4S,5S)-tetrahydro-2H-pyran-2,3,4,5-tetrayl tetrabutyrate (8 g, 18.58 mmol, 93.00% yield) was obtained as colorless oil.

To a solution of (2R,3R,4S,5S)-tetrahydro-2H-pyran-2,3,4,5-tetrayl tetrabutyrate (8 g, 18.58 mmol, 1 equiv.) in THF (100 mL) was added MeNH₂ aq. (2.74 g, 35.31 mmol, 40% purity, 1.9 equiv.) at 25° C. The mixture was stirred at 25° C. for 12 hr. TLC indicated new spot formed. The reaction mixture was concentrated under reduced pressure to give a residue. The residue was purified by column chromatography (SiO₂, petroleum ether/ethyl acetate=10/1 to 1/1). The crude product (2S,3R,4S,5S)-2-hydroxytetrahydro-2H-pyran-3,4,5-triyl tributyrate (4 g, 9.43 mmol, 50.77% yield, 85% purity) was obtained as a yellow oil.

To a solution of (2S,3R,4S,5S)-2-hydroxytetrahydro-2H-pyran-3,4,5-triyl tributyrate (600 mg, 1.66 mmol, 1 equiv.) in DCM (5 mL) was added (E)-4-methoxy-4-oxo-but-2-enoic acid (324.89 mg, 2.50 mmol, 1.5 equiv.), DCC (515.25 mg, 2.50 mmol, 505.15 μL, 1.5 equiv.) and DMAP (101.70 mg, 832.41 μmol, 0.5 equiv.) at 25° C. The mixture was stirred at 25° C. for 12 hr. LCMS showed the desired compound was detected. The reaction mixture was filtered and concentrated under reduced pressure to give a residue. The residue was purified by prep-HPLC (column: Waters Xbridge Prep OBD C18 150×40 10 μm; mobile phase: (water+10 mM NH₄HCO₃/ACN); B %: 45%-70%, 11 min) to give a residue. The residue was further purified by prep-HPLC (column: Xtimate C18 150×25 mm Sum; mobile phase: (water+10 mM NH₄HCO₃/ACN); B %: 50%-80%, 10 min). And then the product was separated by SFC (column: DAICEL CHIRALCEL OD-H 250 mm×30 mm Sum; mobile phase: 0.1% NH₃, H₂O, MeOH; B %: 20%-20%, 1.5 min) to give the title compound (26 mg, 55.03 μmol, 21.67% yield) as a yellow oil. LCMS: (M+18)⁺490.2. ¹H NMR (d4-methanol, 400 MHz): δ 7.0 (m, 1H), 6.4 (m, 1H), 5.3 (m, 3H), 4.2 (m, 1H), 3.8 (m, 3H), 2.4 (m, 6H), 1.5 (m, 6H), 0.9 (m, 9H) ppm.

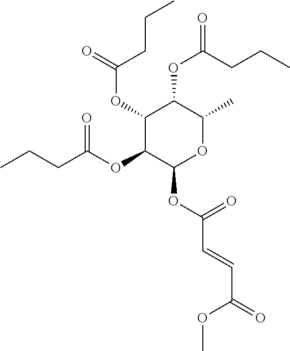 [SMILES: CCCC(=O)O[C@H]1[C@H](OC(=O)CCC)[C@H](OC(=O)/C=C/C(=O)OC)O[C@@H](C)[C@H]1OC(=O)CCC]

### Compound 13: methyl((2S,3S,4R,5R,6S)-3,4,5-tris(butyryloxy)-6-methyltetrahydro-2H-pyran-2-yl) fumarate

To a solution of (2R,3S,4R,5S,6S)-6-methyltetrahydro-2H-pyran-2,3,4,5-tetraol (3.00 g, 18.28 mmol, 1 equiv.) in pyridine (30 mL) was added butanoyl butanoate (17.35 g, 109.68 mmol, 17.94 mL, 6 equiv.) at 25° C. The mixture was stirred at 25° C. for 12 hr. LCMS showed the desired compound was detected. The reaction mixture was concentrated under reduced pressure to give a residue. The reaction mixture was diluted with H₂O (30 mL) and extracted with ethyl acetate 60 mL (20 mL×3). The combined organic layers were washed with brine 20 mL, dried over anhydrous sodium sulfate, filtered and concentrated under reduced pressure to give a residue. Compound (2S,3S,4R,5R,6S)-6-methyltetrahydro-2H-pyran-2,3,4,5-tetrayl tetrabutyrate (8 g, crude) was obtained as colorless oil.

To a solution of (2S,3S,4R,5R,6S)-6-methyltetrahydro-2H-pyran-2,3,4,5-tetrayl tetrabutyrate (8 g, 18.00 mmol, 1 equiv.) in THF (100 mL) was added MeNH₂ aq. (2.66 g, 34.19 mmol, 40% purity, 1.9 equiv.) at 25° C. The mixture was stirred at 25° C. for 12 hr. TLC indicated new spot formed. The reaction mixture was concentrated under reduced pressure to give a residue. The residue was purified by column chromatography (SiO₂, Petroleum ether/Ethyl acetate=10/1). Compound (2R,3S,4R,5R,6S)-2-hydroxy-6-methyltetrahydro-2H-pyran-3,4,5-triyl tributyrate (5 g, crude) was obtained as yellow oil.

To a solution of (2R,3S,4R,5R,6S)-2-hydroxy-6-methyltetrahydro-2H-pyran-3,4,5-triyl tributyrate (500.00 mg, 1.34 mmol, 1 equiv.) in DCM (5 mL) was added DCC (413.29 mg, 2.00 mmol, 405.18 μL, 1.5 equiv.), DMAP (81.57 mg, 667.69 μmol, 0.5 equiv.) and (E)-4-methoxy-4-oxo-but-2-enoic acid (260.60 mg, 2.00 mmol, 1.5 equiv.) at 25° C. The mixture was stirred at 25° C. for 12 hr. LCMS showed the desired compound was detected. The reaction mixture was filtered and concentrated under reduced pressure to give a residue. The residue was purified by prep-HPLC (column: Xtimate C18 150×25 mm 5 μm; mobile phase: water+10 mM NH₄HCO₃/ACN; B %: 65%-80%, 10 min) to give a residue. The residue was purified by SFC (column: DAICEL CHIRALPAK AD-H 250 mm×30 mm, 5 μm); mobile phase: 0.1% NH₃, H₂O, IPA; B %: 15%-15%, 2 min) to give residue (62 mg, 121.95 μmol, 29.66% yield, 95.69% purity) as a yellow solid. The residue was purified by prep-HPLC (column: HUAPU C8 Extreme BDS 150×30 5 μm; mobile phase: water+10 mM NH₄HCO₃/ACN; B %: 55%-75%, 10 min). The title compound (23 mg, 45.24 μmol, 35.50% yield, 95.69% purity) was obtained as a colorless oil. ¹H NMR (CDCl₃, 400 MHz): δ 6.9 (s, 2H), 6.4 (m, 1H), 5.4 (m, 3H), 4.3 (m, 1H), 3.8 (s, 3H), 2.4 (m, 2H), 2.2 (m, 4H), 1.7 (m, 2H), 1.5 (m, 8H), 1.0 (d, 3H), 0.9 (m, 9H) ppm. LCMS: (M+18)⁺504.3.

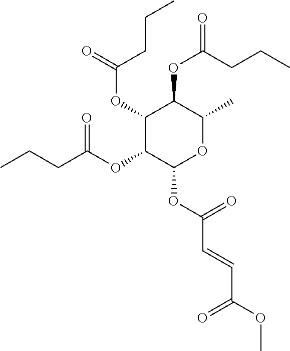 [SMILES: CCCC(=O)O[C@@H]1[C@@H](OC(=O)CCC)[C@H](C)O[C@H](OC(=O)/C=C/C(=O)OC)[C@@H]1OC(=O)CCC]

### Compound 14: methyl((2R,3R,4R,5S,6S)-3,4,5-tris(butyryloxy)-6-methyltetrahydro-2H-pyran-2-yl) fumarate

To a solution of (2S,3R,4R,5R,6S)-6-methyltetrahydro-2H-pyran-2,3,4,5-tetraol (1 g, 6.09 mmol, 1 equiv.) in pyridine (10 mL) was added butanoyl butanoate (5.78 g, 36.55 mmol, 5.98 mL, 6 equiv.) at 25° C. The mixture was stirred at 25° C. for 12 h. Spots on a thin layer chromatogram (TLC) indicated formation of a new compound. The reaction mixture was concentrated under reduced pressure to give a residue. The reaction mixture was diluted with saturated sodium bicarbonate solution (40 mL) and extracted with ethyl acetate (40 mL). The combined organic layers were washed with brine (20 mL), dried over anhydrous sodium sulfate, filtered and concentrated under reduced pressure to give a residue. (2R,3R,4R,5S,6S)-6-methyltetrahydro-2H-pyran-2,3,4,5-tetrayl tetrabutyrate (3.5 g, crude) was obtained as yellow oil.

To a solution of (2R,3R,4R,5S,6S)-6-methyltetrahydro-2H-pyran-2,3,4,5-tetrayl tetrabutyrate (3.5 g, 7.87 mmol, 1 equiv.) in THF (20 mL) was added MeNH₂ aq. (1.10 g, 14.17 mmol, 40% purity, 1.8 equiv.) at 25° C. The mixture was stirred at 25° C. for 12 hr. Spots on a thin layer chromatogram (TLC) indicated formation of a new compound. The reaction mixture was concentrated under reduced pressure to give a residue. The residue was purified by column chromatography (SiO₂, Petroleum ether/ethyl acetate=10/1 to 1/1). Compound (2S,3R,4R,5S,6S)-2-hydroxy-6-methyltetrahydro-2H-pyran-3,4,5-triyl tributyrate (1.8 g, 4.81 mmol, 61.06% yield) was obtained as yellow oil.

To a solution of (2S,3R,4R,5S,6S)-2-hydroxy-6-methyltetrahydro-2H-pyran-3,4,5-triyl tributyrate (600 mg, 1.60 mmol, 1 equiv.) in DCM (10 mL) was added DCC (495.95 mg, 2.40 mmol, 486.23 μL, 1.5 equiv.), DMAP (97.89 mg, 801.23 μmol, 0.5 equiv.) and (E)-4-methoxy-4-oxo-but-2-enoic acid (312.72 mg, 2.40 mmol, 1.5 equiv.) at 25° C. The mixture was stirred at 25° C. for 5 hr. LCMS showed the desired compound was detected. The reaction mixture was filtered and concentrated under reduced pressure to give a residue. The residue was purified by prep-HPLC (column: Waters Xbridge Prep OBD C18 150×40 μm; mobile phase: water+10 mM NH₄HCO₃/ACN; B %: 50%-75%, 11 min) to provide the title compound (70 mg, crude) as a yellow solid, that was further purified by prep-TLC (SiO₂, petroleum ether/ethyl acetate=3:1) to give the purified title compound (25 mg, 47.79 μmol, 33.21% yield, 93% purity) as colorless oil. ¹H NMR (CDCl₃, 400 MHz): δ 6.95.1 (m, 2H), 3.8 (s, 3H), 3.6 (m, 1H) 2.4 (t, 2H), 2.2 (m, 4H) 1.6 (m, 6H), 1.3 (d, 3H), 1.0 (m, 9H) ppm. LCMS: (M+18)⁺: 504.2.

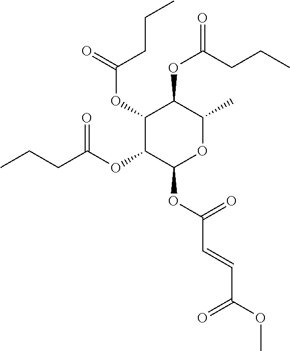 [SMILES: CCCC(=O)O[C@@H]1[C@@H](OC(=O)CCC)[C@H](C)O[C@@H](OC(=O)/C=C/C(=O)OC)[C@@H]1OC(=O)CCC]

### Compound 15: methyl((2S,3R,4R,5S,6S)-3,4,5-tris(butyryloxy)-6-methyltetrahydro-2H-pyran-2-yl) fumarate

To a solution of (2R,3R,4R,5R,6S)-6-methyltetrahydro-2H-pyran-2,3,4,5-tetraol (1 g, 6.09 mmol, 1 equiv.) in pyridine (10 mL) was added butanoyl butanoate (5.78 g, 36.55 mmol, 5.98 mL, 6 equiv.) at 25° C. The mixture was stirred at 25° C. for 12 hr. Spots on a thin layer chromatogram (TLC) indicated formation of a new compound. The reaction mixture was concentrated under reduced pressure to give a residue. The reaction mixture was diluted with saturated sodium bicarbonate solution (40 mL) and extracted with ethyl acetate (40 mL). The combined organic layers were washed with brine (20 mL), dried over anhydrous sodium sulfate, filtered and concentrated under reduced pressure to give (2S,3R,4R,5S,6S)-6-methyltetrahydro-2H-pyran-2,3,4,5-tetrayl tetrabutyrate (3.5 g, crude) as yellow oil.

To a solution of (2S,3R,4R,5S,6S)-6-methyltetrahydro-2H-pyran-2,3,4,5-tetrayl tetrabutyrate (3.5 g, 7.87 mmol, 1 equiv.) in THF (20 mL) was added MeNH₂ aq. (1.10 g, 14.17 mmol, 40% purity, 1.8 equiv.) at 25° C. The mixture was stirred at 25° C. for 12 hr. Spots on a thin layer chromatogram (TLC) indicated formation of a new compound. The reaction mixture was concentrated under reduced pressure to give a residue. The residue was purified by column chromatography (SiO₂, petroleum ether/ethyl acetate=10/1 to 1/1). (2R,3R,4R,5S,6S)-2-hydroxy-6-methyltetrahydro-2H-pyran-3,4,5-triyl tributyrate (1.8 g, 4.81 mmol, 61.06% yield) was obtained as yellow oil.

To a solution of (2R,3R,4R,5S,6S)-2-hydroxy-6-methyltetrahydro-2H-pyran-3,4,5-triyl tributyrate (600 mg, 1.60 mmol, 1 equiv.) in DCM (10 mL) was added DCC (495.95 mg, 2.40 mmol, 486.23 μL, 1.5 equiv.), DMAP (97.89 mg, 801.23 μmol, 0.5 equiv.) and (E)-4-methoxy-4-oxo-but-2-enoic acid (312.72 mg, 2.40 mmol, 1.5 equiv.) at 25° C. The mixture was stirred at 25° C. for 5 hr. LCMS showed the desired compound was detected. The reaction mixture was filtered and concentrated under reduced pressure to give a residue. The residue was purified by prep-HPLC (column: Waters Xbridge Prep OBD C18 150×40 μm; mobile phase: water+10 mM NH₄HCO₃/ACN; B %: 50%-75%, 11 min) to afford the title compound (210 mg, 353.95 μmol, 22.09/yield, 82% purity) as a yellow solid. LCMS: (M+18)⁺: 504.2. ¹H NMR (CDCl₃, 400 MHz): δ 6.9, (m, 2H), 6.1 (s, 1H), 5.4 m, 2H), 5.2 (m, 1H), 3.9 (m, 1H), 3.8 (s, 3H), 2.2 (m, 6H), 1.6 (m, 6H), 1.2 (m, 3H), 0.9 (m, 9H) ppm.

Compound 15-d15 was synthesized in a similar manner as described herein, with the exception that d5-butyric acid was used in combination with, e.g., the EDCI coupling conditions.

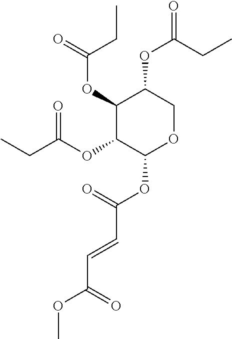 [SMILES: CCC(=O)O[C@@H]1[C@@H](OC(=O)CC)[C@@H](OC(=O)/C=C/C(=O)OC)OC[C@H]1OC(=O)CC]

### Compound 16: methyl((2R,3R,4S,5R)-3,4,5-tris(propionyloxy)tetrahydro-2H-pyran-2-yl) fumarate

A mixture of (2S,3R,4S,5R)-tetrahydro-2H-pyran-2,3,4,5-tetraol (10 g, 66.61 mmol, 1 equiv.) and propanoyl propanoate (52.01 g, 399.65 mmol, 51.50 mL, 6 equiv.) in pyridine (50 mL) was stirred at 25° C. for 12 h. TLC indicated (2S,3R,4S,5R)-tetrahydro-2H-pyran-2,3,4,5-tetraol was consumed completely and two new spots formed. The reaction mixture was concentrated under reduced pressure to give a residue. Then, the reaction mixture was diluted with H₂O (25 mL) and extracted with EtOAc (10 mL×4). The combined organic layers were washed with brine (10 mL), dried over Na₂SO₄, filtered and concentrated under reduced pressure to give a residue. The residue was purified by column chromatography (SiO₂, petroleum ether/ethyl acetate=10/1 to 5/1). (2R,3R,4S,5R)-tetrahydro-2H-pyran-2,3,4,5-tetrayl tetrapropionate (20 g, 53.42 mmol, 80.20/yield) was obtained as yellow oil.

To a solution of (2R,3R,4S,5R)-tetrahydro-2H-pyran-2,3,4,5-tetrayl tetrapropionate (10 g, 26.71 mmol, 1 equiv.) in THF (100 mL) was added MeNH₂aq. (3.73 g, 48.08 mmol, 40% purity, 1.8 equiv.). The mixture was stirred at 25° C. for 12 h under N₂. TLC indicated starting material was consumed completely and one new spot formed. The reaction mixture was diluted with H₂O (25 mL) and extracted with EtOAc (10 mL×4). The combined organic layers were washed with brine (10 mL), dried over Na₂SO₄, filtered and concentrated under reduced pressure to give a residue. The residue was purified by column chromatography (SiO₂, petroleum ether/ethyl acetate=10/1 to 5/1). (2S,3R,4S,5R)-2-hydroxytetrahydro-2H-pyran-3,4,5-triyl tripropionate (6 g, 18.85 mmol, 70.57% yield) was obtained as a yellow oil.

To a solution of (2S,3R,4S,5R)-2-hydroxytetrahydro-2H-pyran-3,4,5-triyl tripropionate (5 g, 15.71 mmol, 1 equiv.) and (E)-4-methoxy-4-oxo-but-2-enoic acid (3.07 g, 23.56 mmol, 1.5 equiv.) in DCM (50 mL) was added DCC (4.86 g, 23.56 mmol, 4.77 mL, 1.5 equiv.) and DMAP (575.69 mg, 4.71 mmol, 0.3 equiv.). The mixture was stirred at 25° C. for 12 hr. LC-MS showed starting material (5 g, 15.71 mmol, 1 equiv.) was consumed completely and the desired m/z was detected. The reaction mixture was diluted with H₂O (15 mL) and extracted with EtOAc (5 mL×4). The combined organic layers were washed with brine (5 mL), dried over Na₂SO₄, filtered and concentrated under reduced pressure to give a residue. The residue was purified by prep-HPLC (column: Phenomenex Luna C18 200×40 mm 10 μm; mobile phase: water+0.1% (v/v) TFA/ACN; B %: 50%-70%, 10 min). Then the residue was separated by SFC (column: DAICEL CHIRALPAK AD-H 250 mm×30 mm, 5 μm; mobile phase: 0.1% NH₃, H₂O, EtOH; B %: 15%-15%, 3.1 min). The title compound (46 mg, 101.00 μmol, 0.643% yield) was obtained as yellow oil. LCMS: (M+Na)⁺: 453.1. ¹H NMR (CDCl₃, 400 MHz): δ 6.9 (m, 2H), 6.2 (m, 1H), 5.4 (m, 1H), 5.1 (m, 2H), 3.7 (m, 5H), 2.2 (m, 8H), 0.9 (m, 12H) ppm.

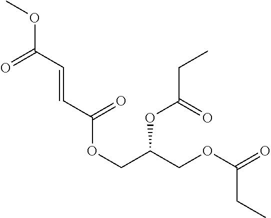 [SMILES: CCC(=O)OC[C@H](COC(=O)/C=C/C(=O)OC)OC(=O)CC]

### Compound 17: (R)-2,3-bis(propionyloxy)propyl methyl fumarate

To a solution of (S)-(2,2-dimethyl-1,3-dioxolan-4-yl)methanol (5 g, 37.83 mmol, 4.67 mL, 1 equiv.) in DCM (50 mL) was added (E)-4-methoxy-4-oxo-but-2-enoic acid (7.38 g, 56.75 mmol, 1.5 equiv.), DCC (11.71 g, 56.75 mmol, 11.48 mL, 1.5 equiv.) and DMAP (2.31 g, 18.92 mmol, 0.5 equiv.) at 25° C. The mixture was stirred at 25° C. for 2 h. Spots on a thin layer chromatogram (TLC) indicated formation of a new compound. The reaction mixture was filtered and the filtrate was concentrated under reduced pressure to give a residue. The residue was purified by column chromatography (SiO₂, petroleum ether/ethyl acetate=10/1). Compound (R)-(2,2-dimethyl-1,3-dioxolan-4-yl)methyl methyl fumarate (8 g, 32.75 mmol, 86.58% yield) was obtained as a white solid.

To a solution of (R)-(2,2-dimethyl-1,3-dioxolan-4-yl)methyl methyl fumarate (500 mg, 2.05 mmol, 1 equiv.) in MeOH (5 mL) was added p-TsOH (60 mg, 348.43 μmol, 0.17 equiv.) at 0° C. The mixture was stirred at 50° C. for 2 h. Spots on a thin layer chromatogram (TLC) indicated formation of a new compound. The reaction mixture was concentrated under reduced pressure to give a residue. The residue was purified by column chromatography (SiO₂, petroleum ether/ethyl acetate=3/1 to 0/1). (R)-2,3-dihydroxypropyl methyl fumarate (330 mg, 1.62 mmol, 78.95% yield) was obtained as a white solid.

To a solution of (R)-2,3-dihydroxypropyl methyl fumarate (330 mg, 1.62 mmol, 1 equiv.) in pyridine (5 mL) was added propanoyl propanoate (841.37 mg, 6.46 mmol, 833.03 μL, 4 equiv.) at 25° C. The mixture was stirred at 25° C. for 12 h. Spots on a thin layer chromatogram (TLC) indicated formation of a new compound. The reaction mixture was concentrated under reduced pressure to give a residue. The residue was purified by column chromatography (SiO₂, petroleum ether/ethyl acetate=I/O to 0/1). The title compound (270 mg, 828.00 μmol, 51.23% yield, 97% purity) was obtained as yellow oil. ¹H NMR (CDCl₃, 400 MHz): δ 6.8 (m, 2H), 5.3 (m, 1H), 4.4 (m, 1H), 4.3 (m, 2H), 3.8 (m, 1H), 3.8 (s, 3H), 2.3 (m, 4H) 1.1 (t, 3H) ppm. LCMS: (M+18)⁺: 334.1.

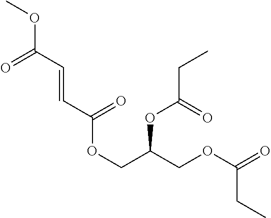 [SMILES: CCC(=O)OC[C@@H](COC(=O)/C=C/C(=O)OC)OC(=O)CC]

### Compound 18: (S)-2,3-bis(propionyloxy)propyl methyl fumarate

To a solution of (R)-(2,2-dimethyl-1,3-dioxolan-4-yl)methanol (5 g, 37.83 mmol, 186.92 μL, 1 equiv.) in DCM (50 mL) was added (E)-4-methoxy-4-oxo-but-2-enoic acid (7.38 g, 56.75 mmol, 1.5 equiv.), DCC (11.71 g, 56.75 mmol, 11.48 mL, 1.5 equiv.) and DMAP (2.31 g, 18.92 mmol, 0.5 equiv.) at 25° C. The mixture was stirred at 25° C. for 2 h. Spots on a thin layer chromatogram (TLC) indicated formation of a new compound. The reaction mixture was filtered and the filtrate was concentrated under reduced pressure to give a residue. The residue was purified by column chromatography (SiO₂, petroleum ether/ethyl acetate=10/1). (S)-(2,2-dimethyl-1,3-dioxolan-4-yl)methyl methyl fumarate (7.29 g, 29.85 mmol, 78.89% yield) was obtained as a white solid.

To a solution of (S)-(2,2-dimethyl-1,3-dioxolan-4-yl)methyl methyl fumarate (2.00 g, 8.19 mmol, 1 equiv.) in MeOH (30 mL) was added p-TsOH (200 mg, 1.16 mmol, 1.42e-1 equiv.) at 0° C. The mixture was stirred at 50° C. for 3 hr. Spots on a thin layer chromatogram (TLC) indicated formation of a new compound. The reaction mixture was concentrated under reduced pressure to give a residue. The residue was purified by column chromatography (SiO₂, petroleum ether/ethyl acetate=2:1 to 0:1). (S)-2,3-dihydroxypropyl methyl fumarate (1 g, 4.90 mmol, 59.81% yield) was obtained as a white solid.

To a solution of (S)-2,3-dihydroxypropyl methyl fumarate (500 mg, 2.45 mmol, 1 equiv.) in pyridine (10 mL) was added propanoyl propanoate (1.27 g, 9.80 mmol, 1.26 mL, 4 equiv.) at 25° C. The mixture was stirred at 25° C. for 12 h. Spots on a thin layer chromatogram (TLC) indicated formation of a new compound. The reaction mixture was concentrated under reduced pressure to give a residue. The residue was purified by column chromatography (SiO₂, petroleum ether/ethyl acetate=10/1 to 0/1). The title compound (655 mg, 1.99 mmol, 81.18% yield, 96% purity) was obtained as yellow oil. ¹H NMR (CDCl₃, 400 MHz): δ 6.8 (m, 2H), 5.3 (m, 1H), 4.4 (m, 1H), 4.3 (m, 2H), 3.8 (m, 1H), 3.8 (s, 3H), 2.3 (q, 4H) 1.1 (t, 3H) ppm. LCMS: (M+18)⁺: 334.1.

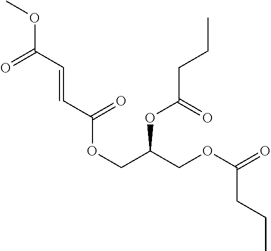 [SMILES: CCCC(=O)OC[C@@H](COC(=O)/C=C/C(=O)OC)OC(=O)CCC]

### Compound 19: (S)-2,3-bis(butyryloxy)propyl methyl fumarate

To a solution of (R)-(2,2-dimethyl-1,3-dioxolan-4-yl)methanol (5 g, 37.83 mmol, 186.92 μL, 1 equiv.) in DCM (50 mL) was added (E)-4-methoxy-4-oxo-but-2-enoic acid (7.38 g, 56.75 mmol, 1.5 equiv.), DCC (11.71 g, 56.75 mmol, 11.48 mL, 1.5 equiv.) and DMAP (2.31 g, 18.92 mmol, 0.5 equiv.) at 25° C. The mixture was stirred at 25° C. for 2 h. Spots on a thin layer chromatogram (TLC) indicated formation of a new compound. The reaction mixture was filtered and the filtrate was concentrated under reduced pressure to give a residue. The residue was purified by column chromatography (SiO₂, petroleum ether/ethyl acetate=10:1) to yield (S)-(2,2-dimethyl-1,3-dioxolan-4-yl)methyl methyl fumarate (7.29 g, 29.85 mmol, 78.89% yield) as a white solid.

To a solution (S)-(2,2-dimethyl-1,3-dioxolan-4-yl)methyl methyl fumarate (2.00 g, 8.19 mmol, 1 equiv.) in MeOH (30 mL) was added p-TsOH (200 mg, 1.16 mmol, 1.42e-1 equiv.) at 0° C. The mixture was stirred at 50° C. for 3 hr. Spots on a thin layer chromatogram (TLC) indicated formation of a new compound. The reaction mixture was concentrated under reduced pressure to give a residue. The residue was purified by column chromatography (SiO₂, petroleum ether/ethyl acetate=2:1 to 0:1). (S)-2,3-dihydroxypropyl methyl fumarate (1 g, 4.90 mmol, 59.81% yield) was obtained as white solid.

To a solution of (S)-2,3-dihydroxypropyl methyl fumarate (500 mg, 2.45 mmol, 1 equiv.) in pyridine (6 mL) was added butanoyl butanoate (1.55 g, 9.80 mmol, 1.60 mL, 4 equiv.) at 25° C. The mixture was stirred at 25° C. for 12 h. Spots on a thin layer chromatogram (TLC) indicated formation of a new compound. The reaction mixture was concentrated under reduced pressure to give a residue. The residue was purified by column chromatography (SiO₂, petroleum ether/ethyl acetate=10/0 to 0/1). The title compound (606 mg, 1.69 mmol, 68.92% yield, 95.9/6 purity) was obtained as yellow oil. ¹H NMR (CDCl₃, 400 MHz): δ 6.8 (m, 2H), 5.3 (m, 1H), 4.4 (m, 1H), 4.3 (m, 2H), 4.1 (m, 1H), 3.8 (s, 3H), 2.3 (m, 4H), 1.6 (m, 4H), 1.1 (t, 3H) ppm. LCMS: (M+18): 334.1.

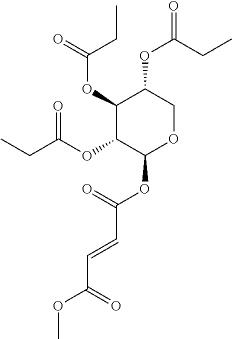 [SMILES: CCC(=O)O[C@@H]1[C@@H](OC(=O)CC)[C@H](OC(=O)/C=C/C(=O)OC)OC[C@H]1OC(=O)CC]

### Compound 20: methyl((2S,3R,4S,5R)-3,4,5-tris(propionyloxy)tetrahydro-2H-pyran-2-yl) fumarate

A mixture of (2R,3R,4S,5R)-tetrahydro-2H-pyran-2,3,4,5-tetraol (10 g, 66.61 mmol, 1 equiv.) and propanoyl propanoate (52.01 g, 399.65 mmol, 51.50 mL, 6 equiv.) in pyridine (50 mL) was stirred at 25° C. for 12 h. TLC indicated starting material was consumed completely and two new spots formed. The reaction mixture was concentrated under reduced pressure to give a residue. Then, the reaction mixture was diluted with H₂O (25 mL) and extracted with EtOAc (10 mL×4). The combined organic layers were washed with brine (10 mL), dried over Na₂SO₄, filtered and concentrated under reduced pressure to give a residue. The residue was purified by column chromatography (SiO₂, petroleum ether/ethyl acetate=10/1 to 5/1). (2S,3R,4S,5R)-tetrahydro-2H-pyran-2,3,4,5-tetrayl tetrapropionate (20 g, 53.42 mmol, 80.20/6 yield) was obtained as yellow oil.

To a solution of (2S,3R,4S,5R)-tetrahydro-2H-pyran-2,3,4,5-tetrayl tetrapropionate (10 g, 26.71 mmol, 1 equiv.) in THF (100 mL) was added MeNH₂aq. (3.73 g, 48.08 mmol, 40% purity, 1.8 equiv.). The mixture was stirred at 25° C. for 12 h under N₂. TLC indicated starting material was consumed completely and one new spot formed. The reaction mixture was diluted with H₂O (25 mL) and extracted with EtOAc (10 mL×4). The combined organic layers were washed with brine (10 mL), dried over Na₂SO₄, filtered and concentrated under reduced pressure to give a residue. The residue was purified by column chromatography (SiO₂, petroleum ether/ethyl acetate=10/1 to 5/1). (2R,3R,4S,5R)-2-hydroxytetrahydro-2H-pyran-3,4,5-triyl tripropionate (6 g, 18.85 mmol, 70.57% yield) was obtained as a yellow oil.

To a solution of (2R,3R,4S,5R)-2-hydroxytetrahydro-2H-pyran-3,4,5-triyl tripropionate (5 g, 15.71 mmol, 1 equiv.) and (E)-4-methoxy-4-oxo-but-2-enoic acid (3.07 g, 23.56 mmol, 1.5 equiv.) in DCM (50 mL) was added DCC (4.86 g, 23.56 mmol, 4.77 mL, 1.5 equiv.) and DMAP (575.69 mg, 4.71 mmol, 0.3 equiv.). The mixture was stirred at 25° C. for 12 h. The desired m/z was detected by LC-MS. The reaction mixture was diluted with H₂O (15 mL) and extracted with EtOAc (5 mL×4). The combined organic layers were washed with brine (5 mL), dried over Na₂SO₄, filtered and concentrated under reduced pressure to give a residue. The residue was purified by prep-HPLC (column: Phenomenex Luna C18 200×40 mm 10 μm; mobile phase: water+0.1% (v/v) TFA/ACN; B %: 50%-70%, 10 min). Then the residue was separated by SFC (column: DAICEL CHIRALPAK AD-H 250 mm×30 mm, 5 μm; B %: 15%-15%, 3.1 min). The title compound (30 mg, 53.67 μmol, 0.342/6 yield) was obtained as white solid. LCMS: (M+Na): 453.1. ¹H NMR (d6-DMSO, 400 MHz): δ 6.8 (m, 2H), 5.9 (m, 1H), 5.3 (m, 1H), 4.9 (m, 2H), 4.0 (m, 1H), 3.7 (m, 4H), 2.2 (m, 8H), 0.9 (m, 12H) ppm.

Compound 20-d9 was synthesized in a similar manner as described herein, with the exception that d3-propionic acid was used in combination with, e.g., the EDCI coupling conditions.

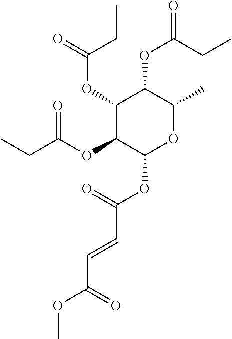 [SMILES: CCC(=O)O[C@H]1[C@H](OC(=O)CC)[C@@H](OC(=O)/C=C/C(=O)OC)O[C@@H](C)[C@H]1OC(=O)CC]

### Compound 21: methyl((2R,3S,4R,5R,6S)-6-methyl-3,4,5-tris(propionyloxy)tetrahydro-2H-pyran-2-yl) fumarate

L-fucopyranose was dissolved to form a 0.5 M mixture of dichloromethane and pyridine (50% mixture), and then propionic anhydride (6 equiv.) was added to the solution at 0° C. The mixture was stirred at room temperature for 8 h. The resulting mixture was neutralized with 1 M HCl and purified by flash column chromatography. The resulting oil was dissolved in 0.1 M dry THF and treated with 1.5 equiv. of methyl amine in THF. The mixture was stirred at room temperature for 5 h, concentrated in vacuo and was purified by column chromatography over silica gel using ethyl acetate-n-hexane (50:50) as eluent. The resulting viscus oil was dissolved in dry tetrahydrofuran (THF), and then dicyclohexylcarbodiimide (DCC, 1.2 equiv.) was added to the solution at 0° C. The mixture was stirred at room temperature for 5 h. The resulting mixture was filtered and concentrated in vacuo. The crude product was purified by column chromatography over silica gel using ethyl acetate-n-hexane (40/60) as eluent to give methyl((2R,3S,4R,5R,6S)-6-methyl-3,4,5-tris(propionyloxy)tetrahydro-2H-pyran-2-yl) fumarate as a waxy solid. ¹H NMR (400 MHz, chloroform-d) δ 6.98-6.77 (m, 1H), 5.77 (d, J=8.3 Hz, 1H), 5.40 (dd, J=10.4, 8.3 Hz, 1H), 5.31 (dd, J=3.5, 1.1 Hz, 1H), 5.13 (dd, J=10.4, 3.4 Hz, 1H), 4.07-3.96 (m, 1H), 2.49 (qd, J=7.7, 3.3 Hz, 1H), 2.33-2.18 (m, 2H), 1.32-1.17 (m, 6H), 1.08 (td, J=7.6, 3.5 Hz, 4H) ppm.

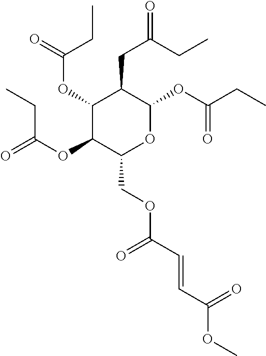 [SMILES: CCC(=O)C[C@H]1[C@H](OC(=O)CC)O[C@H](COC(=O)/C=C/C(=O)OC)[C@@H](OC(=O)CC)[C@@H]1OC(=O)CC]

### Compound 22: methyl(((2R,3R,4S,5R,6S)-3,4,5,6-tetrakis(propionyloxy)tetrahydro-2H-pyran-2-yl)methyl) fumarate

A mixture of (3R,4S,5S,6R)-6-(hydroxymethyl)tetrahydropyran-2,3,4,5-tetrol (20 g, 111.01 mmol, 1 equiv.) and [chloro(diphenyl)methyl]benzene (30.95 g, 111.01 mmol, 1 equiv.) in pyridine (100 mL) was degassed and purged with N₂₃ times. Then the mixture was stirred at 15° C. for 10 h under N₂ atmosphere. TLC indicated (3R,4S,5S,6R)-6-(hydroxymethyl)tetrahydropyran-2,3,4,5-tetrol was consumed completely and three new spots formed. (3R,4S,5S,6R)-6-(trityloxymethyl)tetrahydropyran-2,3,4,5-tetrol (˜111 mmol) as a crude solution in pyridine was used directly in the next step.

To the above solution of (3R,4S,5S,6R)-6-(trityloxymethyl)tetrahydropyran-2,3,4,5-tetrol (˜111 mmol, 1 equiv.) in pyridine was added propionic anhydride (72.23 g, 555.00 mmol, 71.51 mL, 5 equiv.) at 15° C., then the mixture was heated to 65° C. and stirred at 65° C. for 10 h under N₂ atmosphere. TLC revealed three major spots with lower polarity. The reaction mixture was diluted with H₂O (500 mL) and extracted with EtOAc (150 mL×3). The combined organic layers were washed with brine (50 mL), dried over Na₂SO₄, filtered, and the filtrate was concentrated under reduced pressure to give a residue. The residue was purified by column chromatography (SiO₂, petroleum ether/ethyl acetate=10/1 to 3/1). [(2R,3R,4S,5R)-4,5,6-tri(propanoyloxy)-2-(trityloxymethyl)tetrahydropyran-3-yl]propanoate (29 g, 44.84 mmol, 40.40% yield) was obtained as a colorless oil.

A solution of [(2R,3R,4S,5R)-4,5,6-tri(propanoyloxy)-2-(trityloxymethyl)tetrahydropyran-3-yl]propanoate (8 g, 12.37 mmol, 1 equiv.) in HOAc (60 mL) and H₂O (30 mL) was stirred at 65° C. for 2.5 under N₂ atmosphere. TLC indicated [(2R,3R,4S,5R)-4,5,6-tri(propanoyloxy)-2-(trityloxymethyl)tetrahydropyran-3-yl]propanoate was consumed completely, and two new spots formed.

The reaction mixture was diluted with H₂O (100 mL) and extracted with EtOAc (40 mL×3). The combined organic layers were washed with brine (30 mL), dried over Na₂SO₄, filtered, and the filtrate was concentrated under reduced pressure to give colorless oil. The residue was purified by column chromatography (SiO₂, petroleum ether/ethyl acetate=10/1 to 3/1). [(2R,3R,4S,5R)-2-(hydroxymethyl)-4,5,6-tri(propanoyloxy)tetrahydropyran-3-yl]propanoate (3.1 g, 7.67 mmol, 61.97% yield) was obtained as a colorless oil.

A mixture of (E)-4-methoxy-4-oxo-but-2-enoic acid (1.50 g, 11.50 mmol, 1.5 equiv.), DCC (2.37 g, 11.50 mmol, 2.33 mL, 1.5 equiv.), DMAP (468.24 mg, 3.83 mmol, 0.5 equiv.) in DCM (100 mL) was stirred at 15° C. for 0.5 h. [(2R,3R,4S,5R)-2-(hydroxymethyl)-4,5,6-tri(propanoyloxy)tetrahydropyran-3-yl]propanoate (3.1 g, 7.67 mmol, 1 equiv.) was added to the mixture and then the mixture was stirred at 15° C. for 9.5 h under N₂ atmosphere. The reaction mixture was filtered and the filtrate was concentrated under reduced pressure to give a residue. The residue was purified by column chromatography (SiO₂, petroleum ether/ethyl acetate=3/1 to 1/1). After column chromatography, the crude product was purified by recrystallization with petroleum ether/EtOAc=30/1 (10 mL) at 20° C. The compound methyl(((2R,3R,4S,5R,6S)-3,4,5,6-tetrakis(propionyloxy)tetrahydro-2H-pyran-2-yl)methyl) fumarate (770 mg, 1.48 mmol, 19.28% yield, 99.13% purity) was obtained as a white solid from the filter cake after filtration. 1H NMR (400 MHz, Chloroform-d): δ 6.88 (t, J=1.0 Hz, 2H), 5.75 (dd, J=8.3, 1.3 Hz, 1H), 5.35-5.25 (m, 1H), 5.23-5.10 (m, 2H), 4.35-4.26 (m, 2H), 3.90 (d, J=9.9 Hz, 1H), 3.82 (d, J=1.3 Hz, 3H), 2.49-2.19 (m, 8H), 1.19-1.01 (m, 12H) ppm. LCMS (M+18)⁺: 534.2.

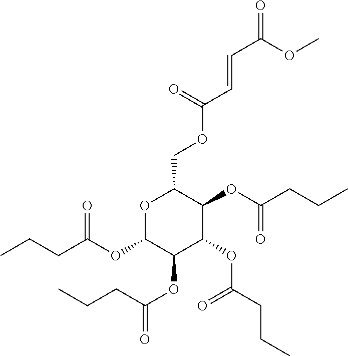 [SMILES: CCCC(=O)O[C@@H]1O[C@H](COC(=O)/C=C/C(=O)OC)[C@@H](OC(=O)CCC)[C@H](OC(=O)CCC)[C@H]1OC(=O)CCC]

### Compound 23: methyl(((2R,3R,4S,5R,6S)-3,4,5,6-tetrakis(butyryloxy)tetrahydro-2H-pyran-2-yl)methyl) fumarate

To a solution of [(2R,3R,4S,5R)-4,5,6-tri(butanoyloxy)-2-(trityloxymethyl)tetrahydropyran-3-yl]butanoate (8 g, 11.38 mmol, 1 equiv.) in HOAc (50 mL) was added HBr (2.79 g, 11.38 mmol, 1.87 mL, 33% purity, 1 equiv.). The mixture was stirred at 15° C. for 0.5 h. TLC indicated reactant was consumed completely. The reaction mixture was filtered and the filtrate was concentrated under reduced pressure to give a residue. The residue was purified by column chromatography (SiO₂, petroleum ether/ethyl acetate=20/1 to 0/1). [(2R,3R,4S,5R)-4,5,6-tri(butanoyloxy)-2-(hydroxymethyl)tetrahydropyran-3-yl]butanoate (2.5 g, 5.43 mmol, 47.69% yield) was obtained as a colorless oil.

To a solution of (E)-4-methoxy-4-oxo-but-2-enoic acid (1.41 g, 10.86 mmol, 2 equiv.) and DCC (1.68 g, 8.14 mmol, 1.5 equiv.) in DCM (20 mL) was added DMAP (331.61 mg, 2.71 mmol, 0.5 equiv.) was stirred at 15° C. for 10 min. Then [(2R,3R,4S,5R)-4,5,6-tri(butanoyloxy)-2-(hydroxymethyl)tetrahydropyran-3-yl]butanoate (2.5 g, 5.43 mmol, 1 equiv.) was added to the mixture and the mixture was stirred at 15° C. for 12 h. TLC indicated reactant was consumed completely. The reaction mixture filtered and the filtrate was concentrated under reduced pressure to give a residue. The residue was purified by column chromatography (SiO₂, petroleum ether/ethyl acetate=20/1 to 5/1). The product methyl(((2R,3R,4S,5R,6S)-3,4,5,6-tetrakis(butyryloxy)tetrahydro-2H-pyran-2-yl)methyl) fumarate (1 g, 1.75 mmol, 32.17% yield) was obtained as a colorless oil. SFC separation (Neu-IPA; B %: 40%-40%, 4 min) was performed to provide methyl(((2R,3R,4S,5R,6S)-3,4,5,6-tetrakis(butyryloxy)tetrahydro-2H-pyran-2-yl)methyl) fumarate (900 mg) as a white solid. ¹H NMR (400 MHz, chloroform-d); δ 6.87 (s, 2H), 5.70 (d, J=8.3 Hz, 1H), 5.42 (dd, J=10.3, 8.2 Hz, 1H), 5.05 (dd, J=10.3, 3.1 Hz, 1H), 4.55-4.33 (m, 2H), 4.08 (d, J=4.0 Hz, 1H), 3.95 (t, J=6.3 Hz, 1H), 3.81 (s, 3H), 2.40-2.19 (m, 8H), 1.69-1.56 (m, 8H), 1.04-0.81 (m, 12H) ppm. LCMS (M+Na)⁺: 595.1.

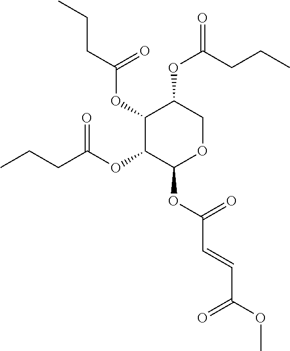 [SMILES: CCCC(=O)O[C@H]1[C@H](OC(=O)/C=C/C(=O)OC)OC[C@@H](OC(=O)CCC)[C@H]1OC(=O)CCC]

### Compound 24: methyl((2S,3R,4R,5R)-3,4,5-tris(butyryloxy)tetrahydro-2H-pyran-2-yl) fumarate

D-(−)-ribose was dissolved to provide a 0.5 M mixture in dichloromethane and pyridine (50/50 mixture), and then propionic anhydride (6 equiv.) was added to the solution at 0° C. The mixture was stirred at room temperature for 8 h. The resulting mixture was neutralized with 1 M HCl and purified by flash column chromatography. The resulting oil was dissolved in dry THF and treated with 1.5 equiv. of methyl amine in THF. The mixture was stirred at room temperature for 5 h, concentrated in vacuo, and purified over silica gel using ethyl acetate/n-hexane (50/50) as eluent. The resulting viscus oil was dissolved in dichloromethane and pyridine (50/50) mixture and then 2 equiv. of MMF was added and the mixture cooled to 0° C. 2 equiv. of N-(3-dimethylaminopropyl)-N′-ethylcarbodiimide hydrochloride (EDCI) was added to the solution followed by the addition of 0.1 equiv. DMAP and the mixture was stirred at room temperature for 5 h. The resulting mixture was filtered and concentrated in vacuo. The crude product was purified by column chromatography over silica gel using ethyl acetate/n-hexane (40/60) as eluent to give methyl((2S,3R,4R,5R)-3,4,5-tris(butyryloxy)tetrahydro-2H-pyran-2-yl) fumarate as a waxy solid. ¹H NMR (400 MHz, chloroform-d): δ 7.01-6.76 (m, 2H), 6.10 (d, J=5.0 Hz, 1H), 5.54 (t, J=3.4 Hz, 1H), 5.25-5.02 (m, 2H), 4.11-3.86 (m, 2H), 3.82 (s, 3H), 2.42-2.22 (m, 6H), 1.66 (dqd, J=8.3, 7.4, 5.8 Hz, 6H), 1.05-0.76 (m, 9H) ppm. LCMS (M+Na)⁺: 495.1.

Compound 24-d15 was synthesized in a similar manner as described herein, with the exception that d5-butyric acid was used in combination with, e.g., the EDCI coupling conditions.

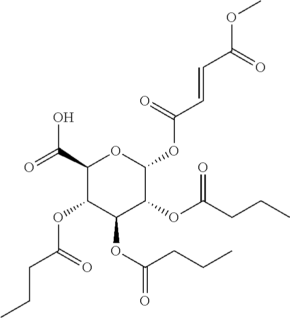 [SMILES: CCCC(=O)O[C@@H]1[C@@H](OC(=O)CCC)[C@@H](OC(=O)/C=C/C(=O)OC)O[C@H](C(=O)O)[C@H]1OC(=O)CCC]

### Compound 25: (2S,3S,4S,5R,6R)-3,4,5-tris(butyryloxy)-6-(((E)-4-methoxy-4-oxobut-2-enoyl)oxy)tetrahydro-2H-pyran-2-carboxylic acid

To a solution of benzyl(2S,3S,4S,5R)-3,4,5,6-tetra(butanoyloxy)tetrahydropyran-2-carboxylate (25 g, 44.28 mmol, 1 equiv.) in THF (30 mL) was added aq. MeNH₂ (5.04 g, 48.71 mmol, 30% purity, 1.1 equiv.). The mixture was stirred at 15° C. for 12 h. TLC indicated reactant was consumed completely. The reaction mixture was concentrated under reduced pressure to give a residue. The residue was purified by column chromatography (SiO₂, petroleum ether/ethyl acetate=30/1 to 7/1). benzyl(2S,3S,4S,5R)-3,4,5-tri(butanoyloxy)-6-hydroxy-tetrahydropyran-2-carboxylate (14 g, 28.31 mmol, 63.94% yield, purity) was obtained as a yellow oil.

To this solution of benzyl(2S,3S,4S,5R)-3,4,5-tri(butanoyloxy)-6-hydroxy-tetrahydropyran-2-carboxylate (5 g, 10.11 mmol, 1 equiv.) in THF (30 mL) was added Pd/C (1 g, 10% purity). The suspension was degassed and purged with H₂3 times. The mixture was stirred under H₂ (15 psi) at 15° C. for 4 h. TLC indicated reactant was consumed completely. The reaction mixture was filtered, and the filtrate was concentrated under reduced pressure to give a residue. (2S,3S,4S,5R)-3,4,5-tri(butanoyloxy)-6-hydroxy-tetrahydropyran-2-carboxylic acid (4 g, 9.89 mmol, 97.83% yield) was obtained as a white solid was used into the next step without further purification.

To a solution of (E)-4-methoxy-4-oxo-but-2-enoic acid (643.40 mg, 4.95 mmol, 2 equiv.) and DCC (765.29 mg, 3.71 mmol, 750.29 μL, 1.5 equiv.) in DCM (10 mL) was added DMAP (151.05 mg, 1.24 mmol, 0.5 equiv.). The resultant mixture was stirred at 15° C. for 10 min. Then (2S,3S,4S,5R)-3,4,5-tri(butanoyloxy)-6-hydroxy-tetrahydropyran-2-carboxylic acid (1 g, 2.47 mmol, 1 equiv.) was added to the mixture and the mixture was stirred at 15° C. for 12 h. LC-MS detected the desired compound. The reaction mixture was filtered, and the filtrate was concentrated under reduced pressure to give a residue. The residue was purified by prep-HPLC (water+0.05% HCl (v/v)/ACN). (2S,3S,4S,5R,6R)-3,4,5-tris(butyryloxy)-6-(((E)-4-methoxy-4-oxobut-2-enoyl)oxy)tetrahydro-2H-pyran-2-carboxylic acid (96 mg, 184.01 μmol, 7.44% yield, 99% purity) was obtained as a yellow solid. ¹H NMR (400 MHz, chloroform-d): δ 6.89 (d, J=1.5 Hz, 2H), 6.46 (d, J=3.7 Hz, 1H), 5.51 (t, J=9.9 Hz, 1H), 5.24 (t, J=9.9 Hz, 1H), 5.11 (dd, J=10.2, 3.6 Hz, 1H), 4.40 (d, J=10.2 Hz, 1H), 3.79 (s, 3H), 2.32-2.05 (m, 6H), 1.66-1.42 (m, 6H), 0.95-0.66 (m, 9H) ppm. LCMS (M−H)+: 514.8.

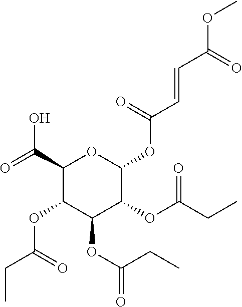 [SMILES: CCC(=O)O[C@@H]1[C@@H](OC(=O)CC)[C@@H](OC(=O)/C=C/C(=O)OC)O[C@H](C(=O)O)[C@H]1OC(=O)CC]

### Compound 26: (2S,3S,4S,5R,6R)-6-(((E)-4-methoxy-4-oxobut-2-enoyl)oxy)-3,4,5-tris(propionyloxy)tetrahydro-2H-pyran-2-carboxylic acid

To a solution of benzyl(2S,3S,4S,5R)-3,4,5,6-tetra(propanoyloxy)tetrahydropyran-2-carboxylate (5 g, 9.83 mmol, 1 equiv.) in THF (20 mL) was added aq. MeNH₂ (1.12 g, 10.82 mmol, 30% purity, 1.1 equiv.). The mixture was stirred at 15° C. for 12 h. LCMS detected the desired compound. The reaction mixture was concentrated under reduced pressure to give a residue. The residue was purified by column chromatography (SiO₂, petroleum ether/EtOAc=20/1 to 3/1). benzyl(2S,3S,4S,5R)-6-hydroxy-3,4,5-tri(propanoyloxy)tetrahydropyran-2-carboxylate (3.6 g, 6.76 mmol, 68.78% yield, 85% purity) was obtained as a yellow oil.

To a solution of benzyl(2S,3S,4S,5R)-6-hydroxy-3,4,5-tri(propanoyloxy)tetrahydropyran-2-carboxylate (3.6 g, 7.96 mmol, 1 equiv.) in THF (5 mL) was added Pd/C (300 mg, 10% purity). The suspension was degassed and purged with H₂3 times. The mixture was stirred under H₂ (15 psi) at 15° C. for 4 h. TLC indicated reactant was consumed completely. The mixture was filtered, and the filtrate was concentrated under reduced pressure to give a residue. (2S,3S,4S,5R)-6-hydroxy-3,4,5-tri(propanoyloxy)tetrahydropyran-2-carboxylic acid (3.6 g, crude) was obtained as a white solid that was used into the next step without further purification.

To a solution of (E)-4-methoxy-4-oxo-but-2-enoic acid (718.12 mg, 5.52 mmol, 2 equiv.) in DCM (10 mL) was added DCC (854.17 mg, 4.14 mmol, 837.42 μL, 1.5 equiv.) and DMAP (168.59 mg, 1.38 mmol, 0.5 equiv.). (2S,3S,4S,5R)-6-hydroxy-3,4,5-tri(propanoyloxy)tetrahydropyran-2-carboxylic acid (1 g, 2.76 mmol, 1 equiv.) was added to the mixture at 15° C. The mixture was stirred at 15° C. for 12 h. LCMS showed the desired compound was detected. The reaction mixture was filtered, and the filtrate was concentrated under reduced pressure to give a residue. The residue was purified by prep-HPLC (water+0.05% HCl (v/v)/ACN). (2S,3S,4S,5R,6R)-6-(((E)-4-methoxy-4-oxobut-2-enoyl)oxy)-3,4,5-tris(propionyloxy)tetrahydro-2H-pyran-2-carboxylic acid (170 mg, 358.34 μmol, 12.98% yield) was obtained as a yellow solid. ¹H NMR (400 MHz, Chloroform-d) δ 6.96 (d, J=1.3 Hz, 2H), 6.52 (d, J=3.7 Hz, 1H), 5.56 (t, J=9.8 Hz, 1H), 5.32 (t, J=9.9 Hz, 1H), 5.19 (dd, J=10.2, 3.7 Hz, 1H), 4.49 (d, J=10.2 Hz, 1H), 3.85 (s, 3H), 2.43-2.15 (m, 6H), 1.09 (p, J=7.7 Hz, 9H) ppm. LCMS (M−H)⁻: 472.8.

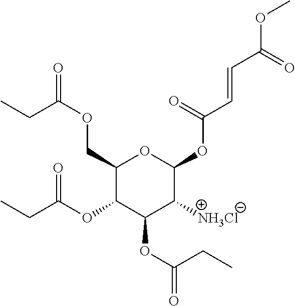

### Compound 27: (2S,3R,4R,5S,6R)-2-(((E)-4-methoxy-4-oxobut-2-enoyl)oxy)-4,5-bis(propionyloxy)-6-((propionyloxy)methyl)tetrahydro-2H-pyran-3-aminium chloride

N-Boc-D-glucosamine was dissolved in a 50/50 mixture of dichloromethane and pyridine, and propionic anhydride (˜6 equiv.) was added to the solution at 0° C. The mixture was stirred at room temperature for 8 h. The resulting mixture was neutralized with 1 M HCl and purified by flash column chromatography. The resulting oil was dissolved in 0.1 M dry THF and treated with 1.5 eq of methyl amine in THF. The mixture was stirred at room temperature for 5 h, concentrated in vacuo, and purified by column chromatography over silica gel using ethyl acetate/n-hexane (50/50) as eluent. The resulting viscus oil was dissolved in dry tetrahydrofuran (THF). Then DMAP and MMF was added and the mixture cooled to 0° C. Dicyclohexylcarbodiimide (DCC, 1.2 eq mmol) was added to the solution followed by stirring at room temperature for 5 h. The resulting mixture was filtered and concentrated in vacuo. The crude product was purified by column chromatography over silica gel using ethyl acetate/n-hexane (40/60) as eluent. Resulting compound was dissolved in methanol and 2 equiv. of a hydrogen chloride solution (in dioxane) was added. The resulting mixture was filtered purified by reverse-phase column chromatography to yield the titled compound (2S,3R,4R,5S,6R)-2-(((E)-4-methoxy-4-oxobut-2-enoyl)oxy)-4,5-bis(propionyloxy)-6-((propionyloxy)methyl)tetrahydro-2H-pyran-3-aminium chloride as a white solid. ¹H NMR (400 MHz, methanol-d4) δ 6.89 (ddd, J=141.2, 15.5, 0.7 Hz, 2H), 5.38 (dd, J=10.8, 9.3 Hz, 1H), 5.14 (d, J=3.4 Hz, 1H), 4.44-4.19 (m, 3H), 4.15-4.00 (m, 1H), 3.77 (d, J=0.7 Hz, 3H), 2.52-2.09 (m, 6H), 1.21-0.68 (m, 9H) ppm. LCMS (M+Na)⁺: 482.1.

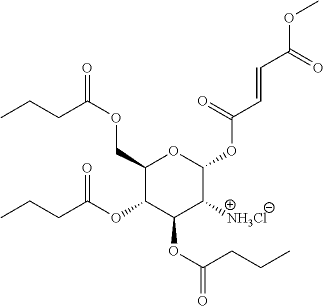

### Compound 28: (2R,3R,4R,5S,6R)-4,5-bis(butyryloxy)-6-((butyryloxy)methyl)-2-(((E)-4-methoxy-4-oxobut-2-enoyl)oxy)tetrahydro-2H-pyran-3-aminium chloride

N-Boc-D-glucosamine was dissolved in a 50/50 mixture of dichloromethane and pyridine. Butyric anhydride (˜6 equiv.) was added to the solution at 0° C. The mixture was stirred at room temperature for 8 h. The resulting mixture was neutralized with 1 M HCl and purified by flash column chromatography. The resulting oil was dissolved in 0.1 M dry THF and treated with 1.5 eq of methyl amine in THF. The mixture was stirred at room temperature for 5 h, concentrated in vacuo and purified by column chromatography over silica gel using ethyl acetate/n-hexane (50/50) as eluent. The resulting viscus oil was dissolved in dry THF, and then DMAP and MMF were added and the mixture cooled to 0° C. Dicyclohexylcarbodiimide (DCC, 1.2 eq mmol) was added to the solution followed by stirring at room temperature for 5 h. The resulting mixture was filtered and concentrated in vacuo. The crude product was purified by column chromatography over silica gel using ethyl acetate/n-hexane (40/60) as eluent. Resulting compound was dissolved in methanol and 2 equiv. of hydrogen chloride solution (in dioxane) was added. The resulting compound was filtered purified by reverse-phase column chromatography to yielded the titled compound (2R,3R,4R,5S,6R)-4,5-bis(butyryloxy)-6-((butyryloxy)methyl)-2-(((E)-4-methoxy-4-oxobut-2-enoyl)oxy)tetrahydro-2H-pyran-3-aminium chloride as a white solid. ¹H NMR (400 MHz, methanol-d4): δ 7.07 (d, J=15.5 Hz, 1H), 6.71 (d, J=15.5 Hz, 1H), 5.47-5.29 (m, 1H), 5.13 (d, J=3.5 Hz, 1H), 5.07 (t, J=9.7 Hz, 1H), 4.39-4.16 (m, 4H), 4.13-4.06 (m, 1H), 3.77 (s, 3H), 2.47-2.10 (m, 6H), 1.76-1.39 (m, 6H), 1.13-0.63 (m, 9H) ppm. LCMS (M+Na)⁺: 524.4.

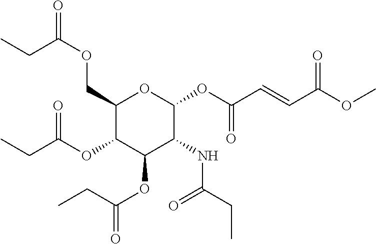 [SMILES: CCC(=O)N[C@H]1[C@@H](OC(=O)/C=C/C(=O)OC)O[C@H](COC(=O)CC)[C@@H](OC(=O)CC)[C@@H]1OC(=O)CC]

### Compound 29: methyl((2R,3R,4R,5S,6R)-3-propionamido-4,5-bis(propionyloxy)-6-((propionyloxy)methyl)tetrahydro-2H-pyran-2-yl) fumarate

D-(+)-glucosamine hydrochloride was dissolved in a 50/50 mixture of dichloromethane and pyridine. Propionic anhydride (˜6 equiv.) and DMAP (0.1 equiv.) were added to the solution at 0° C. The mixture was stirred at room temperature for 8 h. The resulting mixture was neutralized by 1 M HCl and purified by flash column chromatography. The resulting oil was dissolved in 0.1 M dry THF and treated with 2 equiv. of methyl amine in THF. The mixture was stirred at room temperature for 5 h, concentrated in vacuo, and purified by column chromatography over silica gel using ethyl acetate/n-hexane (50/50) as eluent. The resulting hemiacetal was dissolved in dry dichloromethane (0.5 M) and pyridine. 2 equiv. of MMF was added and the mixture cooled to 0° C. 2 equiv. of N-(3-dimethylaminopropyl)-N′-ethylcarbodiimide hydrochloride (EDCI) was added to the solution followed by the addition of 0.1 equiv. DMAP and the mixture was stirred at room temperature for 5 h. The resulting mixture was filtered and concentrated in vacuo. The crude product was purified by column chromatography over silica gel using ethyl acetate/n-hexane (40/60) to obtain the targeted compound methyl((2R,3R,4R,5S,6R)-3-propionamido-4,5-bis(propionyloxy)-6-((propionyloxy)methyl)tetrahydro-2H-pyran-2-yl) fumarate as a waxy solid. ¹H NMR (400 MHz, chloroform-d): δ 7.02-6.87 (m, 2H), 6.31 (d, J=3.5 Hz, 1H), 5.58 (d, J=8.7 Hz, 1H), 5.32-5.21 (m, 2H), 4.52 (ddd, J=11.8, 8.1, 3.4 Hz, 1H), 4.25 (dd, J=12.5, 4.3 Hz, 1H), 4.19-3.95 (m, 4H), 3.85 (d, J=1.0 Hz, 3H), 2.44-2.06 (m, 8H), 1.25 (td, J=7.1, 1.0 Hz, 3H), 1.09 (dt, J=16.6, 7.7 Hz, 9H) ppm. LCMS (M+Na)⁺: 538.1.

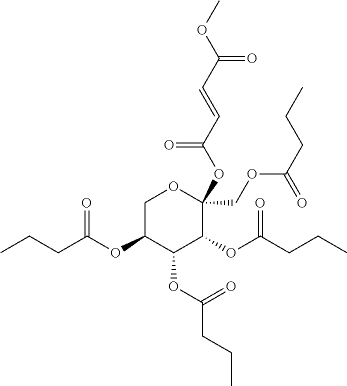 [SMILES: CCCC(=O)OC[C@@]1(OC(=O)/C=C/C(=O)OC)OC[C@H](OC(=O)CCC)[C@@H](OC(=O)CCC)[C@H]1OC(=O)CCC]

### Compound 30: methyl((2S,3R,4R,5S)-3,4,5-tris(butyryloxy)-2-((butyryloxy)methyl)tetrahydro-2H-pyran-2-yl) fumarate

D-(−)-tagatose was dissolved in 50/50 mixture of dichloromethane and pyridine. Butyric anhydride (˜6 equiv.) and DMAP (0.1 equiv.) was added to the solution at 0° C. The mixture was stirred at room temperature for 8 h. The resulting mixture was neutralized by 1 M HCl and purified by flash column chromatography. The resulting oil was dissolved in dry THF and treated with 2 equiv. of methyl amine in THF. The mixture was stirred at room temperature for 5 h, concentrated in vacuo, and purified by column chromatography over silica gel using ethyl acetate/n-hexane (50/50) as eluent. The resulting hemiacetal was dissolved in dry DCM and pyridine. MMF was then added and the mixture 0° C. N-(3-dimethylaminopropyl)-N′-ethylcarbodiimide hydrochloride (EDCI) was added to the solution followed by the addition of DMAP and the mixture was stirred at room temperature for 5 h. The resulting mixture was filtered and concentrated in vacuo. The crude product was purified by column chromatography over silica gel using ethyl acetate/n-hexane (40/60) to obtain the targeted compound methyl((2S,3R,4R,5S)-3,4,5-tris(butyryloxy)-2-((butyryloxy)methyl)tetrahydro-2H-pyran-2-yl) fumarate as a waxy solid. ¹H NMR (400 MHz, chloroform-d): δ 6.91 (d, J=4.4 Hz, 2H), 5.57 (d, J=3.2 Hz, 1H), 5.44-5.17 (m, 2H), 4.85 (d, J=12.3 Hz, 1H), 4.40 (d, J=12.3 Hz, 1H), 4.12 (dd, J=11.2, 5.8 Hz, 1H), 3.83 (s, 3H), 3.48 (t, J=10.9 Hz, 1H), 2.38 (t, J=7.4 Hz, 2H), 2.23 (dq, J=10.8, 7.5 Hz, 6H), 1.74-1.49 (m, 8H), 1.06-0.83 (m, 12H) ppm. LCMS (M+Na)⁺: 595.3.

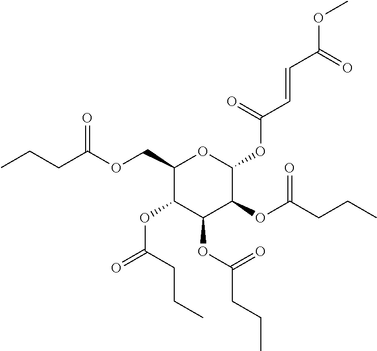 [SMILES: CCCC(=O)OC[C@H]1O[C@H](OC(=O)/C=C/C(=O)OC)[C@@H](OC(=O)CCC)[C@@H](OC(=O)CCC)[C@@H]1OC(=O)CCC]

### Compound 31: methyl((2R,3S,4S,5R,6R)-3,4,5-tris(butyryloxy)-6-((butyryloxy)methyl)tetrahydro-2H-pyran-2-yl) fumarate

D-(+)-mannose was dissolved in a 50/50 mixture of dichloromethane and pyridine. Butyric anhydride (˜6 equiv.) and DMAP (0.1 equiv.) was added to the solution at 0° C. The mixture was stirred at room temperature for 8 h. The resulting mixture was neutralized by 1 M HCl and purified by flash column chromatography. The resulting oil was dissolved in 0.1 M dry THF and treated with 2 eq of methyl amine in THF. The mixture was stirred at room temperature for 5 h, concentrated in vacuo, and purified by column chromatography over silica gel using ethyl acetate/n-hexane (50/50) as eluent. The resulting hemiacetal was dissolved in dry dichloromethane (DCM) and pyridine followed by the addition of MMF and cooling to 0° C. N-(3-dimethylaminopropyl)-N′-ethylcarbodiimide hydrochloride (EDCI) was added to the solution followed by the addition of 0.1 eq of DMAP and the mixture was stirred at room temperature for 5 h. The resulting mixture was filtered and concentrated in vacuo. The crude product was purified by column chromatography over silica gel using ethyl acetate/n-hexane (40/60) to obtain the targeted compound methyl((2R,3S,4S,5R,6R)-3,4,5-tris(butyryloxy)-6-((butyryloxy)methyl)tetrahydro-2H-pyran-2-yl) fumarate as a waxy solid. ¹H NMR (400 MHz, chloroform-d): δ 7.04-6.83 (m, 2H), 6.25-6.13 (m, 1H), 5.51-5.28 (m, 3H), 4.30-3.99 (m, 4H), 3.84 (d, J=1.0 Hz, 3H), 2.50-2.14 (m, 8H), 1.80-1.51 (m, 8H), 1.07-0.82 (m, 12H) ppm. LCMS (M+Na)⁺: 572.1.

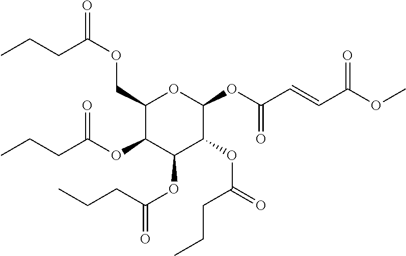 [SMILES: CCCC(=O)OC[C@H]1O[C@@H](OC(=O)/C=C/C(=O)OC)[C@H](OC(=O)CCC)[C@@H](OC(=O)CCC)[C@H]1OC(=O)CCC]

### Compound 32: methyl((2S,3R,4S,5S,6R)-3,4,5-tris(butyryloxy)-6-((butyryloxy)methyl)tetrahydro-2H-pyran-2-yl) fumarate

D-(+)-galactose was dissolved in a 50/50 mixture of dichloromethane and pyridine. Butyric anhydride (˜6 equiv.) and DMAP (0.1 equiv.) was added to the solution at 0° C. The mixture was stirred at room temperature for 8 h. The resulting mixture was neutralized by 1 M HCl and purified by flash column chromatography. The resulting oil was dissolved in 0.1 M dry THF and treated with 2 eq of methyl amine in THF. The mixture was stirred at room temperature for 5 h, concentrated in vacuo and was purified by column chromatography over silica gel using ethyl acetate/n-hexane (50/50) as eluent. The resulting hemiacetal was dissolved in dry DCM and pyridine followed by the addition of 2 equiv. MMF and cooling to 0° C. 2 equiv. of N-(3-dimethylaminopropyl)-N′-ethylcarbodiimide hydrochloride (EDCI) was added to the solution followed by the addition of 0.1 eq DMAP and the mixture was stirred at room temperature for 5 h. The resulting mixture was filtered and concentrated in vacuo. The crude product was purified by column chromatography over silica gel using ethyl acetate/n-hexane (40/60) to obtain the title compound as a waxy solid. ¹H NMR (400 MHz, chloroform-d): δ 6.92 (s, 2H), 6.48 (d, J=2.7 Hz, 2H), 5.55 (t, J=2.0 Hz, 1H), 5.38 (t, J=2.3 Hz, 1H), 4.42-4.29 (m, 2H), 4.10 (dd, J=9.1, 6.9 Hz, 2H), 3.84 (s, 3H), 2.47-2.11 (m, 8H), 1.75-1.47 (m, 8H), 1.05-0.81 (m, 12H) ppm. LCMS (M+Na)⁺: 595.3.

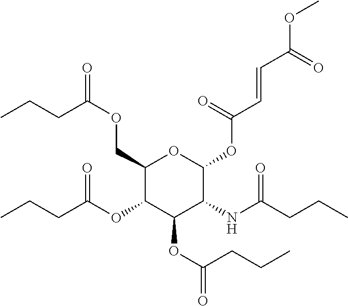 [SMILES: CCCC(=O)N[C@H]1[C@@H](OC(=O)/C=C/C(=O)OC)O[C@H](COC(=O)CCC)[C@@H](OC(=O)CCC)[C@@H]1OC(=O)CCC]

### Compound 33: (2R,3R,4R,5S,6R)-3-butyramido-4,5-bis(butyryloxy)-6-((butyryloxy)methyl)tetrahydro-2H-pyran-2-yl methyl fumarate

D-(+)-glucosamine hydrochloride was dissolved in a 50/50 mixture of dichloromethane and pyridine. Butyric anhydride (˜6 equiv.) and DMAP (0.1 equiv.) was added to the solution at 0° C. The mixture was stirred at room temperature for 8 h. The resulting mixture was neutralized by 1 M HCl and purified by flash column chromatography. The resulting oil was dissolved in 0.1 M dry THF and treated with 2 equiv. of methyl amine in THF. The mixture was stirred at room temperature for 5 h, concentrated in vacuo, and purified by column chromatography over silica gel using ethyl acetate/n-hexane (50/50) as eluent. The resulting hemiacetal was dissolved in dry dichloromethane (DCM) and pyridine followed by the addition of 2 equiv. MMF and cooling to 0° C. 2 equiv. of N-(3-dimethylaminopropyl)-N′-ethylcarbodiimide hydrochloride (EDCI) was added to the solution followed by the addition of 0.1 equiv. DMAP and the mixture was stirred at room temperature for 5 h. The resulting mixture was filtered and concentrated in vacuo. The crude product was purified by column chromatography over silica gel using ethyl acetate/n-hexane (40/60) to obtain the targeted compound (2R,3R,4R,5S,6R)-3-butyramido-4,5-bis(butyryloxy)-6-((butyryloxy)methyl)tetrahydro-2H-pyran-2-yl methyl fumarate as a waxy solid. ¹H NMR (400 MHz, chloroform-d): δ 6.95 (d, J=3.9 Hz, 2H), 6.32 (d, J=3.5 Hz, 1H), 5.60 (d, J=8.5 Hz, 1H), 5.33-5.19 (m, 2H), 4.59-4.42 (m, 1H), 4.26-4.05 (m, 2H), 4.00 (ddd, J=9.8, 4.2, 1.9 Hz, 1H), 3.85 (s, 3H), 2.38-1.96 (m, 8H), 1.72-1.53 (m, 8H), 1.01-0.81 (m, 12H) ppm. LCMS (M+Na)⁺: 595.2.

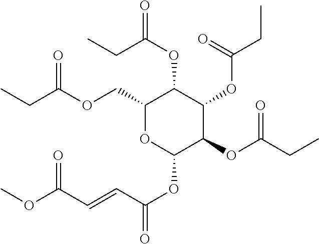 [SMILES: CCC(=O)OC[C@H]1O[C@@H](OC(=O)/C=C/C(=O)OC)[C@H](OC(=O)CC)[C@@H](OC(=O)CC)[C@H]1OC(=O)CC]

### Compound 34: methyl((2S,3R,4S,5S,6R)-3,4,5-tris(propionyloxy)-6-((propionyloxy)methyl)tetrahydro-2H-pyran-2-yl) fumarate

D-(+)-galactose was dissolved in a 50/50 mixture of dichloromethane and pyridine. Propionic anhydride (˜6 equiv.) and DMAP (0.1 equiv.) was added to the solution at 0° C. The mixture was stirred at room temperature for 8 h. The resulting mixture was neutralized by 1 M HCl and purified by flash column chromatography. The resulting oil was dissolved in 0.1 M dry THF and treated with 2 equiv. of methyl amine in THF. The mixture was stirred at room temperature for 5 h, concentrated in vacuo, and purified by column chromatography over silica gel using ethyl acetate/n-hexane (50/50) as eluent. The resulting hemiacetal was dissolved in dry DCM and pyridine followed by the addition of 2 equiv. MMF and cooling to 0° C. 2 equiv. of N-(3-dimethylaminopropyl)-N′-ethylcarbodiimide hydrochloride (EDCI) was added to the solution followed by the addition of 0.1 equiv. DMAP. The mixture was stirred at room temperature for 5 h. The resulting mixture was filtered and concentrated in vacuo. The crude product was purified by column chromatography over silica gel using ethyl acetate/n-hexane (40/60) to yield the title compound as a waxy solid. ¹H NMR (400 MHz, chloroform-d): δ 6.92 (s, 2H), 6.48 (d, J=3.0 Hz, 1H), 5.63-5.50 (m, 1H), 5.46-5.32 (m, 2H), 4.38 (t, J=6.7 Hz, 1H), 4.20-4.04 (m, 2H), 3.84 (s, 3H), 2.52-2.16 (m, 8H), 1.32-1.01 (m, 12H) ppm. LCMS (M+Na)⁺: 539.2.

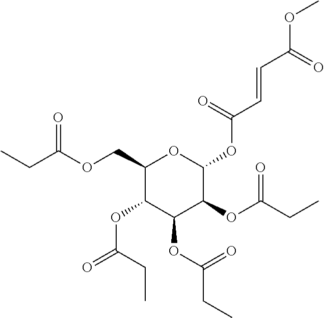 [SMILES: CCC(=O)OC[C@H]1O[C@H](OC(=O)/C=C/C(=O)OC)[C@@H](OC(=O)CC)[C@@H](OC(=O)CC)[C@@H]1OC(=O)CC]

### Compound 35: methyl((2R,3S,4S,5R,6R)-3,4,5-tris(propionyloxy)-6-((propionyloxy)methyl)tetrahydro-2H-pyran-2-yl) fumarate

D-(+)-mannose was dissolved in a 50/50 mixture of dichloromethane and pyridine. Propionic anhydride (˜6 equiv.) and DMAP (0.1 equiv.) was added to the solution at 0° C. The mixture was stirred at room temperature for 8 h. The resulting mixture was neutralized by 1 M HCl and purified by flash column chromatography. The resulting oil was dissolved in 0.1 M dry THF and treated with 2 equiv. of methyl amine in THF. The mixture was stirred at room temperature for 5 h, concentrated in vacuo, and purified by column chromatography over silica gel using ethyl acetate/n-hexane (50/50) as eluent. The resulting hemiacetal was dissolved in dry DCM and pyridine followed by the addition of MMF and cooling to 0° C. N-(3-dimethylaminopropyl)-N′-ethylcarbodiimide hydrochloride (EDCI) was added to the solution followed by the addition of DMAP and the mixture was stirred at room temperature for 5 h. The resulting mixture was filtered and concentrated in vacuo. The crude product was purified by column chromatography over silica gel using ethyl acetate/n-hexane (40/60) to obtain the targeted compound methyl((2R,3S,4S,5R,6R)-3,4,5-tris(propionyloxy)-6-((propionyloxy)methyl)tetrahydro-2H-pyran-2-yl) fumarate as a waxy solid. ¹H NMR (400 MHz, Chloroform-d) δ 7.03-6.83 (m, 2H), 6.19 (d, J=1.9 Hz, 1H), 5.53-5.29 (m, 3H), 4.29 (dd, J=12.4, 4.8 Hz, 1H), 4.16-4.04 (m, 2H), 3.84 (s, 2H), 2.54-2.19 (m, 8H), 1.29-0.90 (m, 12H). LCMS (M+Na)⁺: 539.0 ppm.

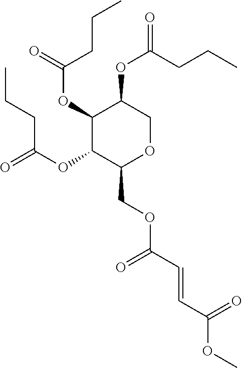 [SMILES: CCCC(=O)O[C@@H]1[C@@H](OC(=O)CCC)[C@@H](OC(=O)CCC)CO[C@H]1COC(=O)/C=C/C(=O)OC]

### Compound 36: 1-methyl(2S,3R,4S,5S)-3,4,5-tris(butanoyloxy)oxan-2-yl(2E)-but-2-enedioate

To a solution of [(3R,4S,5R)-4,5-di(butanoyloxy)-6-hydroxy-tetrahydropyran-3-yl]butanoate (500 mg, 1.39 mmol, 1 equiv.), DCC (429.38 mg, 2.08 mmol, 420.96 μL, 1.5 equiv.) and DMAP (50.85 mg, 416.21 μmol, 0.3 equiv.) in THF (10 mL) was added (E)-4-methoxy-4-oxo-but-2-enoic acid (270.74 mg, 2.08 mmol, 1.5 equiv.). The resultant mixture was stirred at 25° C. for 12 h. LCMS showed the starting reactant was consumed. The mixture reaction was concentrated. The residue was purified by prep-HPLC (water+10 mM NH₄HCO₃/ACN) to obtain 1-methyl(2S,3R,4S,5S)-3,4,5-tris(butanoyloxy)oxan-2-yl(2E)-but-2-enedioate as colorless oil. LCMS (M+Na)⁺: 495.1 at 3.185 min & 495.2 at 3.453 min. ¹H NMR (400 MHz, methanol-d4): δ 6.96 (d, J=5.5 Hz, 2H), 6.43 (d, J=3.5 Hz, 1H), 5.59-5.18 (m, 3H), 4.23 (dd, J=13.5, 1.2 Hz, 1H), 3.85 (s, 4H), 2.52-2.13 (m, 6H), 1.82-1.44 (m, 6H), 1.12-0.71 (m, 9H) ppm.

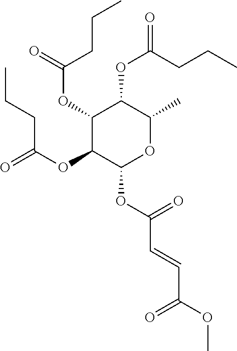 [SMILES: CCCC(=O)O[C@H]1[C@H](OC(=O)CCC)[C@@H](OC(=O)/C=C/C(=O)OC)O[C@@H](C)[C@H]1OC(=O)CCC]

### Compound 37: 1-methyl(2R,3S,4R,5R,6S)-3,4,5-tris(butanoyloxy)-6-methyloxan-2-yl(2E)-but-2-enedioate

To a solution of (3S,4R,5S,6S)-6-methyltetrahydropyran-2,3,4,5-tetrol (10 g, 60.92 mmol, 1 equiv.) in pyridine (100 mL) was added butyric anhydride (57.82 g, 365.51 mmol, 59.79 mL, 6 equiv.). The mixture was stirred at 15° C. for 12 h. TLC showed the starting reactant was consumed and two new spots formed. The mixture was washed with H₂O (100 mL) and extracted with EtOAc (100 mL×3). Then the mixture was concentrated. The residue was purified by column chromatography (SiO₂, petroleum ether/ethyl acetate=50/1). [(2S,3R,4R,5S)-4,5,6-tri(butanoyloxy)-2-methyl-tetrahydropyran-3-yl]butanoate (36.5 g, crude) was obtained as colorless oil.

To a solution of [(2S,3R,4R,5S)-4,5,6-tri(butanoyloxy)-2-methyl-tetrahydropyran-3-yl]butanoate (26 g, 58.49 mmol, 1 equiv.) in THF (200 mL) was added aq. MeNH₂ (10.90 g, 105.28 mmol, 30% purity, 1.8 equiv.). The mixture was stirred at 15° C. for 12 h. TLC showed most starting reactant was consumed and one new spot formed. The mixture was concentrated. The residue was purified by column chromatography (SiO₂, petroleum ether/ethyl acetate=30/1 to 5/1). [(2S,3R,4R,5S)-4,5-di(butanoyloxy)-6-hydroxy-2-methyl-tetrahydropyran-3-yl]butanoate (8.77 g, 23.42 mmol, 40.04% yield) was obtained as yellow oil.

To a solution of [(2S,3R,4R,5S)-4,5-di(butanoyloxy)-6-hydroxy-2-methyl-tetrahydropyran-3-yl]butanoate (8.7 g, 23.24 mmol, 1 equiv.) in DCM (80 mL) was added DCC (7.19 g, 34.85 mmol, 7.05 mL, 1.5 equiv.) and DMAP (1.42 g, 11.62 mmol, 0.5 equiv.). Then (E)-4-methoxy-4-oxo-but-2-enoic acid (4.53 g, 34.85 mmol, 1.5 equiv.) was added to the mixture. The mixture was stirred at 15° C. for 12 h. TLC showed the starting reactant was consumed and two new spots formed. The mixture was concentrated. The residue was purified by column chromatography (SiO₂, petroleum ether/ethyl acetate=50/1 to 10/1) first and then re-purified by prep-HPLC (column: Waters Xbridge Prep OBD C18 150×40 10 μm; mobile phase: water+10 mM NH₄HCO₃/ACN; B %: 50%-70%, 11 min). 1-methyl(2R,3S,4R,5R,6S)-3,4,5-tris(butanoyloxy)-6-methyloxan-2-yl(2E)-but-2-enedioate (227 mg, 461.92 μmol, 1.99/yield, 99% purity) was obtained as colorless oil. LCMS (M+18)⁺: 504.2 at 3.309 min. ¹H NMR (400 MHz, chloroform-d): δ 6.90 (s, 2H), 6.43 (d, J=2.1 Hz, 1H), 5.37 (d, J=1.7 Hz, 3H), 4.28 (q, J=6.5 Hz, 1H), 3.82 (s, 3H), 2.41 (t, J=7.5 Hz, 3H), 2.28-2.07 (m, 3H), 1.79-1.36 (m, 6H), 1.14 (d, J=6.5 Hz, 3H), 1.07-0.73 (m, 9H) ppm.

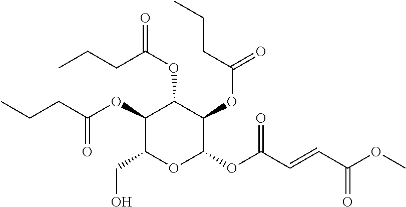 [SMILES: CCCC(=O)O[C@@H]1[C@@H](OC(=O)CCC)[C@H](OC(=O)/C=C/C(=O)OC)O[C@H](CO)[C@H]1OC(=O)CCC]

### Compound 38:1-methyl(2S,3R,4S,5R,6R)-3,4,5-tris(butanoyloxy)-6-(hydroxymethyl)oxan-2-yl(2E)-but-2-enedioate

To a solution of [(2R,3R,4S,5R)-4,5,6-tri(butanoyloxy)-2-(trityloxymethyl)tetrahydropyran-3-yl]butanoate (18 g, 25.61 mmol, 1 equiv.) in THF (100 mL) was added aq. MeNH₂ (2.65 g, 25.61 mmol, 30% purity, 1 equiv.). The mixture was stirred at 15° C. for 18 h. One new spot was observed by TLC. The reaction mixture was concentrated under reduced pressure to give a residue. The residue was purified by column chromatography (SiO₂, petroleum ether/ethyl acetate=30/1 to 7/1). [(2R,3R,4S,5R)-4,5-di(butanoyloxy)-6-hydroxy-2-(trityloxymethyl)tetrahydropyran-3-yl]butanoate (8 g, 12.64 mmol, 49.37% yield) was obtained as a white solid.

To a solution of (E)-4-methoxy-4-oxo-but-2-enoic acid (1.64 g, 12.64 mmol, 2 equiv.) and DCC (1.96 g, 9.48 mmol, 1.5 equiv.) in DCM (20 mL) was added DMAP (386.16 mg, 3.16 mmol, 0.5 equiv.) and the reaction mixture stirred at 15° C. for 10 min. Then [(2R,3R,4S,5R)-4,5-di(butanoyloxy)-6-hydroxy-2-(trityloxymethyl)tetrahydropyran-3-yl]butanoate (4 g, 6.32 mmol, 1 equiv.) was added to the mixture and the mixture was stirred at 15° C. for 12 h. TLC indicated reactant was consumed completely. The reaction mixture was filtered and the filtrate was concentrated under reduced pressure to give a residue. The residue was purified by column chromatography (SiO₂, petroleum ether/ethyl acetate=20/1 to 6/1). O1-methyl O4-[(3R,4S,5R,6R)-3,4,5-tri(butanoyloxy)-6-(trityloxymethyl)tetrahydropyran-2-yl](E)-but-2-enedioate (2.1 g, 2.82 mmol, 44.60% yield) was obtained as a colorless oil. To a solution of O1-methyl O4-[(3R,4S,5R,6R)-3,4,5-tri(butanoyloxy)-6-(trityloxymethyl)tetrahydropyran-2-yl](E)-but-2-enedioate (2.1 g, 2.82 mmol, 1 equiv.) in HOAc (20 mL) was added H₂O (10.00 g, 555.08 mmol, 10 mL, 196.88 equiv.) at 15° C. and the mixture was stirred at 65° C. for 4 h. The reaction mixture was concentrated under reduced pressure to give a residue. The residue was purified by column chromatography (SiO₂, petroleum ether/ethyl acetate=20/1 to 0/1). O1-methyl-O4-[(3R,4S,5R,6R)-3,4,5-tri(butanoyloxy)-6-(hydroxymethyl)tetrahydropyran-2-yl](E)-but-2-enedioate (1.1 g, 2.19 mmol, 77.64% yield) was obtained as a colorless oil. O1-methyl O4-[(3R,4S,5R,6R)-3,4,5-tri(butanoyloxy)-6-(hydroxymethyl)tetrahydropyran-2-yl](E)-but-2-enedioate (1.39 g, 2.79 mmol, 1 equiv.) was purified by SFC (Neu-MeOH; B %: 13%-13%, 7 min). 1-methyl(2S,3R,4S,5R,6R)-3,4,5-tris(butanoyloxy)-6-(hydroxymethyl)oxan-2-yl(2E)-but-2-enedioate (37 mg, 47.86 μmol, 1.72% yield, 65% purity) was obtained as a white solid. LCMS (M+18)⁺: 520.1 at 3.12 min. ¹H NMR (400 MHz, Chloroform-d): δ 6.87-6.65 (m, 1H), 5.74 (d, J=8.2 Hz, 1H), 5.32 (t, J=9.6 Hz, 1H), 5.22-4.95 (m, 1H), 3.83-3.71 (m, 3H), 2.27-2.06 (m, 4H), 1.70-1.40 (m, 5H), 1.02-0.61 (m, 8H) ppm.

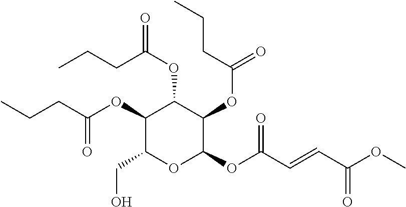 [SMILES: CCCC(=O)O[C@@H]1[C@@H](OC(=O)CCC)[C@@H](OC(=O)/C=C/C(=O)OC)O[C@H](CO)[C@H]1OC(=O)CCC]

### Compound 39: 1-methyl(2R,3R,4S,5R,6R)-3,4,5-tris(butanoyloxy)-6-(hydroxymethyl)oxan-2-yl(2E)-but-2-enedioate

To a solution of [(2R,3R,4S,5R)-4,5,6-tri(butanoyloxy)-2-(trityloxymethyl)tetrahydropyran-3-yl]butanoate (18 g, 25.61 mmol, 1 equiv.) in THF (100 mL) was added aq. MeNH₂ (2.65 g, 25.61 mmol, 30% purity, 1 equiv.). The mixture was stirred at 15° C. for 18 h. TLC indicated one new spot formed. The reaction mixture was concentrated under reduced pressure to give a residue. The residue was purified by column chromatography (SiO₂, petroleum ether/ethyl acetate=30/1 to 7/1). [(2R,3R,4S,5R)-4,5-di(butanoyloxy)-6-hydroxy-2-(trityloxymethyl)tetrahydropyran-3-yl]butanoate (8 g, 12.64 mmol, 49.37% yield) was obtained as a white solid.

To a solution of (E)-4-methoxy-4-oxo-but-2-enoic acid (1.64 g, 12.64 mmol, 2 equiv.) and DCC (1.96 g, 9.48 mmol, 1.5 equiv.) in DCM (20 mL) was added DMAP (386.16 mg, 3.16 mmol, 0.5 equiv.) and the reaction mixture stirred at 15° C. for 10 min. Then [(2R,3R,4S,5R)-4,5-di(butanoyloxy)-6-hydroxy-2-(trityloxymethyl)tetrahydropyran-3-yl]butanoate (4 g, 6.32 mmol, 1 equiv.) was added to the mixture and the mixture was stirred at 15° C. for 12 h. TLC indicated reactant was consumed completely. The reaction mixture was filtered and the filtrate was concentrated under reduced pressure to give a residue. The residue was purified by column chromatography (SiO₂, petroleum ether/ethyl acetate=20/1 to 6/1). O1-methyl O4-[(3R,4S,5R,6R)-3,4,5-tri(butanoyloxy)-6-(trityloxymethyl)tetrahydropyran-2-yl](E)-but-2-enedioate (2.1 g, 2.82 mmol, 44.60% yield) was obtained as a colorless oil. To a solution of O1-methyl O4-[(3R,4S,5R,6R)-3,4,5-tri(butanoyloxy)-6-(trityloxymethyl)tetrahydropyran-2-yl](E)-but-2-enedioate (2.1 g, 2.82 mmol, 1 equiv.) in HOAc (20 mL) was added H₂O (10.00 g, 555.08 mmol, 10 mL, 196.88 equiv.) at 15° C. and the mixture was stirred at 65° C. for 4 h. The reaction mixture was concentrated under reduced pressure to give a residue. The residue was purified by column chromatography (SiO₂, petroleum ether/ethyl acetate=20/1 to 0/1). O1-methyl-O4-[(3R,4S,5R,6R)-3,4,5-tri(butanoyloxy)-6-(hydroxymethyl)tetrahydropyran-2-yl](E)-but-2-enedioate (1.1 g, 2.19 mmol, 77.64% yield) was obtained as a colorless oil. O1-methyl O4-[(3R,4S,5R,6R)-3,4,5-tri(butanoyloxy)-6-(hydroxymethyl)tetrahydropyran-2-yl](E)-but-2-enedioate (1.39 g, 2.79 mmol, 1 equiv.) was purification by SFC (Neu-MeOH; B %: 13%-13%, 7 min). 1-methyl(2R,3R,4S,5R,6R)-3,4,5-tris(butanoyloxy)-6-(hydroxymethyl)oxan-2-yl(2E)-but-2-enedioate (640 mg, 1.22 mmol, 43.89% yield, 96% purity) was obtained as a white solid. LCMS (M+18)⁺: 520.1 at 3.12 min. ¹H NMR (400 MHz, chloroform-d): δ 6.96 (s, 1H), 6.47 (d, J=3.7 Hz, 1H), 5.60 (t, J=10.0 Hz, 1H), 5.26-5.07 (m, 1H), 3.99-3.46 (m, 4H), 2.45-2.10 (m, 4H), 1.82-1.43 (m, 3H), 1.12-0.71 (m, 5H) ppm.

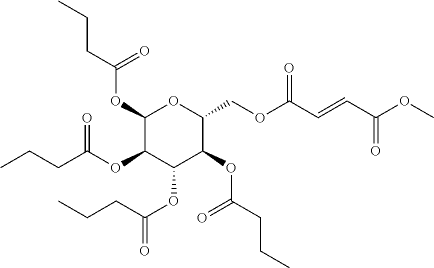 [SMILES: CCCC(=O)O[C@H]1O[C@H](COC(=O)/C=C/C(=O)OC)[C@@H](OC(=O)CCC)[C@H](OC(=O)CCC)[C@H]1OC(=O)CCC]

### Compound 40: 1-methyl 4-[(2R,3R,4S,5R,6R)-3,4,5,6-tetrakis(butanoyloxy)oxan-2-yl]methyl(2E)-but-2-enedioate

To a solution of [(2R,3R,4S,5R)-4,5,6-tri(butanoyloxy)-2-(trityloxymethyl)tetrahydropyran-3-yl]butanoate (8 g, 11.38 mmol, 1 eq) in HOAc (50 mL) was added HBr (2.79 g, 11.38 mmol, 1.87 mL, 33% purity, 1 eq). The mixture was stirred at 15° C. for 0.5 h. TLC indicated reactant was consumed completely. The reaction mixture was filtered and the filtrate was concentrated under reduced pressure to give a residue. The residue was purified by column chromatography (SiO₂, petroleum ether/ethyl acetate 20/1 to 0/1). Compound [(2R,3R,4S,5R)-4,5,6-tri(butanoyloxy)-2-(hydroxymethyl)tetrahydropyran-3-yl]butanoate (2.5 g, 5.43 mmol, 47.69% yield) was obtained as a colorless oil.

To a solution of (E)-4-methoxy-4-oxo-but-2-enoic acid (1.41 g, 10.86 mmol, 2 eq) and DCC (1.68 g, 8.14 mmol, 1.5 eq) in DCM (20 mL) was added DMAP (331.61 mg, 2.71 mmol, 0.5 eq) was stirred at 15° C. for 10 min. Then [(2R,3R,4S,5R)-4,5,6-tri(butanoyloxy)-2-(hydroxymethyl)tetrahydropyran-3-yl]butanoate (2.5 g, 5.43 mmol, 1 eq) was added to the mixture and the mixture was stirred at 15° C. for 12 h. TLC indicated reactant was consumed completely. The reaction mixture filtered and the filtrate was concentrated under reduced pressure to give a residue. The residue was purified by column chromatography (SiO₂, petroleum ether/ethyl acetate=20/1 to 5/1) and subjected to SFC (Neu-IPA; B %: 40%-40%, 4 min) to provide 1-methyl 4-[(2R,3R,4S,5R,6R)-3,4,5,6-tetrakis(butanoyloxy)oxan-2-yl]methyl(2E)-but-2-enedioate (2 g, 3.49 mmol) as a colorless oil. ¹H NMR (400 MHz, chloroform-d): δ 6.81 (s, 2H), 6.29 (d, J=3.6 Hz, 1H), 5.65-5.30 (m, 1H), 5.23-4.86 (m, 2H), 4.30-3.92 (m, 3H), 3.75 (s, 3H), 2.45-2.07 (m, 8H), 1.81-1.40 (m, 8H), 1.02-0.70 (m, 12H). LCMS (M+18)⁺: 590.2 (3.371 min).

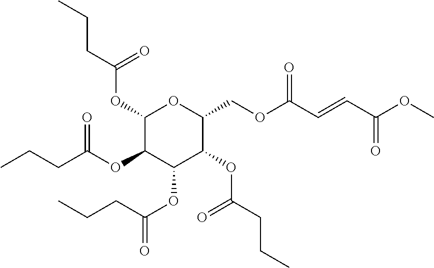 [SMILES: CCCC(=O)O[C@@H]1O[C@H](COC(=O)/C=C/C(=O)OC)[C@H](OC(=O)CCC)[C@H](OC(=O)CCC)[C@H]1OC(=O)CCC]

### Compound 41: 1-methyl 4-[(2R,3S,4S,5R,6S)-3,4,5,6-tetrakis(butanoyloxy)oxan-2-yl]methyl(2E)-but-2-enedioate

(2R,3S,4S,5R)-2,3,4,5,6-pentahydroxyhexanal (5 g, 27.75 mmol, 1 equiv.) and trityl chloride (7.74 g, 27.75 mmol, 1 equiv.) were dissolved in pyridine (100 mL) and the mixture was stirred at 65° C. for 12 h. TLC (petroleum ether/ethyl acetate=0/1, R_{f}=0.1) showed the starting reactant was consumed. The mixture was concentrated. The residue was purified by column chromatography (SiO₂, petroleum ether/ethyl acetate=1/1 to 0/1). (3R,4S,5R,6R)-6-(trityloxymethyl)tetrahydropyran-2,3,4,5-tetrol (7 g, 14.91 mmol, 53.73% yield, 90% purity) was obtained as white solid.

To a solution of (3R,4S,5R,6R)-6-(trityloxymethyl)tetrahydropyran-2,3,4,5-tetrol (7 g, 16.57 mmol, 1 equiv.) in pyridine (100 mL) was added butanoyl butanoate (15.12 g, 95.60 mmol, 15.64 mL, 5.77 equiv.) and the mixture was stirred at 15° C. for 18 h. TLC (petroleum ether/ethyl acetate=3/1, R_{f}=0.6) showed the starting reactant was consumed. The mixture was concentrated. The residue was purified by column chromatography (SiO₂, petroleum ether/ethyl acetate=50/1 to 5/1). [(2R,3S,4S,5R)-4,5,6-tri(butanoyloxy)-2-(trityloxymethyl)tetrahydropyran-3-yl]butanoate was obtained as colorless oil (5 g, 6.40 mmol, 38.64% yield, 90% purity).

To a solution of [(2R,3S,4S,5R)-4,5,6-tri(butanoyloxy)-2-(trityloxymethyl)tetrahydropyran-3-yl]butanoate (5 g, 7.11 mmol, 1 equiv.) in HOAc (50 mL) was added H₂O (25 mL) at 65° C. and the mixture was stirred at 65° C. for 2 h. TLC (petroleum ether/ethyl acetate=1/1, R_{f}=0.5) showed the starting reactant was consumed. The mixture was concentrated. The residue was purified by column chromatography (SiO₂, petroleum ether/ethyl acetate=20/1 to 1/1). [(2R,3S,4S,5R)-4,5,6-tri(butanoyloxy)-2-(hydroxymethyl)tetrahydropyran-3-yl]butanoate (2.7 g, 5.28 mmol, 74.17% yield, 90% purity) was obtained as colorless oil.

To a solution of [(2R,3S,4S,5R)-4,5,6-tri(butanoyloxy)-2-(hydroxymethyl)tetrahydropyran-3-yl]butanoate (2.7 g, 5.86 mmol, 1 equiv.) and (E)-4-methoxy-4-oxo-but-2-enoic acid (1.14 g, 8.79 mmol, 1.5 equiv.) in DCM (30 mL) was added DMAP (214.88 mg, 1.76 mmol, 0.3 equiv.) and DCC (1.81 g, 8.79 mmol, 1.5 equiv.). The mixture was stirred at 15° C. for 12 h. TLC (petroleum ether/ethyl acetate=3/1, R_{f}=0.6) showed the starting reactant was consumed. The mixture was filtered and the filter cake was washed with EtOAc (100 mL). The filtrate was concentrated. The residue was purified by column chromatography (SiO₂, petroleum ether/ethyl acetate=50/1 to 5/1) and by SFC (column: DAICEL CHIRALPAK IC 250 mm×30 mm, 10 μm); mobile phase: Neu-IPA; B %: 45%-45%, 8 min). 1-methyl O4-[[(2R,3S,4S,5R,6S)-3,4,5,6-tetra(butanoyloxy) tetrahydropyran-2-yl]methyl](E)-but-2-enedioate (357 mg, 548.66 μmol, 9.36% yield, 88% purity) was obtained as white solid. LCMS (M)+: 415.2 at 2.351 min. ¹H NMR (400 MHz, chloroform-d): δ 6.86 (d, J=1.3 Hz, 1H), 6.39 (d, J=3.7 Hz, 1H), 5.39 (ddd, J=50.2, 10.7, 3.4 Hz, 2H), 4.57-4.08 (m, 3H), 3.81 (s, 3H), 2.48-2.14 (m, 6H), 1.80-1.49 (m, 6H), 1.11-0.78 (m, 12H) ppm.

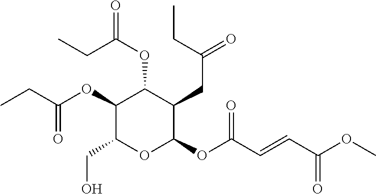 [SMILES: CCC(=O)C[C@H]1[C@@H](OC(=O)/C=C/C(=O)OC)O[C@H](CO)[C@@H](OC(=O)CC)[C@@H]1OC(=O)CC]

### Compound 42: (2R,3R,4S,5R,6R)-6-(hydroxymethyl)-3,4,5-tris(propanoyloxy)oxan-2-yl 1-methyl(2E)-but-2-enedioate

To a mixture of [(2R,3R,4S,5R)-4,5,6-tri(propanoyloxy)-2-(trityloxymethyl)tetrahydropyran-3-yl]propanoate (10 g, 15.46 mmol, 1 equiv.) in THF (50 mL) was added dropwise aq. MeNH₂ (2.40 g, 23.19 mmol, 30% purity, 1.5 equiv.) at 15° C. The resultant mixture was stirred at 15° C. for 10 h under N₂ atmosphere. TLC showed [(2R,3R,4S,5R)-4,5,6-tri(propanoyloxy)-2-(trityloxymethyl)tetrahydropyran-3-yl]propanoate showed was consumed completely. The residue was purified by column chromatography (SiO₂, petroleum ether/ethyl acetate=10/1 to 1/1). [(2R,3R,4S,5R)-6-hydroxy-4,5-di(propanoyloxy)-2-(trityloxymethyl)tetrahydropyran-3-yl]propanoate (3.6 g, 6.09 mmol, 39.42%% yield) was obtained as a colorless oil. A mixture of (E)-4-methoxy-4-oxo-but-2-enoic acid (1.19 g, 9.14 mmol, 1.5 equiv.), DCC (1.89 g, 9.14 mmol, 1.85 mL, 1.5 equiv.) and DMAP (372.30 mg, 3.05 mmol, 0.5 equiv.) in DCM (100 mL) was degassed and purged with N₂ for 3 times at 15° C. The mixture was stirred at 15° C. for 0.5 h. Then, [(2R,3R,4S,5R)-6-hydroxy-4,5-di(propanoyloxy)-2-(trityloxymethyl)tetrahydropyran-3-yl]propanoate (3.6 g, 6.09 mmol, 1 equiv.) was added to the mixture and stirred at 15° C. for 9.5 h. TLC indicated two major new spots with lower polarity was detected. The reaction mixture was filtered and the filtrate was concentrated under reduced pressure to give a residue. The residue was purified by column chromatography (SiO₂, petroleum ether/ethyl acetate=10/1 to 1/1). O1-methyl O4-[(3R,4S,5R,6R)-3,4,5-tri(propanoyloxy)-6-(trityloxymethyl)tetrahydropyran-2-yl](E)-but-2-enedioate (2.2 g, 2.02 mmol, 33.08/yield, 64.40% purity) was obtained as a colorless oil. A mixture of O1-methyl O4-[(3R,4S,5R,6R)-3,4,5-tri(propanoyloxy)-6-(trityloxymethyl)tetrahydropyran-2-yl](E)-but-2-enedioate (2.2 g, 3.13 mmol, 1 equiv.) in AcOH (30 mL) and H₂O (15 mL) was degassed and purged with N₂3 times. Then the mixture was stirred at 65° C. for 4 h under N₂ atmosphere. TLC indicated O1-methyl O4-[(3R,4S,5R,6R)-3,4,5-tri(propanoyloxy)-6-(trityloxymethyl)tetrahydropyran-2-yl](E)-but-2-enedioate was consumed completely and two new spots formed. The reaction mixture was diluted with H₂O (100 mL) and extracted with EtOAc (50 mL×3). The combined organic layers were washed with brine (20 mL), dried over Na₂SO₄, filtered, and the filtrate was concentrated under reduced pressure to give a residue. The residue was purified by column chromatography (SiO₂, petroleum ether/ethyl acetate=3/1 to 1/1). (2R,3R,4S,5R,6R)-6-(hydroxymethyl)-3,4,5-tris(propanoyloxy)oxan-2-yl 1-methyl(2E)-but-2-enedioate (440 mg, 924.38 μmol, 47.29% yield, 96.73% purity) was obtained as a colorless oil. LCMS (M+18)⁺: 478.2. at 3.054 min. ¹H NMR (400 MHz, chloroform-d): δ 6.88 (s, 12H), 6.39 (d, J=3.7 Hz, 1H), 5.50 (t, J=9.9 Hz, 1H), 5.16-4.91 (m, 1H), 3.87 (ddd, J=10.3, 3.9, 2.2 Hz, 1H), 3.78 (s, 3H), 3.59 (dddd, J=58.2, 12.9, 7.0, 3.0 Hz, 1H), 2.40-2.08 (m, 6H), 1.18-0.87 (m, 9H) ppm.

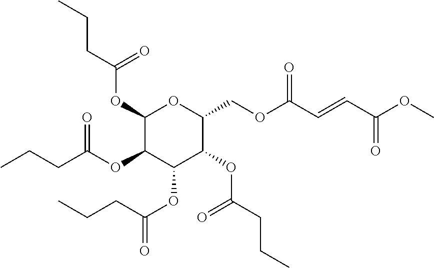 [SMILES: CCCC(=O)O[C@H]1O[C@H](COC(=O)/C=C/C(=O)OC)[C@H](OC(=O)CCC)[C@H](OC(=O)CCC)[C@H]1OC(=O)CCC]

### Compound 43: 1-methyl 4-[(2R,3S,4S,5R,6R)-3,4,5,6-tetrakis(butanoyloxy)oxan-2-yl]methyl(2E)-but-2-enedioate

(2R,3S,4S,5R)-2,3,4,5,6-pentahydroxyhexanal (5 g, 27.75 mmol, 1 equiv.) and trityl chloride (7.74 g, 27.75 mmol, 1 equiv.) was dissolved with pyridine (100 mL) and the mixture was stirred at 65° C. for 12 h. TLC (petroleum ether:ethyl acetate=0/1, R_{f}=0.1) showed the starting reactant was consumed. The mixture was concentrated. The residue was purified by column chromatography (SiO₂, petroleum ether/ethyl acetate=1/1 to 0/1). (3R,4S,5R,6R)-6-(trityloxymethyl)tetrahydropyran-2,3,4,5-tetrol (7 g, 14.91 mmol, 53.73% yield, 90% purity) was obtained as white solid.

To a solution of (3R,4S,5R,6R)-6-(trityloxymethyl)tetrahydropyran-2,3,4,5-tetrol (7 g, 16.57 mmol, 1 equiv.) in pyridine (100 mL) was added butanoyl butanoate (15.12 g, 95.60 mmol, 15.64 mL, 5.77 equiv.) and the mixture was stirred at 15° C. for 18 h. TLC (petroleum ether:ethyl acetate=3/1, R_{f}=0.6) showed the starting reactant was consumed. The mixture was concentrated. The residue was purified by column chromatography (SiO₂, petroleum ether/ethyl acetate=50/1 to 5/1). [(2R,3S,4S,5R)-4,5,6-tri(butanoyloxy)-2-(trityloxymethyl)tetrahydropyran-3-yl]butanoate (5 g, 6.40 mmol, 38.64% yield, 90% purity) was obtained as colorless oil. To a solution of [(2R,3S,4S,5R)-4,5,6-tri(butanoyloxy)-2-(trityloxymethyl)tetrahydropyran-3-yl]butanoate (5 g, 7.11 mmol, 1 equiv.) in HOAc (50 mL) was added H₂O (25 mL) at 65° C. and the mixture was stirred at 65° C. for 2 h. TLC (petroleum ether/ethyl acetate=1/1, R_{f}=0.5) showed the starting reactant was consumed. The mixture was concentrated. The residue was purified by column chromatography (SiO₂, petroleum ether/ethyl acetate=20/1 to 1/1). [(2R,3S,4S,5R)-4,5,6-tri(butanoyloxy)-2-(hydroxymethyl)tetrahydropyran-3-yl]butanoate (2.7 g, 5.28 mmol, 74.17% yield, 90% purity) was obtained as colorless oil.

To a solution of [(2R,3S,4S,5R)-4,5,6-tri(butanoyloxy)-2-(hydroxymethyl)tetrahydropyran-3-yl]butanoate (2.7 g, 5.86 mmol, 1 equiv.) and (E)-4-methoxy-4-oxo-but-2-enoic acid (1.14 g, 8.79 mmol, 1.5 equiv.) in DCM (30 mL) was added DMAP (214.88 mg, 1.76 mmol, 0.3 equiv.) and DCC (1.81 g, 8.79 mmol, 1.5 equiv.). The mixture was stirred at 15° C. for 12 h. TLC (petroleum ether/ethyl acetate=3/1, R_{f}=0.6) showed the starting reactant was consumed. The mixture was filtered, and the filter cake was washed with EtOAc (100 mL). The filtrate was concentrated. The residue was purified by column chromatography (SiO₂, petroleum ether/ethyl acetate=50/1 to 5/1) and by SFC (column: DAICEL CHIRALPAK C 250 mm×30 mm, 10 μm; mobile phase: Neu-IPA; B %: 45%-45%, 8 min). 1-methyl O4-[[(2R,3S,4S,5R,6R)-3,4,5,6-tetra(butanoyloxy) tetrahydropyran-2-yl]methyl](E)-but-2-enedioate (213 mg, 360.83 μmol, 6.15% yield, 97% purity) was obtained as colorless oil. LCMS (M)⁺: 415.1 at 2.327 min. ¹H NMR (400 MHz, chloroform-d): δ 6.87 (d, J=0.9 Hz, 2H), 5.77-5.61 (m, 1H), 5.43 (dd, J=10.3, 8.3 Hz, 1H), 5.04 (td, J=9.5, 8.7, 3.2 Hz, 1H), 4.56-4.31 (m, 2H), 4.09 (s, 3H), 3.95 (t, J=6.3 Hz, 1H), 3.81 (s, 3H), 2.44-2.12 (m, 6H), 1.77-1.43 (m, 6H), 1.05-0.76 (m, 12H) ppm.

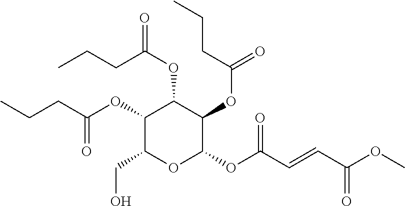 [SMILES: CCCC(=O)O[C@H]1[C@@H](OC(=O)CCC)[C@@H](CO)O[C@@H](OC(=O)/C=C/C(=O)OC)[C@@H]1OC(=O)CCC]

### Compound 44: 1-methyl(2S,3R,4S,5S,6R)-3,4,5-tris(butanoyloxy)-6-(hydroxymethyl)oxan-2-yl(2E)-but-2-enedioate

(2R,3S,4S,5R)-2,3,4,5,6-pentahydroxyhexanal (10 g, 55.51 mmol, 1 equiv.) and trityl chloride (15.47 g, 55.51 mmol, 1 equiv.) was dissolved with pyridine (100 mL) and the mixture was stirred at 65° C. for 12 h. TLC (petroleum ether/ethyl acetate=0/1, R_{f}=0.15) showed the starting reactant was consumed. The mixture was concentrated. The residue was purified by column chromatography (SiO₂, petroleum ether/ethyl acetate=1/1 to 0/1). (3R,4S,5R,6R)-6-(trityloxymethyl)tetrahydropyran-2,3,4,5-tetrol (17 g, 40.24 mmol, 72.49%% yield) was obtained as a white solid. Butanoyl butanoate (36.73 g, 232.18 mmol, 37.98 mL, 5.77 equiv.) was added into a solution of (3R,4S,5R,6R)-6-(trityloxymethyl)tetrahydropyran-2,3,4,5-tetrol (17 g, 40.24 mmol, 1 equiv.) in pyridine (200 mL) and the mixture was stirred at 15° C. for 12 h. TLC (petroleum ether/ethyl acetate=3/1, R_{f}=0.6) showed the starting reactant was consumed. The mixture was concentrated. The residue was purified by column chromatography (SiO₂, petroleum ether/ethyl acetate=20/1 to 3/1). [(2R,3S,4S,5R)-4,5,6-tri(butanoyloxy)-2-(trityloxymethyl)tetrahydropyran-3-yl]butanoate (13 g, 18.50 mmol, 45.97% yield, 90% purity) was obtained as a colorless oil.

To a solution of [(2R,3S,4S,5R)-4,5,6-tri(butanoyloxy)-2-(trityloxymethyl)tetrahydropyran-3-yl]butanoate (7.8 g, 11.10 mmol, 1 equiv.) in THF (20 mL) was added aq. MeNH₂ (1.72 g, 16.65 mmol, 30% purity, 1.5 equiv.) and the mixture was stirred at 15° C. for 12 h. TLC showed the starting reactant was consumed. The mixture was concentrated. The residue was purified by column chromatography (SiO₂, petroleum ether/ethyl acetate=20/1 to 5/1). [(2R,3S,4S,5R)-4,5-di(butanoyloxy)-6-hydroxy-2-(trityloxymethyl)tetrahydropyran-3-yl]butanoate (2.2 g, 3.13 mmol, 28.20/yield, 90% purity) was obtained as white solid.

To a solution of [(2R,3S,4S,5R)-4,5-di(butanoyloxy)-6-hydroxy-2-(trityloxymethyl)tetrahydropyran-3-yl]butanoate (2.2 g, 3.48 mmol, 1 equiv.) and (E)-4-methoxy-4-oxo-but-2-enoic acid (678.52 mg, 5.22 mmol, 1.5 equiv.) in THF (20 mL) was added DCC (1.08 g, 5.22 mmol, 1.05 mL, 1.5 equiv.) and DMAP (127.43 mg, 1.04 mmol, 0.3 equiv.). The reaction mixture was stirred at 15° C. for 12 h. TLC (petroleum ether:ethyl acetate=3/1, R_{f}=0.6) showed the starting reactant was consumed. The mixture was filtered and the filter cake was washed with EtOAc (100 mL), then the filtrate was concentrated. The residue was purified by column chromatography (SiO₂, petroleum ether/ethyl acetate=50/1 to 5/1). O1-methyl O4-[(3R,4S,5S,6R)-3,4,5-tri(butanoyloxy)-6-(trityloxymethyl)tetrahydropyran-2-yl](E)-but-2-enedioate (0.7 g, 845.84 μmol, 24.33% yield, 90% purity) was obtained as colorless oil.

To a solution of O1-methyl O4-[(3R,4S,5S,6R)-3,4,5-tri(butanoyloxy)-6-(trityloxymethyl)tetrahydropyran-2-yl](E)-but-2-enedioate (0.7 g, 939.82 μmol, 1 equiv.) in HOAc (10 mL) was added H₂O (5 mL) at 65° C., and then stirred at 65° C. for 2 h. TLC (petroleum ether/ethyl acetate=3/1, R_{f}=0.3) showed the starting reactant was consumed and LCMS showed the same result. The mixture was filtered and the filtrate was concentrated. The residue was purified by column chromatography (SiO₂, petroleum ether/ethyl acetate=50/1 to 5/1) first. Then the residue was re-purified by prep-HPLC (column: Nano-micro Kromasil C18 100×40 mm 10 μm; mobile phase: water+0.1% (v/v) TFA/ACN; B %: 48%-68%, 9 min). The residue was separated by prep-SFC (column: Phenomenex-Cellulose-2 250 mm×30 mm, μm; mobile phase: Neu-ACN; B %: 40%-40%, 10 min). 1-methyl(2S,3R,4S,5S,6R)-3,4,5-tris(butanoyloxy)-6-(hydroxymethyl)oxan-2-yl(2E)-but-2-enedioate (55 mg, 101.79 μmol, 10.83% yield, 93% purity) was obtained as a white solid. LCMS (M)+: 415.1 at 3.706 min. ¹H NMR (400 MHz, chloroform-d): δ 6.88-6.67 (m, 2H), 5.77 (dd, J=10.2, 8.2 Hz, 1H), 5.55-5.37 (m, 2H), 5.20 (dd, J=10.4, 3.4 Hz, 1H), 4.02-3.85 (m, 1H), 3.81 (d, J=2.9 Hz, 3H), 3.74 (dt, J=11.8, 6.6 Hz, 1H), 3.51 (dt, J=11.8, 7.0 Hz, 1H), 2.56-2.10 (m, 9H), 1.80-1.46 (m, 9H), 1.09-0.74 (m, 12H) ppm.

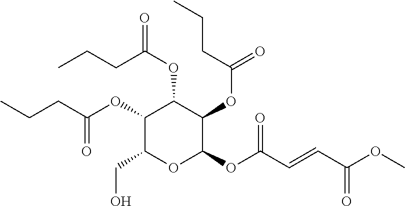 [SMILES: CCCC(=O)O[C@H]1[C@@H](OC(=O)CCC)[C@@H](CO)O[C@H](OC(=O)/C=C/C(=O)OC)[C@@H]1OC(=O)CCC]

### Compound 45: 1-methyl(2R,3R,4S,5S,6R)-3,4,5-tris(butanoyloxy)-6-(hydroxymethyl)oxan-2-yl(2E)-but-2-enedioate

(2R,3S,4S,5R)-2,3,4,5,6-pentahydroxyhexanal (10 g, 55.51 mmol, 1 equiv.) and trityl chloride (15.47 g, 55.51 mmol, 1 equiv.) was dissolved with pyridine (100 mL) and the mixture was stirred at 65° C. for 12 h. TLC (petroleum ether:ethyl acetate=0/1, R_{f}-0.15) showed the starting reactant was consumed. The mixture was concentrated. The residue was purified by column chromatography (SiO₂, petroleum ether/ethyl acetate=1/1 to 0/1). (3R,4S,5R,6R)-6-(trityloxymethyl)tetrahydropyran-2,3,4,5-tetrol (17 g, 40.24 mmol, 72.49%% yield) was obtained as a white solid.

Butanoyl butanoate (36.73 g, 232.18 mmol, 37.98 mL, 5.77 equiv.) was added into a solution of (3R,4S,5R,6R)-6-(trityloxymethyl)tetrahydropyran-2,3,4,5-tetrol (17 g, 40.24 mmol, 1 equiv.) in pyridine (200 mL) and the mixture was stirred at 15° C. for 12 h. TLC (petroleum ether/ethyl acetate=3/1, R_{f}=0.6) showed the starting reactant was consumed. The mixture was concentrated. The residue was purified by column chromatography (SiO₂, petroleum ether/ethyl acetate=20/1 to 3/1). [(2R,3S,4S,5R)-4,5,6-tri(butanoyloxy)-2-(trityloxymethyl)tetrahydropyran-3-yl]butanoate (13 g, 18.50 mmol, 45.97% yield, 90% purity) was obtained as a colorless oil.

To a solution of [(2R,3S,4S,5R)-4,5,6-tri(butanoyloxy)-2-(trityloxymethyl)tetrahydropyran-3-yl]butanoate (7.8 g, 11.10 mmol, 1 equiv.) in THF (20 mL) was added aq. MeNH₂ (1.72 g, 16.65 mmol, 30% purity, 1.5 equiv.) and the mixture was stirred at 15° C. for 12 h. TLC showed the starting reactant was consumed. The mixture was concentrated. The residue was purified by column chromatography (SiO₂, petroleum ether/ethyl acetate=20/1 to 5/1). [(2R,3S,4S,5R)-4,5-di(butanoyloxy)-6-hydroxy-2-(trityloxymethyl)tetrahydropyran-3-yl]butanoate (2.2 g, 3.13 mmol, 28.20/yield, 90% purity) was obtained as white solid. To a solution of [(2R,3S,4S,5R)-4,5-di(butanoyloxy)-6-hydroxy-2-(trityloxymethyl)tetrahydropyran-3-yl]butanoate (2.2 g, 3.48 mmol, 1 equiv.) and (E)-4-methoxy-4-oxo-but-2-enoic acid (678.52 mg, 5.22 mmol, 1.5 equiv.) in THF (20 mL) was added DCC (1.08 g, 5.22 mmol, 1.05 mL, 1.5 equiv.) and DMAP (127.43 mg, 1.04 mmol, 0.3 equiv.). The reaction mixture was stirred at 15° C. for 12 h. TLC (petroleum ether/ethyl acetate=3/1, R_{f}=0.6) showed the starting reactant was consumed. The mixture was filtered and the filter cake was washed with EtOAc (100 mL), then the filtrate was concentrated. The residue was purified by column chromatography (SiO₂, petroleum ether/ethyl acetate=50/1 to 5/1). O1-methyl O4-[(3R,4S,5S,6R)-3,4,5-tri(butanoyloxy)-6-(trityloxymethyl)tetrahydropyran-2-yl](E)-but-2-enedioate (0.7 g, 845.84 μmol, 24.33% yield, 90% purity) was obtained as colorless oil.

To a solution of O1-methyl O4-[(3R,4S,5S,6R)-3,4,5-tri(butanoyloxy)-6-(trityloxymethyl)tetrahydropyran-2-yl](E)-but-2-enedioate (0.7 g, 939.82 μmol, 1 equiv.) in HOAc (10 mL) was added H₂O (5 mL) at 65° C., and then stirred at 65° C. for 2 h. TLC (petroleum ether/ethyl acetate=3/1, R_{f}=0.3) and LCMS showed the starting reactant was consumed. The mixture was filtered and the filtrate was concentrated. The residue was purified by column chromatography (SiO₂, petroleum ether/ethyl acetate=50/1 to 5/1) first. Then the residue was re-purified by prep-HPLC (column: Nano-micro Kromasil C18 100×40 mm 10 μm; mobile phase: water+0.1% (v/v) TFA/ACN; B %: 48%-68%, 9 min). The residue was separated by prep-SFC (column: Phenomenex-Cellulose-2 250 mm×30 mm, 10 μm); mobile phase: Neu-ACN; B %: 40%-40%, 10 min). 1-methyl(2R,3R,4S,5S,6R)-3,4,5-tris(butanoyloxy)-6-(hydroxymethyl)oxan-2-yl(2E)-but-2-enedioate (73 mg, 139.46 μmol, 14.84% yield, 96% purity) was obtained as white solid. ¹H NMR (400 MHz, chloroform-d): δ 6.92-6.64 (m, 2H), 6.41 (d, J=2.8 Hz, 1H), 5.62-5.42 (m, 3H), 5.30 (s, 1H), 4.20 (t, J=6.6 Hz, 1H), 3.81 (s, 3H), 3.78-3.61 (m, 2H), 3.47 (dt, J=11.8, 7.0 Hz, 1H), 2.52-2.01 (m, 6H), 1.77-1.48 (m, 6H), 1.07-0.66 (m, 12H) ppm.

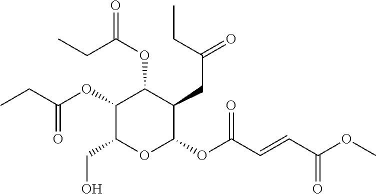 [SMILES: CCC(=O)C[C@H]1[C@H](OC(=O)/C=C/C(=O)OC)O[C@H](CO)[C@H](OC(=O)CC)[C@@H]1OC(=O)CC]

### Compound 46: (2S,3R,4S,5S,6R)-6-(hydroxymethyl)-3,4,5-tris(propanoyloxy)oxan-2-yl 1-methyl(2E)-but-2-enedioate

A mixture of (2R,3S,4S,5R)-2,3,4,5,6-pentahydroxyhexanal (20 g, 111.01 mmol, 1 equiv.) and trityl chloride (30.95 g, 111.01 mmol, 1 equiv.) in pyridine (100 mL) was degassed and purged with N₂3 times. Then the mixture was stirred at 65° C. for 5 h under N₂ atmosphere. TLC indicated (2R,3S,4S,5R)-2,3,4,5,6-pentahydroxyhexanal was consumed completely and three new spots formed. The reaction product, (3R,4S,5R,6R)-6-(trityloxymethyl)tetrahydropyran-2,3,4,5-tetrol (111.01 mmol, 100 mL) in pyridine as crude solution was used for next step directly.

To a solution of (3R,4S,5R,6R)-6-(trityloxymethyl)tetrahydropyran-2,3,4,5-tetrol (111.01 mmol, 100 mL, 1 equiv.) in a pyridine solution was added propanoyl propanoate (36.96 g, 284.04 mmol, 36.6 mL, 6 equiv.) at 15° C. Then the mixture was stirred at 15° C. for 10 h under N₂ atmosphere. TLC indicated (3R,4S,5R,6R)-6-(trityloxymethyl)tetrahydropyran-2,3,4,5-tetrol was consumed completely and three spots formed. The reaction mixture was quenched by addition H₂O (300 mL) at 15° C. and extracted with EtOAc 300 mL (100 mL×3). The combined organic layers were washed with brine (50 mL), dried over Na₂SO₄, filtered, and then the filtrate was concentrated under reduced pressure to give a residue. The residue was purified by column chromatography (SiO₂, petroleum ether/ethyl acetate=10/1 to 1/1). Compound [(2R,3S,4S,5R)-4,5,6-tri(propanoyloxy)-2-(trityloxymethyl)tetrahydropyran-3-yl]propanoate (30 g, 46.39 mmol, 97.99%% yield) was obtained as colorless oil.

To a solution of [(2R,3S,4S,5R)-4,5,6-tri(propanoyloxy)-2-(trityloxymethyl)tetrahydropyran-3-yl]propanoate (10 g, 15.46 mmol, 1 equiv.) in THF (100 mL) was added MeNH₂ (2.40 g, 23.19 mmol, 30% purity, 1.5 equiv., in H₂O). The mixture was stirred at 15° C. for 10 h. TLC indicated [(2R,3S,4S,5R)-4,5,6-tri(propanoyloxy)-2-(trityloxymethyl)tetrahydropyran-3-yl]propanoate remained, and one new spot formed. The reaction mixture was concentrated under reduced pressure to give a residue. The residue was purified by column chromatography (SiO₂, petroleum ether/ethyl acetate=50/1 to 0/1) to give [(2R,3S,4S,5R)-6-hydroxy-4,5-di(propanoyloxy)-2-(trityloxymethyl)tetrahydropyran-3-yl]propanoate (3.7 g, 6.26 mmol, 40.51% yield) as a white solid.

To a solution of (E)-4-methoxy-4-oxo-but-2-enoic acid (1.22 g, 9.40 mmol, 1.5 equiv.) and DCC (1.94 g, 9.40 mmol, 1.90 mL, 1.5 equiv.) in DCM (37 mL) was added DMAP (382.64 mg, 3.13 mmol, 0.5 equiv.) and [(2R,3S,4S,5R)-6-hydroxy-4,5-di(propanoyloxy)-2-(trityloxymethyl)tetrahydropyran-3-yl]propanoate (3.7 g, 6.26 mmol, 1 equiv.). The mixture was stirred at 15° C. for 10 h under N₂. TLC indicated [(2R,3S,4S,5R)-6-hydroxy-4,5-di(propanoyloxy)-2-(trityloxymethyl)tetrahydropyran-3-yl]propanoate remained, and one new spot was detected. LCMS showed desired mass was detected. The reaction mixture was filtered, and the filtrate was concentrated under reduced pressure to give a residue. The residue was purified by column chromatography (SiO₂, petroleum ether/ethyl acetate=50/1 to 0/1) and prep-HPLC (water+0.1% (v/v) TFA/ACN) to give peak 1 for O1-methyl O4-[(2S,3R,4S,5S,6R)-3,4,5-tri(propanoyloxy)-6-(trityloxymethyl)tetrahydropyran-2-yl](E)-but-2-enedioate (1.6 g, 2.28 mmol, 36.35% yield) as a yellow solid and peak 2 for O1-methyl O4-[(2R,3R,4S,5S,6R)-3,4,5-tri(propanoyloxy)-6-(trityloxymethyl)tetrahydropyran-2-yl](E)-but-2-enedioate (1.6 g, 2.28 mmol, 36.35% yield) as a yellow solid.

O1-methyl O4-[(2S,3R,4S,5S,6R)-3,4,5-tri(propanoyloxy)-6-(trityloxymethyl)tetrahydropyran-2-yl](E)-but-2-enedioate (500 mg, 711.50 μmol, 1 equiv.) was added in HOAc (10 mL) at 15° C., then H₂O (5.00 g, 277.54 mmol, 5 mL) was added to the mixture at 65° C. and the mixture was stirred at 65° C. for 3 h. LC-MS showed the desired compound was detected. The reaction mixture was concentrated under reduced pressure to give a residue. The residue was purified by prep-HPLC (water+0.05% HCl (v/v)/ACN). (2S,3R,4S,5S,6R)-6-(hydroxymethyl)-3,4,5-tris(propanoyloxy)oxan-2-yl 1-methyl(2E)-but-2-enedioate (150 mg, 325.78 μmol, 45.79/yield) was obtained as a white solid. LCMS (M+18)⁺478.3 at 1.187 min. ¹H NMR (400 MHz, chloroform-d): δ 6.94-6.69 (m, 2H), 5.73 (d, J=8.3 Hz, 1H), 5.48-5.31 (m, 2H), 5.10 (dd, J=10.4, 3.4 Hz, 1H), 3.88 (td, J=6.5, 1.1 Hz, 1H), 3.75 (s, 3H), 3.67 (dt, J=11.7, 6.4 Hz, 1H), 3.46 (dt, J=11.8, 6.8 Hz, 1H), 2.41 (qd, J=7.5, 2.3 Hz, 2H), 2.28-2.01 (m, 4H), 1.26-0.90 (m, 9H) ppm.

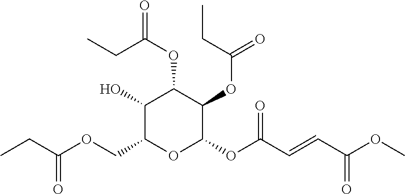 [SMILES: CCC(=O)OC[C@H]1O[C@@H](OC(=O)/C=C/C(=O)OC)[C@H](OC(=O)CC)[C@@H](OC(=O)CC)[C@H]1O]

### Compound 47: (2S,3R,4S,5S,6R)-5-hydroxy-3,4-bis(propanoyloxy)-6-[(propanoyloxy)methyl]oxan-2-yl 1-methyl(2E)-but-2-enedioate

A mixture of (2R,3S,4S,5R)-2,3,4,5,6-pentahydroxyhexanal (20 g, 111.01 mmol, 1 equiv.) and trityl chloride (30.95 g, 111.01 mmol, 1 equiv.) in pyridine (100 mL) was degassed and purged with N₂3 times, and then the mixture was stirred at 65° C. for 5 h under N₂ atmosphere. TLC indicated (2R,3S,4S,5R)-2,3,4,5,6-pentahydroxyhexanal was consumed completely and three new spots formed. The reaction mixture yielded (3R,4S,5R,6R)-6-(trityloxymethyl)tetrahydropyran-2,3,4,5-tetrol (111.01 mmol, 100 mL) in pyridine as crude solution and was used for next step directly.

To a solution of (3R,4S,5R,6R)-6-(trityloxymethyl)tetrahydropyran-2,3,4,5-tetrol (111.01 mmol, 100 mL, 1 equiv.) in pyridine was added propanoyl propanoate (36.96 g, 284.04 mmol, 36.6 mL, 6 equiv.) at 15° C. Then the mixture was stirred at 15° C. for 10 h under N₂ atmosphere. TLC indicated (3R,4S,5R,6R)-6-(trityloxymethyl)tetrahydropyran-2,3,4,5-tetrol was consumed completely and formation of three new spots. The reaction mixture was quenched by addition of H₂O (300 mL) at 15° C. and extracted with EtOAc 300 mL (100 mL×3). The combined organic layers were washed with brine (50 mL), dried over Na₂SO₄, filtered, and then the filtrate was concentrated under reduced pressure to give a residue. The residue was purified by column chromatography (SiO₂, petroleum ether/ethyl acetate=10/1 to 1/1). [(2R,3S,4S,5R)-4,5,6-tri(propanoyloxy)-2-(trityloxymethyl)tetrahydropyran-3-yl]propanoate (30 g, 46.39 mmol, 97.99%% yield) was obtained as colorless oil.

To a solution of [(2R,3S,4S,5R)-4,5,6-tri(propanoyloxy)-2-(trityloxymethyl)tetrahydropyran-3-yl]propanoate (10 g, 15.46 mmol, 1 equiv.) in THF (100 mL) was added MeNH₂ (2.40 g, 23.19 mmol, 30% purity, 1.5 equiv., in H₂O). The mixture was stirred at 15° C. for 10 h. TLC indicated [(2R,3S,4S,5R)-4,5,6-tri(propanoyloxy)-2-(trityloxymethyl)tetrahydropyran-3-yl]propanoate remained, and one new spot formed. The reaction mixture was concentrated under reduced pressure to give a residue. The residue was purified by column chromatography (SiO₂, petroleum ether/ethyl acetate=50/1 to 0/1) to give [(2R,3S,4S,5R)-6-hydroxy-4,5-di(propanoyloxy)-2-(trityloxymethyl)tetrahydropyran-3-yl]propanoate (3.7 g, 6.26 mmol, 40.51% yield) as a white solid.

To a solution of (E)-4-methoxy-4-oxo-but-2-enoic acid (1.22 g, 9.40 mmol, 1.5 equiv.) and DCC (1.94 g, 9.40 mmol, 1.90 mL, 1.5 equiv.) in DCM (37 mL) was added DMAP (382.64 mg, 3.13 mmol, 0.5 equiv.) and [(2R,3S,4S,5R)-6-hydroxy-4,5-di(propanoyloxy)-2-(trityloxymethyl)tetrahydropyran-3-yl]propanoate (3.7 g, 6.26 mmol, 1 equiv.). The mixture was stirred at 15° C. for 10 h under N₂. TLC indicated [(2R,3S,4S,5R)-6-hydroxy-4,5-di(propanoyloxy)-2-(trityloxymethyl)tetrahydropyran-3-yl]propanoate remained, and one new spot was detected. LCMS showed desired mass was detected. The reaction mixture was filtered and the filtrate was concentrated under reduced pressure to give a residue. The residue was purified by column chromatography (SiO₂, petroleum ether/ethyl acetate=50/1 to 0/1) and prep-HPLC (water+0.1% (v/v) TFA/ACN) to give O1-methyl O4-[(2S,3R,4S,5S,6R)-3,4,5-tri(propanoyloxy)-6-(trityloxymethyl)tetrahydropyran-2-yl](E)-but-2-enedioate (1.6 g, 2.28 mmol, 36.35% yield) as a yellow solid and O1-methyl O4-[(2R,3R,4S,5S,6R)-3,4,5-tri(propanoyloxy)-6-(trityloxymethyl)tetrahydropyran-2-yl](E)-but-2-enedioate (1.6 g, 2.28 mmol, 36.35% yield) as a yellow solid. O1-methyl O4-[(2S,3R,4S,5S,6R)-3,4,5-tri(propanoyloxy)-6-(trityloxymethyl)tetrahydropyran-2-yl](E)-but-2-enedioate (500 mg, 711.50 μmol, 1 equiv.) was added in HOAc (10 mL) at 15° C., Then H₂O (5.00 g, 277.54 mmol, 5 mL) was added to the mixture at 65° C. and the mixture was stirred at 65° C. for 3 h. LCMS showed the desired compound was detected. The reaction mixture was concentrated under reduced pressure to give a residue. The residue was purified by prep-HPLC (water+0.05% HCl (v/v)/ACN). (2S,3R,4S,5S,6R)-5-hydroxy-3,4-bis(propanoyloxy)-6-[(propanoyloxy)methyl]oxan-2-yl 1-methyl(2E)-but-2-enedioate (8 mg, 17.38 μmol, 2.96% yield) was obtained as a colorless oil. LCMS (M+18)⁺: 478.2. ¹H NMR (400 MHz, chloroform-d): δ 6.95-6.58 (m, 2H), 5.70 (d, J=8.3 Hz, 1H), 5.42 (dd, J=10.3, 8.3 Hz, 1H), 5.00 (dd, J=10.2, 3.2 Hz, 1H), 4.33 (dd, J=11.6, 6.1 Hz, 1H), 4.20 (dd, J=11.6, 6.5 Hz, 1H), 4.10-3.95 (m, 1H), 3.87 (td, J=6.3, 1.1 Hz, 1H), 3.74 (s, 3H), 2.43-2.05 (m, 6H), 1.04 (dt, J=23.9, 7.6 Hz, 9H) ppm.

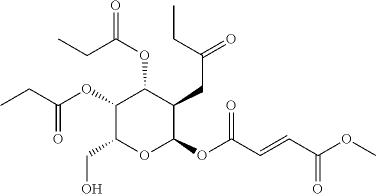 [SMILES: CCC(=O)C[C@H]1[C@@H](OC(=O)/C=C/C(=O)OC)O[C@H](CO)[C@H](OC(=O)CC)[C@@H]1OC(=O)CC]

### Compound 48: (2R,3R,4S,5S,6R)-6-(hydroxymethyl)-3,4,5-tris(propanoyloxy)oxan-2-yl 1-methyl(2E)-but-2-enedioate

A mixture of (2R,3S,4S,5R)-2,3,4,5,6-pentahydroxyhexanal (20 g, 111.01 mmol, 1 equiv.) and trityl chloride (30.95 g, 111.01 mmol, 1 equiv.) in pyridine (100 mL) was degassed and purged with N₂3 times. Then the mixture was stirred at 65° C. for 5 h under N₂ atmosphere. TLC indicated (2R,3S,4S,5R)-2,3,4,5,6-pentahydroxyhexanal was consumed completely and three new spots formed. The reaction yielded (3R,4S,5R,6R)-6-(trityloxymethyl)tetrahydropyran-2,3,4,5-tetrol as a crude pyridine solution (111.01 mmol, 100 ml)) and was used for in the next step directly.

To a pyridine solution of (3R,4S,5R,6R)-6-(trityloxymethyl)tetrahydropyran-2,3,4,5-tetrol (111.01 mmol, 100 mL, 1 equiv.) was added propanoyl propanoate (36.96 g, 284.04 mmol, 36.6 mL, 6 equiv.) at 15° C., and then the mixture was stirred at 15° C. for 10 h under N₂ atmosphere. TLC indicated (3R,4S,5R,6R)-6-(trityloxymethyl)tetrahydropyran-2,3,4,5-tetrol was consumed completely and three spots formed. The reaction mixture was quenched by addition H₂O (300 mL) at 15° C. and extracted with EtOAc 300 mL (100 mL×3). The combined organic layers were washed with brine (50 mL), dried over Na₂SO₄, filtered, and the filtrate was concentrated under reduced pressure to give a residue. The residue was purified by column chromatography (SiO₂, petroleum ether/ethyl acetate=10/1 to 1/1). [(2R,3S,4S,5R)-4,5,6-tri(propanoyloxy)-2-(trityloxymethyl)tetrahydropyran-3-yl]propanoate (30 g, 46.39 mmol, 97.99% yield) was obtained as colorless oil.

To a solution of [(2R,3S,4S,5R)-4,5,6-tri(propanoyloxy)-2-(trityloxymethyl)tetrahydropyran-3-yl]propanoate (10 g, 15.46 mmol, 1 equiv.) in THF (100 mL) was added MeNH₂ (2.40 g, 23.19 mmol, 30% purity, 1.5 equiv., in H₂O). The mixture was stirred at 15° C. for 10 h. TLC indicated [(2R,3S,4S,5R)-4,5,6-tri(propanoyloxy)-2-(trityloxymethyl)tetrahydropyran-3-yl]propanoate remained, and one new spot formed. The reaction mixture was concentrated under reduced pressure to give a residue. The residue was purified by column chromatography (SiO₂, petroleum ether/ethyl acetate=50/1 to 0/1) to give [(2R,3S,4S,5R)-6-hydroxy-4,5-di(propanoyloxy)-2-(trityloxymethyl)tetrahydropyran-3-yl]propanoate (3.7 g, 6.26 mmol, 40.51% yield) as a white solid.

To a solution of (E)-4-methoxy-4-oxo-but-2-enoic acid (1.22 g, 9.40 mmol, 1.5 equiv.) and DCC (1.94 g, 9.40 mmol, 1.90 mL, 1.5 equiv.) in DCM (37 mL) was added DMAP (382.64 mg, 3.13 mmol, 0.5 equiv.) and [(2R,3S,4S,5R)-6-hydroxy-4,5-di(propanoyloxy)-2-(trityloxymethyl)tetrahydropyran-3-yl]propanoate (3.7 g, 6.26 mmol, 1 equiv.). The mixture was stirred at 15° C. for 10 h under N₂. TLC indicated [(2R,3S,4S,5R)-6-hydroxy-4,5-di(propanoyloxy)-2-(trityloxymethyl)tetrahydropyran-3-yl]propanoate remained, and one new spot was detected. LCMS showed desired mass was detected. The reaction mixture was filtered and the filtrate was concentrated under reduced pressure to give a residue. The residue was purified by column chromatography (SiO₂, petroleum ether/ethyl acetate=50/1 to 0/1) and prep-HPLC (water+0.1% (v/v) TFA/ACN) to give O1-methyl O4-[(2S,3R,4S,5S,6R)-3,4,5-tri(propanoyloxy)-6-(trityloxymethyl)tetrahydropyran-2-yl](E)-but-2-enedioate as a yellow solid and peak 2 for O1-methyl O4-[(2R,3R,4S,5S,6R)-3,4,5-tri(propanoyloxy)-6-(trityloxymethyl)tetrahydropyran-2-yl](E)-but-2-enedioate (1.6 g, 2.28 mmol, 36.35% yield) as a yellow solid.

O1-methyl O4-[(2R,3R,4S,5S,6R)-3,4,5-tri(propanoyloxy)-6-(trityloxymethyl)tetrahydropyran-2-yl](E)-but-2-enedioate (1.60 g, 2.28 mmol, 1 equiv.) was added in HOAc (20 mL) was at 15° C., then H₂O (10.00 g, 555.08 mmol, 10 mL, 243.80 equiv.) was added to the mixture at 65° C. The mixture was stirred at 65° C. for 3 h. LC-MS showed the desired compound was detected. The reaction mixture was concentrated under reduced pressure to give a residue. The residue was purified by prep-HPLC (water+0.05% HCl (v/v)/ACN) to get 200 mg crude product which was further separated by prep-TLC (SiO₂, petroleum ether/EtOAc=3/1) and purified again by SFC (Neu-IPA) to get pure (2R,3R,4S,5S,6R)-6-(hydroxymethyl)-3,4,5-tris(propanoyloxy)oxan-2-yl 1-methyl(2E)-but-2-enedioate (23 mg, 49.45 μmol, 75.90/yield, 99/6 purity) as a colorless oil. LCMS (M+18)⁺: 478.2 at 2.724 min. ¹H NMR (400 MHz, chloroform-d): δ 6.85 (s, 2H), 6.42 (d, J=3.1 Hz, 1H), 5.54-5.25 (m, 3H), 4.16 (t, J=6.5 Hz, 1H), 3.78 (s, 3H), 3.62 (dt, J=12.3, 6.3 Hz, 1H), 3.43 (dt, J=11.7, 6.7 Hz, 1H), 2.51-1.95 (m, 6H), 1.23-0.89 (m, 9H) ppm.

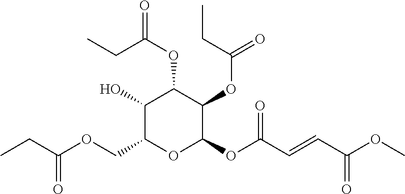 [SMILES: CCC(=O)OC[C@H]1O[C@H](OC(=O)/C=C/C(=O)OC)[C@H](OC(=O)CC)[C@@H](OC(=O)CC)[C@H]1O]

### Compound 49: (2R,3R,4S,5S,6R)-5-hydroxy-3,4-bis(propanoyloxy)-6-[(propanoyloxy)methyl]oxan-2-yl 1-methyl(2E)-but-2-enedioate

A mixture of (2R,3S,4S,5R)-2,3,4,5,6-pentahydroxyhexanal (20 g, 111.01 mmol, 1 equiv.) and trityl chloride (30.95 g, 111.01 mmol, 1 equiv.) in pyridine (100 mL) was degassed and purged with N₂3 times, and then the mixture was stirred at 65° C. for 5 h under N₂ atmosphere. TLC indicated (2R,3S,4S,5R)-2,3,4,5,6-pentahydroxyhexanal was consumed completely and three new spots formed. The crude reaction mixture (3R,4S,5R,6R)-6-(trityloxymethyl)tetrahydropyran-2,3,4,5-tetrol (111.01 mmol, 100 mL)) in pyridine was used for next step directly.

To a crude pyridine solution of (3R,4S,5R,6R)-6-(trityloxymethyl)tetrahydropyran-2,3,4,5-tetrol (111.01 mmol, 100 mL, 1 equiv.) was added propanoyl propanoate (36.96 g, 284.04 mmol, 36.6 mL, 6 equiv.) at 15° C., and then the mixture was stirred at 15° C. for 10 h under N₂ atmosphere. TLC indicated (3R,4S,5R,6R)-6-(trityloxymethyl)tetrahydropyran-2,3,4,5-tetrol was consumed completely and three spots formed. The reaction mixture was quenched by addition of H₂O (300 mL) at 15° C. and extracted with EtOAc 300 mL (100 mL×3). The combined organic layers were washed with brine (50 mL), dried over Na₂SO₄, filtered, and the filtrate was concentrated under reduced pressure to give a residue. The residue was purified by column chromatography (SiO₂, petroleum ether/ethyl acetate=10/1 to 1/1). [(2R,3S,4S,5R)-4,5,6-tri(propanoyloxy)-2-(trityloxymethyl)tetrahydropyran-3-yl]propanoate (30 g, 46.39 mmol, 97.99% yield) was obtained as colorless oil.

To a solution of [(2R,3S,4S,5R)-4,5,6-tri(propanoyloxy)-2-(trityloxymethyl)tetrahydropyran-3-yl]propanoate (10 g, 15.46 mmol, 1 equiv.) in THF (100 mL) was added MeNH₂ (2.40 g, 23.19 mmol, 30% purity, 1.5 equiv., in H₂O). The mixture was stirred at 15° C. for 10 h. The reaction mixture was filtered and the filtrate was concentrated under reduced pressure to give a residue. The reaction mixture was concentrated under reduced pressure to give a residue. The residue was purified by prep-HPLC (water+0.05% HCl (v/v)/ACN). O4-[(2R,3R,4S,5S,6R)-5-hydroxy-3,4-di(propanoyloxy)-6-(propanoyloxymethyl)tetrahydropyran-2-yl]O1-methyl(E)-but-2-enedioate (140 mg, 304.06 μmol, 13.35% yield, 100% purity) was obtained as a colorless oil. LCMS (M+18)⁺: 478.2 at 2.724 min. ¹H NMR (400 MHz, chloroform-d): δ 6.85 (s, 2H), 6.40 (d, J=3.7 Hz, 1H), 5.44 (dd, J=10.8, 3.8 Hz, 1H), 5.25 (dd, J=10.7, 2.9 Hz, 1H), 4.34 (td, J=9.1, 6.0 Hz, 1H), 4.13 (dq, J=9.4, 4.9, 3.5 Hz, 3H), 3.77 (s, 3H), 2.50-2.06 (m, 8H), 1.19-0.95 (m, 9H) ppm.

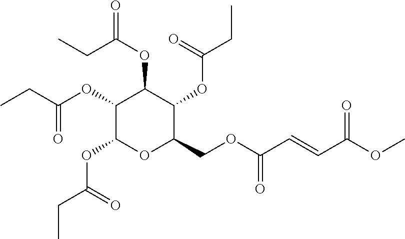 [SMILES: CCC(=O)O[C@H]1O[C@H](COC(=O)/C=C/C(=O)OC)[C@@H](OC(=O)CC)[C@H](OC(=O)CC)[C@H]1OC(=O)CC]

### Compound 50: 1-methyl 4-[(2R,3R,4S,5R,6R)-3,4,5,6-tetrakis(propanoyloxy)oxan-2-yl]methyl(2E)-but-2-enedioate

A mixture of (3R,4S,5S,6R)-6-(hydroxymethyl)tetrahydropyran-2,3,4,5-tetrol (20 g, 111.01 mmol, 1 equiv.) and [chloro(diphenyl)methyl]benzene (30.95 g, 111.01 mmol, 1 equiv.) in pyridine (100 mL) was degassed and purged with N₂₃ times. Then the mixture was stirred at 15° C. for 10 h under N₂ atmosphere. TLC indicated (3R,4S,5S,6R)-6-(hydroxymethyl)tetrahydropyran-2,3,4,5-tetrol was consumed completely and three new spots formed. (3R,4S,5S,6R)-6-(trityloxymethyl)tetrahydropyran-2,3,4,5-tetrol (crude, ˜111 mmol) in pyridine as a crude solution was used directly in next step.

To the above solution of (3R,4S,5S,6R)-6-(trityloxymethyl)tetrahydropyran-2,3,4,5-tetrol (˜111 mmol, 1 equiv.) in pyridine was added propionic anhydride (72.23 g, 555.00 mmol, 71.51 mL, 5 equiv.) at 15° C. Then the mixture was heated to 65° C. and stirred at 65° C. for 10 h under N₂ atmosphere. TLC indicated three major spots with lower polarity were detected. The reaction mixture was diluted with H₂O (500 mL) and extracted with EtOAc (150 mL×3). The combined organic layers were washed with brine (50 mL), dried over Na₂SO₄, filtered, and the filtrate was concentrated under reduced pressure to give a residue. The residue was purified by column chromatography (SiO₂, petroleum ether/ethyl acetate=10/1 to 3/1). [(2R,3R,4S,5R)-4,5,6-tri(propanoyloxy)-2-(trityloxymethyl)tetrahydropyran-3-yl]propanoate (29 g, 44.84 mmol, 40.40% yield) was obtained as a colorless oil. A solution of [(2R,3R,4S,5R)-4,5,6-tri(propanoyloxy)-2-(trityloxymethyl)tetrahydropyran-3-yl]propanoate (8 g, 12.37 mmol, 1 equiv.) in HOAc (60 mL) and H₂O (30 mL) was stirred at 65° C. for 2.5 h under N₂ atmosphere. TLC indicated [(2R,3R,4S,5R)-4,5,6-tri(propanoyloxy)-2-(trityloxymethyl)tetrahydropyran-3-yl]propanoate was consumed completely, and two new spots formed. The reaction mixture was diluted with H₂O (100 mL) and extracted with EtOAc (40 mL×3). The combined organic layers were washed with brine (30 mL), dried over Na₂SO₄, filtered, and the filtrate was concentrated under reduced pressure to give colorless oil. The residue was purified by column chromatography (SiO₂, petroleum ether/ethyl acetate=10/1 to 3/1). [(2R,3R,4S,5R)-2-(hydroxymethyl)-4,5,6-tri(propanoyloxy)tetrahydropyran-3-yl]propanoate (3.1 g, 7.67 mmol, 61.97% yield) was obtained as a colorless oil.

A mixture of (E)-4-methoxy-4-oxo-but-2-enoic acid (1.50 g, 11.50 mmol, 1.5 equiv.), DCC (2.37 g, 11.50 mmol, 2.33 mL, 1.5 equiv.), DMAP (468.24 mg, 3.83 mmol, 0.5 equiv.) in DCM (100 mL) was stirred at 15° C. for 0.5 h. [(2R,3R,4S,5R)-2-(hydroxymethyl)-4,5,6-tri(propanoyloxy)tetrahydropyran-3-yl]propanoate (3.1 g, 7.67 mmol, 1 equiv.) was added to the mixture and then the mixture was stirred at 15° C. for 9.5 h under N₂ atmosphere. LC-MS detected the desired compound. The reaction mixture was filtered and the filtrate was concentrated under reduced pressure to give a residue. The residue was purified by column chromatography (SiO₂, petroleum ether/ethyl acetate=3/1 to 1/1). After column chromatography, the crude product was purified by re-crystallization with petroleum ether/EtOAc=30/1 (10 mL) at 20° C. 1-methyl 4-[(2R,3R,4S,5R,6R)-3,4,5,6-tetrakis(propanoyloxy)oxan-2-yl]methyl(2E)-but-2-enedioate (112 mg, 210.34 μmol, 43.46% yield, 97.00% purity) was obtained as a white solid. LCMS (M+18)⁺: 534.2 at 2.574 min. ¹H NMR (400 MHz, chloroform-d): δ 6.82 (d, J=2.3 Hz, 2H), 6.29 (d, J=3.5 Hz, 1H), 5.44 (t, J=9.9 Hz, 1H), 5.19-4.96 (m, 2H), 4.36-4.01 (m, 3H), 3.75 (s, 3H), 2.48-2.06 (m, 8H), 1.26-0.89 (m, 12H) ppm.

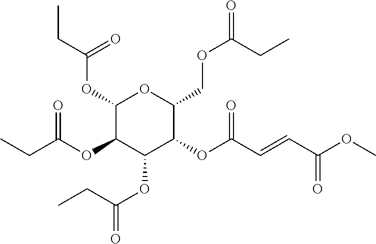 [SMILES: CCC(=O)OC[C@H]1O[C@@H](OC(=O)CC)[C@H](OC(=O)CC)[C@@H](OC(=O)CC)[C@H]1OC(=O)/C=C/C(=O)OC]

### Compound 51: 1-methyl(2R,3S,4S,5R,6S)-4,5,6-tris(propanoyloxy)-2-[(propanoyloxy)methyl]oxan-3-yl(2E)-but-2-enedioate

To a solution of [(2R,3S,4S,5R)-4,5,6-tri(propanoyloxy)-2-(trityloxymethyl)tetrahydropyran-3-yl]propanoate (5 g, 7.73 mmol, 1 equiv.) in HOAc (30 mL) was added H₂O (15.00 g, 832.41 mmol, 15 mL, 107.67 equiv.) at 15° C. and the mixture was stirred at 65° C. for 5 h. TLC indicated reactant was consumed completely. The reaction mixture was filtered and the filtrate was concentrated under reduced pressure to give a residue. The residue was purified by column chromatography (SiO₂, petroleum ether/ethyl acetate=20/1 to 0/1). [(2R,3S,4S,5R)-2-(hydroxymethyl)-4,5,6-tri(propanoyloxy)tetrahydropyran-3-yl]propanoate (1.4 g, 3.46 mmol, 44.78% yield) was obtained as a colorless oil.

To a solution of (E)-4-methoxy-4-oxo-but-2-enoic acid (900.76 mg, 6.92 mmol, 2 equiv.) and DCC (1.07 g, 5.19 mmol, 1.5 equiv.) in DCM (15 mL) was added DMAP (211.46 mg, 1.73 mmol, 0.5 equiv.). The resultant mixture was stirred at 15° C. for 10 min. Then [(2R,3S,4S,5R)-2-(hydroxymethyl)-4,5,6-tri(propanoyloxy)tetrahydropyran-3-yl]propanoate (1.4 g, 3.46 mmol, 1 equiv.) was added to the mixture and the mixture was stirred at 15° C. for 12 h. TLC indicated reactant was consumed completely. The reaction mixture was filtered and the filtrate was concentrated under reduced pressure to give a residue. The residue was purified by column chromatography (SiO₂, petroleum ether/ethyl acetate=20/1 to 1/1). O1-methyl O4-[[(2R,3S,4S,5R)-3,4,5,6-tetra(propanoyloxy)tetrahydropyran-2-yl]methyl](E)-but-2-enedioate (600 mg, 813.18 μmol, 23.49% yield, 70% purity) was obtained as a colorless oil. 1-methyl O4-[[(2R,3S,4S,5R)-3,4,5,6-tetra(propanoyloxy)tetrahydropyran-2-yl]methyl](E)-but-2-enedioate (600 mg, 1.16 mmol, 1 equiv.) was further purified by SFC separation (column: DAICEL CHIRALPAK IC 250 mm×30 mm, 10 μm); mobile phase: Neu-IPA; B %: 20%-20%, 8 min). 1-methyl(2R,3S,4S,5R,6S)-4,5,6-tris(propanoyloxy)-2-[(propanoyloxy)methyl]oxan-3-yl(2E)-but-2-enedioate (170 mg, 325.85 μmol, 28.05% yield, 99% purity) was obtained as a colorless oil. LCMS (M+18)⁺: 534.2 at 3.128. ¹H NMR (400 MHz, chloroform-d): δ 6.87 (d, J=2.1 Hz, 2H), 5.68 (d, J=8.3 Hz, 1H), 5.47 (d, J=3.4 Hz, 1H), 5.40-5.17 (m, 1H), 5.09 (dd, J=10.4, 3.4 Hz, 1H), 4.19-3.96 (m, 3H), 3.77 (s, 3H), 2.50-2.00 (m, 8H), 1.19-0.84 (m, 12H) ppm.

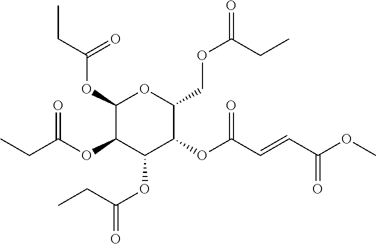 [SMILES: CCC(=O)OC[C@H]1O[C@H](OC(=O)CC)[C@H](OC(=O)CC)[C@@H](OC(=O)CC)[C@H]1OC(=O)/C=C/C(=O)OC]

### Compound 52: 1-methyl(2R,3S,4S,5R,6R)-4,5,6-tris(propanoyloxy)-2-[(propanoyloxy)methyl]oxan-3-yl(2E)-but-2-enedioate

To a solution of [(2R,3S,4S,5R)-4,5,6-tri(propanoyloxy)-2-(trityloxymethyl)tetrahydropyran-3-yl]propanoate (5 g, 7.73 mmol, 1 equiv.) in HOAc (30 mL) was added H₂O (15.00 g, 832.41 mmol, 15 mL, 107.67 equiv.) at 15° C. and the mixture was stirred at 65° C. for 5 h. TLC indicated reactant was consumed completely. The reaction mixture was filtered, and the filtrate was concentrated under reduced pressure to give a residue. The residue was purified by column chromatography (SiO₂, petroleum ether/ethyl acetate=20/1 to 0/1). [(2R,3S,4S,5R)-2-(hydroxymethyl)-4,5,6-tri(propanoyloxy)tetrahydropyran-3-yl]propanoate (1.4 g, 3.46 mmol, 44.78% yield) was obtained as a colorless oil.

To a solution of (E)-4-methoxy-4-oxo-but-2-enoic acid (900.76 mg, 6.92 mmol, 2 equiv.) and DCC (1.07 g, 5.19 mmol, 1.5 equiv.) in DCM (15 mL) was added DMAP (211.46 mg, 1.73 mmol, 0.5 equiv.) was stirred at 15° C. for 10 min. Then [(2R,3S,4S,5R)-2-(hydroxymethyl)-4,5,6-tri(propanoyloxy)tetrahydropyran-3-yl]propanoate (1.4 g, 3.46 mmol, 1 equiv.) was added to the mixture and the mixture was stirred at 15° C. for 12 h. TLC indicated reactant was consumed completely. The reaction mixture was filtered and the filtrate was concentrated under reduced pressure to give a residue. The residue was purified by column chromatography (SiO₂, petroleum ether/ethyl acetate=20/1 to 1/1). O1-methyl O4-[[(2R,3S,4S,5R)-3,4,5,6-tetra(propanoyloxy)tetrahydropyran-2-yl]methyl](E)-but-2-enedioate (600 mg, 813.18 μmol, 23.49%% yield, 70% purity) was obtained as a colorless oil. 1-methyl O4-[[(2R,3S,4S,5R)-3,4,5,6-tetra(propanoyloxy)tetrahydropyran-2-yl]methyl](E)-but-2-enedioate (600 mg, 1.16 mmol, 1 equiv.) was further subjected to SFC separation (column: DAICEL CHIRALPAK IC 250 mm×30 mm, 10 μm); mobile phase: Neu-IPA; B %: 20%-20%, 8 min). O1-methyl O4-[(2R,3S,4S,5R,6R)-4,5,6-tri(propanoyloxy)-2-(propanoyloxymethyl)tetrahydropyran-3-yl](E)-but-2-enedioate (45 mg, 84.51 μmol, 7.28% yield, 97% purity) was obtained as a colorless oil. LCMS: (M+18)⁺: 534.2 at 3.159 min. ¹H NMR (400 MHz, chloroform-d): δ 6.94-6.71 (m, 2H), 6.18 (d, J=26.4 Hz, 1H), 5.46 (dt, J=7.6, 3.9 Hz, 1H), 4.99 (dd, J=5.0, 1.7 Hz, 1H), 4.44-3.94 (m, 3H), 3.76 (d, J=4.5 Hz, 3H), 2.43-2.00 (m, 8H), 1.20-0.86 (m, 12H) ppm.

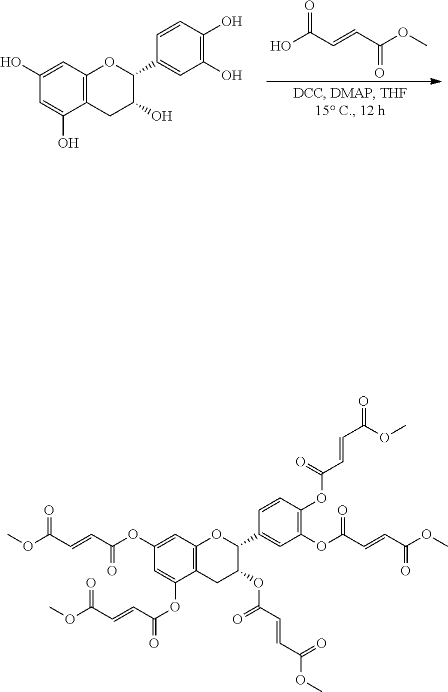

### Compound 53: 04-[2-[(E)-4-methoxy-4-oxo-but-2-enoyl]oxy-4-[(2R,3R)-3,5,7-tris[[(E)-4-methoxy-4-oxo-but-2-enoyl]oxy]chroman-2-yl]phenyl]O1-methyl(E)-but-2-enedioate

To a solution of (2R,3R)-2-(3,4-dihydroxyphenyl)chromane-3,5,7-triol (100 mg, 344.51 μmol, 1 equiv.) and (E)-4-methoxy-4-oxo-but-2-enoic acid (313.74 mg, 2.41 mmol, 7 equiv.) in THF (5 mL) was added DCC (426.49 mg, 2.07 mmol, 418.13 μL, 6 equiv.) and DMAP (2.10 mg, 17.23 μmol, 0.05 equiv.). The mixture was stirred at 15° C. for 12 h. LC-MS detected the desired compound. The reaction mixture was concentrated under reduced pressure to give a residue. The residue was purified by prep-HPLC (water+0.04% (v/v) HCl/ACN) to afford the title compound as a yellow solid (23 mg, 26.77 μmol, 7.77%% yield, 99% purity). LCMS (M+H)⁺: 851.2.

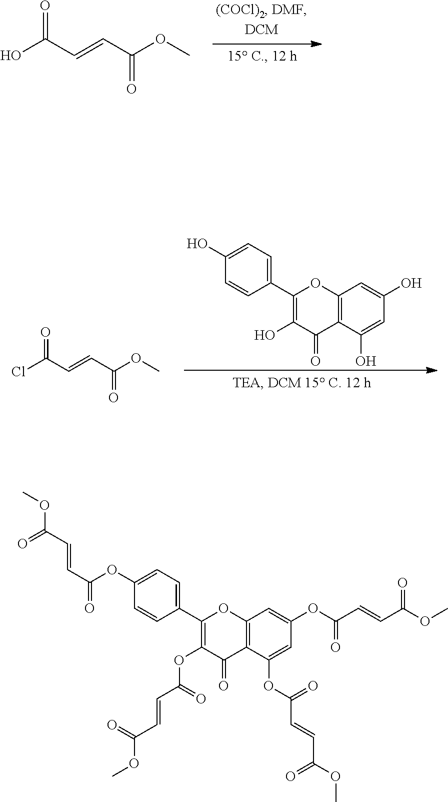

### Compound 54: 01-methyl O4-[4-[3,5,7-tris[[(E)-4-methoxy-4-oxo-but-2-enoyl]oxy]-4-oxo-chromen-2-yl]phenyl](E)-but-2-enedioate

To a solution of (E)-4-methoxy-4-oxo-but-2-enoic acid (1 g, 7.69 mmol, 1 equiv.) in DCM (5 mL) was added DMF (95.00 mg, 1.30 mmol, 0.1 mL) and (COCl)₂ (3.90 g, 30.75 mmol, 2.69 mL, 4 equiv.). The mixture was stirred at 15° C. for 12 h. TLC indicated (E)-4-methoxy-4-oxo-but-2-enoic acid was consumed completely. The reaction mixture was concentrated under reduced pressure to give a residue.

The crude product methyl (E)-4-chloro-4-oxo-but-2-enoate (260 mg, crude) as a white solid was used into the next step without further purification.

To a solution of 3,5,7-trihydroxy-2-(4-hydroxyphenyl)chromen-4-one (100 mg, 349.36 μmol, 1 eq) in DCM (5 mL) was added Et₃N (176.76 mg, 1.75 mmol, 243.14 μL, 5 equiv.) and methyl (E)-4-chloro-4-oxo-but-2-enoate (260 mg, 1.75 mmol, 5 equiv.). The mixture was stirred at 15° C. for 12 h. LC-MS showed the desired compound was detected. The reaction mixture was concentrated under reduced pressure to give a residue. The residue was purified by prep-HPLC (water+0.05% (v/v) HCl/ACN). The title compound was obtained as a white solid (52 mg, 69.37 μmol, 19.86% yield, 98% purity). LCMS (M+H)⁺: 735.2.

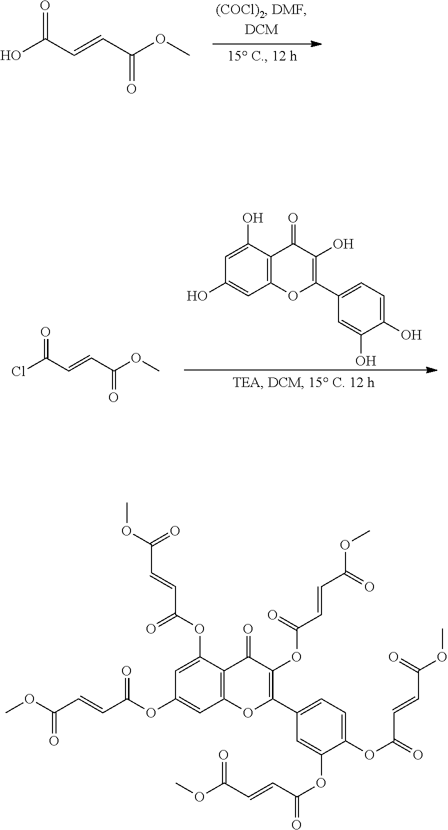

### Compound 55: 04-[2-[(E)-4-methoxy-4-oxo-but-2-enoyl]oxy-4-[3,5,7-tris[[(E)-4-methoxy-4-oxo-but-2-enoyl]oxy]-4-oxo-chromen-2-yl]phenyl]O1-methyl(E)-but-2-enedioate

To a solution of (E)-4-methoxy-4-oxo-but-2-enoic acid (300 mg, 2.31 mmol, 1 equiv.) in DCM (5 mL) was added DMF (95.00 mg, 1.30 mmol, 0.1 mL, 0.56 equiv.) and (COCl)₂ (1.17 g, 9.22 mmol, 807.41 μL, 4 equiv.). The mixture was stirred at 15° C. for 12 h. The reaction mixture was concentrated under reduced pressure to give a methyl (E)-4-chloro-4-oxo-but-2-enoate (260 mg, crude) as a white solid. To a solution of 2-(3,4-dihydroxyphenyl)-3,5,7-trihydroxy-chromen-4-one (100 mg, 330.87 μmol, 1 equiv.) in DCM (5 mL) was added Et₃N (174.10 mg, 1.72 mmol, 239.48 μL, 5.2 equiv.) and methyl (E)-4-chloro-4-oxo-but-2-enoate (255.57 mg, 1.72 mmol, 5.2 equiv.). The mixture was stirred at 15° C. for 12 h. LCMS detected the desired compound. The reaction mixture was concentrated under reduced pressure to give a residue. The residue was purified by prep-HPLC (water+0.05% (v/v) HCl/ACN). The title compound was obtained as a white solid (16 mg, 18.18 μmol, 5.49% yield, 98% purity). LCMS (M+H)+: 863.0.

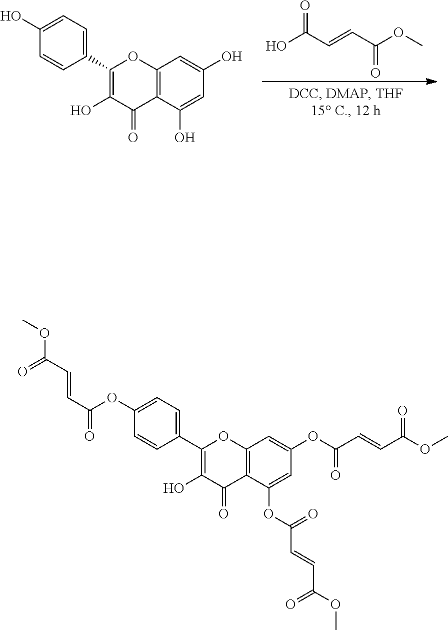

### Compound 56: 04-[4-[3-hydroxy-5,7-bis[[(E)-4-methoxy-4-oxo-but-2-enoyl]oxy]-4-oxo-chromen-2-yl]phenyl]O1-methyl(E)-but-2-enedioate

To a solution of 3,5,7-trihydroxy-2-(4-hydroxyphenyl)chromen-4-one (0.2 g, 698.72 μmol, 1 equiv.) and (E)-4-methoxy-4-oxo-but-2-enoic acid (545.42 mg, 4.19 mmol, 6 equiv.) in THF (5 mL) was added DCC (720.83 mg, 3.49 mmol, 706.69 μL, 5 equiv.) and DMAP (4.27 mg, 34.94 μmol, 0.05 equiv.). The mixture was stirred at 15° C. for 12 h. LCMS detected the desired compound. The mixture was filtered and concentrated under reduced pressure to give a residue. The residue was purified by prep-HPLC (water+0.05% (v/v) HCl/ACN) to afford the title compound as a yellow solid (40 mg, 133.82 μmol, 19.15% yield, 98% purity). LCMS (M+H)+: 623.1.

### Example 2: In Vitro DMPK Degradation Assays

Conjugates disclosed herein may be stable under a range of physiological pH levels and cleaved selectively at a desired site of action (for example, in the GI tract, e.g., in the stomach, small intestine, or large intestine) by enzymes present in the local microenvironment. Conjugates are tested for chemical stability at a range of pH levels as well as their ability to be degraded in representative in vitro systems. Data for select conjugates are shown below.

Assay 1. Stability of conjugates in Simulated Gastric Fluid (SGF). This assay was used to assess the stability of a conjugate in a stomach.

Medium was prepared by dissolving 2 g of sodium chloride in 0.6 L in ultrapure water (MilliQ®, Millipore Sigma, Darmstadt, Germany). The pH was adjusted to 1.6 with 1N hydrochloric acid, and the volume was then adjusted to 1 L with purified water.

60 mg FaSSIF powder (Biorelevant™, London, UK) were dissolved in 500 mL buffer (above). Pepsin was added (0.1 mg/mL) (Millipore Sigma, Darmstadt, Germany), and the solution was stirred. The resulting SGF media were used fresh for each experiment.

Test compounds were dissolved in DMSO stock to 1 mM. An aliquot of the DMSO stock solution was removed and diluted in the SGF Media in 15 mL falcon tubes to generate a total compound concentration of 1 μM. A 1 mL aliquot was immediately removed and diluted once with 1 volume of acetonitrile for TO timepoint. The mixture was sealed and mixed at 37° C. in an incubator. Aliquots (1 mL) were removed at regular intervals and immediately quenched by the addition of 1 volume of acetonitrile. The resulting samples were analyzed by LC/MS to determine degradation rates in SGF.

Assay 2. Stability of conjugates in Simulated Intestinal Fluid (SIF). This assay was used to assess the stability of a conjugate in a small intestine.

Phosphate buffer was prepared by dissolving 0.42 g of sodium hydroxide pellets and 3.95 g of monobasic sodium phosphate monohydrate and 6.19 g of sodium chloride in ultrapure water (MilliQ®, Millipore Sigma, Darmstadt, Germany). The pH was adjusted to 6.7 using aq. HCl and aq. NaOH, as necessary, and the solution was diluted with ultrapure water to produce 1 L of the pH 6.7 buffer.

112 mg FaSSIF powder (Biorelevant™, London, UK) was dissolved in 50 mL of the pH 6.7 buffer. 2 to 3 mL of the resulting solution were then added to 500 mg pancreatin (Millipore Sigma, Darmstadt, Germany). The resulting mixture was agitated by finger tapping the vessel containing the mixture until milky suspension formed. At this time, the remainder of the 50 mL FaSSiF/pH 6.7 buffer solution was added. The resulting suspension was flipped upside down 10 times to produce SIF, which was used fresh.

Test compounds were dissolved in DMSO stock to 1 mM. An aliquot of the DMSO stock solution was removed and diluted in the SIF media in 15 mL falcon tubes to produce a mixture with a tested compound concentration of 1 μM. A 1 mL aliquot was immediately removed and diluted once with 1 volume of acetonitrile for TO timepoint. The mixture was sealed and agitated at 37° C. in an incubator. Aliquots (1 mL) were removed at regular intervals and immediately quenched by the addition of 1 volume of acetonitrile. The resulting samples were analyzed by LC/MS to determine degradation rates

Assay 3. In vitro Colonic Material Stability Assay. This assay was used to assess the stability of a conjugate in a large intestine. All experiments were performed in an anaerobic chamber containing 90% nitrogen, 5% hydrogen and 5% carbon dioxide. Colonic material was resuspended as a slurry (15% w/v final concentration) in pre-reduced, anaerobically sterilized dilution blanks (Anaerobe Systems AS-908). The colonic material was then inoculated into 96 well plates containing YCFAC media (Anaerobe Systems AS-680) or other suitable media (6.7 μL slurry into 1 mL total media). Compounds or groups of compounds were added to each individual well to reach a final analyte concentration of 1 or 10 μM, and the material was mixed by pipetting. Sample was removed after set timepoints (0, 120, 240, 480, 1440, 2880 minutes after initiation of the assay), quenched with acetonitrile containing internal standard, and analyzed by LC/MS.

TABLE 1

| | Assay 1 (SGF) | Assay 2 (SIF) | Assay 3 |
|---|---|---|---|
| | (% Remaining | (% @ Remaining | (% Remaining |
| Compound | @ 1 hour) | 4 hours) | at 24 h) |
| 1 | C | | C |
| 2 | C | B | C |
| 3 | C | B | C |
| 4 | B | C | B |
| 5 | C | C | A |
| 6 | C | A | |
| 7 | C | A | C |
| 8 | C | A | C |
| 9 | C | A | C |
| 10 | B | B | B |
| 11 | B | B | B |
| 12 | C | A | C |
| 13 | B | A | C |
| 14 | C | A | C |
| 15 | B | A | C |
| 16 | | | C |
| 20 | C | A | C |
| 21 | C | B | |
| 22 | C | A | |
| 23 | B | A | A |
| 24 | C | A | |
| 26 | C | A | |
| 27 | | C | B |
| 28 | | B | B |
| 36 | C | A | C |
| 38 | C | A | A |
| 39 | C | A | A |
| 40 | B | A | A |
| 44 | C | A | |
| 45 | C | A | |
| 47 | C | A | |
| 48 | C | A | |
| 49 | C | A | |
| 50 | C | A | |
| 51 | C | A | |
| 52 | C | A | |
| 53 | C | A | |
| 54 | C | A | |
| 55 | C | A | |
| 56 | C | A | |

In Table 1, A: <25% of the tested compound remaining; B: 25-75% of the tested compound remaining; and C: >75% of the tested compound remaining.

Compounds that are stable in assay 1 and unstable in assay 2 can deliver bioactives to the small intestine. Compounds that are stable in assays 1 and 2 and unstable in assay 3 can deliver bioactives to the large intestine.

### Example 3: In Vitro Biotransformation and Detection of Monomethyl Fumarate Assay

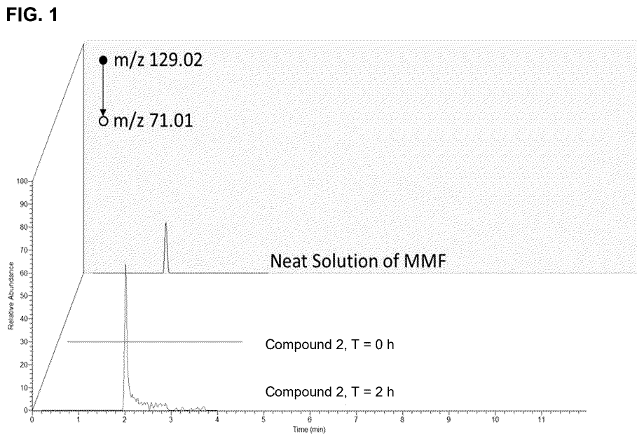

Stock solution of compound 2 was prepared at 10 mM in DMSO. FaSSIF was made by mixing sodium taurocholate (3.0 mM) Lecithin (0.75 mM), and pancreatin (10 mg/mL) in prepared solution of sodium phosphate monobasic (28.4 mM), sodium hydroxide (8.7 mM), sodium chloride (105.9 mM), pH 6.5. Compound 2, was added to FaSSIF to final concentration of 100 μM. Release of monomethyl fumaric acid (MMF) was monitored via UHPLC-MSMS and comparing the retention time and corresponding fragmentation of released MMF to retention time and fragmentation of a neat solution of MMF that was analyzed separately with the same method described below. Release of MMF was measured at 0 h and 2 h time points. At both time points samples were centrifuged at 14000 rpm for 10 minutes at 4° C. Supernatants were then transferred to HPLC vials and analyzed immediately. The results of this assay are illustrated in FIG. 1.

This data demonstrates that monomethyl fumarate is actively released from compound 2 in simulated intestinal fluid and suggests it will also be released in the small intestine of a subject.

### Example 4: In Vivo EAE Model of Multiple Sclerosis

### Study 1

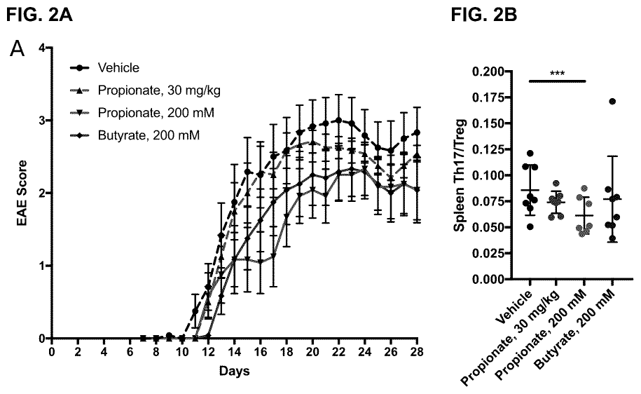

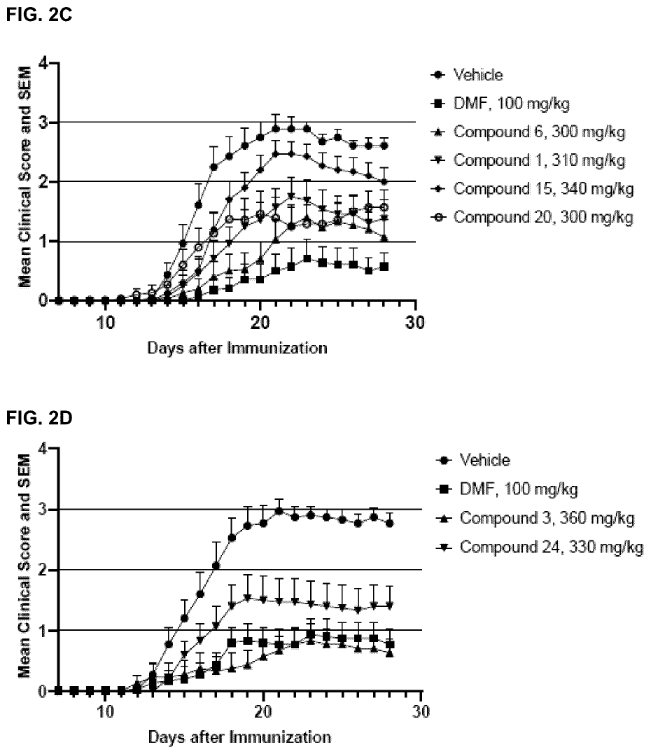

For experimental autoimmune encephalomyelitis (EAE) study, 8- to 11-week-old C57BL/6J mice were anesthetized and subcutaneously injected with 200 mg MOG₃₅₋₅₅ and 200 mg CFA. Pertussis toxin (200 ng/mouse) was applied i.p. on days 0. Daily clinical evaluation was performed via a 5-point scale, and the clinical progression was observed over 28 days (FIG. 2A). Animals received either 200 ml of vehicle (methyl-cellulose) (black line), or sodium propionate (5 μM, BID) (dotted red line) daily via oral gavage, or 200 mM of sodium propionate (solid red line) or sodium butyrate (200 mM) propionate added in drinking water. At the end of the study, flow cytometry analysis was performed on the spleen (n=8 per group). The T_{H}17 cell (defined as CD3⁺, IL7⁺)/regulatory T cells (Treg; defined as CD3⁺, Foxp3⁺) ratio was significantly reduced (P<0.05) in mice received 200 mM of sodium propionate in drinking water (FIG. 2B). Statistical analysis was performed with GraphPad Prism (GraphPad Software). Unpaired t test was used to assess significance between the control (Vehicle) and each treatment group.

Reduction in EAE score suggests that treatment with compounds of the invention would be efficacious in reducing signs and symptoms in patients with multiple sclerosis and. T_{H}17/Treg ratio was modified to a more tolerogenic state consistent with the suggestion that propionate may reduce systemic inflammation and would be efficacious at treating multiple sclerosis.

### Study 2

For experimental autoimmune encephalomyelitis (EAE) studies, 8- to 11-week old C57BL/6J mice were anesthetized and subcutaneously injected with 200 mg MOG₃₅₋₅₅ and 200 mg CFA. Pertussis toxin (200 ng/mouse) was applied intraperitoneally on day 0. Daily clinical evaluation as performed using a 5-point scale and the clinical progression was observed over 28 days (FIGS. 2C and 2D). Animals were orally administered either 200 mL of vehicle (methyl-cellulose), approximately 100 mg/kg of dimethylfumarate (DMF), or an amount of conjugate which provided an approximately equimolar amount of DMF. Reduction in EAE score suggests that treatment with compounds of the invention can be efficacious in reducing signs and symptoms in patients with multiple sclerosis.

### Example 5: Monomethylfumarate and Short Chain Fatty Acid Pharmacokinetic Studies

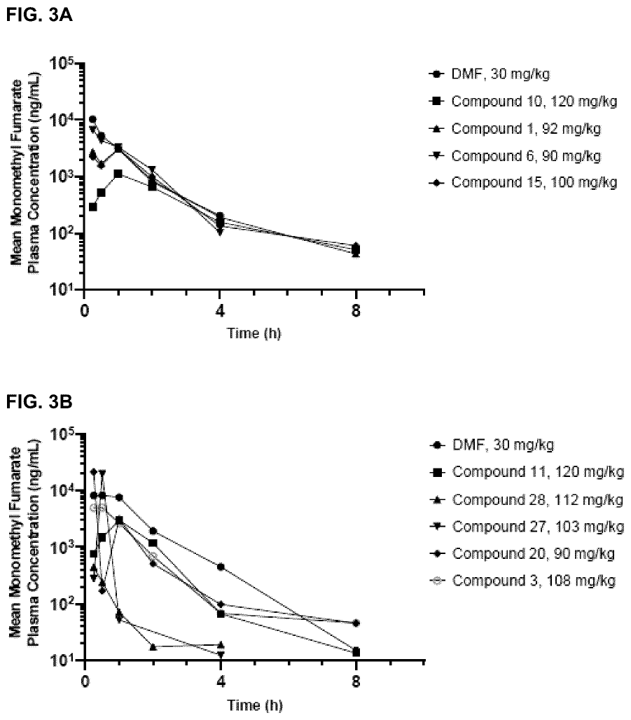

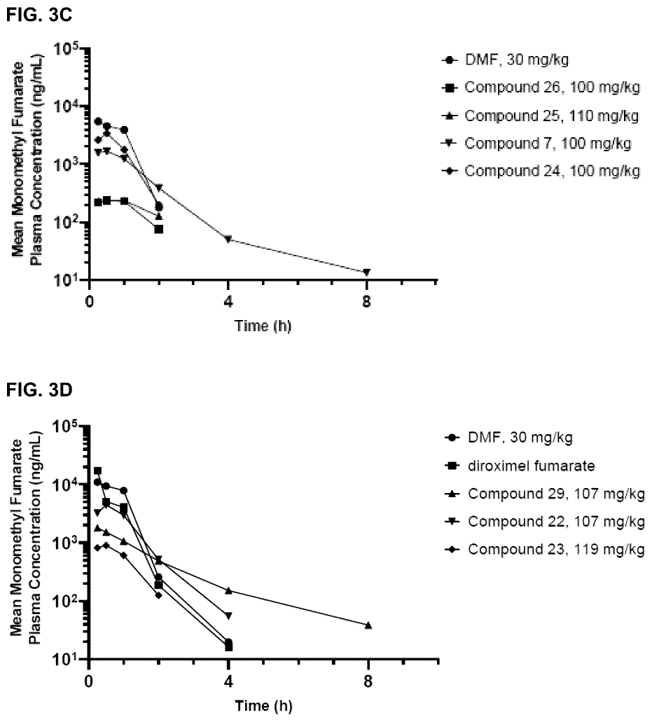

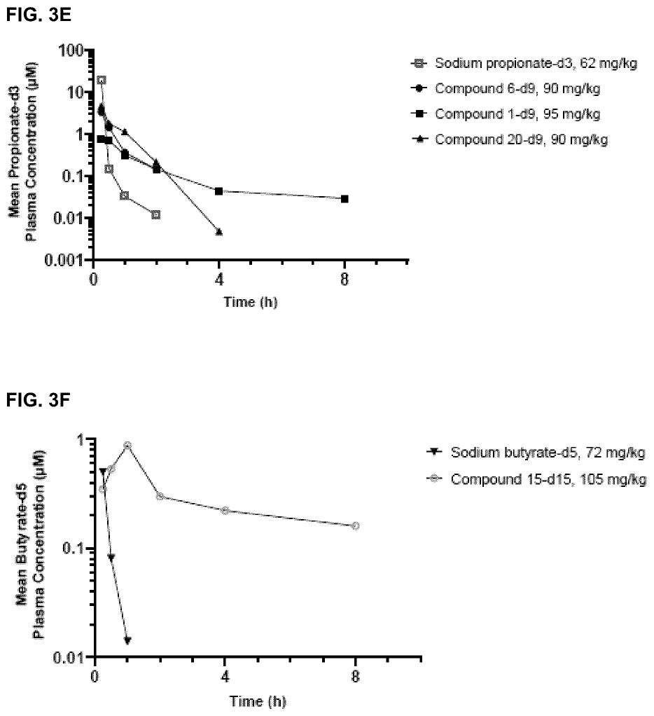

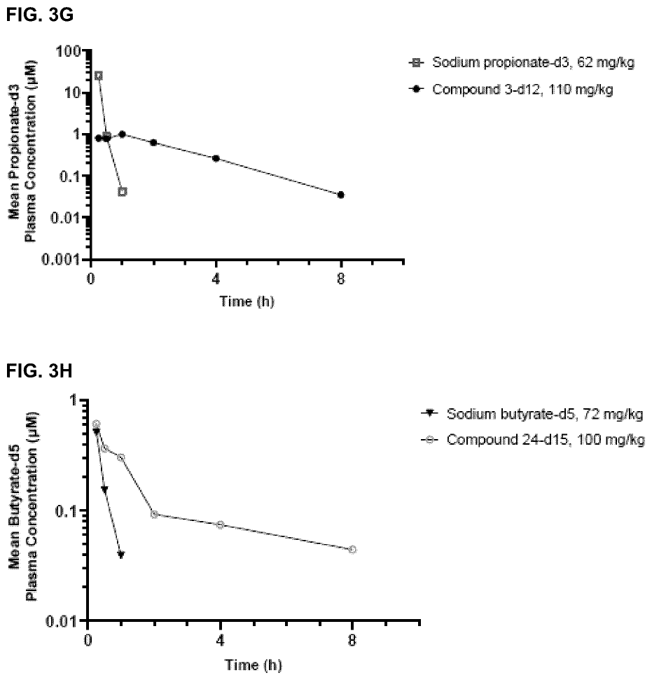

For monomethylfumarate pharmacokinetic studies, 9- to 10-week old male Sprague Dawley rats were orally administered a single dose of dimethylfumarate or compounds of the invention (suspension, 1% (w/v) methyl cellulose in deionized water). The amount of compound dosed was normalized to provide approximately equimolar amounts of monomethylfumarate. Approximately 150 μL whole blood samples were collected at 15 and 30 m; and 1, 2, 4, 8, and 24 h post-dosing from the jugular or tail vein. 100 μL of samples were added to K₂EDTA tubes pre-filled with 300 μL of 100 mM tiopronin in 100 mM ammonium bicarbonate (pH 9.0). Samples were vortex-mixed for approximately 5 min at ambient temperature in order to trap free fractions of monomethyl fumarate. Samples were subsequently analyzed by LC-MS/MS for mean plasma concentration of monomethyl fumarate (FIGS. 3A-3D). Certain pharmacokinetic parameters are provided below in Table 2.

TABLE 2

| | | | | |
|---|---|---|---|---|
| | Dose | Tₘₐₓ | Cₘₐₓ | AUCₗₐₛₜ |
| Compound | (mg/kg) | (h) | (ng/mL) | (h × ng/mL) |

Experiment 1 (FIG. 3A)

| | | | | |
|---|---|---|---|---|
| DMF | 30 | 0.25 | 10300 | 8330 |
| Compound 10 | 120 | 1.00 | 1120 | 2310 |
| Compound 1 | 92 | 0.75 | 3280 | 5040 |
| Compound 6 | 90 | 0.33 | 6730 | 7250 |
| Compound 15 | 100 | 1.00 | 3100 | 5170 |

Experiment 2 (FIG. 3B)

| | | | | |
|---|---|---|---|---|
| DMF | 30 | 0.58 | 9063 | 15042 |
| Compound 11 | 120 | 1.00 | 2968 | 4592 |
| Compound 28 | 112 | 0.25 | 441 | 261 |
| Compound 27 | 103 | 0.50 | 19658 | 7487 |
| Compound 20 | 90 | 0.25 | 21290 | 8890 |
| Compound 3 | 108 | 0.42 | 5276 | 6408 |

Experiment 3 (FIG. 3C)

| | | | | |
|---|---|---|---|---|
| DMF | 30 | 0.25 | 5475 | 6112 |
| Compound 26 | 100 | 0.58 | 283 | 349 |
| Compound 25 | 110 | 0.75 | 287 | 381 |
| Compound 7 | 100 | 0.42 | 1739 | 2611 |
| Compound 24 | 100 | 0.50 | 3408 | 3359 |

Experiment 4 (FIG. 3D)

| | | | | |
|---|---|---|---|---|
| DMF | 30 | 0.25 | 10928 | 12490 |
| Diroximel Fumarate | 53 | 0.25 | 17251 | 9381 |
| Compound 29 | 107 | 0.33 | 1985 | 2958 |
| Compound 22 | 107 | 0.67 | 4757 | 5502 |
| Compound 23 | 100 | 0.42 | 900 | 1163 |

For short chain fatty acid (SCFA) pharmacokinetic studies, 9- to 10-week old male Sprague Dawley rats were orally administered a single dose of deuterated SCFA (sodium propionate-d3 or sodium butyrate-d5) or compounds of the invention comprising deuterated SCFA (suspension, 1% (w/v) methyl cellulose in deionized water). Deuterated SCFA analogs of compounds of the invention (e.g. Compound 3-d12, and Compound 6-d9) were synthesized in a similar manner as previously described, but instead deuterated SCFA was coupled to a sugar using EDCI coupling conditions. For SCFA pharmacokinetic studies, the amount of compound dosed was normalized to provide approximately equimolar amounts of monomethylfumarate. Approximately 150 μL whole blood samples were collected at 15 and 30 min; and 1, 2, 4, 8, and 24 h post-dosing from the jugular or tail vein. 100 μL of samples were added to K₂EDTA tubes pre-filled with 300 μL of 100 mM tiopronin in 100 mM ammonium bicarbonate (pH 9.0). Samples were vortex-mixed for approximately 5 minutes at ambient temperature in order to trap free fractions of SCFA. Samples were subsequently analyzed by LC-MS/MS for mean plasma concentration of SCFA (FIGS. 3E-3H).

Pharmacokinetic studies suggest that compounds of the invention can be metabolized in vivo to provide comparable amounts of monomethylfumarate in plasma when compared to administration of dimethylfumarate only. Pharmacokinetic studies also suggest that compounds of the invention can be metabolized in vivo to provide increased bioavailability of SCFA in plasma (e.g. propionate or butyrate) relative to administration of SCFA only. Further, SCFA pharmacokinetic studies demonstrate extended systematic exposure within a physiological range of each metabolite.

### Example 6: Gastrointestinal Exposure to Monomethylfumarate and Propionate

### Study 1: Gastrointestinal (GI) Exposure to Propionate

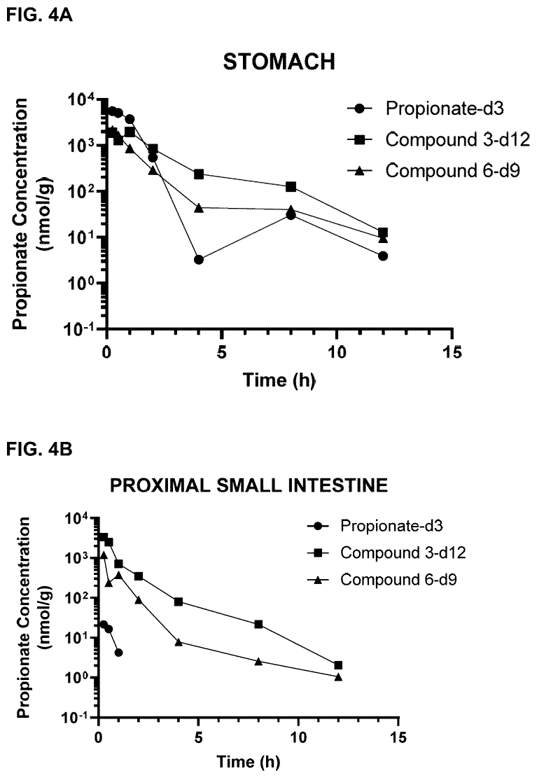

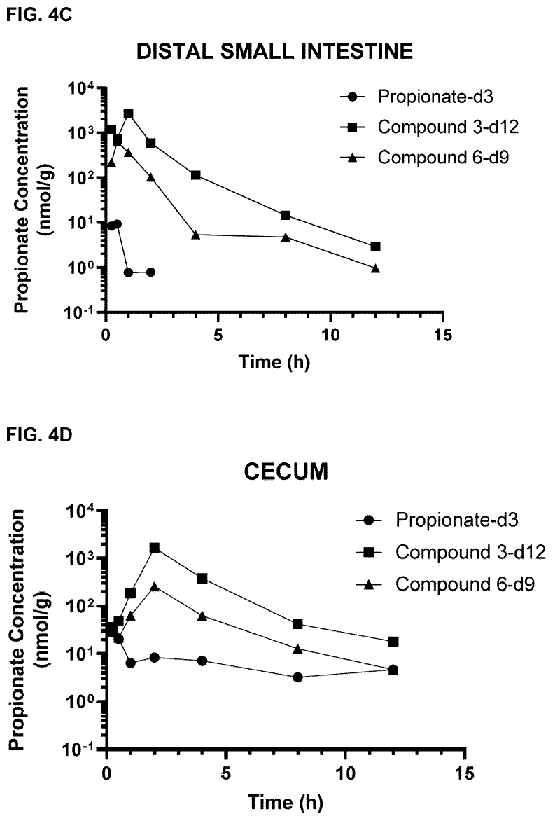

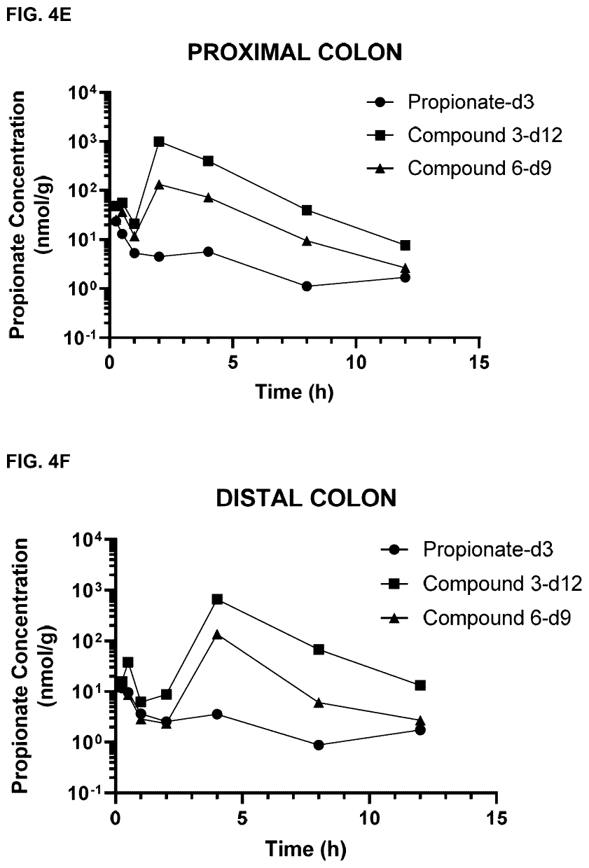

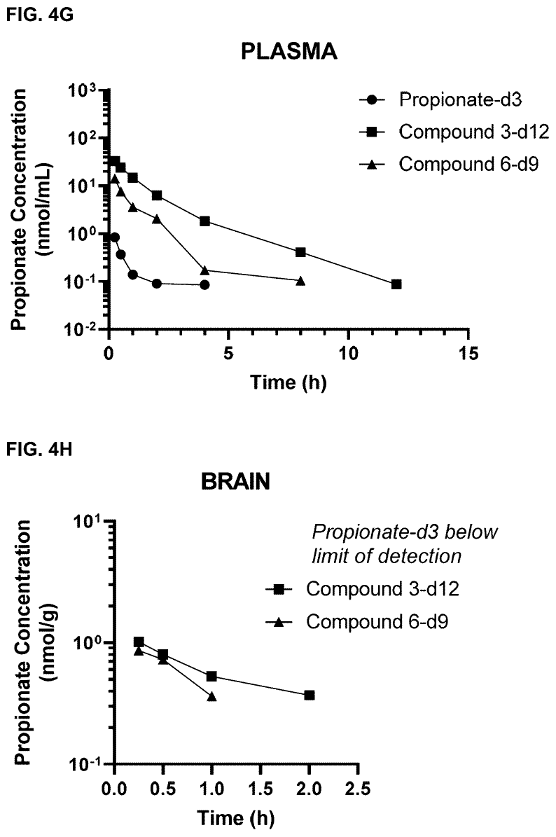

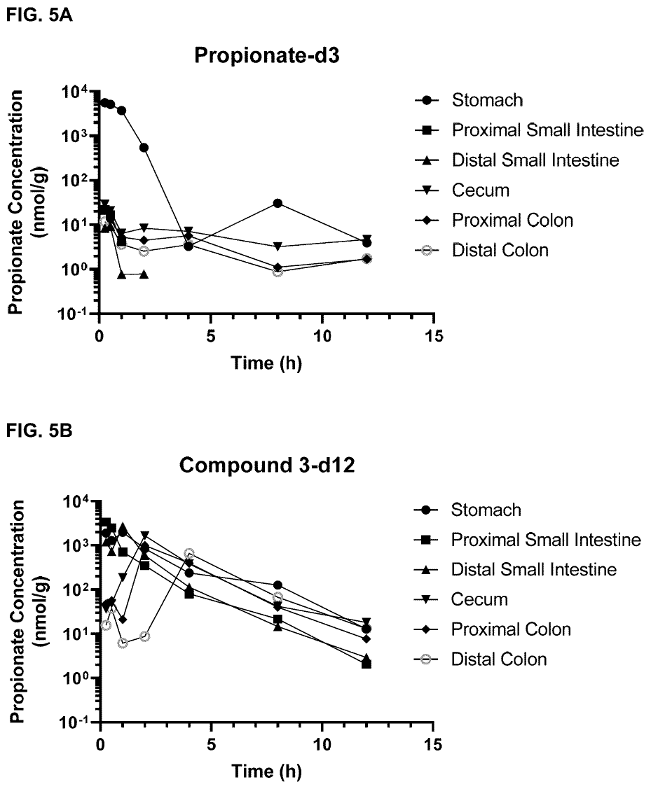

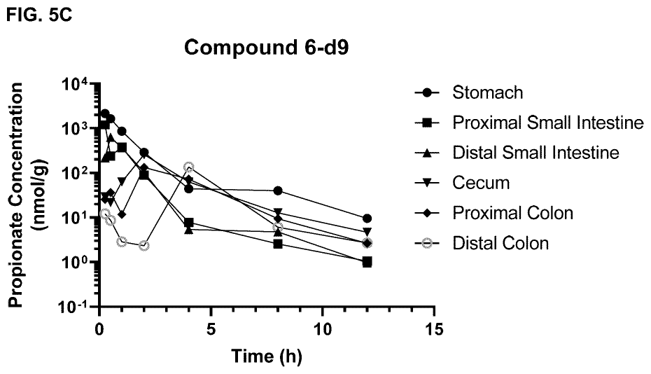

In order to measure propionate concentrations along the GI track, CD-1 mice were orally administered a single dose of deuterated sodium propionate, Compound 3 comprising deuterated propionate, or Compound 6 comprising deuterated propionate (Propionate-d3, Compound 3-d12, and Compound 6-d9, respectively; suspension, 1% (w/v) methyl cellulose in deionized water). The amounts of compound dosed were normalized to provide approximately equimolar amounts of monomethylfumarate (Propionate-d3 at 62 mg/kg, Compound 3-d12 at 110 mg/kg, and Compound 6-d9 at 91 mg/kg). Whole blood samples and GI digesta samples were collected pre-dosing; 15 and 30 min; and 1, 2, 4, 8, and 12 h after dosing. Blood samples were collected in K₂EDTA tubes and stored on wet ice no more than 30 min and then further processed to plasma. GI samples were placed into separate labeled, pre-weighted collection tubes and frozen before analysis. Brain samples were homogenized prior to analysis. Samples were subsequently worked up and analyzed by LC-MS/MS for deuterated propionate concentration. Propionate concentrations versus time for different tissues are depicted in FIGS. 4A-4H and 5A-5C. Certain pharmacokinetic parameters are summarized in Table 3 below.

TABLE 3

| | Propionate-d3 | Tₘₐₓ | Cₘₐₓ | AUCₗₐₛₜ |
|---|---|---|---|---|
| Tissue | derived from . . . | (h) | (nmol/g) | (h × nmol/g) |
| Stomach | Propionate-d3 | 0.25 | 5540 | 7020 |
| | Compound 3-d12 | 1.00 | 1970 | 4950 |
| | Compound 6-d9 | 0.25 | 2140 | 2550 |
| Proximal | Propionate-d3 | 0.25 | 22 | 13 |
| Intestine | Compound 3-d12 | 0.25 | 3370 | 3160 |
| | Compound 6-d9 | 0.25 | 1190 | 841 |
| Distal | Propionate-d3 | 0.50 | 9.2 | 6.6 |
| Intestine | Compound 3-d12 | 1.00 | 2690 | 3880 |
| | Compound 6-d9 | 0.50 | 630 | 759 |
| Cecum | Propionate-d3 | 0.25 | 29 | 76 |
| | Compound 3-d12 | 2.00 | 1660 | 4010 |
| | Compound 6-d9 | 2.00 | 256 | 696 |
| Proximal | Propionate-d3 | 0.25 | 23 | 46 |
| Colon | Compound 3-d12 | 2.00 | 995 | 2910 |
| | Compound 6-d9 | 2.00 | 133 | 489 |
| Distal | Propionate-d3 | 0.25 | 12 | 31 |
| Colon | Compound 3-d12 | 4.00 | 660 | 2310 |
| | Compound 6-d9 | 4.00 | 135 | 447 |
| Brain | Propionate-d3 | 0.25 | below quantitative | |
| | | | detection limit | |
| | Compound 3-d12 | 0.25 | 1.02 | 1.18 |
| | Compound 6-d9 | 0.25 | 0.86 | 0.62 |

Data from these experiments suggest compounds of the invention can be metabolized in vivo to provide large amounts of short chain fatty acid (SCFA) to different regions of the gut (See FIGS. 4A-4H, parameter AUCₗₐₛₜ). Further, in the intestines the amount of SCFA derived from compounds of the invention is higher than that from administration of SCFA only. Even further, compounds of the invention can be metabolized in vivo to deliver SCFA to the brain; administration of SCFA only does not result in detectable amounts of SCFA in the brain (see, e.g., FIG. 4H and Table 2, Brain).

Data (Tₘₐₓ) suggests compounds of the invention can reach throughout the intestine and release higher levels of propionate, especially when compared to delivery of only sodium propionate. The highest concentrations (Cₘₐₓ) of propionate-d3 derived from Compound 3-d12 and Compound 6-d9 were observed in the intestines (proximal and distal). The highest concentrations of gavash-administered propionate-d3 is observed in the stomach with relatively lower concentrations in other regions of the gut.

### Study 2: Gastrointestinal (GI) Exposure to Monomethylfumarate (MMF)

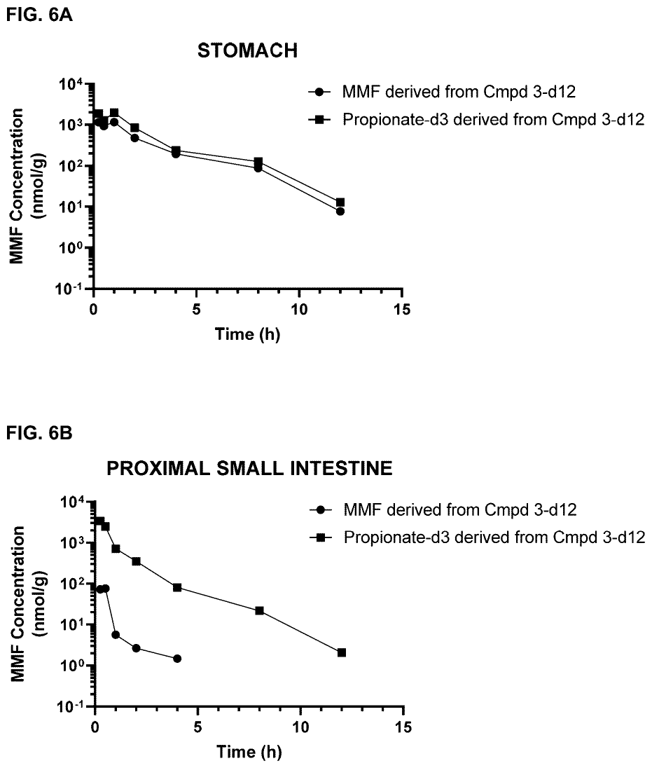

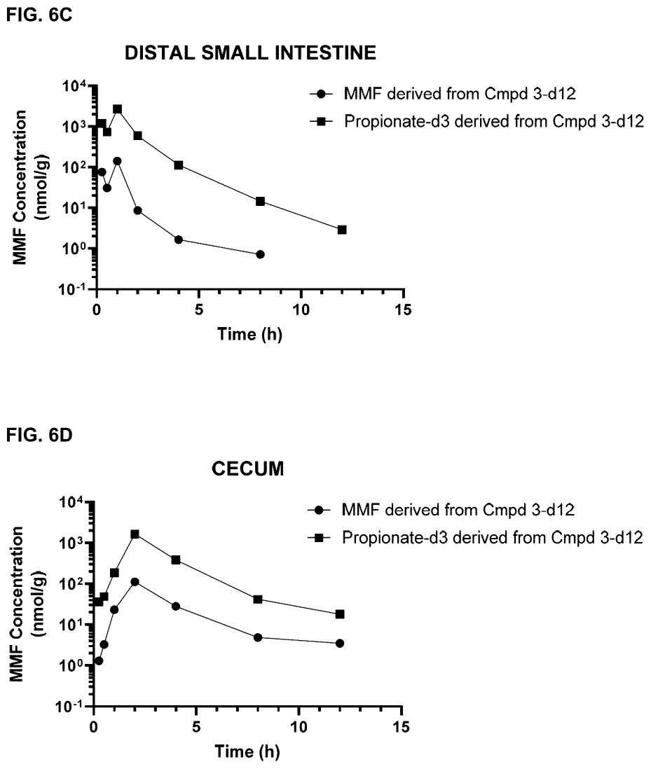

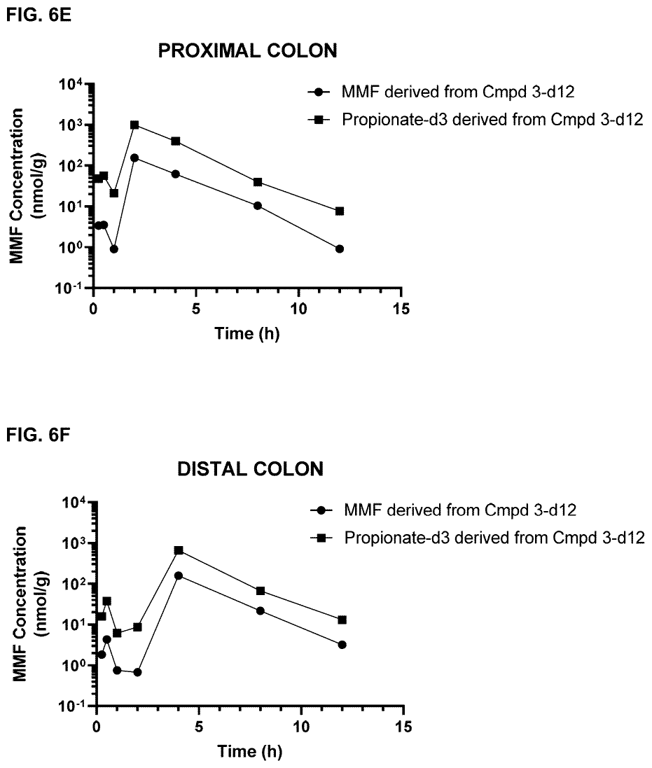

Samples from Example 6, Study 1 were also analyzed by LC-MS/MS for monomethylfumarate concentrations. MMF concentrations versus time for different tissues are depicted in FIGS. 6A-6F. Certain pharmacokinetic parameters are summarized in Table 4 below.

TABLE 4

| | Tₘₐₓ | Cₘₐₓ | AUCₗₐₛₜ |
|---|---|---|---|
| Tissue | (h) | (nmol/g) | (h × nmol/g) |
| Stomach | 1.00 | 1170 | 3180 |
| Proximal | 0.50 | 76.1 | 56.4 |
| Intestine | | | |
| Distal | 1.00 | 142 | 156 |
| Intestine | | | |
| Cecum | 2.00 | 112 | 297 |
| Proximal | 2.00 | 154 | 465 |
| Colon | | | |
| Distal | 4.00 | 157 | 568 |
| Colon | | | |

These data suggest that compounds of the invention are sufficiently stable to be able to deliver monomethyl fumarate to regions in the gut, including particularly the colon.

### Study 3: Gastrointestinal (GI) Exposure to Monomethylfumarate (MMF)

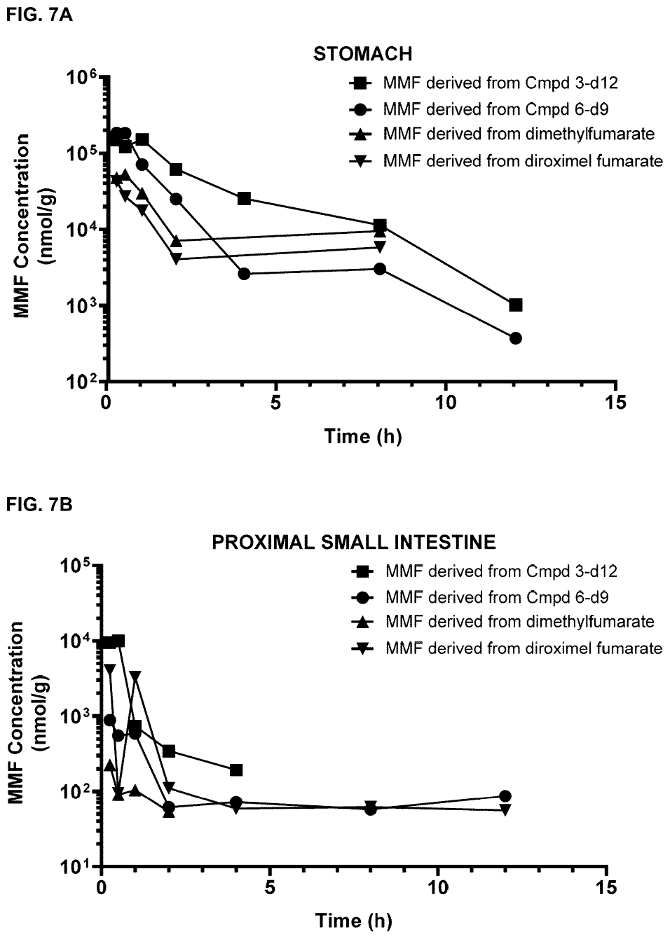

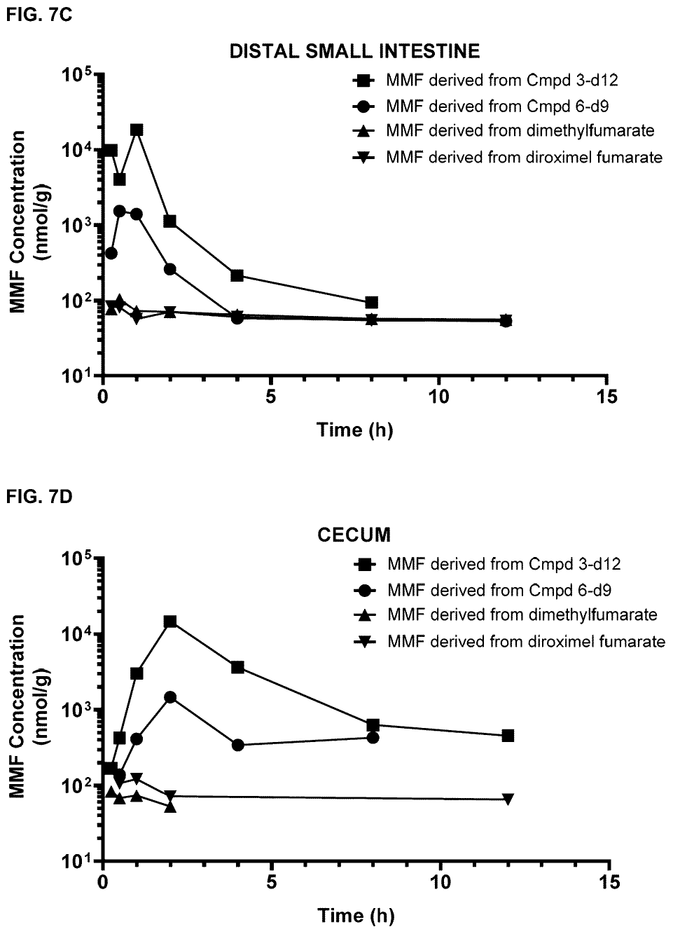

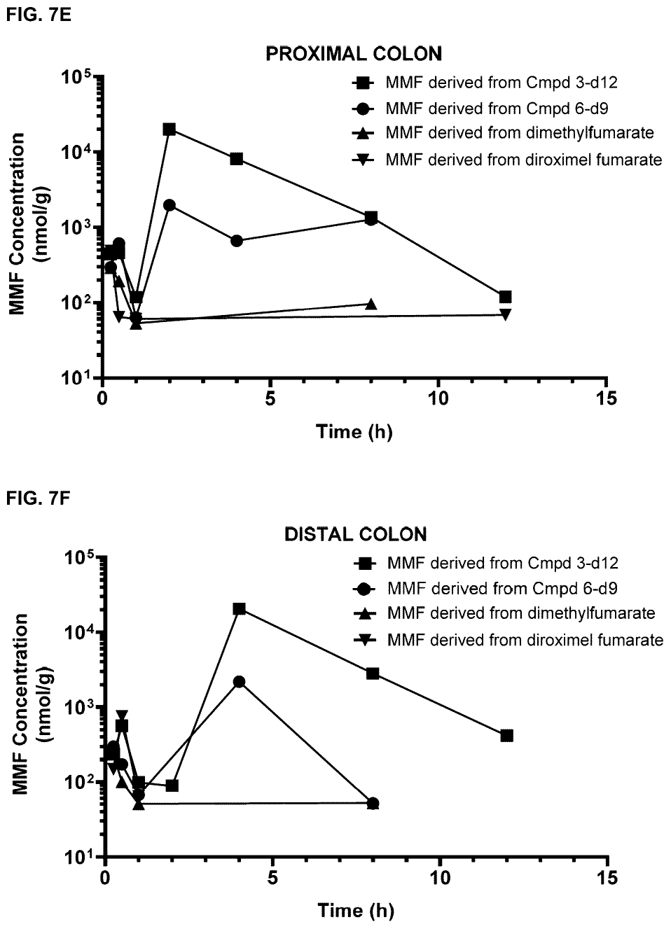

CD-1 mice were orally administered a single dose of Compound 3 comprising deuterated propionate (Compound 3-d12), Compound 6 comprising deuterated propionate (Compound 6-d9), dimethylfumarate, or diroximel fumarate (all as suspensions, 1% (w/v) methyl cellulose in deionized water). The amounts of compound dosed were normalized to provide approximately equimolar amounts of MMF in vivo (Compound 3-d12 at 110 mg/kg, Compound 6-d9 at 91 mg/kg, dimethylfumarate at 30 mg/kg, and diroximel fumarate at 53 mg/kg). Whole blood samples and GI digest samples were collected pre-dosing; at 15 and 30 min; and at 1, 2, 4, 8, and 12 h after dosing. Blood samples were collected in K₂EDTA tubes, stored on wet ice, and then further processed to plasma. GI samples were placed into separately labeled, pre-weighted collection tubes and frozen before analysis. Samples were subsequently worked up and analyzed by LC-MS/MS for MMF concentrations. MMF concentrations versus time for different tissues are depicted in FIGS. 7A-7F.

Data from these experiments suggest that compounds of the invention can be metabolized in vivo and provide MMF to regions of the gut. Further, compounds of the invention can deliver higher amounts of MMF to regions of the gut when compared to dimethylfumarate or diroximel fumarate.

### Example 7: Neutrophil Chemokine Production Assay

A volume 25 mL of human blood was layered over 15 mL of Histopaque®-1077 and centrifuged at 500 g, RT, for 30 min with no break applied to the centrifuge. The PBMC band and Histopaque®-1077 layer were removed leaving behind the bottom red layer which was mixed with 40 mL of 1× red blood cell (RBC) lysis buffer (Sigma-Aldrich) was and split into two 50 mL tubes. The volume for both fractions was brought to 50 mL with RBC lysis buffer, mixed by inversion, and then incubated at RT for 10 min. Solutions were centrifuged at 250 g, for 10 min at RT and the supernatant liquids removed. The reddish pellets were re-suspended in 1 mL of RBC lysis buffer and combined. The cell suspension was incubated for 5 min at RT in RBC lysis buffer. After incubation 45 mL of Hanks Balanced Salt Solution with no calcium, magnesium or phenol red (HBSS-) was added, the cell suspension was spun (250 g for 10 min at RT), and supernatant liquids were removed. The white pellet was re-suspended in 1 mL of HBSS-, and cell counts were determined. The neutrophil cell suspension was brought to a concentration of 1.11e6 cells/mL in RPMI complete (Sigma-Aldrich), and 180 μL of cell suspension was transferred to all wells within a sterile 96-well tissue culture treated plate resulting in 2.0×10⁵ cells/well. Test compounds were brought to a 20× concentration in RPMI with 2% DMSO, and 10 μL of compound solutions were added to wells respective for each compound and incubated for 30 min. After incubation, 10 μL of 2 μg/mL LPS solution in RPMI complete was added to each well except for control wells, which received an additional 10 μL of media. Cells were incubated for 12 h (37° C., 5% CO₂), after which plates were centrifuged at 250 g, RT, for 5 min and supernatant liquids were obtained and stored at −80° C. until analyzed via Luminex® Multiplex Assay for various chemokines and cytokines. Three data points were acquired from two different blood donors and averaged. Statistical analysis was performed using a two-tailed t-test comparing chemokine/cytokines production in the presence of each individual compound to the DMSO+LPS positive control. The results of this assay are summarized in Table 5.

TABLE 5

| | | % IL-8 | % MIP-1α | % MIP-1β |
|---|---|---|---|---|
| | | (Vehicle + | (Vehicle + | (DMSO + |
| | Conc. | LPS = | LPS = | LPS = |
| Compound | (μM) | 100%) | 100%) | 100%) |
| acetate | 500.0 | − | + | + |
| acetate | 1000.0 | − | ++ | + |
| acetate | 3000.0 | +++ | +++ | +++ |
| L-arabinose | 500.0 | − | − | − |
| L-arabinose | 1000.0 | − | − | − |
| epigallocatechin | 0.1 | − | − | − |
| gallate | | | | |
| epigallocatechin | 1.0 | − | − | − |
| gallate | | | | |
| quercetin | 0.1 | − | − | − |
| quercetin | 1.0 | − | − | − |
| (R) 1,3-butanediol | 100.0 | − | − | + |
| (R) 1,3-butanediol | 500.0 | − | − | + |
| β-hydroxybutyric | 200.0 | − | − | + |
| acid | | | | |
| β-hydroxybutyric | 2000.0 | + | ++ | + |
| acid | | | | |
| resveratrol | 10.0 | − | + | ++ |
| butyrate | 500.0 | ++ | +++ | +++ |
| butyrate | 1000.0 | ++ | +++ | +++ |
| propionate | 500.0 | − | +++ | +++ |
| propionate | 1000.0 | ++ | +++ | +++ |
| propionate | 3000.0 | +++ | +++ | +++ |

Vehicle + LPS = 100%
− = >90% Vehicle
+ = <90% Vehicle
++ = <70% Vehicle
+++ = <50% Vehicle

Neutrophils are often the first response from the innate immune system. There is a link between neutrophil presence and disease activity in, e.g., ulcerative colitis. IL-8, MIP1a and MIP1b are important chemokines produced from neutrophils. This work shows compounds of Table 5 reduced neutrophil production of specified markers and therefore may be useful in a variety of autoimmune disorders including multiple sclerosis and psoriasis. Examples of multiple sclerosis include primary progressive multiple sclerosis, secondary progressive multiple sclerosis, or relapsing-remitting multiple sclerosis. Additional indications include obstructive sleep apnea, chronic lymphocytic leukemia, small lymphocytic leukemia, Systemic Sclerosis-Pulmonary Hypertension, Glioblastoma Multiforme, Cutaneous T Cell Lymphoma, rheumatoid arthritis, Psoriatic Arthritis, lupus and Progressive Multifocal Leukoencephalopathy.

### Example 8: Investigating AhR Activation in Caco-2 Cells Through CYP1A1 mRNA Expression

Caco-2 cells from American Type Culture Collection (ATCC) were plated in a sterile tissue culture treated 96-well plate (ThermoFisher) at 8.0×10⁵ cells per well, and grown overnight at 37° C., 5% CO₂ in DMEM complete (Gibco) in order to achieve confluence. After the incubation medium was aspirated off of the Caco-2 monolayers, tissues were then washed with 200 μL of warmed PBS solution, and subsequently 190 μL of pre-warmed growth medium was added to each well. Compounds of interest were diluted at a 20× concentration in growth medium containing 2% DMSO, and 10 μL of compound solutions were added to respective wells in triplicate. Compounds where incubated overnight at 37° C., 5% CO₂. 2-(1′H-indole-3′-carbonyl)-thiazole-4-carboxylic acid methyl ester (ITE) was used as the positive control for AhR activation at 1 and 100 μM concentrations. At the end of the incubation, medium was aspirated off of the Caco-2 cells, and washed with 100 μL of cold PBS solution. RNA was extracted via the TaqMan™ Gene Expression Cells-to-C_{T}™ Kit (ThermoFisher) according to the manufacturer's protocol. The QuantStudio 6 Flex (Applied Biosciences) was used to analyze mRNA levels of CYP1A1 using GAPDH as the endogenous control. TaqMan™ probe sets for both genes were acquired from ThermoFisher. Samples were run in triplicate and data was analyzed using the QuantStudio software and reported as linear (Table 6) and log 2(ΔΔC_{T}) values. Statistical analysis was performed using a two-tailed t-test comparing CYP1A1 levels in the presence of each individual compound to the vehicle negative control.

Activation of aryl hydrocarbon receptor (AhR) has been with associated with immune modulation and active compounds (+, ++, +++) may be beneficial in treating a variety of inflammatory and autoimmune diseases, e.g., ulcerative colitis, multiple sclerosis, rheumatoid arthritis.

TABLE 6

| | Conc. | Average CYP1A1 |
|---|---|---|
| | (μM) | mRNA levels |
| vehicle control | N/A | − |
| acetate | 1000.0 | − |
| acetate | 3000.0 | − |
| L-arabinose | 1000.0 | − |
| epigallocatechin gallate | 0.1 | − |
| epigallocatechin gallate | 1.0 | − |
| quercetin | 0.1 | − |
| quercetin | 1.0 | + |
| butanediol | 500.0 | − |
| β-hydroxybutyric acid | 2000.0 | − |
| resveratrol | 100.0 | − |
| butyrate | 1000.0 | − |
| butyrate | 3000.0 | − |
| propionate | 1000.0 | − |
| propionate | 3000.0 | − |
| indole-3-acetic acid | 500.0 | − |
| indole-3-acetic acid | 1000.0 | − |
| indole-3-butyric acid | 500.0 | − |
| indole-3-butyric acid | 1000.0 | − |
| indole-3-propionic acid | 500.0 | − |
| indole-3-propionic acid | 1000.0 | − |
| indole | 1000.0 | + |
| indole-3-aldehyde | 1000.0 | + |
| indole-3-carbinol | 1000.0 | + |
| indole-3-acetic acid | 500.0 | +++ |
| indole-3-acetic acid | 1000.0 | ++ |
| indole-3-carboxylic acid | 1000.0 | − |
| indole-3-acrylic acid | 1000.0 | +++ |
| indole-3-pyruvic acid | 1000.0 | +++ |
| ITE 1 μM | 1.0 | +++ |

Vehicle = baseline;
− = <2-fold Vehicle;
+ = >2-fold Vehicle;
++ = >5-fold Vehicle;
+++ = >10-fold Vehicle

### Example 9: Human Caco-2 Barrier Integrity Assay

### Study 1

Caco-2 colonocytes were maintained at 37° C. and 5% CO₂ in Dulbecco's Modified Eagle Medium (DMEM) and supplemented with 10% FBS, 1% NEAA, 1% penicillin-streptomycin. At 70-80% confluency, cells were trypsinized and seeded in 0.4 cm² transwell collagen I coated membranes with supplemented DMEM in bath apical and basolateral compartments. Cells were seeded at a density of 200,000 cells per well and maintained for 10 days to form a polarized barrier with a TransEpithelial Electrical Resistance (TEER) reading above 1000Ω. On the first day of the assay, initial TEER readings were taken and cytokines were added to the basolateral media (50 ng/mL TNFα, 25 ng/mL IFNγ and 10 ng/mL IL-1β) to reduce barrier integrity while compounds diluted in (dimethyl-sulfoxide) DMSO were added to the apical media in triplicate. After 48 hours, TEER readings were taken again and viability was measured by CellTiter 96 AQᵤₑₒᵤₛ One Solution Cell Proliferation Assay (Promega). The percent change in TEER over the 48 hours was determined and normalized to the 0.1% DMS control (Table 7). None of the compounds reduced proliferation and therefor did not alter call viability.

TABLE 7

| | % Change in |
|---|---|
| | TEER from DMSO |
| No treatment | ++ (170%) |
| DMSO + cytokines | − (100%) |
| acetate 1 mM + cytokines | − |
| acetate 3 mM + cytokines | − |
| arabinose 0.5 mM + cytokines | − |
| arabinose 1 mM + cytokines | − |
| epigallocatechin gallate 100 nM + cytokines | − |
| epigallocatechin gallate 1 μM + cytokines | − |
| quercetin 100 nM + cytokines | − |
| quercetin 1 μM + cytokines | + |
| (R) 1,3-butanediol 100 μM + cytokines | − |
| (R) 1,3-butanediol 0.5 mM + cytokines | − |
| β-hydroxybutyrate 200 μM + cytokines | + |
| β-hydroxybutyrate 2 mM + cytokines | − |
| resveratrol 10 μM + cytokines | − |
| resveratrol 100 μM + cytokines | +++ |
| butyrate 1 mM + cytokines | − |
| butyrate 3 mM + cytokines | − |
| butyrate 5 mM + cytokines | ++ |
| propionate 1 mM + cytokines | + |
| propionate 3 mM + cytokines | ++ |

Statistical changes in TEER were determined by way ANOVA and compared to DMSO.
<125%: −
125%> <150%: +
150%> <200%: ++
200%>: +++

Barrier function and integrity are important features of a variety of diseases and can be a hallmark of a damaged GI tract. Inflammation can drive a reduction of barrier function. By improving barrier function and therefore TEER, reduced translocation of bacteria and bacterial products occurs, thus reducing inflammation and damage to the GI tract and systemic immune systems. Results from this assay suggest that active compounds (+, ++, +++) may be effective for the treatment of auto-immune diseases. Exemplary indications include: Multiple sclerosis and psoriasis, primary progressive multiple sclerosis, secondary progressive multiple sclerosis, or relapsing-remitting multiple sclerosis. Additional indications include obstructive sleep apnea, chronic lymphocytic leukemia, small lymphocytic leukemia, Systemic Sclerosis-Pulmonary Hypertension, Glioblastoma Multiforme, Cutaneous T Cell Lymphoma, rheumatoid arthritis, Psoriatic Arthritis, lupus and Progressive Multifocal Leukoencephalopathy, Parkinson's.

### Study 2

Caco-2 colonocytes were maintained at 37′C and 5% CO₂ in Dulbecco's Modified Eagle Medium (DMEM) and supplemented with 10% FBS, 1% NEAA, 1% penicillin-streptomycin. At 70-80/confluency, cells were trypsinized and seeded in 0.4 cm² transwell collagen I coated membranes with supplemented DMEM in both apical and basolateral compartments. Cells were seeded at a density of 200,000 cells per well and maintained for 10 days to form a polarized barrier with a TransEpithelial Electrical Resistance (TEER) reading above 1000Ω. On the first day of the assay, initial TEER readings were taken and dimethylfumarate and propionic acid were added to the apical media at various concentrations in order to affect barrier integrity. TEER readings were taken again every 24 hours and viability was measured by CellTiter 96® AQᵤₑₒᵤₛ One Solution Cell Proliferation Assay (Promega). Percent change in TEER over the 72 hours was determined and normalized (Table 8).

TABLE 8

| Compound | % Change in TEER |
|---|---|
| Dimethylfumarate (10 mM) | −28% |
| | (relative to control) |
| Dimethylfumarate (10 mM) + | +12% |
| propionic acid (10 mM) | (relative to dimethylfumarate only) |

Relative to administration of dimethylfumarate alone, a combination of propionate and dimeth fumarate improved barrier integrity, a hallmark of gastrointestinal health. Propionate can restore gastrointestinal barrier dysfunction that is caused by dimethylfumarate.

### Example 10: Human Regulatory T Cell Differentiation Assay

Peripheral blood mononuclear cells (PBMCs) from whole blood donated by health volunteers were separated by Ficoll-Paque gradient centrifugation and naïve CD4⁺ T cells were subsequently isolated using magnet beads (EasySep™ Human Naïve CD4⁺ T Cell Isolation Kit, Cambridge, MA). For regulatory T cell (Treg) differentiation assay, naïve CD4⁺ T cells were cultured (1-10×10⁴ cells) in CTS OpTmizer medium for 6 days and stimulated with 5 ng/ml TGF-β, 100 U/ml IL-2, and ImmunoCult™ Human CC3/CD28/CD2 T Cell Activator; Stemcell #10990) with/without our Compounds. Cell viability was determined using a viability dye (eBioscience Fixable Viability Dye eFluor 780: ThermoFisher 65-0865-14) at 1:500 dilution. The cells were gated for Treg, defined as Live, CD11c⁻, CD14⁻, CD19⁻, CD8⁻, CD4⁺, CD3⁺, CD25⁺, FOXP3⁺. Percent (%) Tregs were calculated as percentage of CD4⁺, CD25⁺, FOXP3⁺ cells over total CD4⁺ T cells. Statistical analysis was performed with GraphPad Prism Software Using One-Way ANOVA. The results of this assay are summarized in Table 9.

TABLE 9

| | Treg induction | Cell viability |
|---|---|---|
| Treatment | % DMSO | % DMSO |
| acetic acid 1 mM | + | = |
| acetic acid 3 mM | ++ | = |
| L-arabinose 0.5 mM | = | = |
| L-arabinose 1 mM | = | = |
| epigallocatechin gallate 100 nM | = | = |
| epigallocatechin gallate 1 μM | = | = |
| quercetin 100 nM | = | = |
| quercetin 1 μM | = | = |
| (R)-1,3-butanediol 100 uM | = | = |
| (R)-1,3-butanediol 0.5 mM | = | = |
| sodium β-hydroxybutyrate 2 mM | + | = |
| sodium β-hydroxybutyrate 20 mM | = | − |
| butyric Acid 3 mM | − | − |
| propionic acid 3 mM | ++ | = |
| rosiglitazone 10 μM | = | = |
| rosiglitazone 100 μM | = | − |
| resveratrol 1 μM | + | − |
| resveratrol 10 μM | + | − |
| obeticholic acid 100 μM | + | = |
| DMSO | = (100.0) | = (100%) |

<90%: −
90%> <110%: =
110%> <130%: +
130%>: ++

Table 9 shows compounds that increased the differentiation of naïve CD4⁺ T cells into Tregs (+, ++), or decreased the differentiation of naïve CD4⁺ T cells into Tregs (−). Tregs play an important role in keeping the balance of immune system and compounds that increase Tregs (+, ++) may be useful in the treatment of autoimmune and inflammatory diseases. Examples of multiple sclerosis include primary progressive multiple sclerosis, secondary progressive multiple sclerosis, or relapsing-remitting multiple sclerosis. Additional indications include obstructive sleep apnea, chronic lymphocytic leukemia, small lymphocytic leukemia, Systemic Sclerosis-Pulmonary Hypertension, Glioblastoma Multiforme, Cutaneous T Cell Lymphoma, rheumatoid arthritis, Psoriatic Arthritis, lupus and Progressive Multifocal Leukoencephalopathy, and Parkinson's disease.

### Example 11: Effect of Compound Treatment on Cytokine Release from Human Peripheral Blood Monocytes (PBMCs

Human donor blood (8 mL) was collected in sodium citrate CPT tubes and centrifuged at 1,600×g for 20 minutes at room temperature. Buffy coat containing PBMCs was collected and transferred to a 50 mL conical tube containing 30 mL of RPMI-1640 medium at room temperature (supplemented with penicillin-streptomycin). PBMCs samples were centrifuged at 400×g for 10 minutes at 10° C. The pelleted PBMCs were washed twice in 10 ml of RPMI-1640 medium (supplemented with penicillin-streptomycin), then resuspended in RPMI-1640 medium (supplemented with penicillin-streptomycin, fetal bovine serum, and L-Glutamine). PBMCs were filtered through a 70 micron mesh to remove any cellular debris. The volume was adjusted to achieve 1.66×100 cells/mL, from which 180 μl (300,000 PBMCs) were added into each well in a 96-well plate (sterile, tissue culture treated, round bottom). PBMCs in a 96-well plate were rested for 30 minutes in a 37° C., 5% CO₂ incubator, then subsequently treated with 10 μl of indicated compound. After 2 hours 10 μL of LPS (O111:B4) 1 mg/mL was added to test wells. After 24 hours of incubation at 37° C., 5% CO₂, 100 μL of cell supernatant was collected and transferred to a 96-well plate (non-tissue treated, flat bottom). The plate was centrifuged at 350×g for 5 minutes at room temperature, and then the clear supernatant transferred to a new 96-well plate (non-tissue treated, flat bottom). The remaining cells were tested for viability using CellTiter-Glo® Luminescent Cell Viability Assay (Promega). The supernatant was analyzed for TNFα, IL-6 and IL-1β (kit LXSAHM-03; R&D Systems), using Luminex Immunoassay Technology (MAGPIX System). Cytokine levels of LPS treated DMSO control samples were set to 100%, and compound treated samples were expressed relative to this (Table 10).

TABLE 10

| | Concen- | TNFα | IL6 | IL1β |
|---|---|---|---|---|
| | tration | % of DMSO | % DMSO | % DMSO |
| Compound | (μM) | control | control | control |
| Propionate | 100 | + | + | + |
| Arabinose | 100 | + | + | = |
| (R) 1,3-butanediol | 100 | = | = | = |
| β-hydroxybutyrate | 100 | − | = | − |
| Butyrate | 100 | ++ | + | − |
| Acetate | 100 | = | = | = |
| Quercetin | 100 | + | + | + |
| Resveratrol | 100 | + | + | = |

(−) >110% DMSO;
(=) 90%> <110% DMSO
(+) 50%> <90% DMSO
(++) <50% DMSO

Compounds that are active in this assay (+, ++) show anti-inflammatory activity in human monocyte cultures as indicated by the reduction in secreted proinflammatory cytokines. In the context of stimulation of cells with LPS, this triggers a host of proinflammatory responses that are representative of autoimmune disorders. As a result of these pathways being activated, proinflammatory signaling molecules are released (IL-6, IL-1β, and TNFα). Reduction of these cytokines suggests compounds would be efficacious in treating autoimmune diseases. Exemplary indications include: Multiple sclerosis and psoriasis, primary progressive multiple sclerosis, secondary progressive multiple sclerosis, or relapsing-remitting multiple sclerosis. Additional indications include obstructive sleep apnea, chronic lymphocytic leukemia, small lymphocytic leukemia, Systemic Sclerosis-Pulmonary Hypertension, Glioblastoma Multiforme, Cutaneous T Cell Lymphoma, rheumatoid arthritis, Psoriatic Arthritis, lupus and Progressive Multifocal Leukoencephalopathy, Parkinson's.

### OTHER EMBODIMENTS

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are in the claims.

1. A conjugate of monomethyl fumarate and a carrier group or aminocarrier group, or a pharmaceutically acceptable salt thereof, wherein monomethyl fumarate acyl is covalently bonded to the carrier group or the aminocarrier group through a carbon-oxygen bond that is cleavable in vivo.

2. The conjugate of claim 1, or a pharmaceutically acceptable salt thereof, wherein the conjugate comprises a carrier group comprising a core with one or more hydroxyls independently substituted with an acyl.

3. (canceled)

4. The conjugate of claim 2, or a pharmaceutically acceptable salt thereof, wherein the core is a monosaccharide, an aminomonosaccharide, or an acid monosaccharide.

5-10. (canceled)

11. The conjugate of claim 2, or a pharmaceutically acceptable salt thereof, wherein the core is a C₅₋₆ pyranose.

12-13. (canceled)

14. The conjugate of claim 1, or a pharmaceutically acceptable salt thereof, wherein the carbon-oxygen bond that is cleavable in vivo is an ester bond.

15. The conjugate of claim 11, or a pharmaceutically acceptable salt thereof, wherein the carbon-oxygen bond that is cleavable in vivo is a glycosidic bond attached to the anomeric carbon atom of the C₅₋₆ pyranose, a bond attached to position 4 of the C₅₋₆ pyranose, or a bond attached to position 6 of the C₅₋₆ pyranose.

16-17. (canceled)

18. The conjugate of claim 1, or a pharmaceutically acceptable salt thereof, wherein the conjugate comprises a fatty acid acyl that is a short chain fatty acid acyl or a fatty acid acyl that is a medium chain fatty acyl.

19-21. (canceled)

22. A conjugate of monomethyl fumarate and a carrier group, or a pharmaceutically acceptable salt thereof, wherein monomethyl fumarate acyl is covalently bonded to the carrier group through a carbon-oxygen bond that is cleavable in vivo, wherein the carrier group comprises a catechin polyphenol core.

23-27. (canceled)

28. A conjugate of monomethyl fumarate and a carrier group, or a pharmaceutically acceptable salt thereof, wherein monomethyl fumarate acyl is covalently bonded to the carrier group through a carbon-oxygen bond that is cleavable in vivo, wherein
the carrier group comprises a sugar alcohol core of formula:
HOCH₂(CHOH)ₙCH₂OH,
wherein n is 1, 2, 3, or 4; and one or more of the hydroxyl groups is independently substituted with an alkyl, acyl, or a bond to monomethyl fumarate.

29-31. (canceled)

32. The conjugate of claim 1, wherein the conjugate is a compound of the following structure: 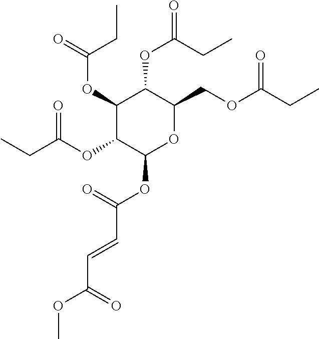 [SMILES: CCC(=O)OC[C@H]1O[C@@H](OC(=O)/C=C/C(=O)OC)[C@H](OC(=O)CC)[C@@H](OC(=O)CC)[C@@H]1OC(=O)CC]
or a pharmaceutically acceptable salt thereof.

33. The conjugate of claim 1, wherein the conjugate is a compound of the following structure: 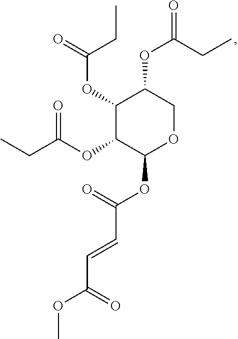 [SMILES: CCC(=O)O[C@H]1[C@H](OC(=O)/C=C/C(=O)OC)OC[C@@H](OC(=O)CC)[C@H]1OC(=O)CC]
or a pharmaceutically acceptable salt thereof.

34-36. (canceled)

37. A pharmaceutical composition comprising:
(i) the conjugate of claim 1, or a pharmaceutically acceptable salt thereof, and
(ii) a pharmaceutically acceptable carrier.

38. A method of treating a subject comprising administering a therapeutically effective amount of the conjugate of claim 1, or a pharmaceutically acceptable salt thereof, to a subject in need thereof.

39. The method of claim 38, wherein the subject is suffering from an autoimmune disorder.

40. (canceled)

41. The method of claim 38, wherein the subject is suffering from multiple sclerosis.

42-48. (canceled)

49. A method of modulating an autoimmunity marker comprising administering a therapeutically effective amount of the conjugate of claim 1, or a pharmaceutically acceptable salt thereof, to a subject in need thereof.

50. The method of claim 49, wherein the autoimmunity marker is for multiple sclerosis, psoriasis, psoriatic arthritis, rheumatoid arthritis, systemic lupus erythematosus, Crohn's disease, Sjogren's syndrome, Behcet's disease, ulcerative colitis, or Guillain-Barré syndrome.

51. The method of claim 38, wherein a CYP1A1 mRNA level, intestinal motility, CD4⁺CD25⁺ Treg cell count, short chain fatty acid level, or mucus secretion is increased following the administration step; or
wherein abdominal pain, gastrointestinal inflammation, gastrointestinal permeability, gastrointestinal bleeding, intestinal motility, or frequency of bowel movements is reduced following the administration step; or
wherein interleukin-8 (IL8) level, macrophage inflammatory protein 1α (MIP-1α) level, macrophage inflammatory protein 1β (MIP-1β) level, NFκB level, inducible nitric oxide synthase (iNOS) level, matrix metallopeptidase 9 (MMP9) level, interferon γ (IFNγ) level, interleukin-17 (IL17) level, intercellular adhesion molecule (ICAM) level, CXCL13 level, 8-iso-prostaglandin F_{2α} (8-iso-PGF2α) level IgA level, calprotectin level, lipocalin-2 level, or indoxyl sulfate level is reduced following the administration step.

52-55. (canceled)

56. The method of claim 38, wherein an Nrf2 expression level, citric acid level, serotonin level, β-hydroxybutyric acid level, docosahexaenoic acid level, putrescine level, N-methyl nicotinic acid level, lauric acid level, or arachidonic acid level is increased following the administration step; or
wherein a L-citrulline level, picolinic acid level, quinolinic acid level, 2-ketoglutaric acid level, L-kynurenine/L-tryptophan ratio, kyunurenic acid level, prostaglandin E2 level, leukotriene B4, linolenic acid level, linoleic acid level, CD8⁺ T cell count, memory B cell count, CD4⁺ EM cell count, cumulative number of new Gd+ lesions, L-phenylalanine level, hippuric acid level, or eicosapentaenoic acid level is reduced following the administration sten.

57-59. (canceled)

60. A method of delivering a monomethyl fumarate moiety to a target site in a subject in need thereof, the method comprising administering to the subject the conjugate of claim 1, or a pharmaceutically acceptable salt thereof.

61. The method of claim 60, wherein the target site is the small intestine, cecum, or colon of the subject.

62-65. (canceled)

66. The method of claim 49, wherein the subject is suffering from multiple sclerosis.

67-71. (canceled)
